(19) United States
(12) Patent Application Publication
(10) Pub. No.: US 2019/0134153 A1
(43) Pub. Date: May 9, 2019

(54) IMMUNOMODULATORY EFFECT OF INHALED KINASE INHIBITOR PEPTIDES IN LUNG

(71) Applicant: MOERAE MATRIX, INC., MORRISTOWN, NJ (US)

(72) Inventor: Cynthia Lander, Mendham, NJ (US)

(21) Appl. No.: 16/005,408

(22) Filed: Jun. 11, 2018

Related U.S. Application Data
(60) Provisional application No. 62/518,426, filed on Jun. 12, 2017.

(51) Int. Cl. A61K 38/17; A61K 45/06; A61K 31/739; A61P 11/00; A61P 37/04

(52) U.S. Cl. CPC A61K 38/177; A61K 45/06; A61P 37/04; A61P 11/00; A61K 31/739

(57) ABSTRACT

The described invention provides a method of treating a subject that is in an immunotolerant state with regard to an immune stimulating agent that is no longer therapeutically effective for treating a disease, disorder or condition of lung. The method includes the steps, in order, of (a) administering (1) a first pharmaceutical formulation formulated for delivery by inhalation containing an immunomodulatory amount of a kinase-inhibiting peptide, and (b) then administering a second pharmaceutical formulation containing a therapeutic amount of the immunostimulatory agent. The the method is effective to resensitize the subject to the immune stimulating agent so that the subject is once again immunoresponsive to it upon its subsequent administration.

### CROSS-REFERENCE TO RELATED APPLICATIONS

This application claims priority to U.S. Provisional Patent Application No. 62/518,426 filed Jun. 12, 2017, entitled “Immunomodulatory Effect of Inhaled Kinase Inhibitor Peptides in Lung,” the contents of which is incorporated herein by reference in its entirety.

### FIELD OF THE INVENTION

This invention relates to, inflammation, cytokine synthesis and expression, kinase mediated signaling pathways and kinase inhibiting peptides.

### BACKGROUND OF THE INVENTION

Anatomy of the Lung

The respiratory system comprises the larynx, trachea, bronchi, bronchioles and alveoli. Rubin's Pathology, Rubin, R and Strayer, D S Eds, 5^{th} Edition, Lippincott Williams & Wilkins, MD (2008), at 484-485.

The trachea is a hollow tube from which the bronchi diverge. The right bronchus diverges at a lesser angle from the trachea than does the left, which is why foreign material is more frequently aspirated on the right side. On entering the lung, the bronchi divide into lobar bronchi, then into segmental bronchi, which supply the 19 lung segments. Id.

The tracheobronchial tree contains cartilage and submucosal mucous glands in the wall. The mucous glands are compound tubular glands with mucous cells and serous cells, which are granular. The lining is pseudostratified epithelium, which appears as layers, although all cells reach the basement membrane. Most cells are ciliated, but there are also mucus-secreting goblet cells and basal cells. The basal cells, which do not reach the surface, are through t to be precursor cells that differentiate to form the more specialized cells of the tracheobronchial epithelium. There are also nonciliated columnar cells (Clara cels), which accumulate and detoxify many inhaled toxic agents. Scattered in the tracheobronchial mucosa are Kulchitsky cells, neuroendocrine cells that contain a variety of hormonally active polypeptides and vasoactive amines. Id.

Distal to the bronchi are the bronchioles, which lack cartilage and mucus-secreting cells. Bronchiolar epithelium becomes thinner with progressive branching, until only one cell layer is present. The terminal bronchiole, the last purely conducting structure free of alveoli, has a circumferential layer of pseudostratified ciliated respiratory epithelium and a smooth muscle wall. Mucous cells gradually disappear from the lining of the bronchioles until they are entirely replaced in the small bronchioles by nonciliated, columnar Clara cells. The terminal bronchioles divide into respiratory bronchioles, which merge into alveolar ducts and alveoli. The acinus, which is the unit of gas exchange in the lung, consists of respiratory bronchioles, alveolar ducts and alveoli. Id.

The alveoli are lined by two types of epithelium: type I cells, which are thin and have a large surface area (both of which facilitates gas exchange) cover 9% of the alveolar surface, but comprise only 40% of alveolar epithelial cells. Type I cells are particularly vulnerable to injury; when they are lost, type II pneumocytes multiply and differentiate to form new type I cells that reconstitute the alveolar surface. Type II cells, which produce surfactant, are 60% of the alveolar lining cells, and are more cuboidal, constitute only 5% of the alveolar surface. Id.

The cytoplasm of epithelial and endothelial cells is spread very thinly on either side of a fused basement membrane, allowing efficient exchange of oxygen and carbon dioxide. An abundant capillary network covers 85% to 95% of the alveolar surface. Away from the site of gas exchange, there is more abundant interstitial connective tissue consisting of collagen, elastin, and proteoglycans. Fibroblasts and myofibroblasts may also be present. This expanded region forms the interstitial space of the alveolar wall, where significant fluid and molecular exchange occurs. Id.

The lung has a dual blood supply: the pulmonary circulation and the bronchial system. Pulmonary arteries accompany the airways in a sheath of connective tissue, the bronchovascular bundle. The more proximal arteries, which are elastic, are succeeded by muscular arteries, the pulmonary arterioles and eventually the pulmonary capillaries. Id.

The smallest veins, which resemble the smallest arteries, join other veins and drain into the lobular septa, connective tissue partitions that subdivide the lung into small respiratory units. The veins then continue in the lobular septa, joining other veins to form a network that is separate from the bronchovascular bundles. Id.

The bronchial arteries arise from the thoracic aorta and nourish the bronchial tree as far as the respiratory bronchioles. They are accompanied by their respective veins, which drain into the azygous or hemizygous veins. Id.

There are no lymphatics in most alveolar walls. The lymphatics commence in alveoli at the periphery of the acinus, which lies along a lobular septum, a bronchovascular bundle or the pleura. The lymphatics of the lobular septa and bronchovascular bundle accompany these structures, and the pleural lymphatics drain toward the hilus through the bronchovascular lymphatics. Id.

Pulmonary collectins belong to the superfamily of Ca2+-dependent lectins (C-type lectins); nine different members have been identified so far: mannose-binding lectin (MBL), conglutinin, SP-A, SP-D, collectin (CL-43, CL-46, CL-P1, CL-L1 and CL-K1, all of which form multimers, which increase their affinity to immune cells and pathogens. SP-A and SP-D, which possess complex oligomeric structures critical to their function, play a critical role in regulating innate immune responses within the lung. NO is capable of modifying these proteins via a number of different mechanisms and with varying effects on their structural organization. Atochina-Vasserman, E N et al (2010) “Chemical and structural modifications of pulmonary collectins and their functional consequences,” Innate Immun. 16(3): 175-82).

### Immune Privilege

Immune privilege is an evolutionary adaptation aimed at protecting especially vulnerable organs from overwhelming inflammation that could abolish their functions and jeopardize the well-being of the individual. Immune privileged status is preserved by local active mechanisms that suppress responses to antigens within the privileged tissues (Id. citing Niederkorn, J Y and Stein-Streilein, J *2010), “History and physiology of immune privilege,” Ocul. Immunol. Inflamm. 18: 19-23).

The best characterized immune privileged structure is the eye. In the eye, one such mechanism is anterior chamber-associated immune deviation (ACAID), referring to a phenomenon in which antigenic material introduced into the anterior chamber of the eye elicits a systemic immune response that results in immune deviation, characterized by the suppression of T cell-mediated immunity, while enabling the production of non-complement-fixing antibodies (Id. citing Kaplan, H J et al. (1975) “Transplantation immunology of the anterior chamber of the eye. II. Immune response to allogeneic cells,” J. Immunol. 115: 805-810; Streilein, J W (2003) “Ocular immune privilege: therapeutic opportunities from an experiment of nature,” Nat. Rev. Immunol. 3: 879-89; Niederkorn, J Y (2006) “See no evil, hear no evil, do no evil: the lessons of immune privilege,” Nat. Immuno. 7: 354-59). ACAID involves the migration of specialized antigen presenting cells from the eye to the thymus and spleen, and is associated with an elevation in regulatory, γδ, and natural killer T cells (Id. citing Streilein, J W (2003) “Ocular immune privilege: therapeutic opportunities from an experiment of nature,” Nat. Rev. Immunol. 3: 879-89; Niederkorn, J Y (2006) “See no evil, hear no evil, do no evil: the lessons of immune privilege,” Nat. Immunol. 7: 354-59). Other mechanisms aimed at maintaining the immune privileged state of the eye include the reduced expression of MHC molecules on ocular cells, and the existence of an intraocular anti-inflammatory environment, mediated by resident cells, and various molecules, both surface-bound and soluble, all of which serve to modulate the activity of infiltrating immune cells, in situ (Streilein, J W (2003) “Ocular immune privilege: therapeutic opportunities from an experiment of nature,” Nat. Rev. Immunol. 3: 879-89; Schewitz-Bowers, L P et al. (2010) “Immune mechanisms of intraocular inflammation,” Expert Rev. Ophthalmol. 5: 43-58; Zhou, R. et al., 2012) “The living eye “disarms” uncommitted autoreactive T cells by converting them to Foxp3(+) regulatory cells following local antigen recognition,” J. Immunol. 188: 1742-50).

### The Lung is an Immune Privileged Organ

The respiratory mucosa is exposed continuously to a wide variety of environmental antigens. Because overzealous host immune responses could be detrimental, causing injury to the lung and interfering with gas exchange, mechanisms specific to the respiratory mucosa exist to limit immune responses and prevent mucosal damage. Some of these mechanisms may include processes that reduce airway inflammation and enhance the development of tolerance to antigen exposure, and some of these mechanisms include rapid clearance of inspired antigen, induction of the development of regulatory/suppressor cells, limitation of costimulatory signals, or induction of functional inactivation in CD4 T cells. Blumenthal, R L et al. (2001) “Human alveolar macrophages induce functional inactivation in antigen-specific CD4 T cells,” J. Allergy Clin. Immunol. 107(2): 258-64) citing Lipscomb, M F et al (1993) “The role of T lymphocytes in pulmonary microbial defense mechanisms,” Arch. Pathol. Lab Med. 117: 1225-32; Brandtzaeg, P et al (1996) “Immune functions and immunopathology of the mucosa of the upper respiratory pathways,” Acta Otolaryngol. 116: 149-59; Chai, J G et al (1999) “Anergic T cells act as suppressor cells in vitro and in vivo,” Eur. J. Immunol. 29: 686-72; Chelen, C J et al (1995) “Human alveolar macrophages present antigen ineffectively due to defective expression of B7 costimulatory cell surface molecules,” J. Clin. Invest. 95: 1415-21; Fireman, E. et al (1993) “Suppressive mechanisms of alveolar macrophages in interstitial lung diseases: role of soluble factors and cell-to-cell contact,” Eur. Respir. J. 6: 956-64; McCombs, C C et al (1982) “Human alveolar macrophages suppress the proliferative response to peripheral blood lymphocytes,” Ches 82: 266-71; Strickland, D. et al (1996) “Regulation of T-cell activation in the lung: alveolar macrophages induce reversible T-cell anergy in vitro associated with inhibition of interleukin-1 receptor signal transduction,” Immunol. 87: 250-58).

Alveolar macrophages (AMs), the most abundant phagocytic cells in the lung, protect the alveolar space from respiratory inflammation. Numerous studies indicate that they do not present antigen effectively to T cells. (Id. citing Chelen, C J et al (1995) “Human alveolar macrophages present antigen ineffectively due to defective expression of B7 costimulatory cell surface molecules,” J. Clin. Invest. 95: 1415-21; Ettensohn, D B et al (1989) “The role of human alveolar macrophages in the allogeneic and autologous mixed leukocyte reactions,” Clin. Exp. Immunol. 75: 432-37; Gant, V A et al (1991) “Normal and sarcoid alveolar macrophages differ in their ability to present antigen and to cluster with autologous lymphocytes,” Clin. Exp. Immuno. 85: 494-99). These studies suggest that AMs might function to limit, rather than initiate, immune responses at the pulmonary mucosal surface.

It has been shown that AMs actively phagocytize foreign materials that reach the lung and mucociliary processes then rapidly remove AMs from the lung (Id. citing Holt, P G, Leivers, S. (1985) “Alveolar macrophages: antigen presentation activity in vivo,” Aut. J. Exp. Biol. Med. Sci. 63 (1): 33-39; Kradin, R L et al (1999) “Pulmonary immunity to Listeria is enhanced by elimination of alveolar macrophages,” Am. J. Respir. Crit. Care Med. 159: 1967-74; Thepen, T et al (1989) “Alveolar macrophage elimination in vivo is associated with an increase in pulmonary immune response in mice,” J. Exp. Med. 170: 499-509); and that AMs fail to upregulate expression of the costimulatory molecules B7-1 (CD80) and B7-2 (CD86) on stimulation with IFN-γ (Id. citing Chelsen, C J et al (1995) “Human alveolar macrophages present antigen ineffectively due to defective expression of B7 costimulatory cell surface molecules,” J. Clin. Invest. 95: 1415-21) suggesting that AMs limit T cell responses in the lung by activating T cells in the absence of co-stimulatory signals. Studies also have shown that elimination of AMs from the lungs, for example, with liposome encapsuled dichloromethylenediphosphonate leads to a significant increase in pulmonary immune responses to antigens encountered in the respiratory tract (Id. citing Thepen, T. et al (1989) “Iveolar macrophage elimination in vivo is associated with an increase in pulmonary immune response in mice,” J. Exp. Med. 170: 499-509).

To more clearly define the mechanisms by which AMs present antigen to T cells and limit pulmonary inflammation and antigen-specific immune responses in the normal lung, the capacity of allogeneic AMs and peripheral blood monocytes to induce proliferation of purified human CD4 T cells and cytokine production was compared. Id. It was shown that AMs actively induce T-cell unresponsiveness (functional inactivation) in an antigen-specific manner and reduce the capacity of CD4 T cells to respond on secondary stimulation. Id. The induction of unresponsiveness was reversed by the addition of CD28 costimulation or IL-2. Id. However, interruption of Fas/Fas ligand interactions or of B7/CTLA-4 interactions did not prevent unresponsiveness, indicating that neither CTLA-4 triggering nor Fas-induced apoptosis was involved in the induction of T-cell unresponsiveness. Id. The study was interpreted to indicate that AMs actively tolerize CD4 T cells in an antigen-specific fashion. It was proposed that AMs mediate a form of immune privilege in the lungs that effectively limits immune responses in the pulmonary compartment, but has little effect on systemic immunity.

### Wound Healing

The term “wound healing” refers to the process by which the body repairs trauma to any of its tissues, especially those caused by physical means and with interruption of continuity. Generally speaking, the body responds to injury with an inflammatory response, which is crucial to maintaining the health and integrity of an organism. If however it goes awry, it can result in tissue destruction.

Wound healing is a dynamic, interactive process involving soluble mediators, blood cells, extracellular matrix, and parenchymal cells. Wound healing generally proceeds through three overlapping dynamic phases: (1) an inflammatory phase, (2) a proliferative phase, and (3) remodeling phase.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of innate cells to invading pathogens. Endogenous danger signals also can influence local innate cells and orchestrate the inflammatory cascade.

The inflammatory phase is triggered by capillary damage, which leads to the formation of a blood clot/provisional matrix composed of fibrin and fibronectin. This provisional matrix fills the tissue defect and enables effector cell influx. Platelets present in the clot release multiple cytokines that participate in the recruitment of inflammatory cells (such as neutrophils, monocytes, and macrophages, amongst others), fibroblasts, and endothelial cells (ECs). The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. For example, chronic allergic airway disease in asthmatics is associated commonly with elevated type-2 helper T cell (Th2) related cytokine profiles (including, but not limited to, interleukin-4 (IL-4), interleukin-5 (IL-5), interleukin-6 (IL-6), interleukin-13 (IL-13), and interleukin-9 (IL-9)), whereas chronic obstructive pulmonary disease and fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)).

The inflammatory phase is followed by a proliferative phase, in which active angiogenesis creates new capillaries, allowing nutrient delivery to the wound site, notably to support fibroblast proliferation. Fibroblasts present in granulation tissue are activated and acquire a smooth muscle cell-like phenotype, then being referred to as myofibroblasts. Myofibroblasts synthesize and deposit extracellular matrix (ECM) components that replace the provisional matrix. They also have contractile properties mediated by α-smooth muscle actin organized in microfilament bundles or stress fibers. Myofibroblastic differentiation of fibroblastic cells begins with the appearance of the protomyofibroblast, whose stress fibers contain only β- and γ-cytoplasmic actins. Protomyofibroblasts can evolve into differentiated myofibroblasts whose stress fibers contain α-smooth muscle actin.

The third healing phase involves gradual remodeling of the granulation tissue and reepithelialization. This remodeling process is mediated largely by proteolytic enzymes, especially matrix metalloproteinases (MMPs) and their inhibitors (TIMPs, tissue inhibitors of metalloproteinases). During the reepithalialization, Type III collagen, the main component of granulation tissue, is replaced gradually by type I collagen, the main structural component of the dermis. Elastin, which contributes to skin elasticity and is absent from granulation tissue, also reappears. Cell density normalizes through apoptosis of vascular cells and myofibroblasts (resolution).

During wound healing, distinct subsets of macrophages infiltrate the site of injury and display different functions corresponding to the changing needs of the tissue along the course of healing; these include the clearing of dead cells and tissue debris at the first stage, and the secretion of anti-inflammatory cytokines and growth factors at the later stage, to aid tissue regrowth and restoration of immune homeostasis (Id. citing Arnold, L. et al. (2007) “inflammatory monocytes recruited after skeletal muscle injury switch into anti-inflammatory macrophages to support myogenesis,” J. Exp. Med. 204: 1057-69; Nahrendorf, M et al. (2007) “The healing myocardium sequentially mobilizes two monocyte subsets with divergent and complementary functions” J. Exp. Med. 204: 3037-47).

### Inflammatory Airway or Lung Tissue Diseases

Idiopathic Pulmonary Fibrosis

Idiopathic Pulmonary fibrosis (IPF, also known as cryptogenic fibrosing alveolitis, CFA, or Idiopathic Fibrosing Interstitial Pneumonia) is defined as a specific form of chronic, progressive fibrosing interstitial pneumonia of uncertain etiology that occurs primarily in older adults, is limited to the lungs, and is associated with the radiologic and histological pattern of usual interstitial pneumonia (UIP) (Raghu G. et al., Am J Respir Crit Care Med., 183(6):788-824, 2011; Thannickal, V. et al., Proc Am Thorac Soc., 3(4):350-356, 2006). It may be characterized by abnormal and excessive deposition of fibrotic tissue in the pulmonary interstitium. On high-resolution computed tomography (HRCT) images, UIP is characterized by the presence of reticular opacities often associated with traction bronchiectasis. As IPF progresses, honeycombing becomes more prominent (Neininger A. et al., J Biol Chem., 277(5):3065-8, 2002). Pulmonary function tests often reveal restrictive impairment and reduced diffusing capacity for carbon monoxide (Thomas, T. et al., J Neurochem., 105(5): 2039-52, 2008). Studies have reported significant increases in TNF-α and IL-6 release in patients with idiopathic pulmonary fibrosis (IPF) (Zhang, Y, et al. J. Immunol. 150(9):4188-4196, 1993), which has been attributed to the level of expression of IL-1β (Kolb, M., et al. J. Clin. Invest, 107(12):1529-1536, 2001). The onset of IPF symptoms, shortness of breath and cough, are usually insidious but gradually progress, with death occurring in 70% of patients within five years after diagnosis. This grim prognosis is similar to numbers of annual deaths attributable to breast cancer (Raghu G. et al., Am J Respir Crit Care Med., 183(6):788-824, 2011).

Previous studies have suggested that superimposed environmental insults may be important in the pathogenesis of idiopathic pulmonary fibrosis. In most reported case series, up to 75 percent of index patients with idiopathic pulmonary fibrosis are current or former smokers. In large epidemiologic studies, cigarette smoking has been strongly associated with idiopathic pulmonary fibrosis. In addition, many of the inflammatory features of idiopathic pulmonary fibrosis are more strongly linked to smoking status than to the underlying lung disease. Thus, cigarette smoking may be an independent risk factor for idiopathic pulmonary fibrosis. Latent viral infections, especially those of the herpes virus family, have also been reported to be associated with idiopathic pulmonary fibrosis.

While pathogenic mechanisms are incompletely understood, the currently accepted paradigm proposes that injury to the alveolar epithelium is followed by a burst of pro-inflammatory and fibroproliferative mediators that invoke responses associated with normal tissue repair. For unclear reasons, these repair processes never resolve and progressive fibrosis ensues. (Selman M, et al., Ann Intern Med, 134(2):136-151, 2001; Noble, P. and Homer R., Clin Chest Med, 25(4):749-58, 2004; Strieter, R., Chest, 128 (5 Suppl 1):526S-532S, 2005).

### Chronic Obstructive Pulmonary Disease

Chronic obstructive pulmonary disease (COPD) is a collective description for lung diseases represented by chronic and relatively irreversible expiratory airflow dysfunction due to some combination of chronic obstructive bronchitis, emphysema, and/or chronic asthma. COPD is caused by a range of environmental and genetic risk factors, including smoking that contributes to the disease.

The prevalence of COPD is increasing worldwide, and COPD has become the fourth leading cause of death in the United States. In the United States, despite the decrease in cigarette smoking in recent decades, both the prevalence of, and the mortality associated with, COPD have increased and are projected to continue to increase for some years yet. Furthermore, COPD is costly, and acute exacerbations, which occur roughly once a year in patients with COPD of moderate or greater severity, constitute the most expensive component.

In COPD, airflow obstruction can occur on the basis of either of two very different pathophysiological processes in the lung: 1) inflammation of the parenchyma resulting in proteolysis of the lung parenchyma and loss of lung elasticity (emphysema); and 2) inflammation, scarring and narrowing of the small airways (“small airway disease”). In an individual patient, one of these processes, which may be controlled by different genetic factors, may predominate although both usually co-exist. Ultimately, both of these processes produce similar patterns of functional impairment: decreased expiratory flow, hyperinflation and abnormalities of gas exchange.

At an early stage of COPD, the following symptoms are found in the lungs of COPD patients: 1) breach of airway epithelium by damaging aerosols, 2) accumulation of inflammatory mucous exudates, 3) infiltration of the airway wall by inflammatory immune cells, 4) airway remodeling/thickening of the airway wall and encroachment on lumenal space, and 5) increased resistance to airflow. During this early stage, smooth muscle contraction and hyper-responsiveness also increase resistance, but the increased resistance is relieved by bronchodilators.

At an advanced stage, COPD patients characteristically develop deposition of fibrous connective tissue in the subepithelial and aventitial compartments surrounding the airway wall. Such peribronchiolar fibrosis contributes to fixed airway obstruction by restricting the enlargement of airway caliber that occurs with lung inflation.

### Emphysema

Emphysema is defined in terms of its pathological features, characterized by abnormal dilatation of the terminal air spaces distal to the terminal bronchioles, with destruction of their wall and loss of lung elasticity. Bullae (blisters larger than 1 cm wide) may develop as a result of overdistention if areas of emphysema are larger than 1 cm in diameter. The distribution of the abnormal air spaces allows for the classification of the two main patterns of emphysema: panacinar (panlobular) emphysema, which results in distension, and destruction of the whole of the acinus, particularly the lower half of the lungs. Centriacinar (centrilobular) emphysema involves damage around the respiratory bronchioles affecting the upper lobes and upper parts of the lower lobes of the lung. Certain forms of emphysema are furthermore known to be associated with fibrosis.

The destructive process of emphysema is predominantly associated with cigarette smoking. Cigarette smoke is an irritant and results in low-grade inflammation of the airways and alveoli. It is known that cigarettes contain over 4,000 toxic chemicals, which affect the balance between the antiprotease and proteases within the lungs, causing permanent damage. Inflammatory cells (macrophages and neutrophils) produce a proteolytic enzyme known as elastase, which destroys elastin, an important component of lung tissue.

The alveoli or air sacs of the lung contain elastic tissue, which supports and maintains the potency of the intrapulmonary airways. The destruction of the alveolar walls allows narrowing in the small airways by loosening the guy ropes that help keep the airways open. During normal inspiration, the diaphragm moves downwards while the rib cage moves outwards, and air is drawn into the lungs by the negative pressure that is created. On expiration, as the rib cage and diaphragm relax, the elastic recoil of the lung parenchyma pushes air upwards and outwards. With destruction of the lung parenchyma, which results in floppy lungs and loss of the alveolar guy ropes, the small airways collapse and air trapping occurs, leading to hyperinflation of the lungs. Hyperinflation flattens the diaphragm, which results in less effective contraction and reduced alveolar efficiency, which in turn leads to further air trapping. Over time the described mechanism leads to severe airflow obstruction, resulting in insufficient expiration to allow the lungs to deflate fully prior to the next inspiration.

Chronic Asthma

Asthma is defined as a chronic inflammatory condition of the airways, leading to widespread and variable airway obstruction that is reversible spontaneously or with treatment. In some patients with chronic asthma, the disease progresses, leading to irreversible airway obstruction, particularly if the asthma is untreated, either because it has not been diagnosed or mismanaged, or if it is particularly severe. Children with asthma have a one in ten chance of developing irreversible asthma, while the risk for adult-onset asthmatics is one in four. Studies also have found that in both children and adults that asthma might lead to irreversible deterioration in lung function if their asthma was not treated appropriately, particularly with corticosteroid therapy.

Although inhaled glucocorticoids currently are the most effective anti-inflammatory treatment for asthma, a subset of asthmatic subjects is relatively insensitive to this treatment. (Zijlstra, G J et al., (2012) “Interleukin 17A induces glucocorticoid insensitivity in human bronchial epithelial cells,” Eur. Respir. J. 39: 439-45). Glucocorticoids (GCs) exert a broad spectrum of anti-inflammatory effects upon binding to their receptor (GR). For example, the ligated receptor translocates to the nucleus and suppresses pro-inflammatory gene transcription by recruitment of histone deacetylases (HDACs), which induce deacetylation of histones containing inflammatory genes, thereby restricting access of the transcriptional machinery to these genes and inhibiting transcription. GCs also are able to exert anti-inflammatory effects through activation of glucocorticoid response elements (GREs), which are present in the promoter of several anti-inflammatory genes, inducing their transcription.

Reduced sensitivity to glucocorticoids has been clinically associated with neutrophilic airway inflammation, but it is largely unclear which cellular and molecular mechanisms contribute to this insensitivity.

GR phosphorylation is regulated by a variety of Ser/Thr kinases and phosphatases. Protein phosphatase 5 (PP5) has been shown to be essential in driving cytokine-induced GC insensitivity by promoting GR dephosphorylation at S211 in ASM cells (Bouazza, B. et al, “Basal p38 Mitogen-activated protein kinase regulates unliganded glucocorticoid receptor function in airway smooth muscle cells” (2014) Am. J. Respir. Cell Mol. Bio. 50(2): 301-15 citing Bouazza, B. et al. (2012) “Cytokines after glucocorticoid receptor phosphorylation in airway cells: role of phosphatases,” Am. J. Respir. Cell Molec. Biol. 47: 464-73). However, the nature of kinases responsible for GR phosphorylation in ASM cells and in other cell types is controversial. It has been reported that the MAPK pathway is critical in determining the transcriptional activities of GR-mediated GC effects (Id. Citing Inusen, E. et al (2002), “p38 mitogen-activated protein kinase-induced glucocorticoid receptor phosphorylation reduces its activity: role in seroid-in densitive asthma,” J. Allergy Clin. Immuno. 109: 649-57; Itoch, M. et al al (2002) “Nuclear export of glucocorticoid receptor is enhanced by cjun n-terminal kinase-mediated phosphorylation,” Mol. Endocrinol. 16: 2382-92; Miller, A L et al (2005) “p38 mitogen-activated protein kinase (MAPK) is a key mediator in glucocorticoid-induced apoptosis of lymphoid cells: correlation between p38 MAPK activation and site-specific phosphorylation of the uman glucocorticoid receptor at serine 211,” Mol. Endocrino. 19: 1569-83; Rogatsky, I. et al (1998),“ Antagonism of glucocorticoid receptor transcriptional activation by the c-jun N-terminal kinase, ”Proc. Natl Acad. Sci. USA 95: 2050-55; takabe, S. et al, (2008)“ De-phosphorylation of GR at ser203 in nuclei associates with GR nuclear translocation and glut5 gene expression in caco-2 cells,” Arch. Biochem. Biophys. 475: 1-6; Tanaka, T. et al (2006), “Modification of glucocorticoid sensitivity by MAP kinase signaling pathways in glucocorticoid-induced T cell apoptosis,” Exp. Hematol. 34: 1542-52)). Several reports have implicated p38 mitogen-activated protein kinase (MAPK), a Ser/Thr kinase involved in many processes thought to be important in inflammatory diseases (Id. Citing Adcock, I M et al, (2006) “Kinase inhibitors and airway inflammation” Eur. J. Pharmcol. 533: 118-132; Kuma, S. et al (2003) “p38 map kinases: key signaling molecules as therapeutic targets for inflammatory disease,” Nat. Rev. Drug Discov. 2: 717-26; Saklatvala, J. (2004) “The p38 map kinase pathway as a therapeutic target in inflammatory disease,” Curr. Opin. Pharmcol. 4: 372-77), in the pathogenesis of patients with asthma, in particular those with severe disease (Id. Citing Bhavsar, P. et al (2010) “Effect of p38 MAPK inhibition on corticosteroid suppression of cytokine resase in severe asthma,” Eur. Respir. J. 35: 750-56; Chang, P J et al (2012), “Corticosteroid insensitivity of chemokine expression in airway smooth muscle of patients with severe asthma,” J. Allergy Clin. Immunol. 130: 877-85; Chung, K F (2011) “p38 mitogen-activated protein kinase pathways in asthma and COPD,” Chest 139: 1470-79; Mercado, N. et al (2012) “Restoration of corticosteroid sensitivity by p38 mitogen activated protein kinase inhibition in peripheral blood mononuclear cells from severe asthma,” PLoS ONE 7: e41582). Studies have suggested that the p38 MAPK-GR interaction acts as a mechanism driving GC resistance in patients with severe asthma, and direct GR phosphorylation on S226 by p38 MAPK appears to be one possible pathway driving the loss of GC efficacy seen in such patients. Id. p38 MAPK blockade was shown to positively regulate GR nuclear translocation and GR-dependent induction of the steroid-target gene GC-induced leucine zipper in a hormone-independent manner. Id. Moreover, p38 MAPK-dependent regulation of GR functions was associated with a differential action on GR phosphorylation at S203 and S211 residues. Id. Lastly, it was shown that the inactive state of GR in resting conditions is ensured by the absence of the GC ligand and by p38 MAPK-dependent phosphorylation of unliganded GR at specific residues, which appears to be important in determining the overall GC responsiveness of ASM cells. Id.

In the human bronchial epithelial cell line 16HBE, IL-17A was reported to activate the p38, extracellular signal-related kinase (ERK) and phosphoinositide-3-kinase (PI3K) pathways, the latter of which appeared to be involved in IL-17A-induced glucocorticoid insensitivity. (Zijlstra, G J et al., (2012) “Interleukin 17A induces glucocorticoid insensitivity in human bronchial epithelial cells,” Eur. Respir. J. 39: 439-45).

The airway inflammation in asthma over time can lead to remodeling of the airways through increased smooth muscle, disruption of the surface epithelium, increased collagen deposition and thickening of the basement membrane.

Increased Smooth Muscle

Increased airway smooth muscle (ASM) mass is the most prominent feature of airway remodeling (N. Carroll, J. Elliot, A. Morton, and A. James, “The structure of large and small airways in nonfatal and fatal asthma,” American Review of Respiratory Disease, vol. 147, no. 2, pp. 405-410, 1993), with ASM mass increasing disproportionately compared to the increase in total wall thickness (E. Tagaya and J. Tamaoki, “Mechanisms of airway remodeling in asthma,” Allergology International, vol. 56, no. 4, pp. 331-340, 2007). Airway remodeling has been documented in both fatal and nonfatal asthma (A. J. James, “Relationship between airway wall thickness and airway hyperesponsiveness,” in Airway Wall Remodeling in Asthma, A. G. Stewart, Ed., pp. 1-27, CRC Press, Boca Raton, Fla., USA, 1997), and correlates with both disease severity and duration, being greater in fatal than nonfatal cases (N. Carroll, J. Elliot, A. Morton, and A. James, “The structure of large and small airways in nonfatal and fatal asthma,” American Review of Respiratory Disease, vol. 147, no. 2, pp. 405-410, 1993; A. L. James, P. D. Pare, and J. C. Hogg, “The mechanics of airway narrowing in asthma,” American Review of Respiratory Disease, vol. 139, no. 1, pp. 242-246, 1989; K. Kuwano, C. H. Bosken, P. D. Pare, T. R. Bai, B. R. Wiggs, and J. C. Hogg, “Small airways dimensions in asthma and in chronic obstructive pulmonary disease,” American Review of Respiratory Disease, vol. 148, no. 5, pp. 1220-1225, 1993) and greater in older patients than in younger patients with fatal asthma. The increase in ASM mass may be the coordinated result of increased myocyte size (hypertrophy), increased myocyte number (hyperplasia), and differentiation and migration of mesenchymal cells to ASM bundles (S. Beqaj, S. Jakkaraju, R. R. Mattingly, D. Pan, and L. Schuger, “High RhoA activity maintains the undifferentiated mesenchymal cell phenotype, whereas RhoA down-regulation by laminin-2 induces smooth muscle myogenesis,” Journal of Cell Biology, vol. 156, no. 5, pp. 893-903, 2002; S. J. Hirst, J. G. Martin, J. V. Bonacci et al., “Proliferative aspects of airway smooth muscle,” Journal of Allergy and Clinical Immunology, vol. 114, no. 2, pp. S2-S17, 2004; M. Schmidt, G. Sun, M. A. Stacey, L. Mori, and S. Mattoli, “Identification of circulating fibrocytes as precursors of bronchial myofibroblasts in asthma,” Journal of Immunology, vol. 171, no. 1, pp. 380-389, 2003; C. Bergeron, W. Al-Ramli, and Q. Hamid, “Remodeling in asthma,” Proceedings of the American Thoracic Society, vol. 6, no. 3, pp. 301-305, 2009).

Mitogens, chemical compounds that stimulate cell division and trigger mitosis (A. Shifren, C. Witt, C. Christie and M. Castro, “Mechanisms of Remodeling in Asthmatic Airways,” Journal of Allergy, vol. 2012, Article ID 316049, pp. 1-12), play an integral role in the development of increased ASM mass typical of asthmatic airways. Mitogens bind receptor tyrosine kinases (RTK), G protein-coupled receptors (GPCR), and cytokine receptors, all of which are capable of producing increases in ASM mass in cell culture models (E. Tagaya and J. Tamaoki, “Mechanisms of airway remodeling in asthma,” Allergology International, vol. 56, no. 4, pp. 331-340, 2007). The list of mitogens is extensive, and includes TGF-β, IL-1β, IL-6, thromboxanes, leukotrienes, histamine, tryptase, serotonin, vascular endothelial growth factor (VEGF), and numerous others (S. J. Hirst, J. G. Martin, J. V. Bonacci et al., “Proliferative aspects of airway smooth muscle,” Journal of Allergy and Clinical Immunology, vol. 114, no. 2, pp. S2-S17, 2004; A. M. Freyer, S. R. Johnson, and I. P. Hall, “Effects of growth factors and extracellular matrix on survival of human airway smooth muscle cells,” American Journal of Respiratory Cell and Molecular Biology, vol. 25, no. 5, pp. 569-576, 2001; P. H. Howarth, A. J. Knox, Y. Amrani, O. Tliba, R. A. Panettieri, and M. Johnson, “Synthetic responses in airway smooth muscle,” Journal of Allergy and Clinical Immunology, vol. 114, no. 2, supplement 1, pp. S32-S50, 2004). The receptor systems regulate mitogenesis primarily through the phosphoinositide 3′-kinase (PI3K) and extracellular signal-regulated kinase (ERK) signaling pathways (K. Page, J. Li, Y. Wang, S. Kartha, R. G. Pestell, and M. B. Hershenson, “Regulation of cyclin D(1) expression and DNA synthesis by phosphatidylinositol 3-kinase in airway smooth muscle cells,” American Journal of Respiratory Cell and Molecular Biology, vol. 23, no. 4, pp. 436-443, 2000; M. J. Orsini, V. P. Krymskaya, A. J. Eszterhas, J. L. Benovic, R. A. Panettieri, and R. B. Penn, “MAPK superfamily activation in human airway smooth muscle: mitogenesis requires prolonged p42/p44 activation,” American Journal of Physiology, vol. 277, no. 3, pp. L479-L488, 1999). The PI3K and ERK pathways activate transcription factors which phosphorylate D-type cyclins facilitating cell cycle progression (E. Tagaya and J. Tamaoki, “Mechanisms of airway remodeling in asthma,” Allergology International, vol. 56, no. 4, pp. 331-340, 2007). Almost all of these mitogens have been identified in airway biopsies and bronchoalveolar lavage (BAL) fluid from asthmatic patients or are detected in asthmatic airway cell cultures (R. M. Pascual and S. P. Peters, “Airway remodeling contributes to the progressive loss of lung function in asthma: an overview,” Journal of Allergy and Clinical Immunology, vol. 116, no. 3, pp. 477-486, 2005).

ASM cells are often noted in close proximity to the airway epithelium (A. Shifren, C. Witt, C. Christie and M. Castro, “Mechanisms of Remodeling in Asthmatic Airways,” Journal of Allergy, vol. 2012, Article ID 316049, pp. 1-12). This epithelial-muscle distance was measured at 67 μm in asthmatics compared to 135 μm in controls (L. Benayoun, A. Druilhe, M. C. Dombret, M. Aubier, and M. Pretolani, “Airway structural alterations selectively associated with severe asthma,” American Journal of Respiratory and Critical Care Medicine, vol. 167, no. 10, pp. 1360-1368, 2003). It has been postulated that mesenchymal airway cells differentiate into ASM with subsequent migration of the new ASM cells into muscle bundles (J. M. Madison, “Migration of airway smooth muscle cells,” American Journal of Respiratory Cell and Molecular Biology, vol. 29, no. 1, pp. 8-11, 2003). Whether these phenomena occur in vivo is unknown, but reports indicate that cultured human ASM cells migrate in response to mitogenic stimuli (M. Hoshino, M. Takahashi, and N. Aoike, “Expression of vascular endothelial growth factor, basic fibroblast growth factor, and angiogenin immunoreactivity in asthmatic airways and its relationship to angiogenesis,” Journal of Allergy and Clinical Immunology, vol. 107, no. 2, pp. 295-301, 2001). Many of the mitogens involved in cell proliferation, including TGF-β, IL-1β, and VEGF, also induce ASM cell migration (R. M. Pascual and S. P. Peters, “Airway remodeling contributes to the progressive loss of lung function in asthma: an overview,” Journal of Allergy and Clinical Immunology, vol. 116, no. 3, pp. 477-486, 2005; E. Tagaya and J. Tamaoki, “Mechanisms of airway remodeling in asthma,” Allergology International, vol. 56, no. 4, pp. 331-340, 2007).

Disruption of Surface Epithelium

Epithelial cell shedding, ciliated cell loss, and goblet cell hyperplasia have all been described in asthmatic airways (N. Carroll, J. Elliot, A. Morton, and A. James, “The structure of large and small airways in nonfatal and fatal asthma,” American Review of Respiratory Disease, vol. 147, no. 2, pp. 405-410, 1993; T. Aikawa, S. Shimura, H. Sasaki, M. Ebina, and T. Takishima, “Marked goblet cell hyperplasia with mucus accumulation in the airways of patients who died of severe acute asthma attack,” Chest, vol. 101, no. 4, pp. 916-921, 1992; B. NAYLOR, “The shedding of the mucosa of the bronchial tree in asthma,” Thorax, vol. 17, pp. 69-72, 1962). Evidence of increased epithelial cell proliferation contributing to thickening of the epithelium and an increased lamina reticularis (also known as subepithelial fibrosis) has been observed in patients with moderate to severe asthma while being absent in patients with mild persistent asthma, chronic bronchitis, and normal controls (L. Cohen, E. Xueping, J. Tarsi et al., “Epithelial cell proliferation contributes to airway remodeling in severe asthma,” American Journal of Respiratory and Critical Care Medicine, vol. 176, no. 2, pp. 138-145, 2007). These studies suggest that thickening of the airway seen in severe asthma may be due, in part, to airway epithelial proliferation.

Goblet cell hyperplasia has been consistently demonstrated in mild, moderate, and severe forms of asthma (H. A. Jenkins, C. Cool, S. J. Szefler et al., “Histopathology of severe childhood asthma: a case series,” Chest, vol. 124, no. 1, pp. 32-41, 2003; C. L. Ordoñez, R. Khashayar, H. H. Wong et al., “Mild and moderate asthma is associated with airway goblet cell hyperplasia and abnormalities in mucin gene expression,” American Journal of Respiratory and Critical Care Medicine, vol. 163, no. 2, pp. 517-523, 2001). Similarly, an increase in the area of airway wall occupied by submucosal mucus glands is a frequent finding in asthmatic airways, and occurs in both fatal and nonfatal forms of asthma (N. Carroll, J. Elliot, A. Morton, and A. James, “The structure of large and small airways in nonfatal and fatal asthma,” American Review of Respiratory Disease, vol. 147, no. 2, pp. 405-410, 1993). Goblet cells produce mucin glycoproteins (MUC), of which thirteen (13) have been identified in human airways (E. Tagaya and J. Tamaoki, “Mechanisms of airway remodeling in asthma,” Allergology International, vol. 56, no. 4, pp. 331-340, 2007). The dominant mucin in humans is MUC5AC, which is expressed in the airways of normal subjects and is upregulated in asthmatic subjects (J. V. Fahy, “Remodeling of the airway epithelium in asthma,” American Journal of Respiratory and Critical Care Medicine, vol. 164, no. 10, pp. S46-51, 2001). Goblet cell hyperplasia has been demonstrated following adoptive transfer of Th2 cells into ovalbumin-challenged mice and is most likely the result of Th2-driven interleukin expression (L. Cohn, J. S. Tepper, and K. Bottomly, “Cutting edge: IL-4-independent induction of airway hyperresponsiveness by Th2, but not Th1, cells,” Journal of Immunology, vol. 161, no. 8, pp. 3813-3816, 1998). IL-13 signals through the STAT-6 signaling pathway (R. J. Homer and J. A. Elias, “Airway remodeling in asthma: therapeutic implications of mechanisms,” Physiology, vol. 20, no. 1, pp. 28-35, 2005) and the effects of IL-13 overexpression in mice are almost completely STAT-6 dependent (D. A. Kuperman, X. Huang, L. L. Koth et al., “Direct effects of interleukin-13 on epithelial cells cause airway hyperreactivity and mucus overproduction in asthma,” Nature Medicine, vol. 8, no. 8, pp. 885-889, 2002).

Epithelial injury is normally followed by upregulation of proteins responsible for tissue repair. Expression of epithelial growth factor receptor (EGFR) and MUC5AC are both markedly upregulated in the epithelium of asthmatic patients (M. Amishima, M. Munakata, Y. Nasuhara et al., “Expression of epidermal growth factor and epidermal growth factor receptor immunoreactivity in the asthmatic human airway,” American Journal of Respiratory and Critical Care Medicine, vol. 157, no. 6, pp. 1907-1912, 1998; S. M. Puddicombe, R. Polosa, A. Richter et al., “Involvement of the epidermal growth factor receptor in epithelial repair in asthma,” FASEB Journal, vol. 14, no. 10, pp. 1362-1374, 2000), and have been shown to co-localize in goblet cells (K. Takeyama, J. V. Fahy, and J. A. Nadel, “Relationship of epidermal growth factor receptors to goblet cell production in human bronchi,” American Journal of Respiratory and Critical Care Medicine, vol. 163, no. 2, pp. 511-516, 2001). Immunoreactivity to EGFR and the total area of MUC5AC staining show a positive correlation in both asthmatics and control subjects. Furthermore, activation of EGFR has been shown to upregulate both mucin production and goblet cell generation in human epithelial cells in vitro (M. Amishima, M. Munakata, Y. Nasuhara et al., “Expression of epidermal growth factor and epidermal growth factor receptor immunoreactivity in the asthmatic human airway,” American Journal of Respiratory and Critical Care Medicine, vol. 157, no. 6, pp. 1907-1912, 1998).

### Increased Collagen Deposition and Thickening of the Basement Membrane

The original report of airway remodeling described the phenomenon of basement membrane thickening (H. L. Huber and K. K. Koessler, “The pathology of bronchial asthma,” Archives of Internal Medicine, vol. 30, no. 6, pp. 689-760, 1922). Electron microscopy has subsequently shown that thickening occurs just below the true basement membrane in a zone known as the lamina reticularis (W. R. Roche, J. H. Williams, R. Beasley, and S. T. Holgate, “Subepithelial fibrosis in the bronchi of asthmatics,” Lancet, vol. 1, no. 8637, pp. 520-524, 1989). The lamina reticularis is a collagenous layer 4-5 μm thick in control subjects. In asthmatics, thickness of the lamina reticularis has been documented at between 7 and 23 μm (R. J. Homer and J. A. Elias, “Consequences of long-term inflammation: airway remodeling,” Clinics in Chest Medicine, vol. 21, no. 2, pp. 331-343, 2000). Thickening is the result of extracellular matrix deposition, primarily collagens 1, III, and V (R. J. Homer and J. A. Elias, “Airway remodeling in asthma: therapeutic implications of mechanisms,” Physiology, vol. 20, no. 1, pp. 28-35, 2005). In addition, abnormalities of noncollagenous matrix, including elastin, fibronectin, tenascin, lumican, and proteoglycans, have also been described (W. R. Roche, J. H. Williams, R. Beasley, and S. T. Holgate, “Subepithelial fibrosis in the bronchi of asthmatics,” Lancet, vol. 1, no. 8637, pp. 520-524, 1989; J. Huang, R. Olivenstein, R. Taha, Q. Hamid, and M. Ludwig, “Enhanced proteoglycan deposition in the airway wall of atopic asthmatics,” American Journal of Respiratory and Critical Care Medicine, vol. 160, no. 2, pp. 725-729, 1999; A. Laitinen, A. Altraja, M. Kampe, M. Linden, I. Virtanen, and L. A. Laitinen, “Tenascin is increased in airway basement membrane of asthmatics and decreased by an inhaled steroid,” American Journal of Respiratory and Critical Care Medicine, vol. 156, no. 3, pp. 951-958, 1997).

Myofibroblasts are believed to be key effectors of subepithelial fibrosis. Myofibroblasts are specialized cells with phenotypic characteristics of both fibroblasts and myocytes (E. Tagaya and J. Tamaoki, “Mechanisms of airway remodeling in asthma,” Allergology International, vol. 56, no. 4, pp. 331-340, 2007). They express α-smooth muscle actin, produce inflammatory mediators, and are major producers of extracellular matrix proteins necessary for tissue repair and remodeling.

Transforming growth factor- (TGF-) β mediates the effects of IL-13 overexpressing mice (Chun Geun Lee, R. J. Homer, Z. Zhu et al., “Interleukin-13 induces tissue fibrosis by selectively stimulating and activating transforming growth factor β1,” Journal of Experimental Medicine, vol. 194, no. 6, pp. 809-821, 2001). TGF-β is a cytokine produced by multiple lung cells including epithelial cells, macrophages, fibroblasts, lymphocytes, and eosinophils (E. Tagaya and J. Tamaoki, “Mechanisms of airway remodeling in asthma,” Allergology International, vol. 56, no. 4, pp. 331-340, 2007). TGF-β induces fibroblasts to express α-smooth muscle actin and assume a myofibroblast phenotype (V. Batra, A. I. Musani, A. T. Hastie et al., “Bronchoalveolar lavage fluid concentrations of transforming growth factor (TGF)-β1, TGF-β2, interleukin (IL)-4 and IL-13 after segmental allergen challenge and their effects on α-smooth muscle actin and collagen III synthesis by primary human lung fibroblasts,” Clinical and Experimental Allergy, vol. 34, no. 3, pp. 437-444, 2004). As part of normal wound repair, TGF-β induces expression and secretion of multiple extracellular matrix proteins while also inhibiting their degradation. In many diseases, excessive TGF-β results in an excess of pathologic tissue fibrosis leading to compromised organ function (M. H. Branton and J. B. Kopp, “TGF-β and fibrosis,” Microbes and Infection, vol. 1, no. 15, pp. 1349-1365, 1999). TGF-β expression is increased in asthmatic airways and BAL fluid, compared to controls. In addition, TGF-β levels correlate with the extent of subepithelial fibrosis, airway fibroblast numbers, and disease severity (E. M. Minshall, D. Y. M. Leung, R. J. Martin et al., “Eosinophil-associated TGF-β1 mRNA expression and airways fibrosis in bronchial asthma,” American Journal of Respiratory Cell and Molecular Biology, vol. 17, no. 3, pp. 326-333, 1997; I. Ohno, Y. Nitta, K. Yamauchi et al., “Transforming growth factor β1 (TGFβ1) gene expression by eosinophils in asthmatic airway inflammation,” American Journal of Respiratory Cell and Molecular Biology, vol. 15, no. 3, pp. 404-409, 1996; L. P. Boulet, M. Belanger, and G. Carrier, “Airway responsiveness and bronchial-wall thickness in asthma with or without fixed airflow obstruction,” American Journal of Respiratory and Critical Care Medicine, vol. 152, no. 3, pp. 865-871, 1995). Thus, excess TGF-β production may be pivotal for the development of subepithelial fibrosis.

Matrix metalloproteinases are zinc-dependent endopeptidases capable of degrading extracellular matrix molecules. The dynamic equilibrium between matrix metalloproteinases and their inhibitors is a critical determinant of matrix remodeling (R. Visse and H. Nagase, “Matrix metalloproteinases and tissue inhibitors of metalloproteinases: structure, function, and biochemistry,” Circulation Research, vol. 92, no. 8, pp. 827-839, 2003). The existence of increased subepithelial fibrosis in asthmatic airways suggests that a profibrotic balance exists between the two. In asthma, the most important metalloproteinase molecules are MMP-9 and its inhibitor, tissue inhibitor of metalloproteinase- (TIMP-) 1 (R. J. Homer and J. A. Elias, “Airway remodeling in asthma: therapeutic implications of mechanisms,” Physiology, vol. 20, no. 1, pp. 28-35, 2005). Both MMP-9 and TIMP-1 levels are elevated in airway biopsies and BAL fluid of asthmatic patients (A. M. Vignola, L. Riccobono, A. Mirabella et al., “Sputum metalloproteinase-9/tissue inhibitor of metalloproteinase-1 ratio correlates with airflow obstruction in asthma and chronic bronchitis,” American Journal of Respiratory and Critical Care Medicine, vol. 158, no. 6, pp. 1945-1950, 1998; M. Hoshino, Y. Nakamura, J. Sim, J. Shimojo, and S. Isogai, “Bronchial subepithelial fibrosis and expression of matrix metalloproteinase-9 in asthmatic airway inflammation,” Journal of Allergy and Clinical Immunology, vol. 102, no. 5, pp. 783-788, 1998; G. Mautino, C. Henriquet, C. Gougat et al., “Increased expression of tissue inhibitor of metalloproteinase-1 and loss of correlation with matrix metalloproteinase-9 by macrophages in asthma). However, compared to control subjects, asthmatics have a significantly lower MMP-9 to TIMP-1 ratio, supporting a profibrotic balance (inhibition over degradation). In addition, the lower MMP-9 to TIMP-1 ratios correlate with the degree of airway obstruction (E. A. Kelly and N. N. Jarjour, “Role of matrix metalloproteinases in asthma,” Current Opinion in Pulmonary Medicine, vol. 9, no. 1, pp. 28-33, 2003).

TGF-β is secreted from cells as a latent complex and is targeted to the extracellular matrix by latent TGF-β binding proteins for subsequent activation (M. Hyytiäinen, C. Penttinen, and J. Keski-Oja, “Latent TGF-β binding proteins: extracellular matrix association and roles in TGF-β activation,” Critical Reviews in Clinical Laboratory Sciences, vol. 41, no. 3, pp. 233-264, 2004). MMPs regulate matrix-bound cytokine release (E. A. Kelly and N. N. Jarjour, “Role of matrix metalloproteinases in asthma,” Current Opinion in Pulmonary Medicine, vol. 9, no. 1, pp. 28-33, 2003), and activation of TGF-β is MMP-9 dependent (Chun Geun Lee, R. J. Homer, Z. Zhu et al., “Interleukin-13 induces tissue fibrosis by selectively stimulating and activating transforming growth factor β1,” Journal of Experimental Medicine, vol. 194, no. 6, pp. 809-821, 2001). Therefore, the role of elevated levels of MMP-9 in asthma may be related to TGF-β activation and its downstream fibrotic sequelae (R. J. Homer and J. A. Elias, “Airway remodeling in asthma: therapeutic implications of mechanisms,” Physiology, vol. 20, no. 1, pp. 28-35, 2005).

### The Immune Response

Immune responses are initiated by an individual's encounter with a foreign antigenic substance/immunogen, for example, an infectious agent. The individual rapidly responds with the production of antibody molecules specific for epitopes of the immunogen (humoral response) and with the expansion and differentiation of antigen-specific regulatory and effector T-lymphocytes. The latter include cells that produce cytokines and killer T cells capable of lysing the infected cells (cell-mediated immune response). Generally this initial immune response is sufficient to control and eradicate the foreign substance.

Generally, as a consequence of the initial response, an immunized individual develops a state of immunologic memory. Paul, W. E., “Chapter 1: The immune system: an introduction,” Fundamental Immunology, 4th Edition, Ed. Paul, W. E., Lippicott-Raven Publishers, Philadelphia (1999). If the same (or a closely related) foreign substance is encountered again, a secondary response is made, which generally consists of an enhanced antibody and T-cell response. This is the basis of vaccination.

Inflammation

Inflammation is the physiologic process by which vascularized tissues respond to injury (See, e.g., FUNDAMENTAL IMMUNOLOGY, 4th Ed., William E. Paul, ed. Lippincott-Raven Publishers, Philadelphia (1999) at 1051-1053, incorporated herein by reference). During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. In acute inflammation, removal of the stimulus halts the recruitment of monocytes (which become macrophages under appropriate activation) into the inflamed tissue, and existing macrophages exit the tissue via lymphatics. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents. The classic signs of inflammation are pain (dolor), heat (calor), redness (rubor), swelling (tumor), and loss of function (functio laesa). Histologically, inflammation involves a complex series of events, including dilatation of arterioles, capillaries, and venules, with increased permeability and blood flow; exudation of fluids, including plasma proteins; and leukocytic migration into the inflammatory focus.

The term “chronic inflammation” as used herein refers to inflammation that is of longer duration and which has a vague and indefinite termination. Chronic inflammation takes over when acute inflammation persists, either through incomplete clearance of the initial inflammatory agent (e.g., cigarette smoking) or as a result of multiple acute events occurring in the same location. Chronic inflammation, which includes the influx of lymphocytes and macrophages and fibroblast growth, may result in tissue scarring at sites of prolonged or repeated inflammatory activity. In chronic inflammation, existing macrophages are tethered in place, and proliferation of macrophages is stimulated.

Regardless of the initiating agent, the physiologic changes accompanying acute inflammation encompass four main features: (1) vasodilation, which results in a net increase in blood flow, is one of the earliest physical responses to acute tissue injury; (2) in response to inflammatory stimuli, endothelial cells lining the venules contract, widening the intracellular junctions to produce gaps, leading to increased vascular permeability which permits leakage of plasma proteins and blood cells out of blood vessels; (3) inflammation often is characterized by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli.

During the inflammatory process, soluble inflammatory mediators of the inflammatory response work together with cellular components in a systemic fashion in the attempt to contain and eliminate the agents causing physical distress. The molecular mediators of the inflammatory process (“inflammatory mediators”) are soluble, diffusible molecules that act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1, interleukin-4 (IL-4), interleukin-6 (IL-6), interleukin-8 (IL-8), tumor necrosis factor (TNF), interferon-gamma, interleukin-12 (IL-12), and interleukin-17 (IL-17).

Inflammation is the body's adaptive response to any insult, be it mechanical, biochemical, or immune-mediated. However, inflammation is beneficial only on the condition that it ends in active resolution (Benhar, I. et al. (2012) “The privileged immunity of immune privileged organs: the case of the eye,” Front. Immunol. 3: 296. doi.org/10.3389/firmmu.2012.00296 citing Gronert, K. (2010) “Resolution, the grail for healthy ocular inflammation,” Exp. Eye Res. 91: 478-85).

The early innate immune response involves cells that are needed for cleaning a site of injury. The activity of these cells must be followed by immune cells that terminate the initial response and subsequently contribute to repair. Both stages involve innate immune cells of distinct phenotypes; the cells that contribute to the termination of the local early response are largely monocyte-derived macrophages that acquire and exert a local anti-inflammatory function (Id. citing Kigerl, K. A. et al. (2009), “Identification of two distinct macrophage subsets with divergent effects causing either neurotoxicity or regeneration in the injured mouse spinal cord,” J. Neurosci. 29: 13435-444; Shechter, R. et al. (2009), “Infiltrating blood-derived macrophages are vital cells playing an anti-inflammatory role in recovery from spinal cord injury in mice,” PLoS Med. 6: e1000113; doi: 10.1371/journal.pmed.1000113; London, A. et al. (2011) “Neuroprotection and progenitor cell renewal in the injured adult murine retina requires healing monocyte-derived macrophages,” J. Exp. Med. 208: 23-39; Zhu, B. et al. (2011) “Plasticity of Ly-6C(hi) myeloid cells in T cell regulation” J. Immuol. 187: 2418-32).

The elements of the immune system include cellular immunity, humoral immunity, and the complement system.

### Cells of the Immune System

The immune system consists of lymphocytes, which are the cells that determine the specificity of immunity, and cells that interact with lymphocytes, which play roles in the presentation of antigen and in the mediation of immunologic functions. These cells include the monocyte/macrophages, dendritic cells and closely related Langerhans' cells, natural killer (NK) cells, mast cells, basophils and other members of the myeloid lineage of cells. In addition, a series of specialized epithelial and stromal cells provide the anatomic environment in which immunity occurs, often by secreting critical factors that regulate growth and/or gene activation in cells of the immune system. Such cells also play direct roles in the induction and effector phases of the response. Paul, W. E., “Chapter 1: The immune system: an introduction,” Fundamental Immunology, 4th Edition, Ed. Paul, W. E., Lippicott-Raven Publishers, Philadelphia (1999).

Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Paul, W. E., “Chapter 1: The immune system: an introduction,” Fundamental Immunology, 4th Edition, Ed. Paul, W. E., Lippicott-Raven Publishers, Philadelphia (1999).

Two broad classes of lymphocytes are recognized: the B-lymphocytes (B-cells), which are precursors of antibody-secreting cells, and T-lymphocytes (T-cells),

### B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses. (Paul, W. E., “Chapter 1: The immune system: an introduction,” Fundamental Immunology, 4^{th} Edition, Ed. Paul, W. E., Lippicott-Raven Publishers, Philadelphia (1999)).

Cross-linkage dependent B-cell activation requires that the antigen express multiple copies of the epitope complementary to the binding site of the cell surface receptors because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes. (Paul, W. E., “Chapter 1: The immune system: an introduction,” Fundamental Immunology, 4^{th} Edition, Ed. Paul, W. E., Lippicott-Raven Publishers, Philadelphia (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell. (Paul, W. E., “Chapter 1: The immune system: an introduction,” Fundamental Immunology, 4^{th} Edition, Ed. Paul, W. E., Lippicott-Raven Publishers, Philadelphia (1999)).

During cognate help for antibody production, the CD40 ligand is transiently expressed on activated CD4+ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in the pathogenic autoantibody production in human SLE patients. (Desai-Mehta, A. et al., “Hyperexpression of CD40 ligand by B and T cells in human lupus and its role in pathogenic autoantibody production,” J. Clin. Invest., 97(9): 2063-2073 (1996)).

T-Lymphocytes

T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. Antibody and B-cell receptors evolved to bind to and to protect against microorganisms in extracellular fluids. In contrast, T cells recognize antigens on the surface of other cells and mediate their functions by interacting with, and altering, the behavior of these antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B. et al., Garland Science, NY, 2002).

T-cells are subdivided into two distinct classes based on the cell surface receptors they express. The majority of T cells express T cell receptors (TCR) consisting of α and β chains. A small group of T cells express receptors made of γ and δ chains. Among the α/β T cells are two important sublineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells particularly CD8+ T cells, can develop into cytotoxic T-lymphocytes (CTLs) capable of efficiently lysing target cells that express antigens recognized by the CTLs. (Paul, W. E., “Chapter 1: The immune system: an introduction,” Fundamental Immunology, 4^{th} Edition, Ed. Paul, W. E., Lippicott-Raven Publishers, Philadelphia (1999)).

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes. (Paul, W. E., “Chapter 1: The immune system: an introduction,” Fundamental Immunology, 4^{th} Edition, Ed. Paul, W. E., Lippicott-Raven Publishers, Philadelphia (1999)).

The TCR's ligand (i.e., the peptide/MHC protein complex) is created within antigen-presenting cells (APCs). In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources. (Paul, W. E., “Chapter 1: The immune system: an introduction,” Fundamental Immunology, 4^{th} Edition, Ed. Paul, W. E., Lippicott-Raven Publishers, Philadelphia (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally nine amino acids in length, are bound into the class I MHC molecules and are brought to the cell surface, where they can be recognized by CD8+ T cells expressing appropriate receptors. This gives the T cell system, particularly CD8+ T cells, the ability to detect cells expressing proteins that are different from, or produced in much larger amounts than, those of cells of the remainder of the organism (e.g., vial antigens) or mutant antigens (such as active oncogene products), even if these proteins in their intact form are neither expressed on the cell surface nor secreted. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4^{th} Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching (of the Ig class being expressed) either depend or are enhanced by the actions of T cell-derived cytokines. (Paul, W. E., “Chapter 1: The immune system: an introduction,” Fundamental Immunology, 4^{th} Edition, Ed. Paul, W. E., Lippicott-Raven Publishers, Philadelphia (1999)).

CD4+ T cells tend to differentiate into cells that principally secrete the cytokines IL-4, IL-5, IL-6, and IL-10 (T_{H2} cells) or into cells that mainly produce IL-2, IFN-γ, and lymphotoxin (T_{H1} cells). The T_{H2} cells are very effective in helping B-cells develop into antibody-producing cells, whereas the T_{H1} cells are effective inducers of cellular immune responses, involving enhancement of microbicidal activity of monocytes and macrophages, and consequent increased efficiency in lysing microorganisms in intracellular vesicular compartments. Although the CD4+ T cells with the phenotype of T_{H2} cells (i.e., IL-4, IL-5, IL-6 and IL-10) are efficient helper cells, T_{H1} cells also have the capacity to be helpers. (Paul, W. E., “Chapter 1: The immune system: an introduction,” Fundamental Immunology, 4^{th} Edition, Ed. Paul, W. E., Lippicott-Raven Publishers, Philadelphia (1999)).

### Priming

“Unprimed cells” (also referred to as virgin, naïve, or inexperienced cells) are T cells and B cells that have generated an antigen receptor (TCR for T cells, BCR for B cells) of a particular specificity, but have never encountered the antigen. The term “priming” as used herein refers to the process whereby T cells and B cell precursors encounter the antigen for which they are specific.

For example, before helper T cells and B cells can interact to produce specific antibody, the antigen-specific T cell precursors must be primed. Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. Janeway, C A, Jr., “The priming of helper T cells, Semin. Immunol. 1(1): 13-20 (1989). Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

T Cells Involved in Induction of Cellular Immunity

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. The T_{H1} cells are effective in enhancing the microbicidal action because they produce IFN-γ. By contrast, two of the major cytokines produced by T_{H2} cells, IL-4 and IL-10, block these activities. (Paul, W. E., “Chapter 1: The immune system: an introduction,” Fundamental Immunology, 4^{th} Edition, Ed. Paul, W. E., Lippicott-Raven Publishers, Philadelphia (1999)).

Suppressor or Regulatory T (Treg) Cells

A controlled balance between initiation and downregulation of the immune response is important to maintain immune homeostasis. Both apoptosis and T cell anergy (a tolerance mechanism in which the T cells are intrinsically functionally inactivated following an antigen encounter (Scwartz, R. H., “T cell anergy,” Annu. Rev. Immunol., 21: 305-334 (2003)) are important mechanisms that contribute to the downregulation of the immune response. A third mechanism is provided by active suppression of activated T cells by suppressor or regulatory CD4+ T (Treg) cells. (Reviewed in Kronenberg, M. et al., “Regulation of immunity by self-reactive T cells,” Nature 435: 598-604 (2005)). CD4+ Tregs that constitutively express the IL-2 receptor alpha (IL-2Rα) chain (CD4+ CD25+) are a naturally occurring T cell subset that are anergic and suppressive. (Taams, L. S. et l., “Human anergic/suppressive CD4+CD25+ T cells: a highly differentiated and apoptosis-prone population,” Eur. J. Immunol., 31: 1122-1131 (2001)). Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development. (Battaglia, M. et al., “Rapamycin promotes expansion of functional CD4+CD25+Foxp3+ regulator T cells of both healthy subjects and type 1 diabetic patients,” J. Immunol., 177: 8338-8347 (200)).

Cytotoxic T Lymphocytes (CTL)

The CD8+ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by a series of enzymes produced by activated CTLs, referred to as granzymes. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

Natural Killer (NK Cells)

NK cells belong to the lymphoid lineage. Although NK cells are not configured to recognize specific target antigens, in the way that T cells are configured to recognize target antigens, the ability of NK cells to bind to the constant region of antibodies enables NK cells to specifically kill the cells that are tagged with antibodies. The NK cell's recognition of the constant region of antibodies is mediated by the Fc receptor (of the NK cell) binding to the Fc portion of the antibody. This type of killing is called, antibody-dependent cell cytotoxicity (ADCC). NK cells can also kill cells independent of the mechanism of ADCC, where this killing requires expression of MHC class I to be lost or deficient in the target cell (see, e.g., Caligiuri (2008) Blood 112:461-469). NK cells have been reported to mediate cytotoxicity against cancer stem cells (see, e.g., Jewett and Tseng (2011) J. Cancer. 2:443-457).

Antigen Presenting Cells

Antigen presenting cells (APCs) are cells of the immune system used for presenting antigen to T cells. APCs include dendritic cells, monocytes, macrophages, marginal zone Kupffer cells, microglia, Langerhans cells, T cells, and B cells (see, e.g., Rodriguez-Pinto and Moreno (2005) Eur. J. Immunol. 35:1097-1105). Antigen-presenting cells display three types of protein molecules on their surface that have a role in activating a T cell to become an effector cell: (1) MHC proteins, which present foreign antigen to the T-cell receptor; (2) costimulatory proteins, which bind to complementary receptors on the T cell surface; and (3) cell-cell adhesion molecules, which enable a T cell to bind to the antigen-presenting cell for long enough to become activated. Alberts, B. et al., Molecular Biology of the Cell, 4^{th} Ed., Garland Science, NY (2002), p. 1394. The function of class I and class II MHC molecules is to bind and present antigen-derived peptides to T cells whose receptors can recognize the peptide/MHC complex that is generated. (Paul, W. E., “Chapter 1: The immune system: an introduction,” Fundamental Immunology, 4th Edition, Ed. Paul, W. E., Lippicott-Raven Publishers, Philadelphia (1999)), p. 15.

Dendritic Cells

Dendritic cells are discrete leukocyte population(s) of antigen presenting cells that initiate specific T-lymphocyte activation and proliferation. Their key properties include (1) the ability to take up, process, and present antigen; (2) the ability to migrate selectively through tissues; and (3) the ability to interact with, stimulate and direct T-lymphocyte responses. Hart, D N J, Blood 90(9): 3245-87, 3245 (1997). The encounter with an antigen induces the dendritic cell to mature from an antigen-capturing cell to an antigen-presenting cell that can activate T cells. Alberts, B. et al., Molecular Biology of the Cell, 4^{th} Ed., Garland Science, NY (2002), p. 1394.

DCs exhibit several features necessary for the generation of T-cell-mediated antitumor immunity (Dermime S, et al., British Medical Bulletin (2002) 62: 149-162; Celia M, et al., Curr. Opin. Immunol. (1997) 9: 10-16).

They efficiently capture and take up antigens in peripheral tissues and transport these antigens to the primary and secondary lymphoid organs where they express high levels of MHC class I and II molecules that present the processed peptides to T-cells for the priming of antigen-specific responses. Specifically, a DC acquires polypeptide antigens, where these antigens can be acquired from outside of the DC, or biosynthesized inside of the DC by an infecting organism. The DC processes the polypeptide, resulting in peptides of about ten amino acids in length, transfers the peptides to either MHC class I or MHC class II to form a complex, and shuttles the complex to the surface of the DC. When a DC bearing a MHC class I/peptide complex contacts a CD8+ T-cell, the result is activation and proliferation of the CD8+ T-cell. Regarding the role of MHC class II, when a DC bearing a MHC class II/peptide complex contacts a CD 4+ T-cell, the outcome is activation and proliferation of the CD4+ T-cell (Munz, et al. (2010) Curr. Opin. Immunol. 22:89-93; Monaco (1995) J. Leukocyte Biol. 57:543-547; Robinson, et al (2002) Immunology 105:252-262). Although dendritic cells presenting antigen to a T-cell can “activate” that T-cell, the activated T-cell might not be capable of mounting an effective immune response. Effective immune response by the CD8+ T-cell, for example, often requires prior stimulation of the DC by one or more of a number of interactions. These interactions include direct contact of a CD4+ T-cell to the DC (by way of contact of the CD4+ T-cell's CD40 ligand to the DCs CD40 receptor), or direct contact of a toll-like receptor (TLR) agonist to one of the dendritic cell's toll-like receptors (TLRs).

### Sensor Effector Receptor Systems

Examples of receptor systems that recognize and induce rapid defenses and delayed cellular responses include, without limitation, the complement system, toll-like receptors, NOD receptor family.

### The Complement System

The term “complement” as used herein refers to a system of soluble pattern recognition receptors and effector molecules that detect and destroy microorganisms. In the presence of pathogens or of antibody bound to pathogens, soluble plasma proteins that in the absence of infection circulate in an inactive form becomes activated, so that particular complement proteins interact with each other to form the pathways of complement activation, which are initiated in different ways. The classical pathway is initiated when complement component C1, which comprises a recognition protein (C1q) associated with proteases (C1r and C1s) either recognizes a microbial surface directly or binds to antibodies already bound to a pathogen. The alternative pathway can be initiated by spontaneous hydrolysis and activation of complement component C3, which can then bind directly to microbial surfaces. The lectin pathway is initiated by soluble carbohydrate-binding proteins—mannose-binding lectin (MBL) and the ficolins—that bind to particular carbohydrate structures on microbial surfaces. MBL-associated serine proteases (MASPs), which associate with these recognition proteins, then trigger cleavage of complement proteins and activation of the pathway. These three pathways converge at the step whereby enzymatic activity of a C3 convertase is generated. The C3 convertase is bound covalently to the pathogen surface, where it cleaves C3 to generate large amounts of C3b, the main effector molecule of the complement system, and C3a, a small peptide that binds to specific receptors and helps induce inflammation. Cleavage of C3 is the critical step in complement activation and leads directly or indirectly to all the effector activities of the complement system. All three pathways have the final outcome of killing the pathogen, either directly or by facilitating its phagocytosis, and inducing inflammatory responses that help to fight infection.

Besides acting in innate immunity, the complement system also influences adaptive immunity. For example, opsonization of pathogens (meaning the coating of the surface of a pathogen that makes it more easily ingested by phagocytes) by complement facilitates their uptake by phagocytic APCs that express complement receptors, which enhances presentation of pathogen antigens to T cells. B cells express receptors for complement proteins that enhance their responses to complement-coated antigens. Several complement fragments also can act to influence cytokine production by APCs, thereby influencing the direction and extent of the subsequent adaptive immune response. Janeway's Immunology, 9^{th} Ed. 2017, Garland Science, New York, Chapter 2, 49-51.

### Toll-Like Receptors

Toll-like receptors (TLRs) are sensors for microbes present in extracellular spaces. Some are cell surface receptors (e.g., TLR-1, TLR-2, TLR-5, TLR-6), but others (e.g., TLR3, TLR-7, TLR-8, TLR-9) are located intracellularly in the membrane of endosomes, where they detect pathogens or their components that have been taken into cells by phagocytosis, receptor-mediated endocytosis or micropinocytosis. Id. at 88.

TLR-4, is expressed by several types of immune system cells, including dendritic cells and macrophages, recognizes the LPS of gram negative bacteria by a mechanism that is partly direct and partly indirect. Systemic injection of LPS causes a collapse of the circulatory and respiratory system (shock), due to an overwhelming secretion of cytokines, particularly TNF-α, causing systemic vascular permeability. To recognize LPS, the ectodomain of TLR-4 uses an accessory protein, MD-2, which initially binds to TLR-4 within the cell and is necessary both for the correct trafficking of TLR-4 to the cell surface and for the recognition of LPS. TLR-4 activation involves two other accessory proteins, LPS-binding protein, present in the blood and in extracellular fluid in tissues, and CD14, which is present on the surface of macrophages, neutrophils and dendritic cells. On its own, CD14 can act as a phagocytic receptor, but on macrophages and dendritic cells it also acts as an accessory protein for TLR-4. Id. at 92.

Mammalian TLRs recognize molecules characteristic of bacteria, fungi and viruses, including the lipoteichoic acids of Gram-positive bacterial cell walls, and the lipopolysaccharide (LPS) of the outer membrane of Gram negative bacteria. Although TLRs have limited specificity compared with the antigen receptors of the adaptive immune system, they can recognize elements of most pathogenic microbes and are expressed by many types of cells, including macrophages, dendritic cells, B cells, stromal cells, and certain epithelial cells. Id. at 88.

Signaling by mammalian TLRs in various cell types induces a diverse range of intracellular responses by activating several different signaling pathways that each activate different transcription factors, which, together result in the production of inflammatory cytokines, chemotactic factors, antimicrobial peptides, and the antiviral cytokines interferon α and β. Id. at 92 The outcome of TLR activation can vary depending on the cell type in which it occurs. Id. at 95.

Signaling by mammalian TLRs is activated when binding of a ligand induces formation of a dimer, or induces conformational changes in a preformed TLR dimer. All mammalian TLR proteins have in their cytoplasmic tail a Toll-IL-1 receptor (TIR) domain, which interacts with other T1R-type domains, usually in other signaling molecules, and is also found in the cytoplasmic tail of the receptor for the cytokine interleukin-1-β. Id. at 88 Dimerization brings the cytoplasmic T1R domains together, allowing them to interact with the T1R domains of cytoplasmic adaptor molecules that initiate intracellular signaling. There are four adaptors used by mammalian TLRs: MyD88, MAL (also known as TIRAP), TRIF, and TRAM. The T1R domains of the different TLRs interact with different combinations of these adaptors. Id. at 92-93.

For example, TLR-3 interacts only with TRIF. TLR-21 and TLR2/6 require MyD88/MAL. TLR-4 signaling uses both MyD8/MAL and TRIF/TRAM, which is used during endosomal signaling by TLR-4. The choice of adaptor influences which of the several downstream signals will be activated by the TLR. Id. at 94.

Signaling by most TLRs activates the transcription factor NFκB, several members of the interferon regulatory factor (IRF) transcription factor family through a second pathway, and members of the activator protein 1 (AP-1) family, such as c-Jun, through another signaling pathway involving mitogen-activated protein kinases (MAPKs). NFκB and AP-1 act primarily to induce the expression of proinflammatory cytokines and chemotactic factors. Id. at 94.

The Signaling Pathway Triggered by TLRs that Use MyD88 [See FIG. 2)]

TLR-7, TLR-8 and TLR-9 signal uniquely through MyD88. MyD88 has a T1R domain at its carboxy terminus that associates with the T1R domains in the TLR cytoplasmic tails. At its amino terminus, it has a death domain that associates with a similar death domain present in other intracellular signaling proteins. Both domains are required for signaling. The MyD88 death domain recruits and activates two serine-threonine protein kinases—IRAK4 (IL-1 receptor associated kinase 4) and IRAK1—via their death domains. This IRAK complex recruits enzymes that produce a signaling scaffold, and uses this scaffold to recruit other molecules that are then phosphorylated by the IRAKs. Id. at 94. To form a signaling scaffold, the IRAK complex recruits the enzyme tumor necrosis factor receptor-associated factor 6 (TRAF6), which is an E3 ubiquitin ligase that acts in cooperation with UBC13, an E2 ubiquitin ligase, and its cofactor Uve1A (together called TRIKA1). The combined activity of TRAF-6 and UBC13 is to ligate one ubiquitin molecule to another protein and thereby generate protein polymers. A polyubiquitin polymer, which can be initiated on other proteins, including TRAF-6 itself, can be extended to produce polyubiquitin chains that act as a scaffold that binds to other signaling molecules. Next the scaffold recruits a signaling complex consisting of the polyubiquitin-binding adaptor proteins TAB1, TAB2, and the serine-threonine kinase TAK1. TAK1 is phosphorylated by the IRAK complex, and activated TAK1 propagates signaling by activating certain MAPKs, such as c-Jun terminal kinase (JNK) and mAPK14 (p38 MAPK). These then activate AP-1 family transcription factors that transcribe cytokine genes.

TAK1 also phosphorylates and activates IκB kinase (IKK) complex, which is composed of three proteins: IKKα, IKKβ, and IKKγ (also known as NEMO, for NFκB essential modifier). NEMO binds to polyubiquitin chains, which brings the IKK complex into proximity with TAK1. TAK1 phosphorylates and activates IKKβ, which phosphorylates IκB (inhibitor of κB), a cytoplasmic protein that constitutively binds to transcription factor NFκB. NFκB contains two subunits, p50 and p65. The binding of IκB traps the NFκB proteins in the cytoplasm. Phosphorylation by IKK induces the degradation of IκB, which releases NFκB into the nucleus, where it can drive the transcription of genes for pro-inflammatory cytokines, such as TNF-α, IL-1β, and IL-6. Id. at 94-95.

TLR-3, TLR-7. TLR-8, and TLR-9, the nucleic acid-sensing TLRs-activate members of the IRF family.

IRF proteins reside in the cytoplasm and are inactive until they become phosphorylated on serine and threonine residues in their carboxy termini. They then move to the nucleus as active transcription factors. There are 9 IRF family members, of which IRF3 and IRF7 are particularly important for TLR signaling and expression of antiviral type 1 interferons. For TLR-3, which is expressed by macrophages and conventional dendritic cells, the cytoplasmic T1R domain interacts with adaptor protein TRIF, which interacts with E3 ubiquitin ligase TRAF3, which, like TRAF6, generates a polyubiquitin scaffold. In TLR-3 signaling, this scaffold recruits a multiprotein complex containing the kinases IKKε and TBK1, which phosphorylate IRF3 [FIG. 3]. TLR-4 also triggers this pathway by binding TRIF, but the IRF3 response induced by TLR-4 is relatively weak compared with that induced by TLR-3.

For TLR-7 and TLR-9 signaling in plasmacytoid dendritic cells, the MyD88 T1R domain recruits the IRAK1/IRAK4 complex, which can also physically associate with IRF7, which is highly expressed by plasmacytoid dendritic cells. This allows IRF7 to become phosphorylated by IRAK1, leading to induction of type 1 interferons. Not all IRF factors regulate type 1 interferon genes; IRF5, for example, plays a role in induction of pro-inflammatory cytokines.

### NOD-Like Receptors (NLRs)

Nucleotide-binding oligomerization domain (NOD)-like receptors (NLRs) are innate sensors that detect microbial products or cellular damage in the cytoplasm or activate signaling pathways, and are expressed in cells that are routinely exposed to bacteria, such as epithelial cells, macrophages and dendritic cells.

Some NLRs activate NFκB to initiate the same inflammatory responses as the TLRs, while others trigger a distinct pathway that induces cell death and the production of pro-inflammatory cytokines. Id. at 96 (See FIG. 4,).

Subfamilies of NLRs can be distinguished based on the other protein domains they contain. For example the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins. Id. at 96. NOD1 senses γ-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood. Id. at 96-98.

When NOD1 or NOD2 recognizes its ligand, it recruits the CARD-containing serine-threonine kinase RIP2 (also known as RICK and RIPK2), which associates with the E3 ligases cIAP1, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO. Id. at 97.

Macrophages and dendritic cells express both TLFs and NOD1 and NOD2, and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins. Id. at 97.

Other members of the NOD family, including NLRX1 and NLRC5, have been identified, but their function is less well understood. Id. at 96-98

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would active the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3. Id. at 98-99.

NLR signaling, as exemplified by NLRP3, leads to the generation of pro-inflammatory cytokines and to cell death through formation of an inflammasome, a multiprotein complex (FIG. 4). Activation of the inflammasome proceeds in several stages: (1) Aggregation of NLRP molecules triggers autocleavage of procaspase I, which releases active caspase 1—Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxyterminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly 1L-1β and IL-18, into their active forms, and induces a form of cell death (pyroptosis) associated with inflammation because of the release of these pro-inflammatory cytokines upon cell rupture. Id. at 99-100.

A priming step, which can result from TLR signaling, must first occur in which cells inducer and translate the mRNAs that encode the pro-forms of IL-1, IL-18 or other cytokines for inflammasome activation to produce inflammatory cytokines. For example, the TLR-3 agonist poly I:C can be used experimentally to prime cells for triggering of the inflammasome. Id. at 100.

Inflammasome activation also can involve proteins of the PYHIN family, which have an H inversion (HIN) domain in place of an LRR domain. There are four PYIN proteins in humans. Id. at 100.

A noncanonical inflammasome (caspase I-independent) pathway uses the protease caspase 11, which therefore is both a sensor and an effector molecule, to detect intracellular LPS. Id. at 101.

Besides activating effector functions and cytokine production, another outcome of the activation of innate sensing pathways is the induction of co-stimulatory molecules on tissue dendritic cells and macrophages. B7.1 (CD80) and B7.2 (CD86), for example, which are induced on macrophages and tissue dendritic cells by innate sensors such as TLRs in response to pathogenic recognition, are recognized by specific co-stimulatory receptors expressed by cells of the adaptive immune response, particularly CD4 T cells, and their activation by B7 is an important step in activating adaptive immune responses. Id. at 105.

### Cytokine and Chemokines Coordinate Cellular Immune Responses

Cytokines are small proteins that are released by various cells usually in response to an activating stimulus that induce responses through binding to specific receptors. They can act in an autocrine manner, affecting the behavior of the cell that released the cytokine, in a paracrine manner, affecting adjacent cells, or in an endocrine manner, affecting distant cells. Id. at 107. They include the interleukin-1 family (IL-1), the hematopoietin superfamily, the interferons, and the TNF family.

### The IL-1 Family

The IL-1 family contains 11 members, including IL-1α, IL-1β, and IL-18. Most are produced as inactive proproteins that are cleaved to produce the mature cytokine (except for IL-1α, for which both the proprotein and its cleaved forms are active). IL-1 family receptors, which have T1R domains in their cytoplasmic tails and signal by the NFκB pathway, function in concert with the IL-1 receptor accessory protein (IL1 RAP) which is required for IL-1 signal transduction. Id. at 108.

### The Hematopoietin Superfamily

The hematopoietin superfamily includes non-immune system growth and differentiation factors, such as erythropoietin, growth hormone and GM-CSF), and interleukins with roles in adaptive and innate immunity (e.g. IL-6). IL-3, IL-4, IL-5, IL-13 and GM-CSF are related structurally; they bind to closely related receptors, which belong to the family of class I cytokine receptors. Another subgroup of class I cytokine receptors includes receptors for cytokines IL-2, IL-4, IL-7, IL-9, IL-15 and IL-21. The receptors for these cytokines are tyrosine kinase-associated receptors that form dimers when their cytokine ligand binds. Dimerization initiates intracellular signaling from the tyrosine kinases associated with the cytoplasmic domains of the receptor. Cytokine signaling is complex, because a large variety of different receptor subunit combinations can occur. Id. at 108-109.

### The Interferon Family

The receptor for IFNγ is a member of a small family of heterodimeric cytokine receptors (class II cytokine receptors) with some similarities to the hematopoietin receptor family. Examples include the receptors for IFNα, IFNβ and the IL-10 receptor. Id. at 109.

The IFN α/β Signaling pathway

IFN-alpha and IFN-beta bind to the type I IFN receptor (IFN-alpha/beta receptor) consisting of two subunits, Interferon (alpha, beta and omega) receptor 1 (IFNAR1) and Interferon (alpha, beta and omega) receptor 2 (IFNAR2). (Pestka S, Krause C D, Walter M R (2004) “Interferons, interferon-like cytokines, and their receptors,” Immunological Revs. 202: 8-32). The IFN-alpha/beta receptor lacks intrinsic kinase activity and thus relies on associated Janus kinases (JAK1 and Tyk2) to phosphorylate receptor and signal transducing molecules, such as Signal transducers and activators of transcription 1 (STAT1 and STAT2), after ligand-induced receptor clustering. IFNAR1 is pre-associated with Tyk2, and also binds STAT1 and STAT2. IFNAR2 is pre-associated with JAK1, STAT1 and STAT2. (de Weerd N A, et al (2007), “Type I interferon receptors: biochemistry and biological functions,” J. Biol. Chem. 282(28): 20053-7). The tyrosine phosphorylation of STAT1 and STAT2 by JAK1 and Tyk2 leads to the formation of transcriptional complexes that translocate to the nucleus to induce expression of certain genes. Platanias L C, (2005) “Mechanisms of type-I- and type-II-interferon-mediated signaling,” Nature Reviews Immunology. 2005 5(5):375-86).

The mature ISG Factor-3 complex (ISGF3) is composed of phosphorylated forms of STAT1 and STAT2 and Interferon regulatory factor 9 (IRF9), which does not undergo tyrosine phosphorylation Id. ISGF3 is the only complex that binds specific elements known as IFN-stimulated response elements (ISREs) that are present in the promoters of certain genes, such as promyelocytic leukemia (PML), ISG15 ubiquitin-like modifier (ISG15), Interferon-induced protein with tetratricopeptide repeats 2 (ISG54) and Interferon alpha-inducible protein 6 (IFI6) (Parrington J, et al. (1993), “The interferon-stimulable response elements of two human genes detect overlapping sets of transcription factors,” European J. Biochem./FEBS 214(3):617-26; Au W C, et al., (1995) “Identification of a member of the interferon regulatory factor family that binds to the interferon-stimulated response element and activates expression of interferon-induced genes,” Proc. Natl Acad. Sci. USA 92(25):11657-61; Stadler M, et al. (1995) “Transcriptional induction of the PML growth suppressor gene by interferons is mediated through an ISRE and a GAS element,” Oncogene 11(12):2565-73; Nakaya T, et al., (2001) “Gene induction pathways mediated by distinct IRFs during viral infection,” Biochem. Biophys. Res. Commun. 283(5):1150-6).

In response to IFN-α, STAT1 and STAT2 can also form another transcriptional complex, STAT1/STAT2 heterodimer that exhibits binding to the gamma-activated sequence (GAS) element of the Interferon regulatory factor 1 (IRF1) gene. Ghislain J J, et al, (2001) “The interferon-inducible Stat2:Stat1 heterodimer preferentially binds in vitro to a consensus element found in the promoters of a subset of interferon-stimulated genes,” J. Interferon & Cytokine Research 21(6):379-88; Brierley M M, Fish E N (2005), “Functional relevance of the conserved DNA-binding domain of STAT2,” J. Biol. Chem. 280(13):13029-36). IRF1, in turn, can also induce the transcription of ISG15, ISG54 and IFI6 genes, whereas another IFN-alpha-inducible factor, Interferon regulatory factor 2 (IRF2), is involved in the repression of gene transcription (Pine R, et al., (1990) “Purification and cloning of interferon-stimulated gene factor 2 (ISGF2): ISGF2 (IRF-1) can bind to the promoters of both beta interferon- and interferon-stimulated genes but is not a primary transcriptional activator of either,” Molec. & Cell. Biol. 10(6):2448-57; Nelson N, et al. (1993), “Interferon consensus sequence-binding protein, a member of the interferon regulatory factor family, suppresses interferon-induced gene transcription,” Molec. Molec. & Cell. Biol. 13(1):588-99; Masumi A, Ozato K (2001) “Coactivator p300 acetylates the interferon regulatory factor-2 in U937 cells following phorbol ester treatment,” J. Biol. Chem. 276(24): 20973-80; Meraro D, et al. (2002) “IFN-stimulated gene 15 is synergistically activated through interactions between the myelocyte/lymphocyte-specific transcription factors, PU.1, IFN regulatory factor-8/IFN consensus sequence binding protein, and IFN regulatory factor-4: characterization of a new subtype of IFN-stimulated response element,” J. Immunology 168(12):6224-31).

Arginine methylation of STAT1 by Protein arginine methyltransferase 1 (PRMT1) is an additional posttranslational modification that regulates transcription factor function required for proper IFN-alpha/beta-induced transcription (Fenner J E, et al. (2006) “Suppressor of cytokine signaling 1 regulates the immune response to infection by a unique inhibition of type I interferon activity,” Nature Immunology 7(1):33-9).

A number of negative regulatory molecules limit the extent of type I IFN signaling. Suppressor of cytokine signaling 1 (SOCS1) inhibits type I IFN signaling via interactions with IFNAR1, JAK1 and Tyk2 (Id.). Protein tyrosine phosphatases non-receptor type 6 and 11 (SHP-1 and SHP-2) dephosphorylate JAK1 and STAT1 and suppress their signaling (David M, et al. (1995), “Differential regulation of the alpha/beta interferon-stimulated Jak/Stat pathway by the SH2 domain-containing tyrosine phosphatase SHPTP1,” Molec. & Cell. Biol. 15(12):7050-8; You M, et al. (1999) “Shp-2 tyrosine phosphatase functions as a negative regulator of the interferon-stimulated Jak/STAT pathway,” Molec. & Cell. Biol. 19(3):2416-24). Protein tyrosine phosphatase non-receptor type 1 (PTP-1B) dephosphorylates Tyk2 and modulates signaling responses to IFN-alpha (Myers M P, et al. (2001) “TYK2 and JAK2 are substrates of protein-tyrosine phosphatase 1B,” J. Biol. Chem. 276(51):47771-4). A type I IFN-inducible ubiquitin specific peptidase 18 (UBP43) binds directly to IFNAR2 and blocks the interaction between JAK1 and IFN-alpha/beta receptor (Malakhova O A, et al. (2006) “UBP43 is a novel regulator of interferon signaling independent of its ISG15 isopeptidase activity,” The EMBO J. 25(11): 2358-67).

Recent studies in immune cells have incriminated IFN-γ as a possible trigger of GC insensitivity in severe asthma; however, little is known about the role of IFN-γ in modulating GC effects in other clinically relevant nonimmune cells, such as airway epithelial cells. Using Western blot analysis, all steps of the IFN-γ induced JAK/STAT signaling pathway were found to be GC sensitive, while transfection of cells with reporter plasmid showed IFN-γ-induced STAT1-dependent gene transcription to be GC insensitive. (O'Connell, D et al. (2015) “IFNγ-induced JAK/STAT, but not NFκB, signaling pathway is insensitive to glucocorticoid in airway epithelial cells,” Am. J. Physiol. Lung Cell Mol. Physiol. 309(4): L348-L359). Real-time PCR analysis showed that IFNγ-inducible genes (IIGs) were differentially affected by GC, with CXCD10 being GC sensitive and CXCL11 and IFIT2 being GC insensitive. Id. The differential sensitivity of IIGs to GC was found to be due to their variable dependency to JAK/STAT vx. NFκB signaling pathways, with GC-sensitive IIGs being more NFκB dependent and GC insensitive IIGs being more JAK/STAT dependent. Id.

### The TNF Family

Many members of the TNF family of cytokines are transmembrane proteins; some (e.g., TNF-α) can be released from the membrane in some circumstances. The effects of TNF-α are mediated by either of two TNF receptors. TNF receptor 1 (TNFR-1) is expressed on a wide range of cells, whereas TNFR-II is expressed largely by lymphocytes. TNF family cytokines are produced as trimers; the binding of these cytokines induces the clustering of three identical receptor subunits. Janeway's Immunology, 9^{th} Ed. 2017, Garland Science, New York, at 109.

### Chemokines

Chemokines induce directed chemotaxis in nearby responsive cells, resulting in the movement of the cells towards the source of the chemokine. The signaling pathway stimulated by chemokines causes changes in cell adhesiveness and changes in the cell's cytoskeleton that lead to directed migration. In the immune system, they function mainly as chemoattractants for leukocytes, recruiting monocytes, neutrophils and other effector cells in innate immunity; lymphocytes in adaptive immunity; as well as in lymphocyte development, migration and in angiogenesis. Id. at 112.

Chemokines fall mainly into two groups: the CC chemokines, which have two adjacent cysteine residues near the main terminus, and the CXC chemokines, in which the equivalent cysteine residues are separated by a single amino acid. The two groups of chemokines act on different sets of G-protein coupled receptors. CC chemokines bind to CCR1-10 receptors, while CXC chemokines bind to CXCR1-7 receptors. In general, CXC chemokines with a Glu-Leu-Arg tripeptide motif immediately before the first cysteine promote the migration of neutrophils (e.g., CXCL8, formerly known as IL-8). Members of the chemokine receptor family have a 7-transmembrane structure and signal by interacting with G-proteins. Id. at 109.

### Cytokine Signaling Pathways

The signaling chains of hematopoietin and interferon receptors are noncovalently associated with protein tyrosine kinases of the Janus kinase (JAK) family. Dimerization or clustering of receptor signaling chains brings the JAKS into close proximity, causing phosphorylation of each JAK on a tyrosine residue that stimulates its kinase activity. The activated JAKs then phosphorylate their associated receptors on specific tyrosine residues. This phosphotyrosine, and the amino acid sequence surrounding it, creates a binding site recognized by SH2 domains found in signal transducers and activators of transcription (STATs), members of a family of transcription factors. There are 7 STATs, which reside in the cytoplasm in an inactive form until activated by cytokine receptors. The receptor specificity of each STAT is determined by the recognition of the distinctive phosphotyrosine sequence on each activated receptor by the different SH2 domains within the various STAT proteins, and a cytokine typically activates one type of STAT. The phosphorylated STAT dimer enters the nucleus, where it acts as a transcription factor to initiate the expression of selected genes that can regulate growth and differentiation of particular subsets of lymphocytes. Id. at 109-110.

Cytokine signaling can be terminated by dephosphorylation of the receptor complex, by negative feedback involving suppressor of cytokine signaling (SOCs), specific inhibitors that are induced by STAT activation, or by protein inhibitors of activated STAT (PIAS) proteins. For example, the nonreceptor tyrosine phosphatases SHP-1 and SHP-2 and the transmembrane receptor tyrosine phosphatase CD45 which is expressed on hematopoietic cells, have been implicated in the dephosphorylation of cytokine receptors, JAKs and STATs. Id. at 110-111.

PIAS1 is a known negative regulator of NF-κB signaling, as it interacts with p65 and represses the transcriptional activity of NF-κB. (Heo, K-S, et al, (2013) “Phosphorylation of protein inhibitor of activated STAT1 (PIAS1) by MAPK-activated protein kinase 2 (MK2) inhibits endothelial inflammation via increasing both PIAS1 transrepression and SUMO E3 ligase activity,” Arterioscler. Thromb. Vasc. Biol. 33(2): 321-29). Their results showed that MK2 phosphorylates PIAS1 at Ser533 (S522) and promotes PIAS1 transrepression activity on NF-κB. The canonical NF-κB pathway involving IKKα, IκBα, and p65 NF-κB phosphorylation was unaffected by the expression of adenovirus wild type MK2 or dominant negative MK2; both IKKα and MK2 are proinflammatory. Id. TNF increased endogenous PIAS1 S522 phosphorylation maximally within 10 minutes after TNF stimulation; endogenous PIAS1 S522 phosphorylation was completely inhibited by the depletion of MK2, suggesting a crucial role of endogenous MK2 in TNF-initiated PIAS1-S522 phosphorylation. Id. Moreover, a PIAS1 S522 A mutation significantly enhanced TNF-induced NF-κB activation compared with wild type, suggesting an inhibitory role of endogenous PIAS1 phosphorylation in NF-κB activation. Id. Depletion of MK2 significantly inhibited TNF-induced p53 SUMOylation in endothelial cells, suggesting that TNF-induced PIAS1 S522 phosphorylation can regulate p53 SUMOylation. Id. Small ubiquitin-related modifier (SUMO) is an ubiquitin-like protein that is covalently attached to a variety of target proteins to regulate such cellular processes as nuclear transport, transcription, chromosome segregation and DNA repair. (Gareau, J R, and Lima, C D, (2010) “The SUMO pathway: emerging mechanisms that shape specificity, conjugation and recognition,” Nat. Rev. Mol. Cell Biol. 11(12): 861-71).

Stimulation of TNF receptors recruits adaptor proteins known as TNF receptor-associated factors (TRAFs). Five of the six known TRAFs also function as E3 ubiquitin ligases, which contributes to the ability of most TNF superfamily members to activate the NFκB pathway using the non-canonical NFκB pathway, a pathway distinct from that initiated by antigen receptor stimulation. Janeway's Immunology, 9^{th} Ed. 2017, Garland Science, New York at 286.

Lipopolysaccharide (LPS, Endotoxin))

Lipopolysaccharide, a glycolipid embedded in the outer membrane of Gram negative bacteria, which plays a crucial role in maintaining the structural integrity of the organism, contains three biochemical domains. The O-specific chain is a repetitive glycan polymer that projects outside of the outer membrane onto the surface of bacteria and that contributes to the antigenicity, morphological appearance and antibiotic sensitivity of Gram negative bacteria. Bohannon, J K, et al, “The immunobiology of TLR4 agonists: from endotoxin tolerance to immunoadjuvants,” Shock (2013) 40(6): 451-62. The core domain is a phosphorylated oligosaccharide that links the O-specific chain to the lipid A moiety and is important for maintaining structural viability. Id. Lipid A, a heat-stable phosphorylated glucosamine disaccharide with multiple fatty acid side chains that anchors the LPS molecule into the lipid bilayer of the bacterial outer membrane, is avidly recognized by leukocytes and other cell types and is the major factor that alerts the immune system to the presence of infection with Gram negative organisms. Id.

Cells recognize lipid A via a surface receptor complex composed of the proteins myeloid differentiation factor 2(MD2) and TLR4. Id. LPS binding protein (LBP) and the membrane-bound and soluble forms of CD14 bind LPS in the systemic and interstitial environments and play important roles in facilitating the presentation of LPS to the endotoxin receptor complex. Id. Binding of LPS to MD2 causes conformation changes in TLR4 that facilitate TLR4 dimerization or oligomerization and activation of downstream signaling. Id.

Initial LPS exposure induces robust TLR-4 mediated activation of NF-κB and AP-1; translocation of NF-κB and AP-1 are the major factors that regulate expression of pro-inflammatory gene products in response to LPS. At the same time, inhibitors of TLR-4, NF-κB and AP-1 signaling are induced (e.g., inhibitor of κB (IκB, MAP kinase phosphatase-1, interleukin receptor-associated kinase M (IRAK-M), suppressor of cytokine signaling-1 (SOCS1) and RelB), which attenuate NF-κB and AP-1 activation and translocation and serve to regulate the LPS-induced inflammatory response to prevent uncontrolled expression of pro-inflammatory mediators. Id. Evidence indicates that LPS-induced expression of MAP kinase phosphatase-1, RelB and IRAK-M are induced by activation of the phosphoinositide-3 kinase (PI3K) pathway.

In vivo, infection with gram negative bacteria releases LPS into the blood stream, which activates monocytes. In response, the activated monocytes secret various inflammatory mediators, including, without limitation, Tumor Necrosis Factor-alpha (TNF-α) and Interleukin-6 (IL-6), to combat the infection. A lipopolysaccharide challenge assay evaluates a subject's ability to respond to an inflammatory stimulus by mounting an acute phase response.

Prior exposure to LPS renders the host resistant to shock caused by subsequent LPS challenge; this is called endotoxin tolerance. Id. Suppression of cytokine signaling has been observed during endotoxin tolerance; SOCS-1 is a potent inhibitor of JAK-STAT signaling. Nakagawa et al reported that SOCS-1 is rapidly induced by LPS, that SOCS-1 deficient mice are highly sensitive to LPS-induced inflammatory injury, and that SOCS-1 deficient mice do not develop endotoxin tolerance (Id. citing Nakagawa, R. et al, (2002) “SOCS-1 participates in negative regulation of LPS responses,” Immunity 17(5): 677-87). PI3K activation may facilitate the development of LPS tolerance. (Id. citing Deng, H. et al, (2013) Molecular mechanism responsible for the priming of macrophage activation,” J. Biol. Chem. 288(6): 3897-3906). Monocytes and macrophages harvested from immunocompromised septic patients exhibit suppressed LPS-induced cytokine production, a characteristic that resembles the endotoxin tolerant phenotype. (Id. citing Biswas, S K and Lopez-Collazo, E., “Endotoxin tolerance: new mechanisms, molecules and clinical significance,” Trends Immunol. 30(10): 475-87).

### Lipopolysaccharide (LPS) Signaling

As depicted in FIG. 5, LPS stimulates both pro- and anti-inflammatory pathways. The timing of events downstream of MK2 activation is critical (1) to stimulate inflammation and (2) then ensure the resolution of the inflammation.

MK2 serves as an important kinase in regulation of inflammatory cell activation in the lung. (Qian, F. et al, (2016) “Pivotal role of mitogen-activated protein kinase-activated protein kinase 2 in inflammatory pulmonary diseases,” Curr. Protein Pept. Sci. 17(4): 332-42, citing Gaestel, M. (2005) “MAPKAP kinases—MKs—two's company, three's a crowd,” Nat. Rev. Mol. Cell Biol. 7(2): 120-130)). Exogenous microbial components termed pathogen-associated molecular patterns (PAMPs) or endogenous inflammatory factors released from necrotic cells bind to the germline-encoded pattern recognition receptors (PRRs) including toll-like receptors (TLRs), NOD-like receptors (NLRs), and C-type lectin receptors (CLRs), which triggers the activation of MAPK cascades via the adaptor proteins myeloid differentiation primary-response protein 88 (MyD88) and T1R domain-containing adaptor protein inducing IFNβ (TRIF (Id. citing Qian, C. and Cao, X, (2013), “Regulation of Toll-like receptor signaling pathways in innate immune responses,” Ann. NY Acad. Sci. 1283: 67-74). In canonical signal transduction, p38 MAPK is selectively phosphorylated by MAPKKs (MKK3 and MKK6), which are in turn activated by MAPKKKs including TGFβ-activated kinase 1 (TAK1), apoptosis signal-regulating kinase 1 (ASK1), mixed-lineage kinase 2 (MLK2) or MLK3. The p38 MAPK-mediated signals initiate the activation of several transcriptional factors including CREB, ATF2 and Myc, as well as other kinases including MK2, but also MK3, MNK1/2, and MSK1/2 (Id. citing Obata, T. et al, (2000) Crit. Care Med. 28 (4 Suppl.: N67-N77; Dong, C. et al, (2002) “MAP kinases in the immune response,” Annu. Rev. Immunol. 20: 55-72)). Among these distal kinases, the role of MK2 has been determined to be essential for the regulation of innate immune responses including modulating production of inflammatory cytokines and chemokines, reactive oxygen species (ROS) and nitric oxide (NO). Id. For example, accumulating evidence indicates that MK2 is involved in regulation of cytokine biosynthesis, e.g., TNF-α, by enhancing mRNA stability. MK2 can regulate inflammatory cytokine production in a synergistic manner via phosphorylating TTP and butyrate response factor 1 (BRF1). The p38 MAPK pathway has been demonstrated to mediate the stabilizing effect of LPS. Grutz, G. (2005) “New Insights into the molecular mechanism of interleukin-10 mediated immunosuppression,” J. Leukocyte Biol. 77: 3-15).

TLR-4 is the crucial receptor for LPS signaling. LPS can trigger both MyD88-dependent and independent signaling cascades. Adaptor proteins MyD88 and TIRAP (also known as MyD88 adaptor-like protein or Mal) mediate signaling via IRAK1/4 to TRAF6, which seem to be important to activate early NF-κB and MAPKs. The adaptor proteins TRIF and TRAM, conversely, are responsible for initiating IRF-3 activation and thereby IFN-α/β secretion in the MyD88-independent pathway; this pathway triggers late NF-κB activation. MyD88-dependent early and MyD88-independent late NF-κB activation is thought to contribute to the initiation of transcription of most proinflammatory cytokines (e.g., TNFα, IL-1, IL-6, IL-8 and IL-12). Palsson-McDermott E M and O'Neill L A J (2004) “Signal transduction by the lipopolysaccharide receptor, Toll-like receptor-4,” Immunology 113(2): 153-62).

Production of proinflammatory cytokines is not only controlled by transcriptional means. Indeed, post-transcriptional mechanisms play an important role in the regulation of mRNA stability, protein translation and maturation into the active, secreted forms of several cytokines.

In response to LPS stimulation, deficiency of tristetraprolin (TTP), a zinc finger protein, results in increased half-life of TNF-α mRNA in macrophages, indicating the inhibitory role of TTP on TNF-α mRNA post-transcription, which is strictly regulated by p38 MAPK/MK2 signal transduction. (Qian, F. et al, (2016) “Pivotal role of mitogen-activated protein kinase-activated protein kinase 2 in inflammatory pulmonary diseases,” Curr. Protein Pept. Sci. 17(4): 332-42 Citing Carballo, E. et al, (1998) “Feedback inhibition of macrophage tumor necrosis factor-alpha production by tristetraprolin,” Science 281 (5379): 1001-1005). There are several alternative mechanisms of MK2 inducing TNF-α production other than direct phosphorylation of TTP. For example, MK2 can maintain p38 MAPK protein stability through direct interaction (Id. citing McGuire, V A, et al (2013) “Crosstalk between the Akt and p38 alpha pathways in macrophages downstream of toll-like receptor signaling,” Mol. Cell Biol. 33(21): 4152-65)). Although MK3, another downstream kinase of p38 MAPK, cannot directly regulate TNF-α, MK3 facilitates MK2 enhancement of TNF-α translation. (Id. citing Ronkina, N. et al, (2007), “The mitogen-activated protein kinase (MAPK)-activated protein kinases MK2 and MK3 cooperate in stimulation of tumor necrosis factor biosynthesis and stabilization of p38 MAPK,” Mol. Cell Biol. 27(1): 170-812)). It is possible that MK3 increases binding stability between p38 MAPK and MK2 (Id. citing Ronkina, N. et al (2011)“ Stress induced gene expression: a direct role for MAPKAP kinases in transcriptional activation of immediate early genes,” Nucleic Acids Res. 39(7): 2503-2518)). Furthermore, MK2 can bridge crosstalk between p38 MAPK and Akt signals via forming a complex with p38 MAPK, Akt and Hsp27. (Id). MK2 also is involved in endoplasmic reticulum stress responses. Id.

In addition to its positive role, MK2 also serves as a negative feedback regulatory molecule in macrophage activation. In response to LPS stimulation, MK2 deficient macrophages display decreased expression of TTP (Id. citing Ronkina, N. et al (2007), “The mitogen-activated protein kinase (MAPK)-activated protein kinases MK2 and MK3 cooperate in stimulation of tumor necrosis factor biosynthesis and stabilization of p38 MAPK,” Mol. Cell Biol. 27(1): 170-81; Ronkina, N et al (2010) “MAPKAP kinases MK2 and MK3 in inflammation: complex regulation of TNF biosynthesis via expression and phosphorylation of tristetraprolin,” Biochem. Pharmacol. 80(12): 1915-20)) indicating that MK2 is required for inhibiting TTP expression.

### LPS Stimulates IL-10 Expression and STAT3 Phosphorylation Through IFN and the Interferon α/β Receptor (IFNAR) to Suppress Inflammation.

An overwhelming response to LPS can lead to endotoxin shock and death. The interplay between inflammation and resolution of inflammation therefore is critical to support host defense from pathogens and tissue damage while preventing excess tissue breakdown and multiple system organ failure.

IL10 ultimately plays a role in the resolution of inflammation, and LPS also supports stabilization of anti-inflammatory IL10 mRNA. The general consensus regarding the IL-10 triggered signaling steps is as follows: (1) it is dimeric IL-10 that binds to its heterodimeric receptor (composed of the IL-10R1 and IL-10R2 chains) enabling activation of the tyrosine kinases Jak1 and Tyk2, which are constitutively associated with IL-10R1 and IL-10R2, respectively; (2) activation of the receptor associated Jak molecules catalyzes the phosphorylation of two tyrosine residues within the IL-10R1 cytoplasmic domain, which is followed by the recruitment and tyrosine phosphorylation of STAT3; (3) it is the Tyr705-phosphorylated STAT3 that is considered to be essential for delivering the downstream IL-10-mediated anti-inflammatory signals. (Bassoni, F. et al (2010) “Understanding the molecular mechanisms of the multifaceted IL-10-mediated anti-inflammatory response: Lessons from neutrophils,” Eur. J. Immuno. 40: 2360-68). At least in human monocytes and LPS-conditioned neutrophils, de novo protein synthesis is necessary to allow prolonged activation of STAT3 by IL-10, which, in turn, is obligatory for triggering the anti-inflammatory response. Id.

Through the IFN-β pathway, LPS also stimulates STAT3 activation. Because the activation of STAT3 is delayed, occurs 80-120 minutes post-LPS delivery, and takes a minimum of 6-7 hours to resolve, there is an immediate pro-inflammatory effect followed by suppression of inflammation.

IL10 and activated STAT3 support anti-inflammatory activity. (Ehtling, C. et al. ((2011) “Distinct Functions of the MAPKAP kinases MK2 and MK3: MK2 mediates LPS-induced STAT3 activation by preventing negative regulatory effects,” J. Biol. Chem. 286 (27): 24113-24). Activated STAT3 is critical to the anti-inflammatory activity of IL10; STAT3 activation appears to be triggered via interferon beta (IFN-β) activation and is sustained in the presence of IL10. Id. While MK2 activation supports production of IL6, and IL6 suppresses STAT3 activity via activation of SOC3, MK2 inhibition strongly suppresses IL10 production. (Grutz, G., (2005) “New insights into the molecular mechanism of interleukin-10 mediated immunosuppression,” J. Leucocyte Biol. 77: 3-15)

LPS also activates the transcription factor NF-κB. NF-κB is kept sequestered in the cytoplasm by its inhibitor of κB (IκB). Stimulation with LPS sequentially leads to phosphorylation, ubiquitination, and proteosomal degradation of IκB, which allows NF-κB to be translocated to the nucleus and to bind to promoter regions of genes. (Grutz, G. (2005) “New Insights into the molecular mechanism of interleukin-10 mediated immunosuppression,” J. Leukocyte Biol. 77: 3-15).

### Hyporesponsiveness to Sequential LPS Exposure

The term “endotoxin tolerance” refers to a state of refractoriness to LPS challenge following prior exposure to LPS. Endotoxin tolerance is thought to limit excessive inflammatory responses. (Chen, H. et al, (2007) “Tobacco smoking inhibits expression of proinflammatory cytokines and activation of IL-1R-associated kinase, p38, and NF-κB in alveolar macrophages stimulated with TLR2 and TLR4 agonists,” J. Immunol. 179: 6097-6106). Whereas activation of ERK, JNK, p38 MAPK and induction of NF-κB and proinflammatory cytokines (e.g., TNF-α and IL-12) are inhibited in LPS-tolerant cells, other responses (e.g., IL-10 and IL-1R antagonist (IL-1RA) expression) are not affected. Id. Molecular mechanisms responsible for induction and maintenance of endotoxin tolerance remain poorly understood. Id.

FIG. 6 depicts regulation of the subcellular shuttling of TLR4 by the p110δ isoform of phosphatidylinositol-3-OH kinase (PI3K), which functions as a mediator of recognition of self-molecules by TLRs and dampening of TLR signaling to limit immune responses. After stimulation with LPS, p110δ promotes the transition of TLR4 from an early acting TIRAP-MyD88-associated plasma membrane complex (panel a, which induces proinflammatory cytokines by activating the mitogen-activated protein kinase p38 and signaling via transcription factor NF-κB) into a late-acting endosomal TRAM-TRIF complex (panel b, which induces type I interferons and anti-inflammatory IL-10 by activating p38, NFκB and transcription factor IRF3). Mechanistically, p110δ acts in part by diminishing the plasma-membrane abundance of the TIRAP-anchoring lipid PtdIns(4,5)P2, probably in synergy with PLC-γ, which also triggers the endocytosis of CD14-TLR4 by mobilizing Ca²⁺ (not shown). The turnover of PtdIns(4,5)P2 causes the release of TIRAP into the cytoplasm, where it is degraded by calpains and the proteosome. Inactivation of p110δ shifts the balance toward proinflammatory early signaling, which causes hypersensitivity to endotoxins. (Siegemund, S and Sauer, K (2012) “Balancing pro- and anti-inflammatory TLR4 signaling,” Nature Immunology 13: 1031-33).

LPS hyporesponsiveness is characterized by decreased expression of cytokines following stimulation with LPS. (Faas, M M et al. (2002) “Monocyte intracellular cytokine production during human endotoxaemia with or without a second in vitro LPSA challenge: effect of RWJ-67657, a p38 MAP-kinase inhibitor, on LPS hyporesponsiveness,” Clin. Exp. Immunol 127: 337-343). In a small subject pool, human monocytes in vivo were shown to became hyporesponsive to repeat exposure to LPS in vivo; treatment with a p38 kinase inhibitor reversed that hyporesponsiveness. Id. Briefly, LPS was injected intravenously in two people. Blood monocytes collected 3 hours post LPS challenge showed increased inflammatory cytokines production, but at 6, 12, and 24 hours it was back to normal as compared to pre LPS injection. They then looked at hyporesponsiveness by taking monocytes pre-LPS stimulation and monocytes 3 and 24 hours post LPS stimulation, and looking at their responsiveness to LPS stimulation in vitro. They showed reduced TNF-α and IL-1β expression after in vitro LPS stimulation even 24 hours post initial in vivo LPS challenge as compared to naïve monocytes. The results were different in patients who had a p38 inhibitor infused prior to LPS in vivo. In these individuals, treatment of their blood monocytes with LPS in vitro 6 or 24 hours following the in vivo LPS/p38 challenge resulted in a dose dependent increase in cytokine expression. These data suggest that the monocytes were hyporesponsive to LPS when people were just treated with LPS, but for people treated with LPS and a p38 inhibitor, repeat challenge with LPS resulted in a pro inflammatory response. Id.

Sinestro, A et al ((2007) “Lipopolysaccharide desensitizes monocytes-macrophages to CD40 ligand stimulation,” Immunology: 122: 362-70) performed a similar study to that of Faas, et al., except that they treated with an NSAID (indomethacin). When a second dose of LPS was given to hyporesponsive cells, inflammatory cytokine production was reduced as compared to naïve unstimulated controls. However, treatment of LPS hyporesponsive cells with indomethacin resulted in increased expression of TNF-α and IL-12. This suggests that treatment of hyporesponsive monocytes with an anti-inflammatory drug resets the inflammatory pathway allowing the cells to again respond to pathogen stimulation. Id.

Smoking-induced immunosuppression has been implicated in the pathogenesis of bacterial infections, chronic pulmonary obstructive disease (COPD), asthma and bronchitis, as evidenced by suppression of NK cytotoxicity, inhibition of B cell proliferation and antibody production, and impaired antigen- and mitogen-mediated T cell responses. (See Chen, H. et al., (2007) “Tobacco smoking inhibits expression of proinflammatory cytokines and activation of IL-1R-associated kinase, p38, and NF-κB in alveolar macrophages stimulated with TLR2 and TLR4 agonists,” J. Immunol. 179: 6097-6106). Smoking severely impairs functions of alveolar macrophages and airway epithelial cells, including inhibiting LPS-induced expression of TNF-α, IL-1β, and IL6, NO secretion, microbicidal activity and phagocytosis. Id. Alveolar macrophages and epithelial cells provide the first line of defense against lung infection by recognizing conserved PAMPs expressed by bacteria fungi and viruses through TLRs. Id.

Tobacco and tobacco smoke contain bioactive LPS (Id. citing Hasday, J. D, et al. (1999) “Bacterial endotoxin is an active component of cigarette smoke,” Chest 115: 829-35) at concentrations sufficient for the induction of endotoxin tolerance. (Id. citing Shnyra, A. et al, (1998) “Reprogramming of lipopolysaccharide-primed macrophages is controlled by a counterbalanced production of IL-10 and IL-12,” J. Immunol. 160: 3729-36; Ertel, W. et al, (1995), “Downregulation of proinflammatory cytokine release in whole blood from septic patients,” Blood 85: 1341-47; Benjamin, C F, et al (2004), “The chronic consequences of severe sepsis,” J. Leukocyte Biol. 5: 408-12; Li, L. et al (2000), “Characterization of interleukin-1 receptor-associated kinase in normal and endotoxin-tolerant cells,” J. Biol. Chem. 275: 23340-345; Medvedev, A E, et al (2002), “Dysregulation of LPS-induced Toll-like receptor 4-MyD88 complex formation and II-1 receptor-associated kinase 1 activation in endotoxin-tolerant cells, “J. Immunol. 169: 5209-16; Stedman, R L (1968),” The chemical composition of tobacco and tobacco smoke,” Chem. Rev. 68: 153-207) Thus, it has been hypothesized that repeated exposure of smokers' alveolar macrophages to LPS contained in cigarette smoke may induce macrophage refractoriness to TLR-2 and TLR-4-inducible activation by mechanisms resembling those that operate in endotoxin tolerance and may contribute to smokers' increased susceptibility to pulmonary infection. Id. Gene expression and production of cytokines, chemokines and IFNs in alveolar macrophages and PBMCs obtained from 25 healthy smokers to age and gender matched nonsmoking individuals, and expression of TLR2 and RLR4, coreceptors CD14 and MD-2, and TLR2/4 inducible activation of IRAK-1, p38 MAPK and NF-κB were determined. LPS stimulation of nonsmokers' alveolar macrophages induced marked upregulation of TNF-α, IL-1β, and IL6 mRNA levels and a potent increase in secretion of these proinflammatory cytokines. Id. Pam3Cys, a TLR2 agonist elicited similar levels of IL-1β mRNA and protein expression, but was about 29-60% less potent than LPS in triggering TNF and IL-6 mRNA expression and production. Id. By comparison, LPS- and Pam3Cys-mediated gene expression and production of TNF-α and IL-6 were inhibited by about 50 to 70%, while smokers' macrophages exhibited comparable or higher secretion of proinflammatory TNF-α and IL-6 after stimulation with poly(I:C), a TLR3 agonist for 6 h and 24 h. Id.

Chemokine expression (IL-8 and RANTES) in cells isolated from smokers and nonsmokers were compared after treatment with Pam3Cys, poly(I:C), and LPS. Id. Stimulation of nonsmokers' alveolar macrophages with LPS or Pam3Cys led to robust IL-8 gene expression and secretion, and LPS markedly increased RANTES mRNA expression and secretion in nonsmokers macrophages. Id. Pam3Cys failed to induce these responses, consistent with the inability of TLR2 agonists to activate the MyD88-independent pathway that activates RANTES expression (Id. citing Kagan, J C and R. Medzhitov (2006), “Phosphoinositide-mediated adaptor recruitment controls Toll-like receptor signaling,” Cell 125: 943-55)). Smokers' alveolar macrophages showed reduced expression of IL-8 and RANTES mRNA and protein. Id. In contrast, activation of TLR3 with poly I:C) led to comparable gene expression of IL-8 and RANTES from nonsmokers' and smokers' alveolar macrophages. Id. Similar levels of anti-inflammatory IL-10 and IL-1RA mRNA and secreted protein were detected in smokers' and nonsmokers' alveolar macrophages stimulated with LPS and Pam3Cys. Id.

Collectively, these data indicate severe suppressive effects of tobacco smoking on TLR2- and TLR4-initiated expression of pro-inflammatory cytokines (TNF-α, IL-1β, and IL-6) and chemokines (IL-8 and RANTES) in alveolar macrophages, whereas anti-inflammatory IL-10 and IL-1RA gene expression and secretion are not influenced by smoking. Id. Further they indicate that smoking mediated immunosuppression of the cytokine responses of alveolar macrophages targets TLR2 and TLR4 signaling, but does not affect TLR3-mediated responses. Id.

Because phosphorylation of IRAK-1 is necessary for IRAK-1 activation (Id. citing Burns, K. et al (2003), “Inhibition of interleukin 1 receptor/Toll-like receptor signaling through the alternatively spliced, short form of MyD88 s due to its failure to recruit IRAK-4,” J. Exp. Med. 197: 263-68)) and because dephosphorylation of MAPK correlates with kinase activity (Id. citing Payne, D. et al, (1991), “Identification of the regulatory phosphorylation sites in pp42/mitogen-activated protein kinase (MAP kinase),” EMBO J. 10: 885-92)), phosphorylation of IRAK-1 and p38 was measured as indicators of TLR-inducible kinase activation. Id. LPS and Pam3Cys inducible degradation of IκB-a, a prerequisite for NF-κB nuclear translocation in the classical pathway of NF-κB activation (Id citing Hayden, M. et al, (2006), “NF-κB and the immune response,” Oncogene 25: 6758-80)) and LPS mediated nuclear translocation of the NF-κB subunits p50 and p65 were measured to judge activation of NF-κB. Id. Like LPS-tolerant cells that have been reported to accumulate transcriptionally incompetent p50 in the nucleus and to have decreased proportions of p65 (Id. citing Kastenbauer, S. and Ziegler-Heitbrock, H W (1999),” “NF-κB1 (p50) is upregulated in lipopolysaccharide tolerance and can block tumor necrosis factor gene expression,” Infect. Immun. 67: 1553-59; Bohuslav, J., et al (1998),“ Regulation of an essential innate immune response by the p50 subunit of NF-κB,” J. Clin. Invest. 102: 1645-51; Medvedev, A. E., et al (2000),“ Inhibition of lipopolysaccharide-induced signal transduction in endotoxin-tolerized mouse macrophages: dysregulation of cytokine, chemokine and Toll-like receptor 2 and 4 gene expression,” J. Immunol. 164: 5564-74), smokers' alveolar macrophages also showed increased basal levels of p50 and deficient nuclear translocation and phosphorylation of p65 in response to LPS. Id. Because phosphorylation of p65 regulates its transcriptional activity (Id. citing Hayden, M. et al, (2006), “NF-κB and the immune response,” Oncogene 25: 6758-80), low amounts of nuclear p65 in smokers' alveolar macrophages exhibited deficient LPS-induced phosphorylation and therefore were transcriptionally inactive. Id.

### Kinases

Kinases are a ubiquitous group of enzymes that catalyze the phosphoryl transfer reaction from a phosphate donor (usually adenosine-5′-triphosphate (ATP)) to a receptor substrate. Although all kinases catalyze essentially the same phosphoryl transfer reaction, they display remarkable diversity in their substrate specificity, structure, and the pathways in which they participate. A recent classification of all available kinase sequences (approximately 60,000 sequences) indicates kinases can be grouped into 25 families of homologous (meaning derived from a common ancestor) proteins. These kinase families are assembled into 12 fold groups based on similarity of structural fold. Further, 22 of the 25 families (approximately 98.8% of all sequences) belong to 10 fold groups for which the structural fold is known. Of the other 3 families, polyphosphate kinase forms a distinct fold group, and the 2 remaining families are both integral membrane kinases and comprise the final fold group. These fold groups not only include some of the most widely spread protein folds, such as Rossmann-like fold (three or more parallel β strands linked by two α helices in the topological order β-α-β-α-β), ferredoxin-like fold (a common α+β protein fold with a signature βαββαβ secondary structure along its backbone), TIM-barrel fold (meaning a conserved protein fold consisting of eight α-helices and eight parallel β-strands that alternate along the peptide backbone), and antiparallel β-barrel fold (a beta barrel is a large beta-sheet that twists and coils to form a closed structure in which the first strand is hydrogen bonded to the last), but also all major classes (all α, all β, α+β, α/β) of protein structures. Within a fold group, the core of the nucleotide-binding domain of each family has the same architecture, and the topology of the protein core is either identical or related by circular permutation. Homology between the families within a fold group is not implied.

Group I (23,124 sequences) kinases incorporate protein S/T-Y kinase, atypical protein kinase, lipid kinase, and ATP grasp enzymes and further comprise the protein S/T-Y kinase, and atypical protein kinase family (22,074 sequences). These kinases include: choline kinase (EC 2.7.1.32); protein kinase (EC 2.7.137); phosphorylase kinase (EC 2.7.1.38); homoserine kinase (EC 2.7.1.39); I-phosphatidylinositol 4-kinase (EC 2.7.1.67); streptomycin 6-kinase (EC 2.7.1.72); ethanolamine kinase (EC 2.7.1.82); streptomycin 3′-kinase (EC 2.7.1.87); kanamycin kinase (EC 2.7.1.95); 5-methylthioribose kinase (EC 2.7.1.100); viomycin kinase (EC 2.7.1.103); [hydroxymethylglutaryl-CoA reductase (NADPH2)] kinase (EC 2.7.1.109); protein-tyrosine kinase (EC 2.7.1.112); [isocitrate dehydrogenase (NADP+)] kinase (EC 2.7.1.116); [myosin light-chain] kinase (EC 2.7.1.117); hygromycin-B kinase (EC 2.7.1.119); calcium/calmodulin-dependent protein kinase (EC 2.7.1.123); rhodopsin kinase (EC 2.7.1.125); [beta-adrenergic-receptor] kinase (EC 2.7.1.126); [myosin heavy-chain] kinase (EC 2.7.1.129); [Tau protein] kinase (EC 2.7.1.135); macrolide 2′-kinase (EC 2.7.1.136); I-phosphatidylinositol 3-kinase (EC 2.7.1.137); [RNA-polymerase]-subunit kinase (EC 2.7.1.141); phosphatidylinositol-4,5-bisphosphate 3-kinase (EC 2.7.1.153); and phosphatidylinositol-4-phosphate 3-kinase (EC 2.7.1.154). Group I further comprises the lipid kinase family (321 sequences). These kinases include: I-phosphatidylinositol-4-phosphate 5-kinase (EC 2.7.1.68); I D-myo-inositol-triphosphate 3-kinase (EC 2.7.1.127); inositol-tetrakisphosphate 5-kinase (EC 2.7.1.140); I-phosphatidylinositol-5-phosphate 4-kinase (EC 2.7.1.149); I-phosphatidylinositol-3-phosphate 5-kinase (EC 2.7.1.150); inositol-polyphosphate multikinase (EC 2.7.1.151); and inositol-hexakiphosphate kinase (EC 2.7.4.21). Group I further comprises the ATP-grasp kinases (729 sequences) which include inositol-tetrakisphosphate 1-kinase (EC 2.7.1.134); pyruvate, phosphate dikinase (EC 2.7.9.1); and pyruvate, water dikinase (EC 2.7.9.2).

Group II (17,071 sequences) kinases incorporate the Rossman-like kinases. Group II comprises the P-loop kinase family (7,732 sequences). These include gluconokinase (EC 2.7.1.12); phosphoribulokinase (EC 2.7.1.19); thymidine kinase (EC 2.7.1.21); ribosylnicotinamide kinase (EC 2.7.1.22); dephospho-CoA kinase (EC 2.7.1.24); adenylylsulfate kinase (EC 2.7.1.25); pantothenate kinase (EC 2.7.1.33); protein kinase (bacterial) (EC 2.7.1.37); uridine kinase (EC 2.7.1.48); shikimate kinase (EC 2.7.1.71); deoxycytidine kinase (EC 2.7.1.74); deoxyadenosine kinase (EC 2.7.1.76); polynucleotide 5′-hydroxyl-kinase (EC 2.7.1.78); 6-phosphofructo-2-kinase (EC 2.7.1.105); deoxyguanosine kinase (EC 2.7.1.113); tetraacyldisaccharide 4′-kinase (EC 2.7.1.130); deoxynucleoside kinase (EC 2.7.1.145); adenosylcobinamide kinase (EC 2.7.1.156); polyphosphate kinase (EC 2.7.4.1); phosphomevalonate kinase (EC 2.7.4.2); adenylate kinase (EC 2.7.4.3); nucleoside-phosphate kinase (EC 2.7.4.4); guanylate kinase (EC 2.7.4.8); thymidylate kinase (EC 2.7.4.9); nucleoside-triphosphate-adenylate kinase (EC 2.7.4.10); (deoxy)nucleoside-phosphate kinase (EC 2.7.4.13); cytidylate kinase (EC 2.7.4.14); and uridylate kinase (EC 2.7.4.22). Group II further comprises the phosphoenolpyruvate carboxykinase family (815 sequences). These enzymes include protein kinase (HPr kinase/phosphatase) (EC 2.7.1.37); phosphoenolpyruvate carboxykinase (GTP) (EC 4.1.1.32); and phosphoenolpyruvate carboxykinase (ATP) (EC 4.1.1.49). Group II further comprises the phosphoglycerate kinase (1,351 sequences) family. These enzymes include phosphoglycerate kinase (EC 2.7.2.3) and phosphoglycerate kinase (GTP) (EC 2.7.2.10). Group II further comprises the aspartokinase family (2,171 sequences). These enzymes include carbamate kinase (EC 2.7.2.2); aspartate kinase (EC 2.7.2.4); acetylglutamate kinase (EC 2.7.2.81); glutamate 5-kinase (EC 2.7.2.1) and uridylate kinase (EC 2.7.4.). Group II further comprises the phosphofructokinase-like kinase family (1,998 sequences). These enzymes include 6-phosphofrutokinase (EC 2.7.1.11); NAD(+) kinase (EC 2.7.1.23); I-phosphofructokinase (EC 2.7.1.56); diphosphate-fructose-6-phosphate I-phosphotransferase (EC 2.7.1.90); sphinganine kinase (EC 2.7.1.91); diacylglycerol kinase (EC 2.7.1.107); and ceramide kinase (EC 2.7.1.138). Group II further comprises the ribokinase-like family (2,722 sequences). These enzymes include: glucokinase (EC 2.7.1.2); ketohexokinase (EC 2.7.1.3); fructokinase (EC 2.7.1.4); 6-phosphofructokinase (EC 2.7.1.11); ribokinase (EC 2.7.1.15); adenosine kinase (EC 2.7.1.20); pyridoxal kinase (EC 2.7.1.35); 2-dehydro-3-deoxygluconokinase (EC 2.7.1.45); hydroxymethylpyrimidine kinase (EC 2.7.1.49); hydroxyethylthiazole kinase (EC 2.7.1.50); I-phosphofructokinase (EC 2.7.1.56); inosine kinase (EC 2.7.1.73); 5-dehydro-2-deoxygluconokinase (EC 2.7.1.92); tagatose-6-phosphate kinase (EC 2.7.1.144); ADP-dependent phosphofructokinase (EC 2.7.1.146); ADP-dependent glucokinase (EC 2.7.1.147); and phosphomethylpyrimidine kinase (EC 2.7.4.7). Group II further comprises the thiamin pyrophosphokinase family (175 sequences) which includes thiamin pyrophosphokinase (EC 2.7.6.2). Group II further comprises the glycerate kinase family (107 sequences) which includes glycerate kinase (EC 2.7.1.31).

Group III kinases (10,973 sequences) comprise the ferredoxin-like fold kinases. Group III further comprises the nucleoside-diphosphate kinase family (923 sequences). These enzymes include nucleoside-diphosphate kinase (EC 2.7.4.6). Group III further comprises the HPPK kinase family (609 sequences). These enzymes include 2-amino-4-hydroxy-6-hydroxymethyldihydropteridine pyrophosphokinase (EC 2.7.6.3). Group III further comprises the guanido kinase family (324 sequences). These enzymes include guanidoacetate kinase (EC 2.7.3.1); creatine kinase (EC 2.7.3.2); arginine kinase (EC 2.7.3.3); and lombricine kinase (EC 2.7.3.5). Group III further comprises the histidine kinase family (9,117 sequences). These enzymes include protein kinase (histidine kinase) (EC 2.7.1.37); [pyruvate dehydrogenase (lipoamide)] kinase (EC 2.7.1.99); and [3-methyl-2-oxybutanoate dehydrogenase(lipoamide)] kinase (EC 2.7.1.115).

Group IV kinases (2,768 sequences) incorporate ribonuclease H-like kinases. These enzymes include hexokinase (EC 2.7.1.1); glucokinase (EC 2.7.1.2); fructokinase (EC 2.7.1.4); rhamnulokinase (EC 2.7.1.5); mannokinase (EC 2.7.1.7); gluconokinase (EC 2.7.1.12); L-ribulokinase (EC 2.7.1.16); xylulokinase (EC 2.7.1.17); erythritol kinase (EC 2.7.1.27); glycerol kinase (EC 2.7.1.30); pantothenate kinase (EC 2.7.1.33); D-ribulokinase (EC 2.7.1.47); L-fucolokinase (EC 2.7.1.51); L-xylulokinase (EC 2.7.1.53); allose kinase (EC 2.7.1.55); 2-dehydro-3-deoxygalactonokinase (EC 2.7.1.58); N-acetylglucosamine kinase (EC 2.7.1.59); N-acylmannosamine kinase (EC 2.7.1.60); polyphosphate-glucose phosphotransferase (EC 2.7.1.63); beta-glucoside kinase (EC 2.7.1.85); acetate kinase (EC 2.7.2.1); butyrate kinase (EC 2.7.2.7); branched-chain-fatty-acid kinase (EC 2.7.2.14); and propionate kinase (EC 2.7.2.15).

Group V kinases (1,119 sequences) incorporate TIM β-barrel kinases. These enzymes include pyruvate kinase (EC 2.7.1.40).

Group VI kinases (885 sequences) incorporate GHMP kinases. These enzymes include galactokinase (EC 2.7.1.6); mevalonate kinase (EC 2.7.1.36); homoserine kinase (EC 2.7.1.39); L-arabinokinase (EC 2.7.1.46); fucokinase (EC 2.7.1.52); shikimate kinase (EC 2.7.1.71); 4-(cytidine 5′-diphospho)-2-C-methyl-D-erythriol kinase (EC 2.7.1.148); and phosphomevalonate kinase (EC 2.7.4.2).

Group VII kinases (1,843 sequences) incorporate AIR synthetase-like kinases. These enzymes include thiamine-phosphate kinase (EC 2.7.4.16) and selenide, water dikinase (EC 2.7.9.3).

Group VIII kinases (565 sequences) incorporate riboflavin kinases (565 sequences). These enzymes include riboflavin kinase (EC 2.7.1.26).

Group IX kinases (197 sequences) incorporate dihydroxyacetone kinases. These enzymes include glycerone kinase (EC 2.7.1.29).

Group X kinases (148 sequences) incorporate putative glycerate kinases. These enzymes include glycerate kinase (EC 2.7.1.31).

Group XI kinases (446 sequences) incorporate polyphosphate kinases. These enzymes include polyphosphate kinases (EC 2.7.4.1).

Group XII kinases (263 sequences) incorporate integral membrane kinases. Group XII comprises the dolichol kinase family. These enzymes include dolichol kinases (EC 2.7.1.108). Group XII further comprises the undecaprenol kinase family. These enzymes include undecaprenol kinases (EC 2.7.1.66).

Kinases play indispensable roles in numerous cellular metabolic and signaling pathways, and are among the best-studied enzymes at the structural, biochemical, and cellular level. Despite the fact that all kinases use the same phosphate donor (in most cases, ATP) and catalyze apparently the same phosphoryl transfer reaction, they display remarkable diversity in their structural folds and substrate recognition mechanisms. This probably is due largely to the diverse nature of the structures and properties of their substrates.

### Mitogen-Activated Protein Kinase (MAPK)-Activated Protein Kinases (MK2 and MK3)

FIG. 1 depicts mitogen-activated protein kinase signaling pathways. MAPK signaling activates a three-tiered cascade with MAPK kinase kinases (MAP3K) activating MAPAK kinases (MAP2K) and finally MAPK. The major MAPK pathways involved in inflammatory diseases are extracellular regulating kinase (ERK), p38 MAPK, and c-Jun NH2-terminal kinase (JNK). Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). Inhibitors are shown in green boxes. (Barnes, P J, (2016) “Kinases as novel therapeutic targets in asthma and chronic obstructive pulmonary disease,” Pharmacol. Rev. 68: 788-815).

Different groups of MAPK-activated protein kinases (MAPKAPKs) have been defined downstream of mitogen-activated protein kinases (MAPKs). These enzymes transduce signals to target proteins that are not direct substrates of the MAPKs and, therefore, serve to relay phosphorylation-dependent signaling with MAPK cascades to diverse cellular functions. One of these groups is formed by the three MAPKAPKs: MK2, MK3 (also known as 3pK), and MK5 (also designated PRAK). Mitogen-activated protein kinase-activated protein kinase 2 (also referred to as “MAPKAPK2”, “MAPKAP-K2”, “MK2”) is a kinase of the serine/threonine (Ser/Thr) protein kinase family. MK2 is highly homologous to MK3 (approximately 75% amino acid identity). The kinase domains of MK2 and MK3 are most similar (approximately 35% to 40% identity) to calcium/calmodulin-dependent protein kinase (CaMK), phosphorylase b kinase, and the C-terminal kinase domain (CTKD) of the ribosomal S6 kinase (RSK) isoforms. The MK2 gene encodes two alternatively spliced transcripts of 370 amino acids (MK2A) and 400 amino acids (MK2B). The MK3 gene encodes one transcript of 382 amino acids. The MK2- and MK3 proteins are highly homologous, yet MK2A possesses a shorter C-terminal region. The C-terminus of MK2B contains a functional bipartite nuclear localization sequence (NLS) (Lys-Lys-Xaa-Xaa-Xaa-Xaa-Xaa-Xaa-Xaa-Xaa-Xaa-Xaa-Lys-Arg-Arg-Lys-Lys; SEQ ID NO: 21) that is not present in the shorter MK2A isoform, indicating that alternative splicing determines the cellular localization of the MK2 isoforms. MK3 possesses a similar nuclear localization sequence. The nuclear localization sequence found in both MK2B and MK3 encompasses a D domain (Leu-Leu-Lys-Arg-Arg-Lys-Lys; SEQ ID NO: 22), which was shown to mediate the specific interaction of MK2B and MK3 with p38α and p38β. MK2B and MK3 also possess a functional nuclear export signal (NES) located N-terminal to the NLS and D domain. The NES in MK2B is sufficient to trigger nuclear export following stimulation, a process which may be inhibited by leptomycin B. The sequence N-terminal to the catalytic domain in MK2 and MK3 is proline rich and contains one (MK3) or two (MK2) putative Src homology 3 (SH3) domain-binding sites, which studies have shown, for MK2, to mediate binding to the SH3 domain of c-Abl in vitro. Recent studies suggest that this domain is involved in MK2-mediated cell migration.

MK2B and MK3 are located predominantly in the nucleus of quiescent cells while MK2A is present in the cytoplasm. Both MK2B and MK3 are rapidly exported to the cytoplasm via a chromosome region maintenance protein (CRM1)-dependent mechanism upon stress stimulation. Nuclear export of MK2B appears to be mediated by kinase activation, as phosphomimetic mutation of Thr334 within the activation loop of the kinase enhances the cytoplasmic localization of MK2B. Without being limited by theory, it is thought that MK2B and MK3 may contain a constitutively active nuclear localization signal (NLS) and a phosphorylation-regulated nuclear export signal (NES).

MK2 and MK3 appear to be expressed ubiquitously, with increased relative expression in the heart, lungs, kidney, reproductive organs (mammary and testis), skin and skeletal muscle tissues, as well as in immune-related cells such as white blood cells/leukocytes and dendritic cells.

### Activation of MK2 and MK3 Kinase Activity

Various activators of p38α and p38β potently stimulate MK2 and MK3 activity. p38 mediates the in vitro and in vivo phosphorylation of MK2 on four proline-directed sites: Thr25, Thr222, Ser272, and Thr334. Of these sites, only Thr25 is not conserved in MK3. Without being limited by theory, while the function of phosphorylated Thr25 is unknown, its location between the two SH3 domain-binding sites suggests that it may regulate protein-protein interactions. Thr222 in MK2 (Thr201 in MK3) is located in the activation loop of the kinase domain and has been shown to be essential for MK2 and MK3 kinase activity. Thr334 in MK2 (Thr313 in MK3) is located C-terminal to the catalytic domain and is essential for kinase activity. The crystal structure of MK2 has been resolved and, without being limited by theory, suggests that Thr334 phosphorylation may serve as a switch for MK2 nuclear import and export. Phosphorylation of Thr334 also may weaken or interrupt binding of the C terminus of MK2 to the catalytic domain, exposing the NES and promoting nuclear export.

Studies have shown that while p38 is capable of activating MK2 and MK3 in the nucleus, experimental evidence suggests that activation and nuclear export of MK2 and MK3 are coupled by a phosphorylation-dependent conformational switch that also dictates p38 stabilization and localization, and the cellular location of p38 itself is controlled by MK2 and possibly MK3. Additional studies have shown that nuclear p38 is exported to the cytoplasm in a complex with MK2 following phosphorylation and activation of MK2. The interaction between p38 and MK2 may be important for p38 stabilization since studies indicate that p38 levels are low in MK2-deficient cells and expression of a catalytically inactive MK2 protein restores p38 levels.

### Substrates and Functions

MK2 shares many substrates with MK3. Both enzymes have comparable substrate preferences and phosphorylate peptide substrates with similar kinetic constants. The minimum sequence required for efficient phosphorylation by MK2 was found to be Hyd-Xaa-Arg-Xaa-Xaa-pSer/pThr (SEQ ID NO: 22), where Hyd is a bulky, hydrophobic residue.

Accumulating studies have shown that MK2 phosphorylates a variety of proteins, which include, but are not limited to, 5-Lipooxygenase (ALOX5), Cell Division Cycle 25 Homolog B (CDC25B), Cell Division Cycle 25 Homolog C (CDC25C), Embryonic Lethal, Abnormal Vision, Drosophila-Like 1 (ELAVL1), Heterogeneous Nuclear Ribonucleoprotein A0 (HNRNPA0), Heat Shock Factor protein 1 (HSF1), Heat Shock Protein Beta-1 (HSPB1), Keratin 18 (KRT18), Keratin 20 (KRT20), LIM domain kinase 1 (LIMK1), Lymphocyte-specific protein 1 (LSP1), Polyadenylate-Binding Protein 1 (PABPC1), Poly(A)-specific Ribonuclease (PARN), CAMP-specific 3′,5′-cyclic Phosphodiesterase 4A (PDE4A), RCSD domain containing 1 (RCSD1), Ribosomal protein S6 kinase, 90 kDa, polypeptide 3 (RPS6KA3), TGF-beta activated kinase 1/MAP3K7 binding protein 3 (TAB3), and Tristetraprolin (TTP/ZFP36).

Heat-Shock Protein Beta-1 (also termed HSPB1 or HSP27) is a stress-inducible cytosolic protein that is ubiquitously present in normal cells and is a member of the small heat-shock protein family. The synthesis of HSPB1 is induced by heat shock and other environmental or pathophysiologic stresses, such as UV radiation, hypoxia and ischemia. Besides its putative role in thermoresistance, HSPB1 is involved in the survival and recovery of cells exposed to stressful conditions.

Experimental evidence supports a role for p38 in the regulation of cytokine biosynthesis and cell migration. The targeted deletion of the mk2 gene in mice suggested that although p38 mediates the activation of many similar kinases, MK2 seems to be the key kinase responsible for these p38-dependent biological processes. Loss of MK2 leads (i) to a defect in lipopolysaccharide (LPS)-induced synthesis of cytokines such as tumor necrosis factor alpha (TNF-α), interleukin-6 (IL-6), and gamma interferon (IFN-γ) and (ii) to changes in the migration of mouse embryonic fibroblasts, smooth muscle cells, and neutrophils.

Consistent with a role for MK2 in inflammatory and immune responses, MK2-deficient mice showed increased susceptibility to Listeria monocytogenes infection and reduced inflammation-mediated neuronal death following focal ischemia. Since the levels of p38 protein also are reduced significantly in MK2-deficient cells, it was necessary to distinguish whether these phenotypes were due solely to the loss of MK2. To achieve this, MK2 mutants were expressed in MK2-deficient cells, and the results indicated that the catalytic activity of MK2 was not necessary to restore p38 levels but was required to regulate cytokine biosynthesis.

Knockout or knockdown studies of MK2 provide strong support that activated MK2 enhances stability of IL-6 mRNA through phosphorylation of proteins interacting with the AU-rich 3′ untranslated region of IL-6 mRNA. In particular, it has been shown that MK2 is principally responsible for phosphorylation of hnRNPA0, an mRNA-binding protein that stabilizes IL-6 RNA. In addition, several additional studies investigating diverse inflammatory diseases have found that levels of pro-inflammatory cytokines, such as IL-6, IL-1β, TNF-α and IL-8, are increased in induced sputum from patients with stable chronic obstructive pulmonary disease (COPD) or from the alveolar macrophages of cigarette smokers (Keatings V. et al, Am J Resp Crit Care Med, 1996, 153:530-534; Lim, S. et al., J Respir Crit Care Med, 2000, 162:1355-1360).

Regulation of mRNA Translation.

Previous studies using MK2 knockout mice or MK2-deficient cells have shown that MK2 increases the production of inflammatory cytokines, including TNF-α, IL-1, and IL-6, by increasing the rate of translation of its mRNA. No significant reductions in the transcription, processing, and shedding of TNF-α could be detected in MK2-deficient mice. The p38 pathway is known to play an important role in regulating mRNA stability, and MK2 represents a likely target by which p38 mediates this function. Studies utilizing MK2-deficient mice indicated that the catalytic activity of MK2 is necessary for its effects on cytokine production and migration, suggesting that, without being limited by theory, MK2 phosphorylates targets involved in mRNA stability. Consistent with this, MK2 has been shown to bind and/or phosphorylate the heterogeneous nuclear ribonucleoprotein (hnRNP) A0, tristetraprolin (TTP), the poly(A)-binding protein PABP1, and HuR, a ubiquitously expressed member of the ELAV (Embryonic-Lethal Abnormal Visual in Drosophila melanogaster) family of RNA-binding protein. These substrates are known to bind or copurify with mRNAs that contain AU-rich elements in the 3′ untranslated region, suggesting that MK2 may regulate the stability of AU-rich mRNAs such as TNF-α. It currently is unknown whether MK3 plays a similar role, but LPS treatment of MK2-deficient fibroblasts completely abolished hnRNP A0 phosphorylation, suggesting that MK3 is not able to compensate for the loss of MK2.

MK3 participates with MK2 in phosphorylation of the eukaryotic elongation factor 2 (eEF2) kinase. eEF2 kinase phosphorylates and inactivates eEF2. eEF2 activity is critical for the elongation of mRNA during translation, and phosphorylation of eEF2 on Thr56 results in the termination of mRNA translation. MK2 and MK3 phosphorylation of eEF2 kinase on Ser377 suggests that these enzymes may modulate eEF2 kinase activity and thereby regulate mRNA translation elongation.

### Transcriptional Regulation by MK2 and MK3

Nuclear MK2, similar to many MKs, contributes to the phosphorylation of cAMP response element binding (CREB), Activating Transcription Factor-1 (ATF-1), serum response factor (SRF), and transcription factor ER81. Comparison of wild-type and MK2-deficient cells revealed that MK2 is the major SRF kinase induced by stress, suggesting a role for MK2 in the stress-mediated immediate-early response. Both MK2 and MK3 interact with basic helix-loop-helix transcription factor E47 in vivo and phosphorylate E47 in vitro. MK2-mediated phosphorylation of E47 was found to repress the transcriptional activity of E47 and thereby inhibit E47-dependent gene expression, suggesting that MK2 and MK3 may regulate tissue-specific gene expression and cell differentiation.

### Other Targets of MK2 and MK3

Several other MK2 and MK3 substrates also have been identified, reflective of the diverse functions of MK2 and MK3 in several biological processes. The scaffolding protein 14-3-3ζ is a physiological MK2 substrate. Studies indicate that 14-3-3ζ interacts with a number of components of cell signaling pathways, including protein kinases, phosphatases, and transcription factors. Additional studies have shown that MK2-mediated phosphorylation of 14-3-3ζ on Ser58 compromises its binding activity, suggesting that MK2 may affect the regulation of several signaling molecules normally regulated by 14-3-3ζ.

Additional studies have shown that MK2 also interacts with and phosphorylates the p16 subunit of the seven-member Arp2 and Arp3 complex (p16-Arc) on Ser77. p16-Arc has roles in regulating the actin cytoskeleton, suggesting that MK2 may be involved in this process. Further studies have shown that the small heat shock protein HSPB1, lymphocyte-specific protein LSP-1, and vimentin are phosphorylated by MK2. HSPB1 is of particular interest because it forms large oligomers which may act as molecular chaperones and protect cells from heat shock and oxidative stress. Upon phosphorylation, HSPB1 loses its ability to form large oligomers and is unable to block actin polymerization, suggesting that MK2-mediated phosphorylation of HSPB1 serves a homeostatic function aimed at regulating actin dynamics that otherwise would be destabilized during stress. MK3 also was shown to phosphorylate HSPB1 in vitro and in vivo, but its role during stressful conditions has not yet been elucidated.

It was also shown that HSPB1 binds to polyubiquitin chains and to the 26S proteasome in vitro and in vivo. The ubiquitin-proteasome pathway is involved in the activation of transcription factor NF-kappa B (NF-κB) by degrading its main inhibitor, I kappa B-alpha (IκB-alpha), and it was shown that overexpression of HSPB1 increases NF-kappaB (NF-κB) nuclear relocalization, DNA binding, and transcriptional activity induced by etoposide, TNF-alpha, and Interleukin-1 beta (IL-1β). Additionally, previous studies have suggested that HSPB1, under stress conditions, favors the degradation of ubiquitinated proteins, such as phosphorylated I kappa B-alpha (IκB-alpha); and that this function of HSPB1 accounts for its anti-apoptotic properties through the enhancement of NF-kappa B (NF-κB) activity (Parcellier, A. et al., Mol Cell Biol, 23(16): 5790-5802, 2003).

MK2 and MK3 also may phosphorylate 5-lipoxygenase. 5-lipoxygenase catalyzes the initial steps in the formation of the inflammatory mediators, leukotrienes. Tyrosine hydroxylase, glycogen synthase, and Akt also were shown to be phosphorylated by MK2. Finally, MK2 phosphorylates the tumor suppressor protein tuberin on Ser1210, creating a docking site for 14-3-3ζ. Tuberin and hamartin normally form a functional complex that negatively regulates cell growth by antagonizing mTOR-dependent signaling, suggesting that p38-mediated activation of MK2 may regulate cell growth by increasing 14-3-3ζ binding to tuberin.

Accumulating studies have suggested that the reciprocal crosstalk between the p38 MAPK-pathway and signal transducer and activator of transcription 3 (STAT3)-mediated signal-transduction forms a critical axis successively activated in lipopolysaccharide (LPS) challenge models. It was shown that the balanced activation of this axis is essential for both induction and propagation of the inflammatory macrophage response as well as for the control of the resolution phase, which is largely driven by IL-10 and sustained STAT3 activation (Bode, J. et al., Cellular Signaling, 24: 1185-1194, 2012). Another study has shown that MK2 controls LPS-inducible IFNβ gene expression and subsequent IFNβ-mediated activation of STAT3 by neutralizing negative regulatory effects of MK3 on LPS-induced p65 and IRF3-mediated signaling. The study further showed that in mk2/3 knockout macrophages, IFNβ-dependent STAT3 activation occurs independently from IL-10, because, in contrast to IFNβ, impaired IL-10 expression is not restored upon additional deletion of MK3 in mk2/3 knockout macrophages (Ehlting, C. et al., J. Biol. Chem., 285(27): 24113-24124).

### Kinase Inhibition

The eukaryotic protein kinases constitute one of the largest superfamilies of homologous proteins that are related by virtue of their catalytic domains. Most related protein kinases are specific for either serine/threonine or tyrosine phosphorylation. Protein kinases play an integral role in the cellular response to extracellular stimuli. Thus, stimulation of protein kinases is considered to be one of the most common activation mechanisms in signal transduction systems. Many substrates are known to undergo phosphorylation by multiple protein kinases, and a considerable amount of information on primary sequence of the catalytic domains of various protein kinases has been published. These sequences share a large number of residues involved in ATP binding, catalysis, and maintenance of structural integrity. Most protein kinases possess a well conserved 30-32 kDa catalytic domain.

Studies have attempted to identify and utilize regulatory elements of protein kinases. These regulatory elements include inhibitors, antibodies, and blocking peptides.

### Inhibitors

Enzyme inhibitors are molecules that bind to enzymes thereby decreasing enzyme activity. The binding of an inhibitor may stop a substrate from entering the active site of the enzyme and/or hinder the enzyme from catalyzing its reaction (as in inhibitors directed at the ATP biding site of the kinase). Inhibitor binding is either reversible or irreversible. Irreversible inhibitors usually react with the enzyme and change it chemically (e.g., by modifying key amino acid residues needed for enzymatic activity) so that it no longer is capable of catalyzing its reaction. In contrast, reversible inhibitors bind non-covalently and different types of inhibition are produced depending on whether these inhibitors bind the enzyme, the enzyme-substrate complex, or both.

Enzyme inhibitors often are evaluated by their specificity and potency. The term “specificity” as used in this context refers to the selective attachment of an inhibitor or its lack of binding to other proteins. The term “potency” as used herein refers to an inhibitor's dissociation constant, which indicates the concentration of inhibitor needed to inhibit an enzyme.

Inhibitors of protein kinases have been studied for use as a tool in protein kinase activity regulation. Inhibitors have been studied for use with, for example, cyclin-dependent (Cdk) kinase, MAP kinase, serine/threonine kinase, Src Family protein tyrosine kinase, tyrosine kinase, calmodulin (CaM) kinase, casein kinase, checkpoint kinase (ChkI), glycogen synthase kinase 3 (GSK-3), c-Jun N-terminal kinase (JNK), mitogen-activated protein kinase 1 (MEK), myosin light chain kinase (MLCK), protein kinase A, Akt (protein kinase B), protein kinase C, protein kinase G, protein tyrosine kinase, Raf kinase, and Rho kinase.

### Small-Molecule MK2 Inhibitors

While individual inhibitors that target MK2 with at least modest selectivity with respect to other kinases have been designed, it has been difficult to create compounds with favorable solubility and permeability. As a result, there are relatively few biochemically efficient MK2 inhibitors that have advanced to in vivo pre-clinical studies (Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012; incorporated by reference in its entirety).

The majority of disclosed MK2 inhibitors are classical type I inhibitors as revealed by crystallographic or biochemical studies. As such, they bind to the ATP site of the kinase and thus compete with intra-cellular ATP (estimated concentration 1 mM-5 mM) to inhibit phosphorylation and activation of the kinase. Representative examples of small-molecule MK2 inhibitors include, but are not limited to,

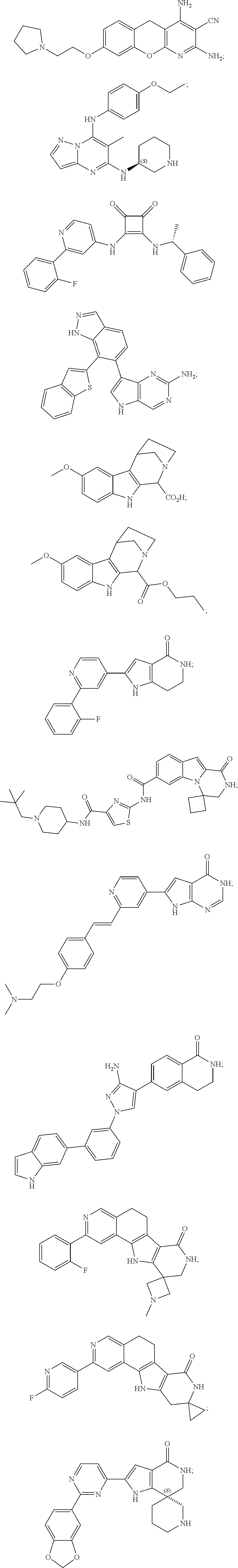

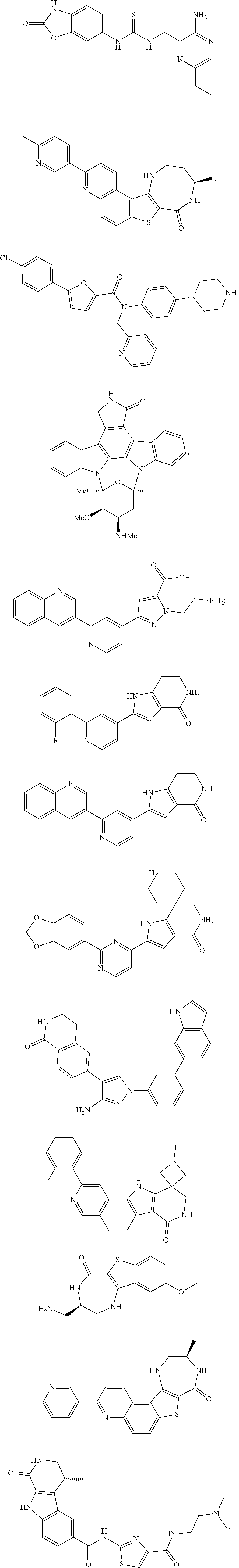

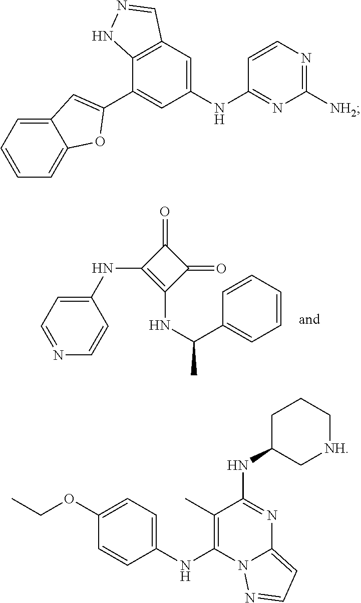 [SMILES: Nc1nccc(Nc2cc(-c3cc4ccccc4o3)c3[nH]ncc3c2)n1 ; C[C@@H](Nc1c(Nc2ccncc2)c(=O)c1=O)c1ccccc1 ; CCOc1ccc(Nc2c(C)c(N[C@H]3CCCNC3)nc3ccnn23)cc1]

### Blocking Peptides

A peptide is a chemical compound that is composed of a chain of two or more amino acids whereby the carboxyl group of one amino acid in the chain is linked to the amino group of the other via a peptide bond. Peptides have been used inter alia in the study of protein structure and function. Synthetic peptides may be used inter alia as probes to see where protein-peptide interactions occur. Inhibitory peptides may be used inter alia in clinical research to examine the effects of peptides on the inhibition of protein kinases, cancer proteins and other disorders.

The use of several blocking peptides has been studied. For example, extracellular signal-regulated kinase (ERK), a MAPK protein kinase, is essential for cellular proliferation and differentiation. The activation of MAPKs requires a cascade mechanism whereby MAPK is phosphorylated by an upstream MAPKK (MEK) which then, in turn, is phosphorylated by a third kinase MAPKKK (MEKK). The ERK inhibitory peptide functions as a MEK decoy by binding to ERK.

Other blocking peptides include autocamtide-2 related inhibitory peptide (AIP). This synthetic peptide is a highly specific and potent inhibitor of Ca2+/calmodulin-dependent protein kinase II (CaMKII). AIP is a non-phosphorylatable analog of autocamtide-2, a highly selective peptide substrate for CaMKII. AIP inhibits CaMKII with an IC50 of 100 nM (IC50 is the concentration of an inhibitor required to obtain 50% inhibition). The AIP inhibition is non-competitive with respect to syntide-2 (CaMKII peptide substrate) and ATP but competitive with respect to autocamtide-2. The inhibition is unaffected by the presence or absence of Ca2+/calmodulin. CaMKII activity is inhibited completely by AIP (1 μM) while PKA, PKC and CaMKIV are not affected.

Other blocking peptides include cell division protein kinase 5 (Cdk5) inhibitory peptide (CIP). Cdk5 phosphorylates the microtubule protein tau at Alzheimer's Disease-specific phospho-epitopes when it associates with p25. p25 is a truncated activator, which is produced from the physiological Cdk5 activator p35 upon exposure to amyloid β peptides. Upon neuronal infections with CIP, CIPs selectively inhibit p25/Cdk5 activity and suppress the aberrant tau phosphorylation in cortical neurons. The reasons for the specificity demonstrated by CIP are not fully understood.

Additional blocking peptides that have been studied include extracellular-regulated kinase 2 (ERK2), ERK3, p38/HOG1, protein kinase C, casein kinase II, Ca2+/calmodulin kinase IV, casein kinase II, Cdk4, Cdk5, DNA-dependent protein kinase (DNA-PK), serine/threonine-protein kinase PAK3, phosphoinositide (PI)-3 kinase, PI-5 kinase, PSTAIRE (the cdk highly conserved sequence), ribosomal S6 kinase, GSK-4, germinal center kinase (GCK), SAPK (stress-activated protein kinase), SEK1 (stress signaling kinase), and focal adhesion kinase (FAK).

### Protein Substrate-Competitive Inhibitors

Most of the protein kinase inhibitors developed to date are ATP competitors. This type of molecule competes for the ATP binding site of the kinase and often shows off-target effects due to serious limitations in its specificity. The low specificity of these inhibitors is due to the fact that the ATP binding site is highly conserved among diverse protein kinases. Non-ATP competitive inhibitors, on the other hand, such as substrate competitive inhibitors, are expected to be more specific as the substrate binding sites have a certain degree of variability among the various protein kinases.

Although substrate competitive inhibitors usually have a weak binding interaction with the target enzyme in vitro, studies have shown that chemical modifications can improve the specific binding affinity and the in vivo efficacy of substrate inhibitors (Eldar-Finkelman, H. et al., Biochim, Biophys. Acta, 1804(3):598-603, 2010). In addition, substrate competitive inhibitors show better efficacy in cells than in cell-free conditions in many cases (van Es, J. et al., Curr. Opin. Gent. Dev. 13:28-33, 2003).

In an effort to enhance specificity and potency in protein kinase inhibition, bisubstrate inhibitors also have been developed. Bisubstrate inhibitors, which consist of two conjugated fragments, each targeted to a different binding site of a bisubstrate enzyme, form a special group of protein kinase inhibitors that mimic two natural substrates/ligands and that simultaneously associate with two regions of given kinases. The principle advantage of bisubstrate inhibitors is their ability to generate more interactions with the target enzyme that could result in improved affinity and selectivity of the conjugates, when compared with single-site inhibitors. Examples of bisubstrate inhibitors include, but are not limited to, nucleotide-peptide conjugates, adenosine derivative-peptide conjugates, and conjugates of peptides with potent ATP-competitive inhibitors.

### Protein Transduction Domains (PTD)/Cell Permeable Proteins (CPP)

The plasma membrane presents a formidable barrier to the introduction of macromolecules into cells. For nearly all therapeutics to exert their effects, at least one cellular membrane must be traversed. Traditional small molecule pharmaceutical development relies on the chance discovery of membrane permeable molecules with the ability to modulate protein function. Although small molecules remain the dominant therapeutic paradigm, many of these molecules suffer from lack of specificity, side effects, and toxicity. Information-rich macromolecules, which have protein modulatory functions far superior to those of small molecules, can be created using rational drug design based on molecular, cellular, and structural data. However, the plasma membrane is impermeable to most molecules of size greater than 500 Da. Therefore, the ability of cell penetrating peptides, such as the basic domain of Trans-Activator of Transcription (Tat), to cross the cell membrane and deliver macromolecular cargo in vivo, can greatly facilitate the rational design of therapeutic proteins, peptides, and nucleic acids.

Protein transduction domains (PTDs) are a class of peptides capable of penetrating the plasma membrane of mammalian cells and of transporting compounds of many types and molecular weights across the membrane. These compounds include effector molecules, such as proteins, DNA, conjugated peptides, oligonucleotides, and small particles such as liposomes. When PTDs are chemically linked or fused to other proteins, the resulting fusion peptides still are able to enter cells. Although the exact mechanism of transduction is unknown, internalization of these proteins is not believed to be receptor-mediated or transporter-mediated. PTDs are generally 10-16 amino acids in length and may be grouped according to their composition, such as, for example, peptides rich in arginine and/or lysine.

The use of PTDs capable of transporting effector molecules into cells has become increasingly attractive in the design of drugs as they promote the cellular uptake of cargo molecules. These cell-penetrating peptides, generally categorized as amphipathic (meaning having both a polar and a nonpolar end) or cationic (meaning of or relating to containing net positively charged atoms) depending on their sequence, provide a non-invasive delivery technology for macromolecules. PTDs often are referred to as “Trojan peptides”, “membrane translocating sequences”, or “cell permeable proteins” (CPPs). PTDs also may be used to assist novel HSPB1 kinase inhibitors to penetrate cell membranes. (see U.S. application Ser. No. 11/972,459, entitled “Polypeptide Inhibitors of HSPB1 Kinase and Uses Therefor,” filed Jan. 10, 2008, and Ser. No. 12/188,109, entitled “Kinase Inhibitors and Uses Thereof,” filed Aug. 7, 2008, the contents of each application are incorporated by reference in their entirety herein).

### Viral PTD Containing Proteins

The first proteins to be described as having transduction properties were of viral origin. These proteins still are the most commonly accepted models for PTD action. The HIV-1 Transactivator of Transcription (Tat) and HSV-1 VP 22 protein are the best characterized viral PTD containing proteins.

Tat (HIV-1 trans-activator gene product) is an 86-amino acid polypeptide, which acts as a powerful transcription factor of the integrated HIV-1 genome. Tat acts on the viral genome, stimulating viral replication in latently infected cells. The translocation properties of the Tat protein enable it to activate quiescent infected cells, and it may be involved in priming of uninfected cells for subsequent infection by regulating many cellular genes, including cytokines. The minimal PTD of Tat is the 9 amino acid protein sequence RKKRRQRRR (TAT49-57; SEQ ID NO: 20). Studies utilizing a longer fragment of Tat demonstrated successful transduction of fusion proteins up to 120 kDa. The addition of multiple Tat-PTDs as well as synthetic Tat derivatives has been demonstrated to mediate membrane translocation. Tat PTD containing fusion proteins have been used as therapeutic moieties in experiments involving cancer, transporting a death-protein into cells, and disease models of neurodegenerative disorders.

The mechanism used by transducing peptides to permeate cell membranes has been the subject of considerable interest in recent years, as researchers have sought to understand the biology behind transduction. Early reports that Tat transduction occurred by a nonendocytic mechanism have largely been dismissed as artifactual though other cell-penetrating peptides might be taken up by way of direct membrane disruption. The recent findings that transduction of Tat and other PTDs occurs by way of macropinocytosis, a specialized form of endocytosis, has created a new paradigm in the study of these peptides. Enhanced knowledge of the mechanism of transduction helped improve transduction efficiency with the ultimate goal of clinical success (Snyder E. and Dowdy, S., Pharm Res., 21(3):389-393, 2004).

The current model for Tat-mediated protein transduction is a multistep process that involves binding of Tat to the cell surface, stimulation of macropinocytosis, uptake of Tat and cargo into macropinosomes, and endosomal escape into the cytoplasm. The first step, binding to the cell surface, is thought to be through ubiquitous glycan chains on the cell surface. Stimulation of macropinocytosis by Tat occurs by an unknown mechanism that might include binding to a cell surface protein or occur by way of proteoglycans or glycolipids. Uptake by way of macropinocytosis, a form of fluid phase endocytosis used by all cell types, is required for Tat and polyarginine transduction. The final step in Tat transduction is escape from macropinosomes into the cytoplasm; this process is likely to be dependent on the pH drop in endosomes that, along with other factors, facilitates a perturbation of the membrane by Tat and release of Tat and its cargo (i.e. peptide, protein or drug etc.) to the cytoplasm (Snyder E. and Dowdy, S., Pharm Res., 21(3):389-393, 2004).

VP22 is the HSV-1 tegument protein, a structural part of the HSV virion. VP22 is capable of receptor independent translocation and accumulates in the nucleus. This property of VP22 classifies the protein as a PTD containing peptide. Fusion proteins comprising full length VP22 have been translocated efficiently across the plasma membrane.

Homeoproteins with Intercellular Translocation Properties

Homeoproteins are highly conserved, transactivating transcription factors involved in morphological processes. They bind to DNA through a specific sequence of 60 amino acids. The DNA-binding homeodomain is the most highly conserved sequence of the homeoprotein. Several homeoproteins have been described as exhibiting PTD-like activity; they are capable of efficient translocation across cell membranes in an energy-independent and endocytosis-independent manner without cell type specificity.

The Antennapedia protein (Antp) is a trans-activating factor capable of translocation across cell membranes; the minimal sequence capable of translocation is a 16 amino acid peptide corresponding to the third helix of the protein's homeodomain (HD). The internalization of this helix occurs at 4° C., suggesting that this process is not endocytosis dependent. Peptides of up to 100 amino acids produced as fusion proteins with AntpHD penetrate cell membranes.

Other homeodomains capable of translocation include Fushi tarazu (Ftz) and Engrailed (En) homeodomain. Many homeodomains share a highly conserved third helix.

### Human PTDs

Human PTDs may circumvent potential immunogenicity issues upon introduction into a human patient. Peptides with PTD sequences include: Hoxa-5, Hox-A4, Hox-B5, Hox-B6, Hox-B7, HOX-D3, GAX, MOX-2, and FtzPTD. These proteins all share the sequence found in AntpPTD. Other PTDs include Islet-1, Interleukin-1 (IL-1), Tumor Necrosis Factor (TNF), and the hydrophobic sequence from Kaposi-fibroblast growth factor or Fibroblast Growth Factor-4 (FGF-4) signal peptide, which is capable of energy-, receptor-, and endocytosis-independent translocation. Unconfirmed PTDs include members of the Fibroblast Growth Factor (FGF) family. FGFs are polypeptide growth factors that regulate proliferation and differentiation of a wide variety of cells. Several publications have reported that basic fibroblast growth factor (FGF-2) exhibits an unconventional internalization similar to that of VP-22, Tat, and homeodomains. It has also been reported that acidic FGF (FGF-1) translocated cell membranes at temperatures as low as 4° C. However, no conclusive evidence exists about the domain responsible for internalization or the translocation properties of fusion proteins (Beerens, A. et al., Curr Gene Ther., 3(5):486-494, 2003).

### Synthetic PTDs

Several peptides have been synthesized in an attempt to create more potent PTDs and to elucidate the mechanisms by which PTDs transport proteins across cell membranes. Many of these synthetic PTDs are based on existing and well documented peptides, while others are selected for their basic residues and/or positive charges, which are thought to be crucial for PTD function. A few of these synthetic PTDs showed better translocation properties than the existing ones (Beerens, A. et al., Curr Gene Ther., 3(5):486-494, 2003). Exemplary Tat-derived synthetic PTDs include, for example, but are not limited to, WLRRIKAWLRRIKA (SEQ ID NO: 12); WLRRIKA (SEQ ID NO: 13); YGRKKRRQRRR (SEQ ID NO: 14); WLRRIKAWLRRI (SEQ ID NO: 15); FAKLAARLYR (SEQ ID NO: 16); KAFAKLAARLYR (SEQ ID NO: 17); and HRRIKAWLKKI (SEQ ID NO: 18).

### Compositions Comprising PTDs Fused to MK2 Inhibitor Peptide Therapeutic Domains (TD)

Several MK2 inhibitor peptides (TD) have been synthesized, fused to synthetic PTDs and the use of compositions comprising these fused polypeptides has been studied. These polypeptides include, but are not limited to, YARAAARQARAKALARQLGVAA (SEQ ID NO: 1; MMI-0100), YARAAARQARAKALNRQLGVA (SEQ ID NO: 19; MMI-0200), FAKLAARLYRKALARQLGVAA (SEQ ID NO: 3; MMI-0300), KAFAKLAARLYRKALARQLGVAA (SEQ ID NO: 4; MMI-0400), HRRIKAWLKKIKALARQLGVAA (SEQ ID NO: 7; MMI-0500), YARAAARDARAKALNRQLAVAA (SEQ ID NO: 23; MMI-0600), and YARAAARQARAKALNRQLAVA (SEQ ID NO: 24; MMI-0600-2). Both in vitro and in vivo studies have shown that these polypeptides can be useful in the treatment of various diseases, disorders and conditions. These include, without limitation, hyperplasia and neoplasm (U.S. Pat. Nos. 8,536,303 and 8,741,849), inflammatory disorders (U.S. application Ser. No. 12/634,476 and U.S. application Ser. No. 13/934,933), adhesions (U.S. application Ser. No. 12/582,516), failure of a vascular graft (U.S. application Ser. No. 13/114,872), improving neurite outgrowth (U.S. application Ser. No. 12/844,815), cutaneous scarring (U.S. application Ser. No. 13/829,876), failure of a coronary artery bypass vascular graft (U.S. application Ser. No. 13/700,087) and interstitial lung disease and pulmonary fibrosis (U.S. application Ser. No. 13/445,759).

### Inhibitory Peptide YARAAARQARAKALARQLGVAA (SEQ ID NO: 1) (MMI-0100)

Inhaled inhibitory peptide YARAAARQARAKALARQLGVAA (SEQ ID NO: 1) (MMI-0100) has attractive PK/PD properties. It shows rapid and complete uptake by cells with high endosome deposition, long intracellular half-life (about 77 hours), allowing for once-daily dosing, with a prolonged functional duration of effect observed in vitro and in vivo, limited plasma exposure (little to no detectable MMI-0100 plasma concentrations, even at high doses, which minimizes potential for adverse drug events and/or drug-drug interactions; and biomarker assays have been developed for use in clinical studies), and efficacy in mice has been observed with μg/kg doses (IP and IH) with a clean safety/toxicity profile.

Preclinical data have demonstrated that MMI-0100 consistently inhibits fibrosis and inflammation in 11 distinct animal models in 4 species delivered systemically or inhaled.

| Disease | Animal Model | Results (vs Placebo) |
|---|---|---|
| Pulmonary | Mouse bleomycin | Prevented fibrosis formation and also abrogated |
| Fibrosis [4, 6] | model | progression of fibrosis after significant fibrosis |
| | (WT and mHAS2 | present. Improved survival in both WT and mHAS2tg |
| | tg) | models |
| LPS Challenge [1, 6] | Mouse LPS | Significantly decreased BAL macrophage |
| | challenge (WT) | concentrations |
| Intimal | Mouse aortic | Reduced intimal thickness, fewer infiltrating |
| Hyperplasia [2] | bypass graft | macrophages in graft tissues |
| Intimal | Porcine | Lower rates of intimal hyperplasia post angioplasty |
| Hyperplasia [1] | angioplasty | |
| Intimal | Rabbit jugular | Reduced intimal thickness, fewer infiltrating |
| Hyperplasia [7] | vein bypass graft | macrophages in graft tissues |
| Myocardial | Mouse model- | Improved cardiac function with 50% decreased |
| Infarction [3, 9] | induced MI | incidence of fibrosis |
| | (IP and IH) | |
| Cutaneous | Mouse skin | Lower rates of scarring and inflammation |
| Scarring [1] | distraction | macroscopically and by histology |
| Surgical | Rat bowel | Decreased number and tenacity of adhesions; |
| Adhesions [5] | anastomosis | healing (integrity of anastomosis) unaffected |
| Familial Cardiac | Transgenic | Decreased cardiac fibrosis; trend towards improved |
| Hypertrophy [1] | murine cardiac | survival; first long-term (6 mos) model |
| | fibrosis | |
| Allergic Asthma [1] | Murine dust mite | Significantly decreased BAL eosinophil |
| | | concentrations |

[1] Data not shown, Moerae Matrix. 2015;
[2] Muto, A., et al., Vascular Pharmacology. 56: p. 46-55 (2012);
[3] Xu L, et al. JMCC. 2014. 77: 86-101;
[4] Vittal R et al. Am J Respir Cell Mol Biol. 2013; 49(1): 47-57;
[5] Ward et al. J Surg Res. 2011; 169: e27-38.;
[6] data not shown;
[7] Evans BC, et al. Science Transl. Med. 2015; 7(291): 291ra95.
[9] Brown DI, et al. Int J Pept Res Ther. 2016.

MMI-0100 blocks key MK2 substrate phosphorylation following TGFβ activation in normal primary human fetal lung fibroblasts. In a murine bleomycin treatment model, it decreases plasma IL-6 and TNFα, modulates matrix remodeling and TFGβ-signaling genes, ameliorates fibrosis, and modulates markers of fibrosis and activated MK2 expression. Vittal R, et al. (2013) Am J Respir Cell Mol Biol. 49(1): 47-57.

In a murine bleomycin prevention model, it yields overall survival outcomes consistent with favorable histologic and biomarker outcomes. Li, Y et a., (2014) JEM 208(7): 1459-71.

The described invention provides two pathologies with an inflammatory component that have been used as model systems to explore the role of MK2 and potential therapeutic indications for nebulized cell permeant peptide inhibitor of MK2 (MMI-0100). In a first aspect, an LPS challenge in smokers, who already exhibit inflammatory changes, was employed to produce an artificial, short-term, but measurable, inflammatory response. In a second aspect, the role of MK2 in the pathogenesis of hereditary cardiomyopathies (CryAB R120G and Bag3 P209L Tg mouse lines) and an inducible CM-specific Tg mouse model of μ-calpain activation (cMyBP-C 40 kDaTg mouse line) using MMI-0100 will be evaluated.

### SUMMARY OF THE INVENTION

The described invention provides a method of treating a subject that is in an immunotolerant state with regard to an immune stimulating agent that is no longer therapeutically effective for treating a disease, disorder or condition of lung comprising, in order, (a) administering (1) a first pharmaceutical formulation formulated for delivery by inhalation containing an immunomodulatory amount of a kinase-inhibiting peptide, and (b) then administering a second pharmaceutical formulation containing a therapeutic amount of the immunostimulatory agent, wherein the method is effective to resensitize the subject to the immune stimulating agent so that the subject is immunoresponsive to the immune stimulating agent upon its subsequent administration. According to one embodiment, the immunotolerant state of the subject is characterized by an attenuated immune response to the immunostimulatory agent, compared to a normal control. According to another embodiment, the immunotolerant state is characterized by one or more of a reduced level of synthesis, expression, or both of pro-inflammatory cytokines, anti-inflammatory cytokines, both pro-inflammatory and anti-inflammatory cytokines, or an altered balance between proinflammatory cytokines and anti-inflammatory cytokines, compared to a control. According to another embodiment, the immunotolerant state is a result of repeated prior exposure to the immunostimulatory agent. According to another embodiment, the immunostimulatory agent is a chemotherapeutic agent. According to another embodiment, the immunostimulatory agent is lipopolysaccharide (LPS). According to another embodiment, the kinase-inhibiting peptide is MMI0100, or a functional equivalent, a peptide mimetic or a variant of MMI0100. According to another embodiment, the immunomodulatory amount of MMI0100 is effective to modulate MK2 signaling. According to another embodiment, the immunomodulatory amount of MMI0100 is effective to modulate the MK2 signaling affecting an MAPK pathway, an NfκB pathway, an IFN α/β pathway or a combination thereof. According to another embodiment, the immunomodulatory amount of MMI100 is effective to modulate one or more of autocrine signaling, paracrine signaling or hormonal signaling in an immune cell population. According to another embodiment, the immunomodulatory amount of MMI0100 is effective to increase activation of a population of inflammatory cells selected from the group consisting of T cells, B cells, NK cells, CT cells, neutrophils, lymphocytes, macrophages, dendritic cells. According to another embodiment, the immunomodulatory amount of MMI0100 is effective to increase one or more of autocrine signaling, paracrine signaling or hormonal signaling by immune cells. According to another embodiment, the autocrine signaling, paracrine signaling or hormonal signaling by one or more immune cells comprises TLR-4 signaling. According to another embodiment, the immune cells are one or more populations selected from T cells, B cells, NK cells, CT cells, neutrophils, lymphocytes, macrophages, dendritic cells. According to another embodiment, as a result of the signaling, the immune cells express, synthesize, or secrete one or more cytokines selected from the group consisting of IL-1α, IL-1β, IL-2, IL-3, IL-4, IL-5, IL-6, IL-7, IL-8, IL-9, IL-10, IL-11, IL-12/IL-23 P40, IL13, IL-17, IL-18, TGF-β, IFN-γ, GM-CSF, CXCL1, CXCL2, and TNF-α. According to another embodiment, a level of cytokines expressed, synthesized or secreted is measurable in a body fluid. According to another embodiment, the body fluid is sputum, blood or both. According to another embodiment, the immunoresponsive immune response comprises restoration of expression, synthesis or both of inflammatory cytokines in immune cells of the lung without affecting immune cells systemically in an amount to cause unwanted systemic side effects. According to another embodiment, the disease, disorder or condition is gram negative bacterial sepsis, cystic fibrosis, COPD, or lung cancer. According to another embodiment, the subject is an immunocompromised subject.

### BRIEF DESCRIPTION OF THE DRAWINGS

FIG. 1 depicts mitogen-activated protein kinase signaling pathways. Taken from Barnes, P J, (2016) “Kinases as novel therapeutic targets in asthma and chronic obstructive pulmonary disease,” Pharmacol. Rev. 68: 788-815.

FIG. 2 shows that TLR signaling can activate the transcription factor NFκB, which induces the expression of pro-inflammatory cytokines. First panel: TLRs signal via their cytoplasmic TIR domains, which are brought into proximity to each other by ligand-induced dimerization of their ectodomains. Some TLRs use the adaptor protein MyD88, and others use the MyD88/MAL pair to initiate signaling. The MyD88 death domain recruits the serine-threonine kinases IRAK1 and IRAK4, in association with the ubiquitin E3 ligase TRAF-6. IRAK undergoes auto-activation and phosphorylates TRAF-6, activating its E3 ligase activity. Second panel: TRAF-6 cooperates with an EG2 ligase (UBC13) and a cofactor (Uve1A) to generate polyubiquitin scaffolds by attachment of ubiquitin through its lysine 63 (K63). This scaffold recruits a complex of proteins composed of the kinase TAK1 and two adaptor proteins, TAB1 and TAB2. TAB1 and TAB2 function to bind to polyubiquitin, bringing TAK1 into proximity with IRAK to become phosphorylated. Third panel: activated TAK1 activates IKKK, the IκB complex. The IKKγ subunit (NEMO) binds to the polyubiquitin scaffold and brings the IKK complex into proximity to TAK1. TAK1 then phosphorylates and activates IKKβ, which then phosphorylates IκB, the cytoplasmic inhibitor of NFκB. Fourth panel: phosphorylated IκB is targeted by a process of ubiquitination (not shown), that leads to its degradation. This releases NFκB, which is composed of two subunits, p50 and p65, into the nucleus, driving the transcription of many genes, including those encoding inflammatory cytokines. TAK1 also stimulates activation of MAPKs JNK and p38, which phosphorylate and activate AP-1 transcription factors (not shown). Taken from Janeway's Immunobiology, 9^{th} Ed., Kenneth Murphy & Casey Weaver, Eds., Garland Sci.: New York (2017) at 95.

FIG. 3 shows that expression of antiviral interferons in response to viral nucleic acids can be stimulated by two different pathways from different TLRs. Left panel: TLR-3 signaling uses the adaptor protein TRIF, which recruits the E3 ligase TRAF3 to generate K63-linked polyubiquitin chains. This scaffold recruits NEMO and TRAF family member-associated NFκB activator (TANK), which associate with the serine-threonine kinases IκB kinase ε (IKKε) and TANK binding kinase 1 (TBK1). TBK phosphorylates transcription factor IRF3, and IRF3 then enters the nucleus and induces expression of type 1 interferon genes. Right panel: TLR-7 signals through MyD88. Here, IRAK1 directly recruits and phosphorylates IRF7, which then enters the nucleus to induce expression of type 1 interferons. Taken from Janeway's Immunobiology, 9^{th} Ed., Kenneth Murphy & Casey Weaver, Eds., Garland Sci.: New York (2017) at 96.

FIG. 4 shows the pathway by which the NLRP3 inflammasome is activated to produce pro-inflammatory cytokines. Cellular damage activates the NLRP3 inflammasome to produce pro-inflammatory cytokines. The LRR domain of NLRP3 associates with chaperones (HSP90 and SGT1) that prevent NLRP3 activation. Damage to cells caused by bacterial pore-forming toxins, reactive oxygen intermediates and disruption of lysosomes or activation of the P2X7 receptor by extracellular ATP allows efflux of K+ ions from the cell; this may dissociate these chaperones from NLRP3 and induce multiple NLRP3 molecules to aggregate through interactions of their NOD domains. The aggregated NLRP3 conformation brings multiple NLRP3 pyrin domains into close proximity, which then interact with the pyrin domains of the adaptor protein ASC (PYCARD). This conformation aggregates the ASC CARD domains, which in turn aggregate the CARD domains of pro-caspase 1. The aggregation of pro-caspase 1 induces proteolytic cleavage of itself to form the active caspase 1, which cleaves the immature forms of pro-inflammatory cytokines, releasing the mature cytokines that are then secreted. Taken from Janeway's Immunobiology, 9^{th} Ed., Kenneth Murphy & Casey Weaver, Eds., Garland Sci.: New York (2017) at 98.

FIG. 5 depicts pro- and anti-inflammatory pathways stimulated by LPS. LPS binding to its receptor complex elicits intra-cellular signal transduction that results in changes of gene expression and in enhanced production of inflammatory cytokines, including IFNβ, IFNγ, IL-1β, IL-6, IL-12 and TNFα. The production of these cytokines essentially requires activation of the p38 MAPK pathway and results in a succession of autocrine/paracrine feedback loops, which in turn modified LPS-induced cytokine expression. The release of IFNβ interferon alpha receptor (IFNAR)1 dependently induces expression of IL-10, which in turn leads to a sustained activation of STAT3, which, in contrast to STAT3 activation induced by other cytokines, such as IL6 or IFNβ, is insensitive to the endogenous inhibitor of STAT3-mediated cytokine signaling suppressor of cytokine signaling (SOCS3). STAT3 induction of SOCS3 also requires the activation of the p38MAPK pathway, which in turn is negatively regulated by the dual specific phosphatase (DUSP)1. Taken from Bode, J G et al, (2012) “The macrophage response towards LPS and its control through the p38MAPK-STAT3 axis,” Cellular Signaling 24: 1185-94.

FIG. 6 depicts a model whereby the p110δ isoform of phosphatidylinositol-3-OH kinase (PI3K) mediates a balance between pro and anti-inflammatory TLR4 signaling in dendritic cells. Taken from Siegemund, S. and Sauer, K. (2012) “Balancing pro- and anti-inflammatory TLR4 signaling,” Nature Immunology 13(11): 1031-33.

FIG. 7 show the study design for the LPS challenge as described, a double blind, placebo controlled two-way crossover for healthy smokers (N=20).

FIG. 8 shows that there is no deleterious effect on lung function (as measured by forced expiratory volume (FEV1) and AUC) following repeat dosing of MMI-0100.

FIG. 9 shows primary endpoint sputum cytokine analysis for cytokines IL-1β, IL-8, TNFα and IL-6 plotted as % ratio MMI/PBO±95% confidence interval (CI) day 5 post-LPS (N=16) with ANOVA.

FIG. 10 shows secondary endpoint sputum cell counts for total cell count, neutrophil count, neutrophil differential (%), macrophage count, and macrophage differential (%) plotted as % ratio MMI/PBO±95% confidence interval (CI) day 5 post-LPS with ANOVA.

FIG. 11 depicts the phosphorylation of MK2 protein (via measurement of STAT1 phosphorylation) in induced sputum macrophages (upper panel) and in induced sputum neutrophils (lower panel) on Day 3 for placebo (n=8) and MMI-0100 (n=8)

FIG. 12 shows the response to MMI-0100 post-LPS challenge by treatment period. Left panel shows the ratio MMI/PBO±95% confidence interval (CI) day 5 post-LPS for cytokines IL-1β, IL-8, TNFα and IL-6 by FAS. Right panel shows the effect of treatment period order (placebo (PBO) first or second) on levels of IL-1β, IL-8, TNFα and IL-6.

FIG. 13 shows the response to placebo post-LPS challenge by treatment period (treatment with placebo first or with placebo second) for the cytokines IL-1β (pg/mL), IL-8 (pg/mL), TNFα (pg/mL) and IL-6 (pg/mL).

FIG. 14 shows sputum IL-1β levels (pg/mL) in non-responders (N=6/6) and Responders (10/10) in the response to MMI-0100 following LPS challenge.

FIG. 15 shows that with respect to sputum IL-1β levels (pg/mL), subjects receiving MMI-0100 first are more likely to display robust LPS challenge responses in the placebo period and to demonstrate anti-inflammatory response to MMI-0100.

FIG. 16 shows the ratio MMI/PBO±95% confidence interval (CI) day 5 post-LPS for blood biomarkers IL-6, IL-8, TNF-α, MMP-2, MMP-8, MMP-12, IL-4, CCL2, CCL5, CXCL1, CXCL5, ICAM, and MUC1.

FIG. 17A shows IL-6 level (pg/mL) in subjects who received MMI-0100 in period 1, followed by placebo in period 2; and FIG. 17B, in subjects who received placebo in period 1, followed by MMI0100 in period 2.

FIG. 18 shows serum IL-6 after 5 days of dosing, pre- and post-LPS challenge. Boxplots and individual concentrations are shown by period and treatment; p-values correspond to Welch's t-test within each treatment period comparing MMI-0100 (N=10) to Placebo (N=10) (Left Panel) and Placebo Period 1 to Placebo Period 2 (Right Panel).

FIG. 19 shows buffy coat pHSP27 after 5 days of dosing, prior to LPS challenge. Boxplots and individual concentrations are shown by period and treatment; p-values correspond to Welch's t-test within each treatment period comparing MMI-0100 (N=10) to Placebo (N=10).

FIGS. 20A and 20B show group-level (FIG. 20A) and by-treatment period (FIG. 20BI) analysis of sputum cytokines post-LPS. Group-level and by-treatment period data depicted as least squares adjusted ratios of MMI-0100 to Placebo estimated from a linear mixed effects model that included treatment, period and sequence as fixed effects and subject within sequence as a random term.

FIGS. 21A and 21B show group-level (FIG. 21A) and by-treatment period (FIG. 21B) sputum supernatant pHSP27 Days 3 and 5 (post-LPS). Group-level and by-treatment period data depicted as least squares adjusted ratios of MMI-0100 to Placebo estimated from a linear mixed effects model that included treatment, period and sequence as fixed effects and subject within sequence as a random term.

FIG. 22 illustrates that proteotoxic peptides are found in a variety of diseases, including heart failure due to multiple causes, including mutations in the human small heat shock proteins CryAB (R120G) and Bag3 P209L. From: McLendon P M, Robbins J. Circ Res 2015; 116:1863-1882.

FIG. 23 shows that daily p38 inhibitor treatment reverses established systolic dysfunction in Bag3 P209L Tg+ hearts at 15 months (unpublished data).

FIG. 24 illustrates signaling through TGFβ, activating p38 and downstream MK2. The MMI-0100 peptide is a cell permeant inhibitor of MK2 that attenuates CryAB R120G-mediated disease.

FIG. 25 shows that MMI-0100 alleviated the interstitial but not perivascular fibrosis induced by the cMyBP-c 40 kDa fragment. Mice were treated with 50 micrograms/kg/day MMI-0100 i.p. (or PBS alone) starting at 4 weeks of age prior to induction of the cMyBP-c 40 kDa protein. A. Masson's trichrome staining of collagen/fibrosis (blue) in non-transgenic mice dosed with PBS (top row), the cMyBP-c 40 kDa transgenic hearts dosed with PBS (2nd row down). Significant reductions in fibrosis are seen in the cMyBP-c 40 kDa transgenic hearts treated with daily MMI-0100 (bottom row). B. MMI-0100 treatment attenuated the development of cardiac hypertrophy, evidenced by reductions in heart weight/body weight. C/D. MMI-0100 treatment improved survival at 10 weeks in both female and male cMyBP-c 40 kDa mice. Unpublished data.

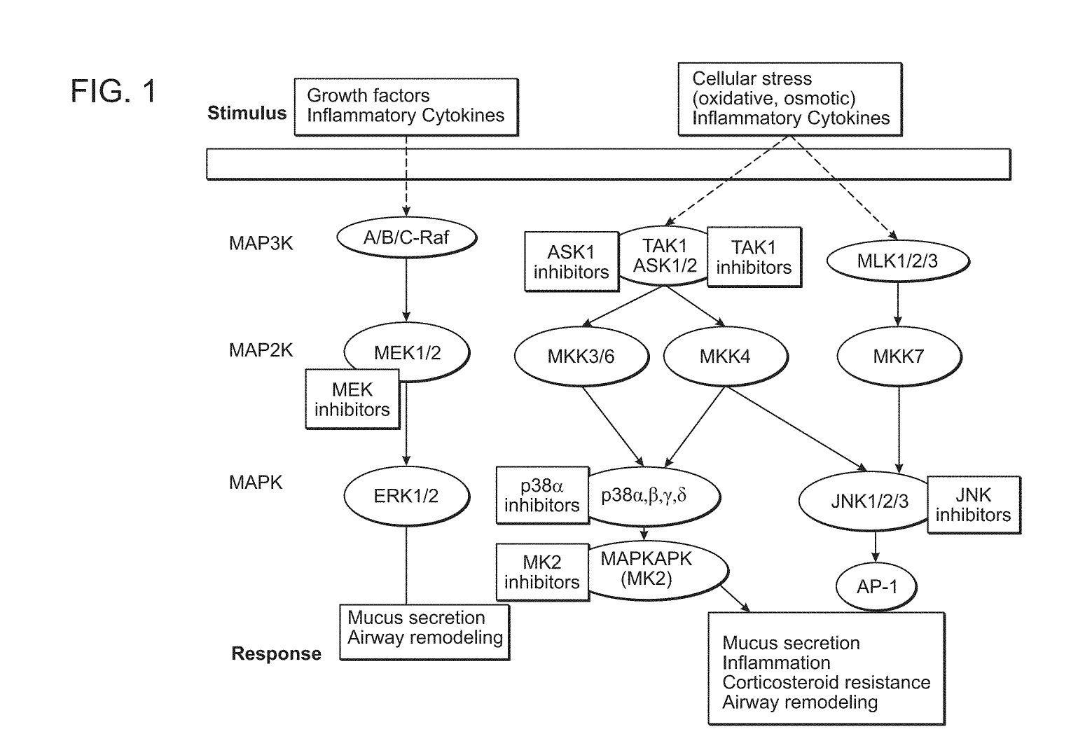

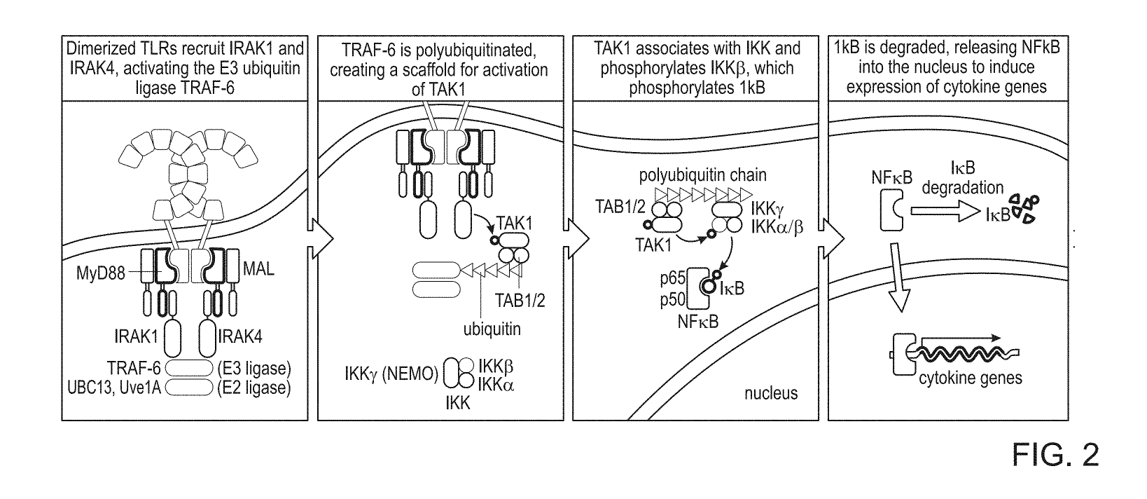

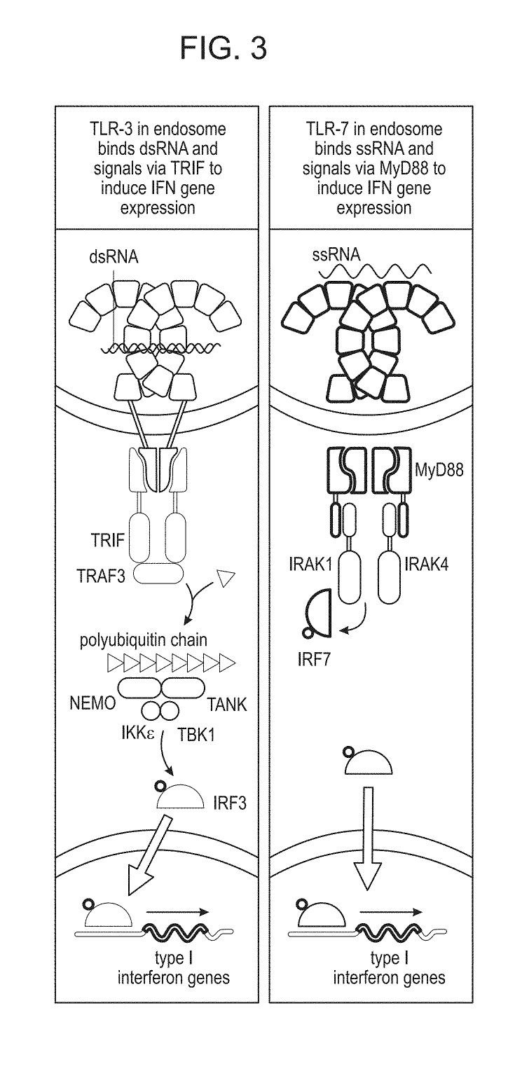

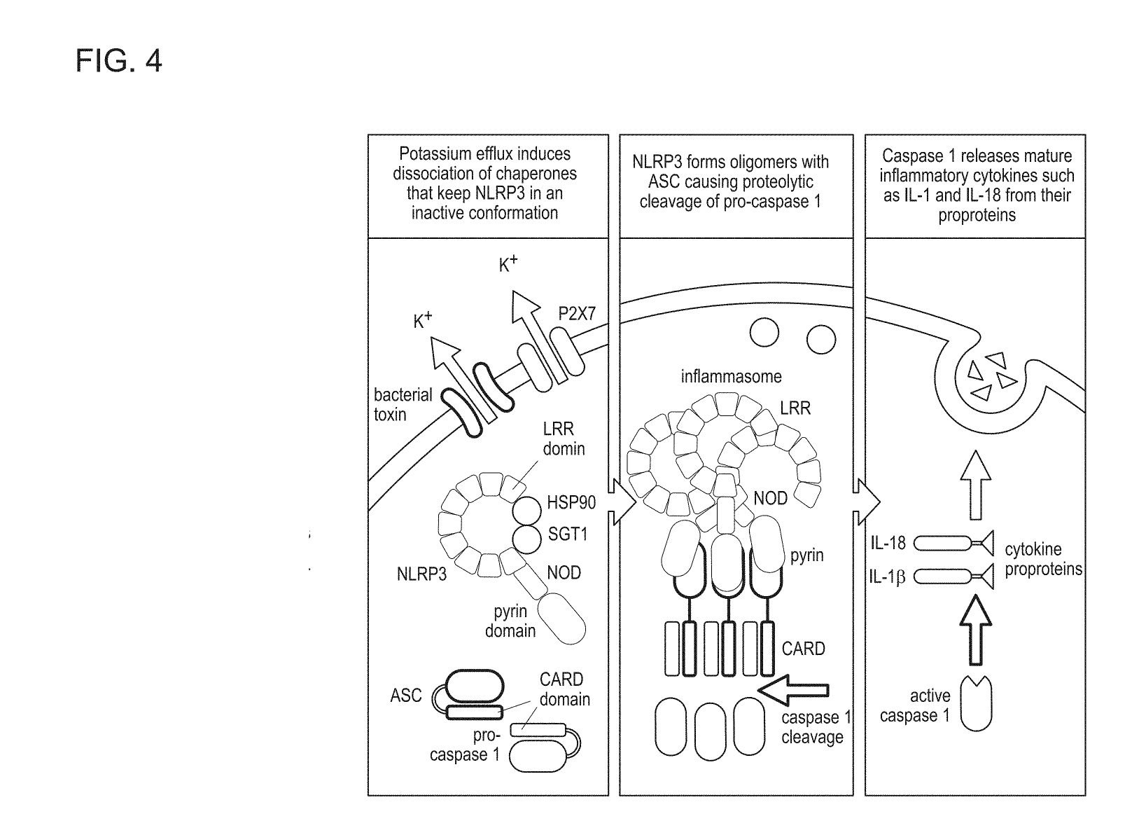

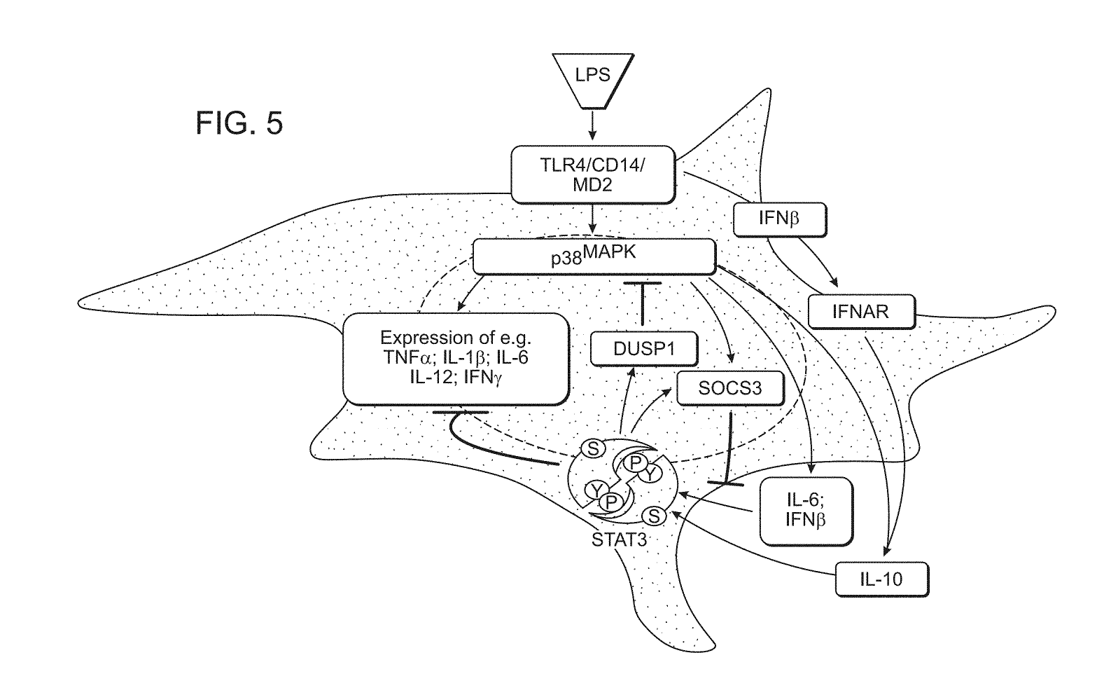

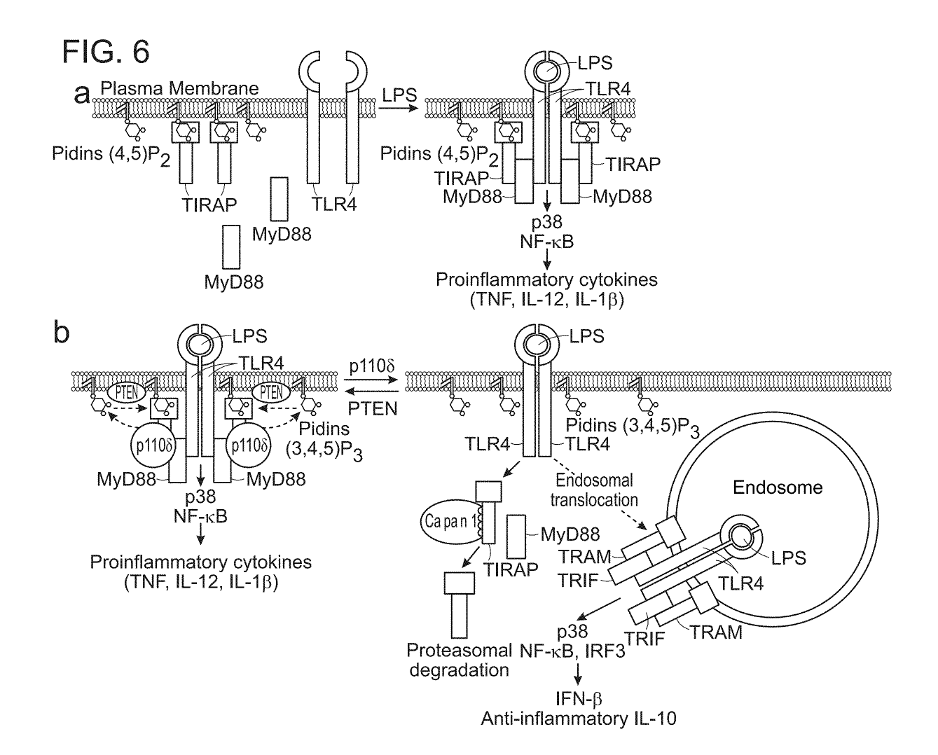

### DETAILED DESCRIPTION OF THE INVENTION

### Glossary

### Definitions

Various terms used throughout this specification shall have the definitions set out herein.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immmunoglobulins (antibodies). The soluble product of an activated T lymphocyte is lymphokines.

The term “administering” as used herein includes in vivo administration, as well as administration directly to tissue ex vivo. Generally, compositions can be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or can be locally administered by means such as, but not limited to, injection, implantation, grafting, topical application, or parenterally.

The term “allogeneic” as used herein refers to being genetically different although belonging to or obtained from the same species.

The term “anergy” as used herein refers to a lack of reaction by the body's defense mechanisms to foreign substances, and consists of a direct induction of peripheral lymphocyte tolerance.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary.

In mammals, there are five classes of antibodies, IgA, IgD, IgE, IgG, and IgM, each with its own class of heavy chain—α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (V_{H}) and light (V_{κ} and V_{λ}) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (V_{H}) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “antigen” and its various grammatical forms refers to any substance that can stimulate the production of antibodies and can combine specifically with them. The terms “epitope” and “antigenic determinant” are used interchangeably herein to refer to an antigenic site on a molecule that an antibody combining site (ACS) recognizes and to which that antibody binds/attaches itself. A given epitope may be primary, secondary, or tertiary-sequence related. Sequential antigenic determinants/epitopes essentially are linear chains. In ordered structures, such as helical polymers or proteins, the antigenic determinants/epitopes essentially would be limited regions or patches in or on the surface of the structure involving amino acid side chains from different portions of the molecule which could come close to one another. These are conformational determinants.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon agregation and mitochondrial localization, induces cytochrome c release. Akt is a critical regulator of GSK-3, and phosphorylation and inactivation of GSK-3 may mediate some of the antiapoptotic effects of Akt.

The term “area under the curve (AUC)” as used herein refers to the area under a plot of plasma concentration of a drug against time after drug administration. The area is determined by the trapazoidal rule: the data points are connected by straight line segments, perpendiculars are erected from the abscissa to each data point, and the sum of the areas of the triangles and trapazoids so constructed is computed. Typically, the area is computed starting at the time the drug is administered and ending when the concentration in plasma is negligible. In practice, the drug concentration is measured at certain discrete points in time and the trapezoidal rule is used to estimate the AUC. The AUC is of use in estimating bioavailability of a drug and in estimating total clearance of a drug.

The term “attenuate” as used herein means to reduce the force, effect, or value of.

The term “autocrine signaling” refers to a type of cell signaling in which a cell secretes signal molecules that act on itself or on other adjacent cells of the same type.

CD3 (TCR complex) is a protein complex composed of four distinct chains. In mammals, the complex contains a CD3γ chain, a CD3δ chain, and two CD3ε chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

The term “chemokine” as used herein refers to a class of chemotactic cytokines that signal leukocytes to move in a specific direction. The terms “chemotaxis” or “chemotactic” refer to the directed motion of a motile cell or part along a chemical concentration gradient towards environmental conditions it deems attractive and/or away from surroundings it finds repellent.

The term “chemoresistance” as used herein refers to the development of a cell phenotype resistant to a variety of structurally and functionally distinct agents. Tumors can be intrinsically resistant prior to chemotherapy, or resistance may be acquired during treatment by tumors that are initially sensitive to chemotherapy. Drug resistance is a multifactorial phenomenon involving multiple interrelated or independent mechanisms. A heterogeneous expression of involved mechanisms may characterize tumors of the same type or cells of the same tumor and may at least in part reflect tumor progression. Exemplary mechanisms that can contribute to cellular resistance include: increased expression of defense factors involved in reducing intracellular drug concentration; alterations in drug-target interaction; changes in cellular response, in particular increased cell ability to repair DNA damage or tolerate stress conditions, and defects in apoptotic pathways.

The term “chemosensitive”, “chemosensitivity” or “chemosensitive tumor” as used herein refers to a tumor that is responsive to a chemotherapy or a chemotherapeutic agent. Characteristics of a chemosensitive tumor include, but are not limit to, reduced proliferation of the population of tumor cells, reduced tumor size, reduced tumor burden, tumor cell death, and slowed/inhibited progression of the population of tumor cells.

The term “chemotherapeutic agent” as used herein refers to chemicals useful in the treatment or control of a disease.

The term “chemotherapy” as used herein refers to a course of treatment with one or more chemotherapeutic agent.

The term “chemotherapy regimen” (“combination chemotherapy”) means chemotherapy with more than one drug in order to benefit from the dissimilar toxicities of the more than one drug. A principle of combination cancer therapy is that different drugs work through different cytotoxic mechanisms; since they have different dose-limiting adverse effects, they can be given together at full doses.

The term “condition”, as used herein, refers to a variety of health states and is meant to include disorders or diseases caused by any underlying mechanism or injury.

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins including interleukin 2 (IL-2), as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1α, IL-1β, IL-2, IL-3, IL-4, IL-5, IL-6, IL-7, IL-8, IL-9, IL-10, IL-11, IL-12/IL-23 P40, IL13, IL-17, IL-18, TGF-β, IFN-γ, GM-CSF, chemokine ligand 1 (CXCL1; GRO-alpha), monocyte chemoattractant protein 1(MCP-1 or CCL2) and TNF-α.

The term “cytometry” as used herein refers to a process in which physical and/or chemical characteristics of single cells, or by extension, of other biological or nonbiological particles in roughly the same size or stage, are measured. In flow cytometry, the measurements are made as the cells or particles pass through the measuring apparatus (a flow cytometer) in a fluid stream. A cell sorter, or flow sorter, is a flow cytometer that uses electrical and/or mechanical means to divert and collect cells (or other small particles) with measured characteristics that fall within a user-selected range of values.

The term “disease” or “disorder,” as used herein, refers to an impairment of health or a condition of abnormal functioning.

The term “drug” as used herein refers to a therapeutic agent or any substance used in the prevention, diagnosis, alleviation, treatment, or cure of disease.

The term “enzymatic activity” as used herein refers to the action of an enzyme (meaning a protein that catalyzes a specific chemical reaction) on its target. It is quantified as the amount of substrate consumed (or product formed) in a given time under given conditions. The term “turnover number” as used herein refers to the number of molecules of substrate that can be converted into product per catalytic site of a given enzyme.

The term “forced expiratory volume” (FEV1) as used herein is the maximal amount of air that can be forcefully exhaled in 1 second. The forced vital capacity (FVC) is the volume of air that can be maximally forcefully exhaled, and therefore contains FEV1. If the FEV1/FVC ratio is <80%, it indicates that an obstructive defect is present.

The term “flow cytometry” as used herein refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multi-parameter, recording several measurements on each cell; therefore, it is possible to identify a homogeneous subpopulation within a heterogeneous population (Marion G. Macey, Flow cytometry: principles and applications, Humana Press, 2007).

The term “hormonal signaling” as used herein refers to signaling via a chemical produced in one part of the body and released into the blood to trigger or regulate particular functions of the body.

The term “immune checkpoint inhibitor” as used herein refers to a type of drug that blocks certain proteins that help keep immune responses in check. These proteins are made by some types of immune system cells (e.g. T cells) and some cancer cells; when these proteins are blocked, the brakes on the immune system are released and T cells can kill cancer cells better. Examples of checkpoint proteins found on T cells or cancer cells include PD-1/PL-L1 and CTLA-4/B7-1/B7-2.

The term “immune privilege” as used herein refers to tissues or organs that do not have a strong inflammatory immune response when challenged. This privileged status is preserved by local active mechanisms that suppress responses to antigens within the privileged tissues.

The terms “immune response” and “immune-mediated” are used interchangeably herein to refer to any functional expression of a subject's immune system, against either foreign or self antigens, whether the consequences of these reactions are beneficial or harmful to the subject.

The term “immune system” as used herein refers to a complex network of cells, tissues, organs, and the substances they make that helps the body fight infections and other diseases. The immune system includes white blood cells and organs and tissues of the lymph system, such as the thymus, spleen, tonsils, lymph nodes, lymph vessels, and bone marrow. The term “immunogenic” as used herein refers to any substance that on is own elicits an immune response.

The term “immune tolerance”, “immunotolerance” or “immunological tolerance” or “tolerance” as used herein refers to a state of unresponsiveness of the immune system to substances that previously had the capacity to elicit an immune response.

The term “immunocompetent” and its other grammatical forms as used herein refers to the ability to produce a normal immune response.

The term “immunocompromised” as used herein refers to having a weakened immune system.

The term “immunomodulatory” and its other grammatical forms as used herein refer(s) to a substance that is capable of augmenting or diminishing an immune response by affecting the expression of chemokines, cytokines and other mediators of immune responses. The term “immunomodulatory agent” as used herein refers to a substance that stimulates or suppresses the immune system. Specific immunomodulating agents affect specific parts of the immune system. Nonspecific immunomodulating agents affect the immune system in a general way. The term “immunomodulatory cell(s)” as used herein refer(s) to cell(s) that are capable of augmenting or diminishing immune responses by expressing chemokines, cytokines and other mediators of immune responses.

The term “immunopotent” as used herein, refers to the ability to activate and guide a naïve immune system to mount a response toward a foreign protein.

The term “immunostimulatory amount” as used herein to describe the disclosed compositions refers to an amount of an immunogenic composition that is effective to increase the ability of the immune system to fight infection and disease, e.g., to stimulate an immune response, for example, as measured by ELISPOT assay (cellular immune response), ICS (intracellular cytokine staining assay), major histocompatibility complex (MHC) tetramer assay to detect and quantify antigen-specific T cells, quantifying the blood population of antigen-specific CD4+ T cells, or quantifying the blood population of antigen specific CD8+ T cells by a measurable amount, when compared to a suitable control.

The term “immunosuppression” as used herein and its other grammatical forms refer to a decrease of the body's immune response and ability of the immune system to fight infections and other diseases. For example, some immunosuppression may be induced with drugs, or may result from disease.

The term “inflammatory cytokines” or “inflammatory mediators” as used herein refers to the molecular mediators of the inflammatory process, which may modulate being either pro- or anti-inflammatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “inhalation” as used herein refers to the act of drawing in a medicated vapor with the breath.

The term “inhalation delivery device” as used herein refers to any device that produces small droplets or an aerosol from a liquid or dry powder aerosol formulation and is used for administration through the mouth in order to achieve pulmonary administration of a drug, e.g., in solution, powder, and the like. Examples of an inhalation delivery device include, but are not limited to, a nebulizer, a metered-dose inhaler, and a dry powder inhaler (DPI).

The term “insufflation” as used herein refers to the act of delivering air, a gas, or a powder under pressure to a cavity or chamber of the body. For example, nasal insufflation relates to the act of delivering air, a gas, or a powder under pressure through the nose.

The term “inhibit” and its various grammatical forms, including, but not limited to, “inhibiting” or “inhibition”, are used herein to refer to reducing the amount or rate of a process, to stopping the process entirely, or to decreasing, limiting, or blocking the action or function thereof. Inhibition can include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor can stop a substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injury,” as used herein, refers to damage or harm to a structure or function of the body caused by an outside agent or force, which can be physical or chemical.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin 2 (IL-2), interleukin-1β (IL-1β), interleukin-6 (IL-6), interleukin-8 (IL-8), and interleukin-12 (IL-12).

Interleukin-6 (IL-6) is a multifunctional cytokine whose major actions include enhancement of immunoglobulin synthesis, activation of T cells, and modulation of acute-phase protein synthesis. Many different types of cells are known to produce IL-6, including monocytes, macrophages, endothelial cells, and fibroblasts, and expression of the IL-6 gene in these cells is known to be regulated by a variety of inducers. Interleukin-1 (IL-1) and tumor necrosis factor-alpha (TNF-α) are two key known inducers of IL-6 gene expression. Other inducers include activators of protein kinase C, calcium ionophore A23187, and various agents causing elevation of intracellular cyclic AMP (cAMP) levels.

The term “kinase” as used herein refers to a type of enzyme that transfers phosphate groups from high-energy donor molecules to specific target molecules or substrates. High-energy donor groups can include, but are not limited, to GTP and ATP.

The term “lipopolysaccharide (LPS)” as used herein refers to is a compound with both lipid and carbohydrate components, derived from the cell wall of gram-negative bacteria. In vivo, infection of gram negative bacteria releases LPS into the blood stream, which activates monocytes. In response, the activated monocytes secret various inflammatory mediators, e.g., Tumor Necrosis Factor-alpha (TNF-α) and Interleukin-6 (IL-6), to combat the infection.

The term “mimetic” as used herein refers to a compound containing chemical moieties that mimic the biological activity of a peptide. For example, if a peptide contains two charged chemical moieties having functional activity, a mimetic places two charged chemical moieties in a spatial orientation and constrained structure so that the charged chemical function is maintained in three-dimensional space. Mimetics may themselves be peptides.

The term “mitogenic compound” as used herein refers to a compound capable of affecting the rate of cell division for at least one cell type under at least one set of conditions suitable for growth or culture.

The term “modify” as used herein means to change, vary, adjust, temper, alter, affect or regulate to a certain measure or proportion in one or more particulars.

The term “modulate” as used herein means to regulate, alter, adapt, or adjust to a certain measure or proportion.

The term “neutrophils” as used herein refers to myeloid cells that are first line phagocytic cells of the innate immune system are also able to produce and release several cytokines and chemokines. The main action exerted by IL-10 on human neutrophils is to influence their ability to express novel proteins, including cytokines. Bazzoni, F. et al. (2010) “Understanding the molecular mechanisms of the multifaceted IL-10 mediated anti-inflammatory response: Lessons from neutrophils,” Eur. J. Immunol. 40: 2360-68). IL-10-R1 conditions neutrophil responsiveness to IL10. Id. Studies have shown that IL-10 even if added concurrently with LPS, needs at least 4 hr to significantly influence LPS-induced mRNA accumulation and extracellular release of cytokines and chemokines, because the neutrophils need to be preliminarily conditioned by pro-inflammatory and anti-inflammatory mediators to express newly formed IL-10R1. Id.

As used herein, the terms “oral” or “orally” refer to the introduction into the body by mouth whereby absorption occurs in one or more of the following areas of the body: the mouth, stomach, small intestine, lungs (also specifically referred to as inhalation), and the small blood vessels under the tongue (also specifically referred to as sublingually).

The term “paracrine signaling” as used herein refers to short-range cell-cell communication via secreted signal molecules that act on adjacent cells.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or infusion techniques. A parenterally administered composition of the described invention is delivered using a needle, e.g., a surgical needle. The term “surgical needle” as used herein, refers to any needle adapted for delivery of fluid (i.e., capable of flow) compositions of the described invention into a selected anatomical structure. Injectable preparations, such as sterile injectable aqueous or oleaginous suspensions, can be formulated according to the known art using suitable dispersing or wetting agents and suspending agents.

The term “pathogen-associated molecular patterns (PAMPs)” as used herein refers to molecules specifically associated with groups of pathogens that are recognized by cells of the innate immune system.

The term “pattern recognition receptor (PRR)” as used herein refers to receptors of the innate immune system that recognize common molecular patterns on pathogen surfaces.

The term “pharmaceutical composition” as used herein refers to a preparation comprising a pharmaceutical product, drug, metabolite, or active ingredient.

The term “proliferate” and its various grammatical forms as used herein refers to an increase in number. The terms “proliferate” and “expand” are used interchangeably herein.

“Rectal” or “rectally” as used herein refers to introduction into the body through the rectum where absorption occurs through the walls of the rectum.

The term “reduced” or “to reduce” as used herein refers to a diminution, a decrease, an attenuation or abatement of the degree, intensity, extent, size, amount, density or number.

The term “regulatory T cells (Tregs)”, formerly known as suppressor T cells, as used herein refers to a subpopulation of T cells which modulate the immune system to maintain tolerance to self-antigens and abrogate autoimmune disease.

The term “stimulate” in any of its grammatical forms as used herein refers to inducing activation or increasing activity.

As used herein, the terms “subject” or “individual” or “patient” are used interchangeably to refer to a member of an animal species of mammalian origin, including humans.

As used herein the term “a subject in need thereof” is used to refer to a patient who (i) is immunotolerant to an immunostimulating agent; (ii) is at risk for becoming immunotolerant to an immunostimulating agent; (iii) will suffer from a disorder that was responsive but is no longer responsive to an otherwise immunostimulating therapeutic agent; (iv) is suffering from a disorder that was responsive but is no longer responsive to an otherwise immunostimulating therapeutic agent; or (iii) has suffered from a disease that was responsive but is no longer responsive to an otherwise immunostimulating therapeutic agent. According to some embodiments, the phrase also is used to refer to a patient who (i) will receive the described treatment; (b) is receiving the described treatment; or (c) has received the described treatment, unless the context and usage of the phrase indicates otherwise.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, “therapeutically effective amount”, an “amount effective”, or “pharmaceutically effective amount” of an active agent is used interchangeably to refer to an amount that is sufficient to provide the intended benefit of treatment. An effective amount of the active agent(s) that can be employed according to the described invention generally ranges from about 0.25 mg/kg body weight to about 160 mg/kg body weight per dose, with three doses given per day. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” refers to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined.

In this case, a target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The terms “T_{H}1” and “T_{H}2” as used herein refers to subsets of effector CD4 T cells characterized by the cytokines they produce. T_{H}1 cells are mainly involved in activating macrophages but can also help stimulate B cells to produce antibody. T_{H}2 cells are involved in stimulating B cells to produce antibody.

The term “T_{H}17” as used herein refers to a subset of CD4 T cells characterized by production of the cytokine IL-17. They help recruit neutrophils to sites of infection.

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacyterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “topical” refers to administration of a composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably.

As used herein the term “treating” includes abrogating, substantially inhibiting, slowing or reversing the progression of a condition, substantially ameliorating clinical symptoms of a condition, or substantially preventing the appearance of clinical symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor necrosis factor (TNF, also referred as TNF-α)” as used herein refers to a cytokine involved in systemic inflammation; it is a member of a group of cytokines that stimulate the acute phase reaction. Studies have shown that TNF-α induces expression of IL-6 via three distinct signaling pathways inside the cell, i.e., 1) NF-κB pathway 2) MAPK pathway, and 3) death signaling pathway.

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants of polypeptide YARAAARQARAKALARQLGVAA (SEQ ID NO: 1) having single or multiple amino acid substitutions, deletions, additions or replacements, but functionally equivalent to SEQ ID NO: 1. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, for example, an epitope for an antibody. The techniques for obtaining such variants, including, but not limited to, genetic (suppressions, deletions, mutations, etc.), chemical, and enzymatic techniques, are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “vehicle” as used herein refers to a substance that facilitates the use of a drug or other material that is mixed with it.

### Pharmaceutical Formulations

According to one embodiment, the described invention provides a pharmaceutical formulation comprising an inhibitor of MK2 kinase. According to another embodiment, the MK2 inhibitor is a polypeptide. According to another embodiment, the polypeptide includes, but is not limited to, MMI-0100 (YARAAARQARAKALARQLGVAA (SEQ ID NO: 1)), its functional equivalents or a mimic thereof.

According to one embodiment, the pharmaceutical formulation comprises a neat spray dried dispersion comprising MMI-0100 (YARAAARQARAKALARQLGVAA; SEQ ID NO: 1) or a functional equivalent thereof, 5% w/w solids. According to another embodiment, the pharmaceutical formulation comprises a neat spray dried dispersion comprising MMI-0100 (YARAAARQARAKALARQLGVAA; SEQ ID NO: 1) or a functional equivalent thereof, 1% w/w solids. According to another embodiment, the pharmaceutical formulation comprises a spray dried dispersion comprising 80/20 MMI-0100 (YARAAARQARAKALARQLGVAA; SEQ ID NO: 1) or a functional equivalent thereof/trehalose. According to another embodiment, the pharmaceutical formulation comprises a spray dried dispersion comprising 92.5/7.5 MMI-0100 (YARAAARQARAKALARQLGVAA; SEQ ID NO: 1) or a functional equivalent thereof/trehalose.

A spray-dried dispersion (SDD) is a single-phase, amorphous molecular dispersion of a drug in a polymer matrix. It is a solid solution with a compound (e.g., drug) molecularly “dissolved” in a solid matrix. SDDs are obtained by dissolving drug and polymer in an organic solvent to obtain a solution and then spray-drying the solution. The use of spray drying for pharmaceutical applications results in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been consistently demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

According to one embodiment, the pharmaceutical formulation comprises MMI-0100 (YARAAARQARAKALARQLGVAA; SEQ ID NO: 1) or a functional equivalent thereof and 0.9% NaCl (saline). According to another embodiment, the pharmaceutical formulation comprises 7 mg/mL, 6 mg/mL, 5 mg/mL, 4 mg/mL, 3 mg/mL, 2 mg/mL, or 1 mg/mL MMI-0100 (YARAAARQARAKALARQLGVAA; SEQ ID NO: 1) or a functional equivalent thereof. According to anther embodiment, the pharmaceutical formulation comprises 0.9 mg/mL, 0.8 mg/mL, 0.7 mg/mL, 0.6 mg/mL, 0.5 mg/mL, 0.4 mg/mL, 0.3 mg/mL, 0.2 mg/mL, or 0.1 mg/mL MMI-0100 (YARAAARQARAKALARQLGVAA; SEQ ID NO: 1) or a functional equivalent thereof. According to another embodiment, the formulation comprising MMI-0100 (YARAAARQARAKALARQLGVAA; SEQ ID NO: 1) or a functional equivalent thereof is a liquid formulation. According to another embodiment, the liquid formulation is aerosolized.

According to one embodiment, the pharmaceutical formulation comprises MMI-0100 (YARAAARQARAKALARQLGVAA; SEQ ID NO: 1) or a functional equivalent thereof and glycerin.

According to one embodiment, the pharmaceutical formulation comprises MMI-0100 (YARAAARQARAKALARQLGVAA; SEQ ID NO: 1) or a functional equivalent thereof and a nano-polyplex polymer. According to another embodiment, the nano-polyplex polymer is poly(acrylic acid) (PAA). According to another embodiment, the nano-polyplex polymer is poly(propylacrylic acid) (PPAA). According to another embodiment, the pharmaceutical formulation comprises a charge ratio (CR) of MMI-0100 (YARAAARQARAKALARQLGVAA; SEQ ID NO: 1) or a functional equivalent thereof to PPAA ([NH₃⁺]_{MK2i}:[COO⁻]_{PPAA}) selected from the group consisting of 10:1, 9:1, 8:1, 7:1, 6:1, 5:1, 4:1, 3:1, 2:1, 1:1, 1:1.5, 1:2, 1:3, 1:4, 1:5, 1:6, 1:7, 1:8, 1:9 and 1:10. According to another embodiment, the pharmaceutical formulation comprises a charge ratio of MMI-0100 (YARAAARQARAKALARQLGVAA; SEQ ID NO: 1) or a functional equivalent thereof to PPAA ([NH₃⁺]_{MK2i}:[COO⁻]_{PPAA}) of 1:3.

According to another embodiment, the functional equivalent of the polypeptide YARAAARQARAKALARQLGVAA (MMI-0100; SEQ ID NO: 1) has a substantial sequence identity to amino acid sequence YARAAARQARAKALARQLGVAA (SEQ ID NO: 1).

According to another embodiment, the functional equivalent of the polypeptide YARAAARQARAKALARQLGVAA (MMI-0100; SEQ ID NO: 1) has at least 80 percent sequence identity to amino acid sequence YARAAARQARAKALARQLGVAA (SEQ ID NO: 1). According to another embodiment, the functional equivalent of the polypeptide YARAAARQARAKALARQLGVAA (MMI-0100; SEQ ID NO: 1) has at least 90 percent sequence identity to amino acid sequence YARAAARQARAKALARQLGVAA (SEQ ID NO: 1). According to another embodiment, the functional equivalent of the polypeptide YARAAARQARAKALARQLGVAA (MMI-0100; SEQ ID NO: 1) has at least 95 percent sequence identity to amino acid sequence YARAAARQARAKALARQLGVAA (SEQ ID NO: 1).

According to another embodiment, the functional equivalent of the polypeptide YARAAARQARAKALARQLGVAA (MMI-0100; SEQ ID NO: 1) is a polypeptide of amino acid sequence YARAAARQARAKALNRQLGVA (MMI-0200; SEQ ID NO: 19)

According to another embodiment, the functional equivalent of the polypeptide YARAAARQARAKALARQLGVAA (MMI-0100; SEQ ID NO: 1) is a polypeptide of amino acid sequence FAKLAARLYRKALARQLGVAA (MMI-0300; SEQ ID NO: 3).

According to another embodiment, the functional equivalent of the polypeptide YARAAARQARAKALARQLGVAA (SEQ ID NO: 1) is a polypeptide of amino acid sequence KAFAKLAARLYRKALARQLGVAA (MMI-0400; SEQ ID NO: 4).

According to another embodiment, the functional equivalent of the polypeptide YARAAARQARAKALARQLGVAA (SEQ ID NO: 1) is a polypeptide of amino acid sequence YARAAARQARAKALARQLAVA (SEQ ID NO: 5).

According to another embodiment, the functional equivalent of the polypeptide YARAAARQARAKALARQLGVAA (SEQ ID NO: 1) is a polypeptide of amino acid sequence YARAAARQARAKALARQLGVA (SEQ ID NO: 6).

According to another embodiment, the functional equivalent of the polypeptide YARAAARQARAKALARQLGVAA (SEQ ID NO: 1) is a polypeptide of amino acid sequence HRRIKAWLKKIKALARQLGVAA (MMI-0500; SEQ ID NO: 7).

According to another embodiment, the functional equivalent of the polypeptide YARAAARQARAKALARQLGVAA (SEQ ID NO: 1) is a polypeptide of amino acid sequence YARAAARQARAKALNRQLAVAA (MMI0600, SEQ ID NO: 23)

According to another embodiment, the functional equivalent of the polypeptide YARAAARQARAKALARQLGVAA (SEQ ID NO: 1) is a polypeptide of amino acid sequence YARAAARQARAKALNRQLAVA (MMI0600-2, SEQ ID NO: 24).

According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has a substantial identity to amino acid sequence KALARQLGVAA (SEQ ID NO: 2).

According to another embodiment, the second polypeptide has at least 70 percent sequence identity to amino acid sequence KALARQLGVAA (SEQ ID NO: 2), and the pharmaceutical formulation is effective to inhibit the kinase activity of Mitogen-Activated Protein Kinase-Activated Protein Kinase 2 (MK2). According to another embodiment, the second polypeptide has at least 80 percent sequence identity to amino acid sequence KALARQLGVAA (SEQ ID NO: 2), and the pharmaceutical formulation is effective to inhibit the kinase activity of Mitogen-Activated Protein Kinase-Activated Protein Kinase 2 (MK2). According to another embodiment, the second polypeptide has at least 90 percent sequence identity to amino acid sequence KALARQLGVAA (SEQ ID NO: 2), and the pharmaceutical formulation is effective to inhibit the kinase activity of Mitogen-Activated Protein Kinase-Activated Protein Kinase 2 (MK2). According to another embodiment, the second polypeptide has at least 95 percent sequence identity to amino acid sequence KALARQLGVAA (SEQ ID NO: 2), and the pharmaceutical formulation is effective to inhibit the kinase activity of Mitogen-Activated Protein Kinase-Activated Protein Kinase 2 (MK2).

According to another embodiment, the second polypeptide is a polypeptide of amino acid sequence KALARQLAVA (SEQ ID NO: 8).

According to another embodiment, the second polypeptide is a polypeptide of amino acid sequence KALARQLGVA (SEQ ID NO: 9).

According to another embodiment, the second polypeptide is a polypeptide of amino acid sequence KALNRQLAVAA (SEQ ID NO: 25)

According to another embodiment, the second polypeptide is a polypeptide of amino acid sequence KALNRQLAVA (SEQ ID NO: 26).

According to another embodiment, the second polypeptide is a polypeptide of amino acid sequence KALARQLGVAA (SEQ ID NO: 10).

According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11), and the second polypeptide is of amino acid sequence KALARQLGVAA (SEQ ID NO: 2).

According to another embodiment, the first polypeptide is a polypeptide of amino acid sequence WLRRIKAWLRRIKA (SEQ ID NO: 12).

According to another embodiment, first polypeptide is a polypeptide of amino acid sequence WLRRIKA (SEQ ID NO: 13).

According to another embodiment, the first polypeptide is a polypeptide of amino acid sequence YGRKKRRQRRR (SEQ ID NO: 14).

According to another embodiment, the first polypeptide is a polypeptide of amino acid sequence WLRRIKAWLRRI (SEQ ID NO: 15).

According to another embodiment, the first polypeptide is a polypeptide of amino acid sequence FAKLAARLYR (SEQ ID NO: 16).

According to another embodiment, the first polypeptide is a polypeptide of amino acid sequence KAFAKLAARLYR (SEQ ID NO: 17).

According to another embodiment, the first polypeptide is a polypeptide of amino acid sequence HRRIKAWLKKI (SEQ ID NO: 18).

According to some embodiments, in order to enhance drug efficacy and to prevent accumulation of the polypeptide YARAAARQARAKALARQLGVAA (SEQ ID NO: 1) or its functional equivalent in non-target tissues, the polypeptide of the present invention of amino acid sequence YARAAARQARAKALARQLGVAA (SEQ ID NO: 1) or its functional equivalent can be linked or associated with a targeting moiety, which directs the polypeptide to a specific cell type or tissue. Examples of the targeting moiety include, but are not limited to, (i) a ligand for a known or unknown receptor or (ii) a compound, a peptide, or a monoclonal antibody that binds to a specific molecular target, e.g., a peptide or carbohydrate, expressed on the surface of a specific cell type.

According to some embodiments, the polypeptide of the described invention is chemically synthesized. Such a synthetic polypeptide, prepared using the well-known techniques of solid phase, liquid phase, or peptide condensation techniques, or any combination thereof, may include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptide may be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers, each incorporated by reference herein in its entirety.

According to some embodiments, the polypeptide of the invention comprises D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Examples of synthetic amino acid substitutions include ornithine for lysine, and norleucine for leucine or isoleucine.

According to some embodiments, the polypeptide may be linked to other compounds to promote an increased half-life in vivo, such as polyethylene glycol or dextran. Such linkage can be covalent or non-covalent as is understood by those of skill in the art. According to some other embodiments, the polypeptide may be encapsulated in a micelle, such as a micelle made of poly(ethyleneglycol)-block-poly(polypropylenglycol) or poly(ethyleneglycol)-block-polylactide. According to some other embodiments, the polypeptide may be encapsulated in degradable nano- or micro-particles composed of degradable polyesters including, but not limited to, polylactic acid, polyglycolide, and polycaprolactone.

According to some embodiments, the pharmaceutical formulation of the described invention may be administered by an inhalation device. Examples of the inhalation device that can be used for administering the pharmaceutical formulation includes, but is not limited to, a nebulizer, a metered-dose inhaler, a dry powder inhaler and an aqueous droplet inhaler.

Nebulizers, which actively aerosolize a liquid formulation and operate continuously once loaded, require either compressed air or an electrical supply. Exemplary nebulizers include, a vibrating mesh nebulizer, a jet nebulizer (also known as an atomizer) and an ultrasonic wave nebulizer. Exemplary vibrating mesh nebulizers include, but are not limited to, Respironics i-Neb, Omron MicroAir, Beurer Nebulizer IH50 and Aerogen Aeroneb. Acorn-I, Acorn-II, AquaTower, AVA-NEB, Cirrhus, Dart, DeVilbiss 646, Downdraft, Fan Jet, MB-5, Misty Neb, Salter Labs 8900, Sidestream, Updraft-II, and Whisper Jet are examples of a jet nebulizer. Exemplary ultrasonic nebulizers include, but are not limited to, an Omron NE-U17 nebulizer and a Beurer Nebulizer IH30.

Metered-dose inhalers (MDI) use a propellant to deliver a fixed volume of liquid solution or suspension to a patient in the form of a spray.

Dry powder inhalers (DPI) contain an active drug mixed with an excipient containing much larger particles (e.g., lactose) to which the drug attaches. During aerosolization, the active drug is stripped from the carrier and inhaled while the the carrier particles impact on the mouth and throat and are ingested. DPIs synchronize drug delivery with inhalation.

According to one embodiment, the polypeptide of the described invention may be in the form of a dispersible dry powder for delivery by inhalation or insufflation (either through the mouth or through the nose, respectively). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the described invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition, such as gelatin or plastic capsules, with a removable portion that allows a stream of gas (e.g., air) to be directed into the container to disperse the dry powder composition. Such containers are exemplified by those shown in U.S. Pat. Nos. 4,227,522; 4,192,309; and 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference in their entireties.

Aqueous droplet inhalers (ADI) deliver a pre-metered dose of liquid formulation without using a propellant. ADIs actively aerosolize liquid producing a soft mist of fine particles. Berodual Respimat® (Boehringer Ingelheim Pharma Gmbh & Co.) is an exemplary aqueous droplet inhaler.

According to one embodiment, the polypeptide of the described invention may be in the form of a nebulization solution. According to another embodiment, the nebulization formulation does not contain mannitol. According to one embodiment, the nebulization solution is delivered by a nebulizer.

According to another embodiment, the polypeptide may be prepared in a solid form (including granules, powders or suppositories) or in a liquid form (e.g., solutions, suspensions, or emulsions).

According to another embodiment, the polypeptide of the described invention may be in the form of a nano-polyplex. According to one embodiment, the nan-polyplex polymer is anionic. According to another embodiment, the nano-polyplex polymer is an endosomolytic polymer. Exemplary nano-polyplex polymers include, but are not limited to, chitosan, polyethyleneimine (PEI), polyethylene oxide (PEO), poly(organophos-phazene), poly(acrylic acid) (PAA) and poly(propylacrylic acid) (PPAA).

According to one embodiment, the formulation of the described invention may be delivered by implanting a biomedical device. The biomedical device includes, but is not limited to, a graft. According to another embodiment, the formulation may be disposed on or in the graft. According to another embodiment, the graft includes, but is not limited to, a vascular graft. According to another embodiment, the formulation may be delivered parenterally. According to another embodiment, the formulation may be delivered topically.

According to another embodiment, the formulation of the described invention comprises a carrier. The carrier can include, but is not limited to, a release agent, such as a sustained release or delayed release carrier. According to such embodiments, the carrier can be any material capable of sustained or delayed release of the polypeptide to provide a more efficient administration, e.g., resulting in less frequent and/or decreased dosage of the polypeptide, improving ease of handling, and extending or delaying effects on diseases, disorders, conditions, syndromes, and the like. Non-limiting examples of such carriers include liposomes, microsponges, microspheres, or microcapsules of natural and synthetic polymers and the like. Liposomes may be formed from a variety of phospholipids, including, but not limited to, cholesterol, stearylamines or phosphatidylcholines.

According to another embodiment, the polypeptide of the invention may be applied in a variety of solutions. A suitable formulation is sterile, dissolves sufficient amounts of the therapeutic polypeptide, preserves stability of the therapeutic polypeptide, and is not harmful for the proposed application. For example, the compositions of the described invention may be formulated as aqueous suspensions wherein the active ingredient(s) is (are) in admixture with excipients suitable for the manufacture of aqueous suspensions.

Such excipients include, without limitation, suspending agents (e.g., sodium carboxymethylcellulose, methylcellulose, hydroxy-propylmethylcellulose, sodium alginate, polyvinylpyrrolidone, gum tragacanth, and gum acacia), dispersing or wetting agents including, a naturally-occurring phosphatide (e.g., lecithin), or condensation products of an alkylene oxide with fatty acids (e.g., polyoxyethylene stearate), or condensation products of ethylene oxide with long chain aliphatic alcohols (e.g., heptadecaethyl-eneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate).

Compositions of the described invention also may be formulated as oily suspensions by suspending the active ingredient in a vegetable oil (e.g., arachis oil, olive oil, sesame oil or coconut oil) or in a mineral oil (e.g., liquid paraffin). The oily suspensions may contain a thickening agent (e.g., beeswax, hard paraffin or cetyl alcohol).

Compositions of the described invention also may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients also may be present.

Compositions of the described invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

According to some embodiments, the pharmaceutical formulations of the described invention are effective to inhibit a kinase activity of Mitogen-Activated Protein Kinase-Activated Protein Kinase 2 (MK2). According to some embodiments, the pharmaceutical formulations of the described invention are effective to inhibit at least 50% of the kinase activity of MK2 kinase. According to some embodiments, the pharmaceutical formulations of the described invention inhibit at least 55% of the kinase activity of MK2 kinase. According to some embodiments, the pharmaceutical formulations of the described invention are effective to inhibit at least 60% of the kinase activity of MK2 kinase. According to some embodiments, the pharmaceutical formulations or the described invention are effective to inhibit at least 65% of the kinase activity of MK2 kinase. According to some embodiments, the pharmaceutical formulations of the described invention are effective to inhibit at least 70% of the kinase activity of MK2 kinase. According to some embodiments, the pharmaceutical formulations of the described invention are effective to inhibit at least 75% of the kinase activity of MK2 kinase. According to some embodiments, the pharmaceutical formulations of the described invention are effective to inhibit at least 80% of the kinase activity of MK2 kinase. According to some embodiments, the pharmaceutical formulations of the described invention are effective to inhibit at least 85% of the kinase activity of MK2 kinase. According to some embodiments, the pharmaceutical formulations of the described invention are effective to inhibit at least 90% of the kinase activity of MK2 kinase. According to some embodiments, the pharmaceutical formulations of the described invention are effective to inhibit at least 95% of the kinase activity of MK2 kinase.

According to another embodiment, the pharmaceutical formulation is effective to inhibit a kinase activity of Mitogen-Activated Protein Kinase-Activated Protein Kinase 3 (MK3). According to some such embodiments, the pharmaceutical formulation is effective to inhibit at least 50% of the kinase activity of MK3 kinase. According to some such embodiments, the pharmaceutical formulation is effective to inhibit at least 55% of the kinase activity of MK3 kinase. According to some such embodiments, the pharmaceutical formulation is effective to inhibit at least 60% of the kinase activity of MK3 kinase. According to another embodiment, the pharmaceutical formulation is effective to inhibit at least 65% of the kinase activity of MK3 kinase. According to another embodiment, the pharmaceutical formulation is effective to inhibit at least 70% of the kinase activity of MK3 kinase. According to another embodiment, the pharmaceutical formulation is effective to inhibit at least 75% of the kinase activity of MK3 kinase. According to another embodiment, the pharmaceutical formulation is effective to inhibit at least 80% of the kinase activity of MK3 kinase. According to another embodiment, the pharmaceutical formulation is effective to inhibit at least 85% of the kinase activity of MK3 kinase. According to another embodiment, the pharmaceutical formulation is effective to inhibit at least 90% of the kinase activity of MK3 kinase. According to another embodiment, the pharmaceutical formulation is effective to inhibit at least 95% of the kinase activity of MK3 kinase.

According to another embodiment, the pharmaceutical formulation is effective to inhibit a kinase activity of calcium/calmodulin-dependent protein kinase I (CaMKI). According to some such embodiments, the pharmaceutical formulation is effective to inhibit at least 50% of the kinase activity of Ca2+/calmodulin-dependent protein kinase I (CaMKI). According to some such embodiments, the pharmaceutical formulation is effective to inhibit at least 55% of the kinase activity of Ca2+/calmodulin-dependent protein kinase I (CaMKI). According to some such embodiments, the pharmaceutical formulation is effective to inhibit at least 60% of the kinase activity of Ca2+/calmodulin-dependent protein kinase I (CaMKI). According to another embodiment, the pharmaceutical formulation is effective to inhibit at least 65% of the kinase activity of Ca2+/calmodulin-dependent protein kinase I (CaMKI). According to another embodiment, the pharmaceutical formulation is effective to inhibit at least 70% of the kinase activity of Ca2+/calmodulin-dependent protein kinase I (CaMKI). According to another embodiment, the pharmaceutical formulation is effective to inhibit at least 75% of the kinase activity of Ca2+/calmodulin-dependent protein kinase I (CaMKI). According to another embodiment, the pharmaceutical formulation is effective to inhibit at least 80% of the kinase activity of Ca2+/calmodulin-dependent protein kinase I (CaMKI). According to another embodiment, the pharmaceutical formulation is effective to inhibit at least 85% of the kinase activity of Ca2+/calmodulin-dependent protein kinase I (CaMKI). According to another embodiment, the pharmaceutical formulation is effective to inhibit at least 90% of the kinase activity of Ca2+/calmodulin-dependent protein kinase I (CaMKI). According to another embodiment, the pharmaceutical formulation is effective to inhibit at least 95% of the kinase activity of Ca2+/calmodulin-dependent protein kinase I (CaMKI).

According to another embodiment, the pharmaceutical formulation is effective to inhibit a kinase activity of BDNF/NT-3 growth factors receptor (TrkB). According to some such embodiments, the pharmaceutical formulation is effective to inhibit at least 50% of the kinase activity of BDNF/NT-3 growth factors receptor (TrkB). According to some such embodiments, the pharmaceutical formulation is effective to inhibit at least 55% of the kinase activity of BDNF/NT-3 growth factors receptor (TrkB). According to some such embodiments, the pharmaceutical formulation is effective to inhibit at least 60% of the kinase activity of BDNF/NT-3 growth factors receptor (TrkB). According to another embodiment, the pharmaceutical formulation is effective to inhibit at least 65% of the kinase activity of BDNF/NT-3 growth factors receptor (TrkB). According to another embodiment, the pharmaceutical formulation is effective to inhibit at least 70% of the kinase activity of BDNF/NT-3 growth factors receptor (TrkB). According to another embodiment, the pharmaceutical formulation is effective to inhibit at least 75% of the kinase activity of BDNF/NT-3 growth factors receptor (TrkB). According to another embodiment, the pharmaceutical formulation is effective to inhibit at least 80% of the kinase activity of BDNF/NT-3 growth factors receptor (TrkB). According to another embodiment, the pharmaceutical formulation is effective to inhibit at least 85% of the kinase activity of BDNF/NT-3 growth factors receptor (TrkB). According to another embodiment, the pharmaceutical formulation is effective to inhibit at least 90% of the kinase activity of BDNF/NT-3 growth factors receptor (TrkB). According to another embodiment, the pharmaceutical formulation is effective to inhibit at least 95% of the kinase activity of BDNF/NT-3 growth factors receptor (TrkB).

According to another embodiment, the pharmaceutical formulation is effective to inhibit a kinase activity of Mitogen-Activated Protein Kinase-Activated Protein Kinase 2 (MK2) and a kinase activity of calcium/calmodulin-dependent protein kinase I (CaMKI).

According to another embodiment, the pharmaceutical formulation is effective to inhibit a kinase activity of Mitogen-Activated Protein Kinase-Activated Protein Kinase 2 (MK2) and a kinase activity of BDNF/NT-3 growth factors receptor (TrkB).

According to another embodiment, the pharmaceutical formulation is effective to inhibit a kinase activity of Mitogen-Activated Protein Kinase-Activated Protein Kinase 2 (MK2), a kinase activity of calcium/calmodulin-dependent protein kinase I (CaMKI), and a kinase activity of BDNF/NT-3 growth factors receptor (TrkB).

According to another embodiment, the pharmaceutical formulation is effective to inhibit at least 65% of the kinase activity of Mitogen-Activated Protein Kinase-Activated Protein Kinase 2 (MK2) and at least 65% of the kinase activity of calcium/calmodulin-dependent protein kinase I (CaMKI).

According to another embodiment, the pharmaceutical formulation is effective to inhibit at least 65% of the kinase activity of Mitogen-Activated Protein Kinase-Activated Protein Kinase 2 (MK2) and at least 65% of the kinase activity of BDNF/NT-3 growth factors receptor (TrkB).

According to another embodiment, the pharmaceutical formulation is effective to inhibit at least 65% of the kinase activity of Mitogen-Activated Protein Kinase-Activated Protein Kinase 2 (MK2), at least 65% of the kinase activity of calcium/calmodulin-dependent protein kinase I (CaMKI), and at least 65% of the kinase activity of BDNF/NT-3 growth factors receptor (TrkB).

According to another embodiment, the pharmaceutical formulation is effective to inhibit the kinase activity of at least one kinase selected from the group of MK2, MK3, CaMKI, TrkB, without substantially inhibiting the activity of one or more other selected kinases from the remaining group listed in Table 1 herein.

TABLE 1
Kinase Profiling Assay

| | MMI-0100 | MMI-0200 | MMI-0300 | MMI-0400 | MMI-0500 |
|---|---|---|---|---|---|
| | (SEQ ID | (SEQ ID | (SEQ ID | (SEQ ID | (SEQ ID |
| | NO: 1) | NO: 19) | NO: 3) | NO: 4) | NO: 7) |
| | (100 μM) | (100 μM) | (100 μM) | (100 μM) | (100 μM) |
| Abl(h) | 136 | 107 | 69 | 84 | 16 |
| Abl (H396P) (h) | 130 | 121 | 101 | 105 | 51 |
| Abl (M351T)(h) | 128 | 119 | 90 | 121 | 61 |
| Abl (Q252H) (h) | 105 | 107 | 82 | 98 | 40 |
| Abl(T315I)(h) | 98 | 108 | 97 | 105 | 16 |
| Abl(Y253F)(h) | 104 | 102 | 86 | 78 | 29 |
| ACK1(h) | 106 | 97 | 104 | 95 | 64 |
| ALK(h) | 118 | 95 | 19 | 16 | 12 |
| ALK4(h) | 124 | 152 | 140 | 130 | 81 |
| Arg(h) | 89 | 82 | 72 | 84 | 22 |
| AMPKα1(h) | 107 | 108 | 71 | 87 | 35 |
| AMPKα2(h) | 121 | 88 | 54 | 58 | 9 |
| ARK5(h) | 108 | 93 | 78 | 69 | 20 |
| ASK1(h) | 100 | 101 | 80 | 69 | −4 |
| Aurora-A(h) | 120 | 107 | 92 | 119 | 110 |
| Aurora-B(h) | 94 | 166 | 128 | 150 | 5 |
| Axl(h) | 81 | 99 | 52 | 41 | 12 |
| Bmx(h) | 62 | 76 | N/D | 26 | 45 |
| BRK(h) | 70 | 127 | 35 | 18 | 41 |
| BrSK1(h) | 100 | 93 | 67 | 76 | 72 |
| BrSK2(h) | 129 | 102 | 83 | 86 | 84 |
| BTK(h) | 112 | 100 | 102 | 94 | 18 |
| BTK(R28H)(h) | 91 | 104 | 74 | 24 | 10 |
| CaMKI(h) | 13 | 21 | 1 | 0 | −1 |
| CaMKIIβ(h) | 58 | 53 | 2 | 11 | 3 |
| CaMKIIγ(h) | 106 | 94 | 5 | 3 | 3 |
| CaMKIδ(h) | 59 | 47 | 10 | 17 | 0 |
| CaMKIIδ(h) | 89 | 2 | 1 | 2 | 1 |
| CaMKIV(h) | 87 | 71 | 17 | 18 | −1 |
| CDK1/cyclinB(h) | 96 | 115 | 73 | 74 | 57 |
| CDK2/cyclinA(h) | 97 | 114 | 86 | 92 | 87 |
| CDK2/cyclinE(h) | 106 | 112 | 94 | 83 | 19 |
| CDK3/cyclinE(h) | 106 | 104 | 94 | 92 | 8 |
| CDK5/p25(h) | 114 | 97 | 89 | 92 | 66 |
| CDK5/p35(h) | 94 | 92 | 79 | 76 | 59 |
| CDK6/cyclinD3(h) | 103 | 100 | 86 | 85 | 23 |
| CDK7/cyclinH/MAT1(h) | 89 | 67 | 65 | 47 | 15 |
| CDK9/cyclin T1(h) | 228 | 103 | 91 | 235 | 6 |
| CHK1(h) | 97 | 115 | 91 | 87 | 65 |
| CHK2(h) | 104 | 105 | 66 | 54 | 13 |
| CHK2(I157T)(h) | 97 | 85 | 43 | 41 | 3 |
| CHK2(R145W)(h) | 97 | 81 | 33 | 31 | 3 |
| CK1γ1(h) | 110 | 98 | 111 | 116 | 109 |
| CK1γ2(h) | 119 | 104 | 123 | 114 | 119 |
| CK1γ3(h) | 105 | 96 | 125 | 115 | 114 |
| CK1δ(h) | 115 | 92 | 92 | 93 | 78 |
| CK2(h) | 90 | 83 | 90 | 101 | 93 |
| CK2α2(h) | 104 | 88 | 105 | 96 | 103 |
| CLK2(h) | 88 | 97 | 103 | 116 | 116 |
| CLK3(h) | 108 | 76 | 61 | 84 | 76 |
| cKit(h) | 95 | 110 | 53 | 43 | 45 |
| cKit(D816V)(h) | 117 | 118 | 60 | 35 | 30 |
| cKit(D816H)(h) | 79 | 106 | 126 | 143 | 194 |
| cKit(V560G)(h) | 94 | 115 | 102 | 124 | 198 |
| cKit(V654A)(h) | 69 | 113 | 134 | 150 | 223 |
| CSK(h) | 70 | 33 | 49 | 16 | 2 |
| c-RAF(h) | 97 | 115 | 107 | 102 | 19 |
| cSRC(h) | 70 | 32 | 26 | 14 | 30 |
| DAPK1(h) | 97 | 113 | 46 | 36 | 0 |
| DAPK2(h) | 41 | 92 | 32 | 16 | 3 |
| DCAMKL2(h) | 146 | 131 | 81 | 70 | 56 |
| DDR2(h) | 105 | 104 | 94 | 95 | 79 |
| DMPK(h) | 60 | 66 | 59 | 54 | 12 |
| DRAK1(h) | 47 | 34 | 14 | 14 | 8 |
| DYRK2(h) | 99 | 142 | 155 | 195 | 127 |
| eEF-2K(h) | 113 | 136 | 91 | 43 | 43 |
| EGFR(h) | 95 | 83 | 21 | 16 | −1 |
| EGFR(L858R)(h) | 76 | 120 | N/D | 52 | 26 |
| EGFR(L861Q)(h) | 53 | 74 | 25 | 22 | 15 |
| EGFR(T790M)(h) | 106 | 113 | 100 | 106 | 70 |
| EGFR(T790M, L858R)(h) | 93 | 108 | 85 | 78 | 53 |
| EphA1(h) | 114 | 136 | 73 | 61 | 40 |
| EphA2(h) | 58 | 95 | 31 | 17 | N/D |
| EphA3(h) | 107 | 117 | 6 | 12 | 33 |
| EphA4(h) | 110 | 127 | 88 | 65 | 48 |
| EphA5(h) | 110 | 123 | 18 | 24 | 42 |
| EphA7(h) | 193 | 220 | 159 | 222 | 189 |
| EphA8(h) | 181 | 133 | 93 | 146 | 337 |
| EphB2(h) | 68 | 128 | 18 | 22 | 70 |
| EphB1(h) | 99 | 95 | 44 | 58 | 37 |
| EphB3(h) | 109 | 128 | 62 | 47 | 79 |
| EphB4(h) | 62 | 131 | 44 | 28 | 38 |
| ErbB4(h) | 73 | 82 | 40 | 0 | 2 |
| FAK(h) | 98 | 110 | 111 | 96 | 94 |
| Fer(h) | 117 | 101 | 130 | 108 | 196 |
| Fes(h) | 44 | 74 | 20 | 16 | 23 |
| FGFR1(h) | 120 | 97 | 55 | 59 | 18 |
| FGFR1(V561M)(h) | 108 | 72 | 74 | 74 | 113 |
| FGFR2(h) | 49 | 73 | 14 | 18 | 12 |
| FGFR2(N549H)(h) | 95 | 104 | 116 | 112 | 105 |
| FGFR3(h) | 73 | 208 | 102 | 0 | 10 |
| FGFR4(h) | 67 | 75 | 28 | 19 | 3 |
| Fgr(h) | 54 | 71 | 60 | 47 | 109 |
| Flt1(h) | 109 | 96 | 69 | 48 | 27 |
| Flt3(D835Y)(h) | 120 | 115 | 80 | 71 | 65 |
| Flt3(h) | 104 | 99 | 84 | 18 | 17 |
| Flt4(h) | 135 | 105 | 83 | 89 | 73 |
| Fms(h) | 89 | 92 | 45 | 37 | 14 |
| Fms(Y969C)(h) | 126 | 88 | 72 | 91 | N/D |
| Fyn(h) | 71 | 75 | 74 | 54 | 83 |
| GCK(h) | 98 | 99 | 70 | 66 | 30 |
| GRK5(h) | 117 | 135 | 136 | 131 | 116 |
| GRK6(h) | 131 | 132 | 147 | 141 | 174 |
| GRK7(h) | 111 | 124 | 122 | 100 | 93 |
| GSK3α(h) | 183 | 119 | 157 | 164 | 175 |
| GSK3β(h) | 113 | 132 | 205 | 202 | 238 |
| Haspin(h) | 127 | 71 | 48 | 36 | 25 |
| Hck(h) | 354 | 107 | 72 | 72 | 78 |
| Hck(h) activated | 58 | 100 | 82 | 81 | 67 |
| HIPK1(h) | 94 | 115 | 74 | 91 | 47 |
| HIPK2(h) | 98 | 102 | 73 | 90 | 38 |
| HIPK3(h) | 105 | 105 | 93 | 105 | 85 |
| IGF-1R(h) | 102 | 49 | 119 | 90 | 117 |
| IGF-1R(h), activated | 126 | 94 | 80 | 77 | 45 |
| IKKα(h) | 108 | 104 | 93 | 87 | 50 |
| IKKβ(h) | 105 | 109 | 84 | 84 | 71 |
| IR(h) | 112 | 90 | 96 | 85 | 95 |
| IR(h), activated | 127 | 105 | 79 | 59 | 90 |
| IRR(h) | 85 | 69 | 8 | 8 | 10 |
| IRAK1(h) | 97 | 101 | 95 | 93 | 5 |
| IRAK4(h) | 100 | 110 | 59 | 59 | 3 |
| Itk(h) | 99 | 98 | 77 | 63 | 7 |
| JAK2(h) | 89 | 131 | 133 | 119 | 49 |
| JAK3(h) | 150 | 117 | 121 | 122 | 95 |
| JNK1α1(h) | 91 | 106 | 97 | 98 | 109 |
| JNK2α2(h) | 114 | 109 | 98 | 96 | 81 |
| JNK3(h) | 104 | 90 | 89 | 70 | 171 |
| KDR(h) | 100 | 110 | 101 | 94 | 15 |
| Lck(h) | 346 | 113 | −2 | 228 | 359 |
| Lck(h) activated | 106 | 90 | 243 | 216 | 76 |
| LIMK1(h) | 103 | 109 | 88 | 92 | 87 |
| LKB1(h) | 111 | 99 | 101 | 89 | 51 |
| LOK(h) | 37 | 67 | 37 | 18 | 7 |
| Lyn(h) | 113 | 98 | 69 | 3 | 31 |
| MAPK1(h) | 108 | 97 | 107 | 100 | 102 |
| MAPK2(h) | 98 | 105 | 98 | 93 | 60 |
| MAPKAP-K2(h) | 19 | 35 | 5 | 5 | 9 |
| MAPKAP-K3(h) | 27 | 39 | 3 | 7 | 9 |
| MEK1(h) | 86 | 116 | 77 | 77 | 21 |
| MARK1(h) | 109 | 102 | 132 | 120 | 110 |
| MELK(h) | 74 | 59 | 16 | 17 | 0 |
| Mer(h) | 47 | 90 | 52 | 50 | 17 |
| Met(h) | 104 | 71 | 65 | 62 | 27 |
| Met(D1246H)(h) | 99 | 139 | 125 | 68 | 150 |
| Met(D1246N)(h) | 114 | 149 | 82 | 31 | 90 |
| Met(M1268T)(h) | 114 | 143 | 255 | 265 | 239 |
| Met(Y1248C)(h) | 77 | 141 | 84 | 36 | 73 |
| Met(Y1248D)(h) | 87 | 118 | 102 | 31 | 218 |
| Met(Y1248H)(h) | 88 | 153 | 117 | 63 | 126 |
| MINK(h) | 96 | 103 | 48 | 52 | 5 |
| MKK6(h) | 74 | 98 | 48 | 44 | 18 |
| MKK7β(h) | 137 | 117 | 100 | 94 | 102 |
| MLCK(h) | 85 | 103 | 2 | 1 | 0 |
| MLK1(h) | 77 | 84 | 40 | 33 | 43 |
| Mnk2(h) | 94 | 106 | 89 | 86 | 6 |
| MRCKα(h) | 98 | 103 | 104 | 97 | 5 |
| MRCKβ(h) | 103 | 102 | 83 | 71 | −10 |
| MSK1(h) | 52 | 50 | 32 | 28 | 8 |
| MSK2(h) | 105 | 88 | 56 | 52 | 14 |
| MSSK1(h) | 82 | 100 | 77 | 75 | 22 |
| MST1(h) | 85 | 72 | 14 | 6 | 3 |
| MST2(h) | 98 | 104 | 19 | 11 | 2 |
| MST3(h) | 104 | 95 | 45 | 36 | 4 |
| mTOR(h) | 102 | 110 | 91 | 93 | 135 |
| mTOR/FKBP12(h) | 117 | 118 | 145 | 125 | 140 |
| MuSK(h) | 85 | 106 | 93 | 93 | 27 |
| NEK2(h) | 102 | 97 | 78 | 61 | 0 |
| NEK3(h) | 100 | 100 | 92 | 85 | 20 |
| NEK6(h) | 109 | 98 | 82 | 85 | 49 |
| NEK7(h) | 97 | 96 | 84 | 87 | 89 |
| NEK11(h) | 102 | 95 | 53 | 33 | 2 |
| NLK(h) | 100 | 106 | 87 | 90 | 19 |
| p70S6K(h) | 89 | 84 | 35 | 33 | 3 |
| PAK2(h) | 71 | 69 | 65 | 59 | 44 |
| PAK4(h) | 92 | 98 | 94 | 89 | 86 |
| PAK3(h) | N/D | 50 | 140 | 121 | 102 |
| PAK5(h) | 97 | 100 | 110 | 117 | 125 |
| PAK6(h) | 121 | 105 | 104 | 100 | 107 |
| PAR-1Bα(h) | 62 | 110 | 113 | 109 | 97 |
| PASK(h) | 81 | 60 | 29 | 28 | 9 |
| PDGFRα(h) | 104 | 108 | 65 | 40 | 40 |
| PDGFRα(D842V)(h) | 103 | 107 | 114 | 118 | 170 |
| PDGFRα(V561D)(h) | 58 | 106 | 82 | 100 | 146 |
| PDGFRβ(h) | 116 | 137 | 81 | 53 | 40 |
| PDK1(h) | 144 | 143 | 135 | 159 | 178 |
| PhKγ2(h) | 62 | 86 | 46 | 38 | 16 |
| Pim-1(h) | 44 | 18 | 8 | 7 | 0 |
| Pim-2(h) | 117 | 74 | 76 | 92 | 46 |
| Pim-3(h) | 98 | 94 | 80 | 80 | 37 |
| PKA(h) | 138 | 110 | 119 | 119 | 118 |
| PKBα(h) | 140 | 110 | 57 | 67 | 30 |
| PKBβ(h) | 284 | 250 | 84 | 98 | 21 |
| PKBγ(h) | 105 | 103 | 20 | 41 | 20 |
| PKCα(h) | 94 | 100 | 89 | 86 | 3 |
| PKCβI(h) | 88 | 98 | 78 | 78 | 1 |
| PKCβII(h) | 102 | 100 | 82 | 75 | 3 |
| PKCγ(h) | 94 | 101 | 89 | 79 | 6 |
| PKCδ(h) | 100 | 101 | 101 | 90 | 61 |
| PKCε(h) | 102 | 98 | 79 | 59 | 23 |
| PKCη(h) | 105 | 101 | 103 | 98 | 45 |
| PKCι(h) | 110 | 97 | 68 | 46 | 7 |
| PKCμ(h) | 79 | 73 | 22 | 14 | 10 |
| PKCθ(h) | 102 | 101 | 88 | 76 | 62 |
| PKCζ(h) | 82 | 98 | 81 | 75 | 7 |
| PKD2(h) | 84 | 78 | 33 | 25 | 10 |
| PKG1α(h) | 82 | 70 | 64 | 58 | 25 |
| PKG1β(h) | 71 | 57 | 50 | 53 | 24 |
| Plk1(h) | 109 | 128 | 115 | 119 | 104 |
| Plk3(h) | 107 | 107 | 127 | 129 | 122 |
| PRAK(h) | 159 | 115 | 128 | 118 | 95 |
| PRK2(h) | 72 | 74 | 33 | 27 | 7 |
| PrKX(h) | 84 | 112 | 61 | 76 | 57 |
| PTK5(h) | 135 | 108 | 132 | 129 | 96 |
| Pyk2(h) | 113 | 127 | 47 | 34 | 46 |
| Ret(h) | 108 | 96 | 140 | 145 | 174 |
| Ret (V804L)(h) | 113 | 100 | 79 | 73 | 20 |
| Ret(V804M)(h) | 92 | 105 | 95 | 87 | 36 |
| RIPK2(h) | 92 | 98 | 97 | 98 | 30 |
| ROCK-I(h) | 99 | 117 | 79 | 73 | 17 |
| ROCK-II(h) | 102 | 85 | 74 | 77 | 2 |
| Ron(h) | 117 | 120 | 93 | 79 | 46 |
| Ros(h) | 107 | 86 | 95 | 99 | 150 |
| Rse(h) | 109 | 88 | 88 | 89 | 63 |
| Rsk1(h) | 86 | 102 | 46 | 54 | 34 |
| Rsk2(h) | 65 | 101 | 51 | 38 | 14 |
| Rsk3(h) | 76 | 109 | 76 | 71 | 23 |
| Rsk4(h) | 99 | 125 | 90 | 91 | 29 |
| SAPK2a(h) | 110 | 107 | 90 | 85 | 52 |
| SAPK2a(T106M)(h) | 101 | 100 | 97 | 99 | 32 |
| SAPK2b(h) | 99 | 95 | 81 | 82 | 42 |
| SAPK3(h) | 106 | 97 | 84 | 79 | 24 |
| SAPK4(h) | 98 | 106 | 96 | 91 | 48 |
| SGK(h) | 128 | 115 | 48 | 54 | 2 |
| SGK2(h) | 103 | 119 | 56 | 98 | −1 |
| SGK3(h) | 95 | 58 | 10 | 8 | −3 |
| SIK(h) | 113 | 102 | 66 | 68 | 40 |
| Snk(h) | 94 | 109 | 114 | 131 | 122 |
| Src(1-530)(h) | 95 | 75 | 23 | 19 | 21 |
| Src(T341M)(h) | 98 | 56 | 70 | 76 | 59 |
| SRPK1(h) | 69 | 93 | 90 | 96 | 80 |
| SRPK2(h) | 92 | 100 | 106 | 97 | 80 |
| STK33(h) | 99 | 98 | 45 | 52 | 16 |
| Syk(h) | 45 | 36 | 24 | 9 | 5 |
| TAK1(h) | 116 | 124 | 122 | 177 | N/D |
| TAO1(h) | 99 | 105 | 82 | 73 | 24 |
| TAO2(h) | 95 | 93 | 70 | 74 | 15 |
| TAO3(h) | 45 | 102 | 77 | 67 | 12 |
| TBK1(h) | 106 | 98 | 37 | 39 | 16 |
| Tec(h) activated | 100 | 77 | 56 | 29 | 33 |
| Tie2(h) | 28 | 53 | 26 | 21 | 22 |
| Tie2(R849W)(h) | 102 | 89 | 117 | 108 | 106 |
| Tie2(Y897S)(h) | 99 | 85 | 83 | 87 | 80 |
| TLK2(h) | 113 | 129 | 114 | 151 | 133 |
| TrkA(h) | 74 | N/D | 25 | 17 | 24 |
| TrkB(h) | 4 | 7 | 5 | 8 | 12 |
| TSSK1(h) | 99 | 98 | 79 | 79 | 46 |
| TSSK2(h) | 107 | 91 | 98 | 94 | 92 |
| Txk(h) | 87 | 98 | 48 | 37 | 10 |
| ULK2(h) | 123 | 132 | 122 | 131 | 124 |
| ULK3(h) | 142 | 164 | 167 | 147 | 177 |
| WNK2(h) | 95 | 94 | 64 | 54 | 8 |
| WNK3(h) | 100 | 97 | 77 | 74 | 9 |
| VRK2(h) | 112 | 109 | 161 | 185 | 169 |
| Yes(h) | 49 | 93 | 67 | 14 | N/D |
| ZAP-70(h) | 79 | 58 | 75 | 33 | 1 |
| ZIPK(h) | 80 | 67 | 28 | 13 | 1 |

N/D: % activity could not be determined as the duplicates.
MMI-0100: YARAAARQARAKALARQLGVAA (SEQ ID NO: 1)
MMI-0200: YARAAARQARAKALNRQLGVA (SEQ ID NO: 19)
MMI-0300: FAKLAARLYRKALARQLGVAA (SEQ ID NO: 3)
MMI-0400: KAFAKLAARLYRKALARQLGVAA (SEQ ID NO: 4)
MMI-0500: HRRIKAWLKKIKALARQLGVAA (SEQ ID NO: 7)

According to some embodiments, inhibitory profiles of MMI-0100 (SEQ ID NO: 1), its functional equivalents, and its mimetics in vivo depend on dosages, routes of administration, and cell types responding to the inhibitors.

According to some embodiments, the pharmaceutical formulation inhibits less than 65% of the kinase activity of the other selected kinase(s). According to some embodiments, the pharmaceutical formulation inhibits less than 60% of the kinase activity of the other selected kinase(s). According to some embodiments, the pharmaceutical formulation inhibits less than 55% of the kinase activity of the other selected kinase(s). According to another embodiment, the pharmaceutical formulation inhibits less than 50% of the kinase activity of the other selected kinase(s). According to some embodiments, the pharmaceutical formulation inhibits less than 45% of the kinase activity of the other selected kinase(s). According to another embodiment, the pharmaceutical formulation inhibits less than 40% of the kinase activity of the other selected kinase(s). According to some embodiments, the pharmaceutical formulation inhibits less than 35% of the kinase activity of the other selected kinase(s). According to some embodiments, the pharmaceutical formulation inhibits less than 30% of the kinase activity of the other selected kinase(s). According to some embodiments, the pharmaceutical formulation inhibits less than 25% of the kinase activity of the other selected kinase(s). According to another embodiment, the pharmaceutical formulation inhibits less than 20% of the kinase activity of the other selected kinase(s). According to another embodiment, the pharmaceutical formulation inhibits less than 15% of the kinase activity of the other selected kinase(s). According to another embodiment, the pharmaceutical formulation inhibits less than 10% of the kinase activity of the other selected kinase(s). According to another embodiment, the pharmaceutical formulation inhibits less than 5% of the kinase activity of the other selected kinase(s). According to another embodiment, the pharmaceutical formulation increases the kinase activity of the other selected kinases.

According to the embodiments of the immediately preceding paragraph, the one or more other selected kinase that is not substantially inhibited is selected from the group of Ca2+/calmodulin-dependent protein kinase II (CaMKII, including its subunit CaMKIIδ), Proto-oncogene serine/threonine-protein kinase (PIM-1), cellular-Sarcoma (c-SRC), Spleen Tyrosine Kinase (SYK), c-Src Tyrosine Kinase (CSK), and Insulin-like Growth Factor 1 Receptor (IGF-1R).

According to some embodiments, kinases that are substantially inhibited (i.e., kinases whose kinase activity is inhibited by at least 65%) by at least one MMI inhibitor (i.e., at least one of MMI-0100 (SEQ ID NO: 1), MMI-0200 (SEQ ID NO: 19), MMI-0300 (SEQ ID NO: 3), MMI-0400 (SEQ ID NO: 4), and MMI-0500 (SEQ ID NO: 7)) of the present invention is selected from the group consisting of: Abelson murine leukemia viral oncogene homolog 1 (Abl), Abelson murine leukemia viral oncogene homolog 1 (T3151) (Abl (T3151)), Abelson murine leukemia viral oncogene homolog 1 (Y253F) (Abl (Y253F)), Anaplastic lymphoma kinase (ALK), Abelson-related gene (Arg), 5′-AMP-activated protein kinase catalytic subunit alpha-1 (AMPKα1), 5′-AMP-activated protein kinase catalytic subunit alpha-2 (AMPKα2), AMPK-related protein kinase 5 (ARK5), Apoptosis signal regulating kinase 1 (ASK1), Aurora kinase B (Aurora-B), AXL receptor tyrosine kinase (Axl), Bone marrow tyrosine kinase gene in chromosome X protein (Bmx), Breast tumor kinase (BRK), Bruton's tyrosine kinase (BTK), Bruton's tyrosine kinase (R28H) (BTK (R28H)), Ca2⁺/calmodulin-dependent protein kinase I (CaMKI), Ca2⁺/calmodulin-dependent protein kinase IIβ (CaMIIβ), Ca2⁺/calmodulin-dependent protein kinase IIγ (CaMKIIγ), Ca2⁺/calmodulin-dependent protein kinase δ (CaMKIδ), Ca2⁺/calmodulin-dependent protein kinase IIδ (CaMKIIδ), Ca2⁺/calmodulin-dependent protein kinase IV (CaMKIV), Cell devision kinase 2 (CDK2/cyclinE), Cell devision kinase 3 (CDK3/cyclinE), Cell devision kinase 6 (CDK6/cyclinD3), Cell devision kinase 7 (CDK7/cyclinH/MAT1), Cell devision kinase 9 (CDK9/cyclin T1), Checkpoint kinase 2 (CHK2), Checkpoint kinase 2 (1157T) (CHK2 (1157T)), Checkpoint kinase 2 (R145W) (CHK2 (R145W)), Proto-oncogene tyrosine-protein kinase cKit (D816V) (cKit (D816V)), C-src tyrosine kinase (CSK), Raf proto-oncogene serine/threonine protein kinase (c-RAF), Proto-oncogene tyrosine-protein kinase (cSRC), Death-associated protein kinase 1 (DAPK1), Death-associated protein kinase 2 (DAPK2), Dystrophia myotonica-protein kinase (DMPK), DAP kinase-related apoptosis-inducing protein kinase 1 (DRAK1), Epidermal growth factor receptor (EGFR), Epidermal growth factor receptor (EGFR L858R), Epidermal growth factor receptor L861Q (EGFR (L861Q)), Eph receptor A2 (EphA2) (EphA2), Eph receptor A3 (EphA3), Eph receptor A5 (EphA5), Eph receptor B2 (EphB2), Eph receptor B4 (EphB4), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), Fibroblast growth factor receptor 4 (FGFR4), Fms-like tyrosine kinase receptor-3 (Flt3), FMS proto-oncogene (Fms), Haploid germ cell-specific nuclear protein kinase (Haspin), Insulin receptor-related receptor (IRR), Interleukin-1 receptor-associated kinase 1 (IRAK1), Interleukin-1 receptor-associated kinase 4 (IRAK4), IL2-inducible T-cell kinase (Itk), Kinase insert domain receptor (KDR), Lymphocyte cell-specific protein-tyrosine kinase (Lck), Lymphocyte-oriented kinase (LOK), Lyn tyrosine protein kinase (Lyn), MAP kinase-activated protein kinase 2 (MK2), MAP kinase-activated protein kinase 3 (MK3), MEK1, Maternal embryonic leucine zipper kinase (MELK), c-Mer proto-oncogene tyrosine kinase (Mer), c-Met proto-oncogene tyrosine kinase (Met), c-Met proto-oncogene tyrosine kinase D1246N (Met (D1246N)), c-Met proto-oncogene tyrosine kinase Y1248D (Met Y1248D), Misshapen/NIK-related kinase (MINK), MAP kinase kinase 6 (MKK6), Myosin light-chain kinase (MLCK), Mixed lineage kinase 1 (MLK1), MAP kinase signal-integrating kinase 2 (MnK2), Myotonic dystrophy kinase-related CDC42-binding kinase alpha (MRCKα), Myotonic dystrophy kinase-related CDC42-binding kinase beta (MRCKβ), Mitogen- and stress-activated protein kinase 1 (MSK1), Mitogen- and stress-activated protein kinase 2 (MSK2), Muscle-specific serine kinase 1 (MSSK1), Mammalian STE20-like protein kinase 1 (MST1), Mammalian STE20-like protein kinase 2 (MST2), Mammalian STE20-like protein kinase 3 (MST3), Muscle, skeletal receptor tyrosine-protein kinase (MuSK), Never in mitosis A-related kinase 2 (NEK2), Never in mitosis A-related kinase 3 (NEK3), Never in mitosis A-related kinase 11 (NEK11), 70 kDa ribosomal protein S6 kinase 1 (p70S6K), PAS domain containing serine/threonine kinase (PASK), Phosphorylase kinase subunit gamma-2 (PhKγ2), Pim-1 kinase (Pim-1), Protein kinase B alpha (PKBα), Protein kinase B beta (PKBβ), Protein kinase B gamma (PKBγ), Protein kinase C, alpha (PKCα), Protein kinase C, beta1 (PKCβ1), Protein kinase C, beta II (PKCβII), Protein kinase C, gamma (PKCγ), Protein kinase C, epsilon (PKCε), Protein kinase C, iota (PCKι), Protein kinase C, mu (PKCμ), Protein kinase C, zeta (PKCζ), protein kinase D2 (PKD2), cGMP-dependent protein kinase 1 alpha (PKG1α), cGMP-dependent protein kinase 1 beta (PKG1β), Protein-kinase C-related kinase 2 (PRK2), Proline-rich tyrosine kinase 2 (Pyk2), Proto-oncogene tyrosine-protein kinase receptor Ret V804L (Ret (V804L)), Receptor-interacting serine-threonine kinase 2 (RIPK2), Rho-associated protein kinase I (ROCK-I), Rho-associated protein kinase II (ROCK-II), Ribosomal protein S6 kinase 1 (Rsk1), Ribosomal protein S6 kinase 2 (Rsk2), Ribosomal protein S6 kinase 3 (Rsk3), Ribosomal protein S6 kinase 4 (Rsk4), Stress-activated protein kinase 2A T106M (SAPK2a, T106M), Stress-activated protein kinase 3 (SAPK3), Serum/glucocorticoid regulated kinase (SGK), Serum/glucocorticoid regulated kinase 2 (SGK2), Serum/glucocorticoid-regulated kinase 3 (SGK3), Proto-oncogene tyrosine-protein kinase Src 1-530 (Src, 1-530), Serine/threonine-protein kinase 33 (STK33), Spleen tyrosine kinase (Syk), Thousand and one amino acid protein 1 (TAO1), Thousand and one amino acid protein 2 (TAO2), Thousand and one amino acid protein 3 (TAO3), TANK-binding kinase 1 (TBK1), Tec protein tyrosine kinase (Tec), Tunica interna endothelial cell kinase 2 (Tie2), Tyrosine kinase receptor A (TrkA), BDNF/NT-3 growth factors receptor (TrkB), TXK tyrosine kinase (Txk), WNK lysine deficient protein kinase 2 (WNK2), WNK lysine deficient protein kinase 3 (WNK3), Yamaguchi sarcoma viral oncogene homolog 1 (Yes), Zeta-chain (TCR) Associated Protein kinase 70 kDa (ZAP-70), and ZIP kinase (ZIPK).

According to some other embodiments, kinases that are substantially inhibited (i.e., kinases whose kinase activity is inhibited by at least 65%) by at least two MMI inhibitors (i.e., at least two of MMI-0100 (SEQ ID NO: 1), MMI-0200 (SEQ ID NO: 19), MMI-0300 (SEQ ID NO: 3), MMI-0400 (SEQ ID NO: 4), and MMI-0500 (SEQ ID NO: 7)) of the present invention is selected from the group consisting of: Anaplastic lymphoma kinase (ALK), Breast tumor kinase (BRK), Bruton's tyrosine kinase (BTK), Ca²⁺/calmodulin-dependent protein kinase I (including CaMKIδ), Ca²⁺/calmodulin-dependent protein kinase II (CaMKII, including CaMKIIβ, CaMKIIδ and CaMKIIγ), Ca²⁺/calmodulin-dependent protein kinase IV (CaMKIV), Checkpoint kinase 2 (CHK2 (R145W)), Proto-oncogene tyrosine-protein kinase cKit (D816V) (cKit (D816V)), C-src tyrosine kinase (CSK), Proto-oncogene tyrosine-protein kinase (cSRC), Death-associated protein kinase 1 (DAPK1), Death-associated protein kinase 2 (DAPK2), DAP kinase-related apoptosis-inducing protein kinase 1 (DRAK1), Epidermal growth factor receptor (EGFR), Epidermal growth factor receptor L861Q (EGFR (L861Q)), Eph receptor A2 (EphA2), Eph receptor A3 (EphA3), Eph receptor A5 (EphA5), Eph receptor B2 (EphB2), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), and Fibroblast growth factor receptor 4 (FGFR4), Fms-like tyrosine kinase receptor-3 (Flt3), Insulin receptor-related receptor (IRR), Lymphocyte-oriented kinase (LOK), Lyn tyrosine protein kinase (Lyn), MAP kinase-activated protein kinase 2 (MK2), MAP kinase-activated protein kinase 3 (MK3), Maternal embryonic leucine zipper kinase (MELK), Myosin light-chain kinase (MLCK), Mitogen- and stress-activated protein kinase (MSK1), Mammalian STE20-like protein kinase 1 (MST1), Mammalian STE20-like protein kinase 2 (MST2), Never in mitosis A-related kinase 11 (NEK11), 70 kDa ribosomal protein S6 kinase 1 (p70S6K), PAS domain containing serine/threonine kinase (PASK), Pim-1 kinase (Pim-1), Protein kinase B, gamma (PKBγ), Protein kinase C, mu (PKCμ), protein kinase D2 (PKD2), Protein-kinase C-related kinase 2 (PRK2), Serum/glucocorticoid-regulated kinase 3 (SGK3), Proto-oncogene tyrosine-protein kinase Src (Src), Spleen tyrosine kinase (Syk), Tec protein tyrosine kinase (Tec), Tunica interna endothelial cell kinase 2 (Tie2), Tyrosine kinase receptor A (TrkA), BDNF/NT-3 growth factors receptor (TrkB), Zeta-chain (TCR) Associated Protein kinase 70 kDa (ZAP-70), and ZIP kinase (ZIPK).

According to some embodiments, the pharmaceutical formulation comprises a small-molecule inhibitor of MK2, including, but not limited to:

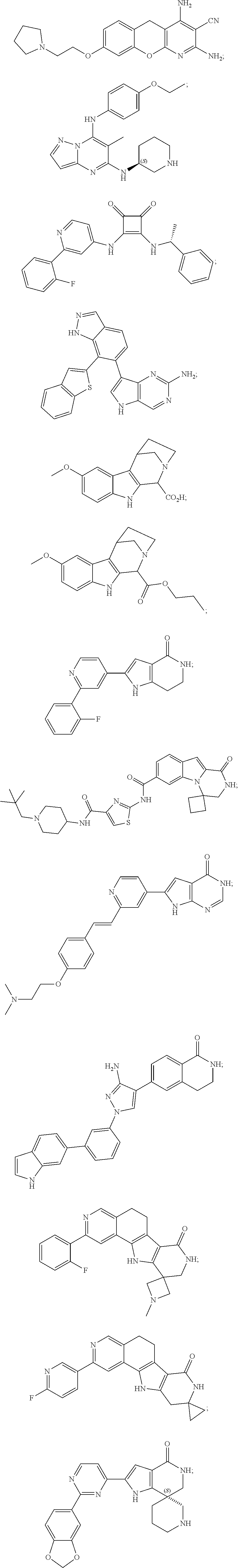

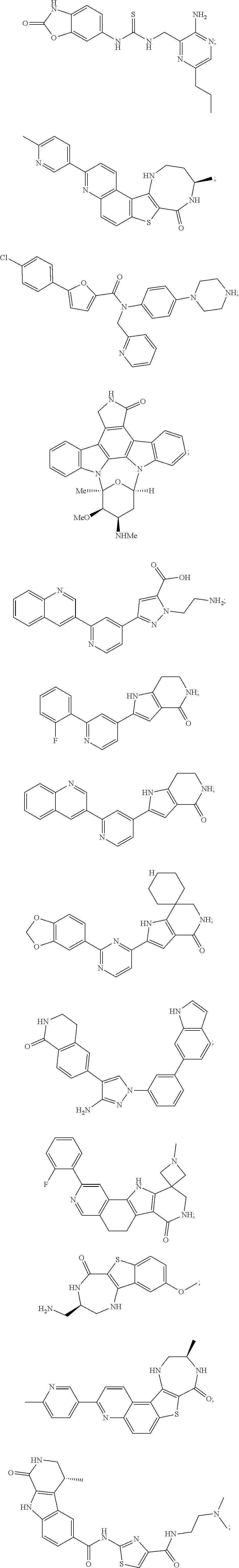

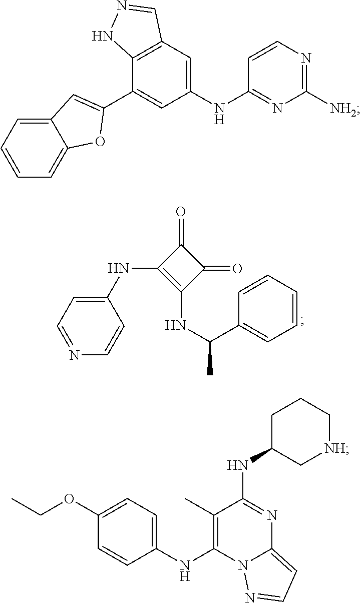 [SMILES: Nc1nccc(Nc2cc(-c3cc4ccccc4o3)c3[nH]ncc3c2)n1 ; C[C@@H](Nc1c(Nc2ccncc2)c(=O)c1=O)c1ccccc1 ; CCOc1ccc(Nc2c(C)c(N[C@H]3CCCNC3)nc3ccnn23)cc1]

or a combination thereof.

According to some embodiments, an immunomodulatory amount of the polypeptide of amino acid sequence YARAAARQARAKALARQLGVAA (SEQ ID NO: 1), its functional equivalents, variants or mimetics, when used before treatment with an immunostimulatory agent to which a subject in need thereof has become tolerized, is effective to re-sensitize the subject to that agent so that the subject becomes immunoresponsive to the immunostimulating agent upon its subsequent administration, thereby converting an otherwise attenuated or suppressed immune response to a robust immune response.

According to some embodiments, the described invention provides a pharmaceutical composition comprising, in order (1) a first pharmaceutical formulation formulated for delivery by inhalation containing an immunomodulatory amount of an MK2 inhibitory peptide, followed by (2) a second pharmaceutical formulation containing a therapeutic amount of an immunostimulatory agent for use in the treatment of a disease, disorder or condition of lung tissue in a subject that is in an immunotolerant state with regard to the immunostimulatory agent that is no longer therapeutically effective, wherein the use is effective so that the subject is resensitized and therefore is immunoresponsive to the immune stimulating agent upon its subsequent administration.

According to some embodiments, the immunotolerant state of the subject is characterized by an attenuated immune response to the immunostimulatory agent, compared to a normal control. According to some embodiments, the immunotolerant state is characterized by one or more of a reduced level of synthesis, expression, or both of pro-inflammatory cytokines, anti-inflammatory cytokines, both pro-inflammatory and anti-inflammatory cytokines, or an altered balance between proinflammatory cytokines and anti-inflammatory cytokines, compared to a control. According to some embodiments the immunotolerant state results from exposure to an immunosuppressive drug. According to some embodiments, the immunotolerant state is a result of repeated prior exposure to the immunostimulatory agent. According to some embodiments, the immunostimulatory agent is a chemotherapeutic agent. According to some embodiments, the immunostimulatory agent is lipopolysaccharide (LPS).

According to some embodiments, the kinase-inhibiting peptide is MMI0100, or a functional equivalent, a peptide mimetic or a variant of MMI0100.

According to some embodiments, the immunoactivating amount of MMI0100 is effective to modulate MK2 signaling.

According to some embodiments, the immunoactivating amount of MMI0100 is effective to modulate the MK2 signaling affecting a MAPK pathway, an NfκB pathway, an IFNα/β pathway or a combination thereof.

According to some embodiments, the immunomodulating amount of the MK2 peptide is effective to modulate one or more of autocrine signaling, paracrine signaling or hormonal signaling in an immune cell population.

According to some embodiments, the immunomodulatory amount of MMI0100 is effective to increase activation of a population of inflammatory cells selected from the group consisting of T cells, B cells, NK cells, CT cells, neutrophils, lymphocytes, macrophages, dendritic cells.

According to some embodiments, the immunomodulatory amount of MMI0100 is effective to increase one or more of autocrine signaling, paracrine signaling or hormonal signaling by immune cells. According to some embodiments, the autocrine signaling, paracrine signaling or hormonal signaling by one or more immune cells comprises TLR-4 signaling. According to some embodiments, the immune cells are one or more populations selected from T cells, B cells, NK cells, CT cells, neutrophils, lymphocytes, macrophages, dendritic cells. According to some embodiments, the robust immune response comprises one or more of autocrine signaling, paracrine signaling or hormonal signaling by immune cells.

According to some embodiments, as a result of the signaling, the immune cells express, synthesize, or secrete one or more cytokines selected from the group consisting of IL-1α, IL-1β, IL-2, IL-3, IL-4, IL-5, IL-6, IL-7, IL-8, IL-9, IL-10, IL-11, IL-12/IL-23 P40, IL13, IL-17, IL-18, TGF-β, IFN-γ, GM-CSF, CXCL1, CXCL2, and TNF-α.

According to some embodiments, a level of cytokines expressed, synthesized or secreted is measurable in a body fluid.

According to some embodiments, the body fluid is sputum, blood or both.

According to some embodiments, the immunoresponsive immune response comprises restoration of expression, synthesis or both of inflammatory cytokines in immune cells of the lung without affecting systemic immune cells in an amount to cause unwanted systemic side effects.

According to some embodiments, the disease, disorder or condition is gram negative bacterial sepsis, cystic fibrosis, COPD, or lung cancer. According to some embodiments, the subject is an immunocompromised subject.

According to some embodiments, the therapeutic amount of the therapeutic inhibitor peptide of the first pharmaceutical formulation is of an amount from about 0.000001 mg/kg body weight to about 100 mg/kg body weight. According to another embodiment, the therapeutic amount of the therapeutic inhibitory peptide of the first pharmaceutical formulation is of an amount from about 0.00001 mg/kg body weight to about 100 mg/kg body weight. According to another embodiment, the therapeutic amount of the therapeutic inhibitory peptide of the first pharmaceutical formulation is of an amount from about 0.0001 mg/kg body weight to about 100 mg/kg body weight. According to another embodiment, the therapeutic amount of the therapeutic inhibitory peptide of the first pharmaceutical formulation is of an amount from about 0.001 mg/kg body weight to about 10 mg/kg body weight. According to another embodiment, the therapeutic amount of the therapeutic inhibitory peptide of the first pharmaceutical formulation is of an amount from about 0.01 mg/kg body weight to about 10 mg/kg body weight. According to another embodiment, the therapeutic amount of the therapeutic inhibitory peptide of the first pharmaceutical formulation is of an amount from about 0.1 mg/kg (or 100 μg/kg) body weight to about 10 mg/kg body weight. According to another embodiment, the therapeutic amount of the therapeutic inhibitory peptide of the first pharmaceutical formulation is of an amount from about 1 mg/kg body weight to about 10 mg/kg body weight. According to another embodiment, the therapeutic amount of the therapeutic inhibitory peptide of the first pharmaceutical formulation is of an amount from about 10 mg/kg body weight to about 100 mg/kg body weight. According to another embodiment, the therapeutic amount of the therapeutic inhibitory peptide of the first pharmaceutical formulation is of an amount from about 2 mg/kg body weight to about 10 mg/kg body weight. According to another embodiment, the therapeutic amount of the therapeutic inhibitory peptide of the first pharmaceutical formulation is of an amount from about 3 mg/kg body weight to about 10 mg/kg body weight. According to another embodiment, the therapeutic amount of the therapeutic inhibitory peptide of the pharmaceutical formulation is of an amount from about 4 mg/kg body weight to about 10 mg/kg body weight. According to another embodiment, the therapeutic amount of the therapeutic inhibitory peptide of the first pharmaceutical formulation is of an amount from about 5 mg/kg body weight to about 10 mg/kg body weight. According to another embodiment, the therapeutic amount of the therapeutic inhibitory peptide of the first pharmaceutical formulation is of an amount from about 60 mg/kg body weight to about 100 mg/kg body weight. According to another embodiment, the therapeutic amount of the therapeutic inhibitory peptide of the first pharmaceutical formulation is of an amount from about 70 mg/kg body weight to about 100 mg/kg body weight. According to another embodiment, the therapeutic amount of the therapeutic inhibitory peptide of the first pharmaceutical formulation is of an amount from about 80 mg/kg body weight to about 100 mg/kg body weight. According to another embodiment, the therapeutic amount of the therapeutic inhibitory peptide of the first pharmaceutical formulation is of an amount from about 90 mg/kg body weight to about 100 mg/kg body weight. According to another embodiment, the therapeutic amount of the therapeutic inhibitor peptide of the first pharmaceutical formulation is of an amount from about 0.000001 mg/kg body weight to about 90 mg/kg body weight. According to another embodiment, the therapeutic amount of the therapeutic inhibitor peptide of the first pharmaceutical formulation is of an amount from about 0.000001 mg/kg body weight to about 80 mg/kg body weight. According to another embodiment, the therapeutic amount of the therapeutic inhibitor peptide of the first pharmaceutical formulation is of an amount from about 0.000001 mg/kg body weight to about 70 mg/kg body weight. According to another embodiment, the therapeutic amount of the therapeutic inhibitor peptide of the pharmaceutical formulation is of an amount from about 0.000001 mg/kg body weight to about 60 mg/kg body weight. According to another embodiment, the therapeutic amount of the therapeutic inhibitor peptide of the first pharmaceutical formulation is of an amount from about 0.000001 mg/kg body weight to about 50 mg/kg body weight. According to another embodiment, the therapeutic amount of the therapeutic inhibitor peptide of the first pharmaceutical formulation is of an amount from about 0.000001 mg/kg body weight to about 40 mg/kg body weight. According to another embodiment, the therapeutic amount of the therapeutic inhibitor peptide is of an amount from about 0.000001 mg/kg body weight to about 30 mg/kg body weight. According to another embodiment, the therapeutic amount of the therapeutic inhibitor peptide of the first pharmaceutical formulation is of an amount from about 0.000001 mg/kg body weight to about 20 mg/kg body weight. According to another embodiment, the therapeutic amount of the therapeutic inhibitor peptide of the pharmaceutical formulation is of an amount from about 0.000001 mg/kg body weight to about 10 mg/kg body weight. According to another embodiment, the therapeutic amount of the therapeutic inhibitor peptide of the first pharmaceutical formulation is of an amount from about 0.000001 mg/kg body weight to about 1 mg/kg body weight. According to another embodiment, the therapeutic amount of the therapeutic inhibitor peptide of the first pharmaceutical formulation is of an amount from about 0.000001 mg/kg body weight to about 0.1 mg/kg body weight. According to another embodiment, the therapeutic amount of the therapeutic inhibitor peptide of the pharmaceutical formulation is of an amount from about 0.000001 mg/kg body weight to about 0.1 mg/kg body weight. According to another embodiment, the therapeutic amount of the therapeutic inhibitor peptide of the first pharmaceutical formulation is of an amount from about 0.000001 mg/kg body weight to about 0.01 mg/kg body weight. According to another embodiment, the therapeutic amount of the therapeutic inhibitor peptide of the first pharmaceutical formulation is of an amount from about 0.000001 mg/kg body weight to about 0.001 mg/kg body weight. According to another embodiment, the therapeutic amount of the therapeutic inhibitor peptide of the first pharmaceutical formulation is of an amount from about 0.000001 mg/kg body weight to about 0.0001 mg/kg body weight. According to another embodiment, the therapeutic amount of the therapeutic inhibitor peptide of the first pharmaceutical formulation is of an amount from about 0.000001 mg/kg body weight to about 0.00001 mg/kg body weight.

According to some other embodiments, the therapeutic dose of the therapeutic inhibitor peptide of the first pharmaceutical formulation ranges from 1 μg/kg/day to 25 μg/kg/day. According to some other embodiments, the therapeutic dose of the therapeutic inhibitor peptide of the pharmaceutical formulation ranges from 1 μg/kg/day to 2 μg/kg/day. According to some other embodiments, the therapeutic dose of the therapeutic inhibitor peptide of the first pharmaceutical formulation ranges from 2 μg/kg/day to 3 μg/kg/day. According to some other embodiments, the therapeutic dose of the therapeutic inhibitor peptide of the first pharmaceutical formulation ranges from 3 μg/kg/day to 4 μg/kg/day. According to some other embodiments, the therapeutic dose of the therapeutic inhibitor peptide of the pharmaceutical ranges from 4 μg/kg/day to 5 μg/kg/day. According to some other embodiments, the therapeutic dose of the therapeutic inhibitor peptide of the first pharmaceutical formulation ranges from 5 μg/kg/day to 6 μg/kg/day. According to some other embodiments, the therapeutic dose of the therapeutic inhibitor peptide of the first pharmaceutical formulation ranges from 6 μg/kg/day to 7 μg/kg/day. According to some other embodiments, the therapeutic dose of the therapeutic inhibitor peptide of the first pharmaceutical formulation ranges from 7 μg/kg/day to 8 μg/kg/day. According to some other embodiments, the therapeutic dose of the therapeutic inhibitor peptide of the first pharmaceutical formulation ranges from 8 μg/kg/day to 9 μg/kg/day. According to some other embodiments, the therapeutic dose of the therapeutic inhibitor peptide of the first pharmaceutical formulation ranges from 9 μg/kg/day to 10 μg/kg/day. According to some other embodiments, the therapeutic dose of the therapeutic inhibitor peptide of the first pharmaceutical formulation ranges from 1 μg/kg/day to 5 μg/kg/day. According to some other embodiments, the therapeutic dose of the therapeutic inhibitor peptide of the first pharmaceutical formulation ranges from 5 μg/kg/day to 10 μg/kg/day. According to some other embodiments, the therapeutic dose of the therapeutic inhibitor peptide of the first pharmaceutical formulation ranges from 10 μg/kg/day to 15 μg/kg/day. According to some other embodiments, the therapeutic dose of the therapeutic inhibitor peptide of the first pharmaceutical formulation ranges from 15 μg/kg/day to 20 μg/kg/day. According to some other embodiments, the therapeutic dose of the therapeutic inhibitor peptide of the first pharmaceutical formulation ranges from 25 μg/kg/day to 30 μg/kg/day. According to some other embodiments, the therapeutic dose of the therapeutic inhibitor peptide of the first pharmaceutical formulation ranges from 30 μg/kg/day to 35 μg/kg/day. According to some other embodiments, the therapeutic dose of the therapeutic inhibitor peptide of the first pharmaceutical formulation ranges from 35 μg/kg/day to 40 μg/kg/day. According to some other embodiments, the therapeutic dose of the therapeutic inhibitor peptide of the first pharmaceutical formulation ranges from 40 μg/kg/day to 45 μg/kg/day. According to some other embodiments, the therapeutic dose of the therapeutic inhibitor peptide of the first pharmaceutical formulation ranges from 45 μg/kg/day to 50 μg/kg/day. According to some other embodiments, the therapeutic dose of the therapeutic inhibitor peptide of the first pharmaceutical formulation ranges from 50 μg/kg/day to 55 μg/kg/day. According to some other embodiments, the therapeutic dose of the therapeutic inhibitor peptide of the first pharmaceutical formulation ranges from 55 μg/kg/day to 60 μg/kg/day. According to some other embodiments, the therapeutic dose of the therapeutic inhibitor peptide of the pharmaceutical formulation ranges from 60 μg/kg/day to 65 μg/kg/day. According to some other embodiments, the therapeutic dose of the therapeutic inhibitor peptide of the first pharmaceutical formulation ranges from 65 μg/kg/day to 70 μg/kg/day. According to some other embodiments, the therapeutic dose of the therapeutic inhibitor peptide of the first pharmaceutical formulation ranges from 70 μg/kg/day to 75 μg/kg/day. According to some other embodiments, the therapeutic dose of the therapeutic inhibitor peptide of the first pharmaceutical formulation ranges from 80 μg/kg/day to 85 μg/kg/day. According to some other embodiments, the therapeutic dose of the therapeutic inhibitor peptide of the first pharmaceutical formulation ranges from 85 μg/kg/day to 90 μg/kg/day. According to some other embodiments, the therapeutic dose of the therapeutic inhibitor peptide of the first pharmaceutical formulation ranges from 90 μg/kg/day to 95 μg/kg/day. According to some other embodiments, the therapeutic dose of the therapeutic inhibitor peptide of the first pharmaceutical formulation ranges from 95 μg/kg/day to 100 μg/kg/day.

According to another embodiment, the therapeutic dose of the therapeutic inhibitor peptide of the first pharmaceutical formulation is 1 μg/kg/day.

According to another embodiment, the therapeutic dose of the therapeutic inhibitor peptide of the first pharmaceutical formulation is 2 μg/kg/day.

According to another embodiment, the therapeutic dose of the therapeutic inhibitor peptide of the first pharmaceutical formulation is 3 μg/kg/day.

According to another embodiment, the therapeutic dose of the therapeutic inhibitor peptide of the first pharmaceutical formulation is 4 μg/kg/day.

According to another embodiment, the therapeutic dose of the therapeutic inhibitor peptide of the pharmaceutical formulation is 5 μg/kg/day.

According to another embodiment, the therapeutic dose of the therapeutic inhibitor peptide of the first pharmaceutical formulation is 6 μg/kg/day.

According to another embodiment, the therapeutic dose of the therapeutic inhibitor peptide of the first pharmaceutical formulation is 7 μg/kg/day.

According to another embodiment, the therapeutic dose of the therapeutic inhibitor peptide of the first pharmaceutical formulation is 8 μg/kg/day.

According to another embodiment, the therapeutic dose of the therapeutic inhibitor peptide of the pharmaceutical formulation is 9 μg/kg/day.

According to another embodiment, the therapeutic dose of the therapeutic inhibitor peptide of the pharmaceutical formulation is 10 μg/kg/day.

The polypeptide of amino acid sequence YARAAARQARAKALARQLGVAA (SEQ ID NO: 1) or a functional equivalent thereof may be administered in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. Pharmaceutically acceptable salts are well-known. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the described invention or may be prepared by separately reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by methods known in the art of pharmacy. Such methods include the step of bringing into association a therapeutic agent(s), or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

According to some embodiments, the carrier is a controlled release carrier. The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations. According to some embodiments, the controlled release of the pharmaceutical formulation is mediated by changes in temperature. According to some other embodiments, the controlled release of the pharmaceutical formulation is mediated by changes in pH.

Injectable depot forms may be made by forming microencapsulated matrices of a therapeutic agent/drug in biodegradable polymers such as, but not limited to, polyesters (polyglycolide, polylactic acid and combinations thereof), polyester polyethylene glycol copolymers, polyamino-derived biopolymers, polyanhydrides, polyorthoesters, polyphosphazenes, sucrose acetate isobutyrate (SAIB), photopolymerizable biopolymers, naturally-occurring biopolymers, protein polymers, collagen, and polysaccharides. Depending upon the ratio of drug to polymer and the nature of the particular polymer employed, the rate of drug release may be controlled. Such long acting formulations may be formulated with suitable polymeric or hydrophobic materials (for example as an emulsion in acceptable oil) or ion exchange resins, or as sparingly soluble derivatives, for example, as a sparingly soluble salt. Depot injectable formulations also are prepared by entrapping the drug in liposomes or microemulsions which are compatible with body tissues.

According to some embodiments, the carrier is a delayed release carrier. According to another embodiment, the delayed release carrier comprises a biodegradable polymer. According to another embodiment, the biodegradable polymer is a synthetic polymer. According to another embodiment, the biodegradable polymer is a naturally occurring polymer.

According to some embodiments, the carrier is a sustained release carrier. According to another embodiment, the sustained-release carrier comprises a biodegradable polymer. According to another embodiment, the biodegradable polymer is a synthetic polymer. According to another embodiment, the biodegradable polymer is a naturally occurring polymer.

According to some embodiments, the carrier is a short-term release carrier. The term “short-term” release, as used herein, means that an implant is constructed and arranged to deliver therapeutic levels of the active ingredient for about 1, 2, 3, 4, 5, 6, 7, 8, 9, 10, 11, 12, 13, 14, 15, 16, 17, 18, 19, 20, 21, 22, or 23 hours. According to some other embodiments, the short term release carrier delivers therapeutic levels of the active ingredient for about 1, 2, 3, or 4 days.

According to some embodiments, the carrier is a long-term release carrier. The term “long-term” release, as used herein, means that an implant is constructed and arranged to deliver therapeutic levels of the active ingredient for at least about 5, 6, 7, 8, 9, 10, 11, 12, 13, 14, 15, 16, 17, 18, 19, 20, 21, 22, 23, 24, 25, 26, 27, 29, 29, 30, 31, 32, 33, 34, 35, 36, 37, 38, 39, 40, 41, 42, 43, 44, 45, 46, 47, 48, 48, 50, 51, 52, 53, 54, 55, 56, 57, 58, 59, or 60 days. According to another embodiment, the long-term-release carrier comprises a biodegradable polymer. According to another embodiment, the biodegradable polymer is a synthetic polymer.

According to some embodiments, the carrier comprises particles. According to some embodiments, formulations as described herein are contained in the particle. According to some embodiments, formulations as described herein are contained on the particle. According to some embodiments, formulations as described herein are contained both in and on the particle.

The formulations also may contain appropriate adjuvants, including, without limitation, preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

According to some embodiments, the polypeptides of the present invention can be covalently attached to polyethylene glycol (PEG) polymer chains. According to some other embodiments, the polypeptides of the present invention are stapled with hydrocarbons to generate hydrocarbon-stapled peptides that are capable of forming stable alpha-helical structure (Schafmeister, C. et al., J. Am. Chem. Soc., 2000, 122, 5891-5892, incorporated herein by reference in its entirety).

According to some other embodiments, the polypeptides of the present invention are encapsulated or entrapped into microspheres, nanocapsules, liposomes, or microemulsions, or comprises d-amino acids in order to increase stability, to lengthen delivery, or to alter activity of the peptides. These techniques can lengthen the stability and release simultaneously by hours to days, or delay the uptake of the drug by nearby cells.

The formulations of therapeutic agent(s) may be administered in pharmaceutically acceptable solutions, which may routinely contain pharmaceutically acceptable concentrations of salt, buffering agents, preservatives, compatible carriers, adjuvants, and optionally other therapeutic ingredients.

According to some embodiments, the pharmaceutical formulation further comprises at least one additional therapeutic agent.

According to some such embodiments, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

According to another embodiment, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to another embodiment, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

According to another embodiment, the anti-inflammatory agent includes, without limitation, Transforming Growth Factor-beta3 (TGF-β3), an anti-Tumor Necrosis Factor-alpha (TNF-α) agent, or a combination thereof.

According to some embodiments, the additional agent is an analgesic agent. According to some embodiments, the analgesic agent relieves pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, but are not limited to, etodolac, indomethacin, sulindac, tolmetin, nabumetone, piroxicam, acetaminophen, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, but are not limited to, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to some embodiments, the second pharmaceutical formulation comprises an anti-infective agent. According to another embodiment, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Other examples of therapeutic agents include, without limitation, rose hip oil, vitamin E, 5-fluorouracil, bleomycin, onion extract, pentoxifylline, prolyl-4-hydroxylase, verapamil, tacrolimus, tamoxifen, tretinoin, colchicine, a calcium antagonist, tranilst, zinc, and a combination thereof.

### Methods/Use

According to another aspect, the described invention provides a therapeutic regimen for use of a pharmaceutical composition containing a first pharmaceutical formulation containing a therapeutic MK2 peptide and a second pharmaceutical formulation containing an immunostimulatory agent for treating a subject in need thereof, wherein the subject in need thereof is in a non-immunoresponsive, immunotolerant state with regard to an immunostimulating agent that is no longer therapeutically effective for treating a disease, disorder or condition of lung, wherein the therapeutic regimen is effective to resensitize the subject to the immune stimulating agent so that the subject is immunoresponsive to the immune stimulating agent upon its subsequent administration. According to some embodiments, the resensitizing is effective to convert an attenuated or suppressed immune response to a robust immune response when compared to a control.

According to some embodiments, the described invention provides use of, in order, (1) a first pharmaceutical formulation formulated for delivery by inhalation containing an immunomodulatory amount of an MK2 inhibitory peptide followed by (2) a second pharmaceutical formulation containing a therapeutic amount of an immunostimulatory agent, in the manufacture of a medicament for the therapeutic and/or prophylactic treatment of a subject that is in an immunotolerant state with regard to an immunostimulating agent that is no longer therapeutically effective for treating medical disease, disorder or condition of lung, wherein the use is effective to re-sensitize the subject to the immunostimulating agent so that the subject becomes immunoresponsive to the immunostimulating agent upon its subsequent administration. According to some embodiments, the resensitizing is effective to convert an attenuated or suppressed immune response to a robust immune response when compared to a control.

According to some embodiments, the described invention provides a method for treating a subject that is in an immunotolerant state with regard to an immunostimulating agent that is no longer therapeutically effective for treating disease, disorder, or condition of lung tissue comprising, in order (a) administering to the lunga first pharmaceutical formulation formulated for delivery by inhalation containing an immunomodulatory amount of a kinase-inhibiting peptide, and (2) then administering a second pharmaceutical formulation containing a therapeutic amount of the immunostimulatory agent, wherein the method is effective to resensitize the subject to the immunostimulatory agent so that the subject is immunoresponsive to it upon its subsequent administration. According to some embodiments, the resensitizing comprises converting an attenuated or suppressed immune response to a robust immune response.

According to some embodiments, the immunotolerant state of the subject is characterized by an attenuated immune response to the immunostimulatory agent, compared to a normal control.

According to some embodiments, the immunotolerant state is characterized by one or more of a reduced level of synthesis, expression, or both of pro-inflammatory cytokines, anti-inflammatory cytokines, both pro-inflammatory and anti-inflammatory cytokines, or an altered balance between proinflammatory cytokines and anti-inflammatory cytokines, compared to a control.

According to some embodiments the immunotolerant state results from exposure to an immunosuppressive drug. According to some embodiments, the immunotolerant state is a result of repeated prior exposure to the immunostimulatory agent.

According to some embodiments, the immunostimulatory agent is a chemotherapeutic agent. According to some embodiments, the immunostimulatory agent is lipopolysaccharide (LPS).

According to some embodiments, the kinase-inhibiting peptide is MMI0100, a functional equivalent, a peptide mimetic or a variant of MMI0100.

According to some embodiments, the immunomodulatory amount of MMI0100 is effective to modulate MK2 signaling.

According to some embodiments, the immunomodulatory amount of MMI0100 is effective to modulate the MK2 signaling affecting an MAPK pathway, an NfκB pathway, an IFNα/β pathway or a combination thereof.

According to some embodiments, the immunomodulatory amount of the MK2 peptide is effective to modulate one or more of autocrine signaling, paracrine signaling or hormonal signaling in an immune cell population.

According to some embodiments, the immunomodulatory amount of MMI0100 is effective to increase activation of a population of inflammatory cells selected from the group consisting of T cells, B cells, NK cells, CT cells, neutrophils, lymphocytes, macrophages, and dendritic cells.

According to some embodiments, the autocrine signaling, paracrine signaling or hormonal signaling by one or more immune cells comprises TLR-4 signaling.

According to some embodiments, the immune cells are one or more populations selected from T lymphocytes, B lymphocytes, NK cells, CT cells, neutrophils, lymphocytes, dendritic cells, and macrophages.

According to some embodiments, as a result of the signaling, the immune cells express, synthesize, or secrete one or more cytokines selected from the group consisting of IL-1α, IL-1β, IL-2, IL-3, IL-4, IL-5, IL-6, IL-7, IL-8, IL-9, IL-10, IL-11, IL-12/IL-23 P40, IL13, IL-17, IL-18, TGF-β, IFN-γ, GM-CSF, CXCL1, CXCL2, and TNF-α.

According to some embodiments, the level of cytokines expressed, synthesized or secreted is measurable in a body fluid.

According to some embodiments, the body fluid is sputum, blood or both.

According to some embodiments, the immunoresponsive immune response comprises restoration of expression, synthesis or both of inflammatory cytokines in immune cells of the lung without affecting systemic immune cells in an amount to cause unwanted side effects.

According to some embodiments, the disease, disorder or condition is gram negative bacterial sepsis, cystic fibrosis, COPD, or lung cancer. According to some embodiments, the subject is an immunocompromised subject.

### Sepsis

Sepsis, the presence of various pathogenic organisms, or their toxins, in the blood or tissues, is a race to the death between invading microbes and the host immune response, with pathogens seeking an advantage by incapacitating various aspects of host immunity. (Hotchkiss, R. S. et al., “Immunosuppression in sepsis: a novel understanding of the disorder and a new therapeutic approach,” Lancet Infect. Dis. 13(3): 260-68).

There are three theories regarding potential inflammatory responses to sepsis. The immune response in sepsis are determined by multiple factors, including pathogen virulence, size of bacterial inoculum, and comorbidities. According to the first, although both proinflammatory and anti-inflammatory responses begin rapidly after sepsis, the initial response in previously healthy patients with severe sepsis is typified by an overwhelming hyperinflammatory phase with fever, hyperdynamic circulation, and shock. Deaths in this early phase of sepsis are generally due to cardiovascular collapse, metabolic derangements, and multiple organ dysfunction. According to the second, many patients who develop sepsis are elderly, with numerous comorbidities that impair immune response. When these individuals develop sepsis, a blunted or absent hyperinflammatory phase is common, and patients rapidly develop impaired immunity and an anti-inflammatory state. According to the third, the immunological response is characterized by cycling between hyperinflammatory and hypoinflammatory states. Here, patients who develop sepsis have an initial hyperinflammatory response followed by a hypoinflammatory state. With the development of a new secondary infection, patients have a repeat hyperinflammatory response and may either recover or reenter the hypoinflammatory state. Patients can die in either state. There is less evidence for the third theory, and the longer the sepsis continues, the more likely a patient is to develop profound immunosuppression. Id.

Sepsis induces multiple overlapping mechanisms of immunosuppression in spleen and lung. Id. LPS-stimulated splenocytes from patients with sepsis had reduced production of both proinflammatory and anti-inflammatory cytokines, less than 10% of that in patients without sepsis. Both spleen and lung showed upregulated expression of selected inhibitory receptors including PD1, expansion of suppressor cells (Treg and myeloid derived suppressor cells) and concomitant downregulation of activation pathways. (Id. citing Boomer, J. S. et al (2011), “Immunosuppression in patients who die of sepsis and multiple organ failure,” JAMA 306: 2594-2605). Severe depletion of immune effector cells, e.g., CD4 T, CD8 T, B and dendritic cells, is a universal finding in all age groups during sepsis. The net effect of these immunological changes is that the host's ability to combat invading pathogens is severely compromised. Two studies showed that patients with sepsis who were treated with granulocyte macrophage colony stimulating factor (GM-CSF), a cytokine that activates and induces production of neutrophils and monocytes or macrophages, had restoration of HLA-DR expression (Id. citing Meisel, C. et al, (2009) “Granulocyte-macrophage colony-stimulating factor to reverse sepsis-associated immunosuppression: a double-blind, randomized, placebo-controlled multicenter trial,” Am. J. Respir. Crit. Care Med. 180: 640-48; Hall, M W et al (2011) “Immunoparalysis and nosocomial infection in children with multiple organ dysfunction syndrome,” Intensive Care Med. 37: 525-32).

### Cystic Fibrosis

Cystic fibrosis (CF, mucovidosis, mucovisidosis) is an inherited autosomal recessive disorder. It is one of the most common fatal genetic disorders in the United States, affecting about 30,000 individuals, and is most prevalent in the Caucasian population, occurring in one of every 3,300 live births. The gene involved in cystic fibrosis, which was identified in 1989, codes for a protein called the cystic fibrosis transmembrane conductance regulator (CFTR). CFTR normally is expressed by exocrine epithelia throughout the body and regulates the movement of chloride ions, bicarbonate ions and glutathione into and out of cells. In cystic fibrosis patients, mutations in the CFTR gene lead to alterations or total loss of CFTR protein function, resulting in defects in osmolarity, pH and redox properties of exocrine secretions. In the lungs, CF manifests itself by the presence of a thick mucus secretion which clogs the airways. In other exocrine organs, such as the sweat glands, CF may not manifest itself by an obstructive phenotype, but rather by abnormal salt composition of the secretions (hence the clinical sweat osmolarity test to detect CF patients). The predominant cause of illness and death in cystic fibrosis patients is progressive lung disease. The thickness of CF mucus, which blocks the airway passages, is believed to stem from abnormalities in osmolarity of secretions, as well as from the presence of massive amounts of DNA, actin, proteases and prooxidative enzymes originating from a subset of inflammatory cells, called neutrophils. Indeed, CF lung disease is characterized by early, hyperactive neutrophil-mediated inflammatory reactions to both viral and bacterial pathogens. The hyperinflammatory syndrome of CF lungs has several underpinnings, among which an imbalance between pro-inflammatory chemokines, chiefly IL-8, and anti-inflammatory cytokines, chiefly IL-10, has been reported to play a major role. See Chmiel et al. Clin Rev Allergy Immunol. 3(1):5-27 (2002). Studies have reported that levels of TNF-a, IL-6 and IL-1β were higher in the bronchoalveolar lavage fluid of cystic fibrosis patients, than in healthy control bronchoalveolar lavage fluid (Bondfield, T. L., et al. Am. J. Resp. Crit. Care Med. 152(1):2111-2118, 1995).

### COPD

Chronic obstructive pulmonary disease (COPD) is a collective description for lung diseases represented by chronic and relatively irreversible expiratory airflow dysfunction due to some combination of chronic obstructive bronchitis, emphysema, and/or chronic asthma. COPD is caused by a range of environmental and genetic risk factors, including smoking that contributes to the disease.

The prevalence of COPD is increasing worldwide, and COPD has become the fourth leading cause of death in the United States. In the United States, despite the decrease in cigarette smoking in recent decades, both the prevalence of, and the mortality associated with, COPD have increased and are projected to continue to increase for some years yet. Furthermore, COPD is costly, and acute exacerbations, which occur roughly once a year in patients with COPD of moderate or greater severity, constitute the most expensive component.

In COPD, airflow obstruction can occur on the basis of either of two very different pathophysiological processes in the lung: 1) inflammation of the parenchyma resulting in proteolysis of the lung parenchyma and loss of lung elasticity (emphysema); and 2) inflammation, scarring and narrowing of the small airways (“small airway disease”). In an individual patient, one of these processes, which may be controlled by different genetic factors, may predominate although both usually co-exist. Ultimately, both of these processes produce similar patterns of functional impairment: decreased expiratory flow, hyperinflation and abnormalities of gas exchange.

At an early stage of COPD, the following symptoms are found in the lungs of COPD patients: 1) breach of airway epithelium by damaging aerosols, 2) accumulation of inflammatory mucous exudates, 3) infiltration of the airway wall by inflammatory immune cells, 4) airway remodeling/thickening of the airway wall and encroachment on lumenal space, and 5) increased resistance to airflow. During this early stage, smooth muscle contraction and hyper-responsiveness also increase resistance, but the increased resistance is relieved by bronchodilators.

At an advanced stage, COPD patients characteristically develop deposition of fibrous connective tissue in the subepithelial and aventitial compartments surrounding the airway wall. Such peribronchiolar fibrosis contributes to fixed airway obstruction by restricting the enlargement of airway caliber that occurs with lung inflation.

### Lung Cancer

Lung cancer has become the number one killer among cancers worldwide (Molina, J. R. et al., Mayo Clin Proc. 2008 May; 83(5): 584-594). Only 15% of all lung cancer patients are alive five (5) years or more after diaganosis (Ettinger, D. S. et al., J Natl Compr Canc Netw 2010; 8: 740-801). The two (2) main types of lung cancer are small cell lung cancer (SCLC) and non-small cell lung cancer (NSCLC), the latter of which accounts for approximately 85% of all cases of lung cancer (Molina, J. R. et al., Mayo Clin Proc. 2008 May; 83(5): 584-594; Navada, S. et al., J Clin Oncol. 2006; 24(18S) suppl: 384S; Sher, T. et al., Mayo Clin Proc. 2008; 83(3): 355-367).

The primary risk factor for lung cancer is smoking, which accounts for more than 85% of all lung cancer-related deaths (Ettinger, D. S. et al., J Natl Compr Canc Netw 2010; 8: 740-801; Doll, R. et al., Br Med J 1976; 2: 1525-1536). The risk for lung cancer increases with the number of cigarettes smoked per day and the number of years spent smoking. In addition to the hazard of first-hand smoke, exposed nonsmokers have an increased relative risk for developing lung cancer (Ettinger, D. S. et al., J Natl Compr Canc Netw 2010; 8: 740-801; Wald N. J. et al., Br Med J 1986; 293: 1217-1222). Radon gas, a radioactive gas that is produced by the decay of radium 226, is the second leading cause of lung cancer. The decay of this isotope leads to the production of substances that emit alpha-particles, which may cause cell damage and therefore increase the potential for malignant transformation (Ettinger, D. S. et al., J Natl Compr Canc Netw 2010; 8: 740-801; Schrump, D. S. et al., DeVita, Hellman, and Rosenberg's Cancer: Principles & Practice of Oncology, 8th Edition. Vol. 1. Philadelphia: Lippincott Williams & Wilkins; 2008:896-946).

DNA damage signaling and checkpoint control pathways are among the most commonly mutated networks in human tumors (Morandell, S. et al., Cell Reports 5, 868-877, Nov. 27, 2013; Negrini, S. et al., Nat Rev Mol Cell Biol 2010; 11: 220-228). The three major DNA damage-responsive cell cycle checkpoints are the G1/S checkpoint, intra S-phase checkpoint, and the G2/M checkpoint. In response to DNA damage, eukaryotic cells activate complex signaling networks that mediate DNA repair and cell cycle arrest or, if the damage is extensive, trigger apoptosis (Ciccia, A. and Elledge, S. J., Mol Cell 2010; 40: 179-204). Three canonical protein kinase pathways in both normal and cancer cells arrest the cell cycle in response to damaged DNA: the Ataxia-Telangiectasia and Rad-3 related through Chk1 (ATR-Chk1) pathway, the Ataxia-Telangiectasia mutated through Chk2 (ATM-Chk2) pathway, and the stress-activated protein kinases p38 mitogen-activated protein kinase (MAPK) and its substrate MAPKAP kinase-2 (MK2) (Morandell, S. et al., Cell Reports 5, 868-877, Nov. 27, 2013). The ATM/Chk2 pathway responds primarily to DNA double strand breaks, while the ATRpChk1 pathway is activated by bulky DNA lesions, and following replication fork collapse during S-phase. In contrast to the DNA damage-specific activation of Chk1 and Chk2, the p38MAPK pathway is a general stress-activated kinase pathway that responds to various cellular stimuli, including cytokines, hyperosmolarity, and UV irradiation.

Tumor suppressor protein p53 is a major downstream effector of the aforementioned DNA damage kinase pathways. In normal cells, p53-dependent signaling results in G1 arrest, mainly mediated by transcriptional upregulation of p21. P21 also appears to play a role in sustaining the G2 checkpoint after gamma-irradiation. If DNA damage is extensive, however, p53-dependent pathways target the damaged cell for apoptotic cell death. The MK2 pathway is critical for arresting the cell cycle after genotoxic stress, including cisplatin-induced DNA crosslinks and topoisomerase-inhibitor-induced DNA strand breaks only in tumor cells that lack functional p53 (Manke, I. A. et al., Mol Cell 2005; 17: 37-48; Reinhardt, H. C. et al., Cancer Cell 2007; 11: 175-189). Both the ATRChk1 pathway and the p38-MK2 pathway are required for effective cell-cycle checkpoint function in the absence of p53 (Reinhardt, H. C. et al., Mol Cell 2010; 40: 34-49).

Morandell et al. (Cell Reports 5, 868-877, Nov. 27, 2013) showed that, in response to genotoxic chemotherapy, MK2 is essential for the survival of NSCLC tumor cells that lack functional p53 but is dispensable in p53-proficient cells. MK2 was found to specifically sensitize p53-deficient tumors to the DNA-damaging agent cisplatin but had no effect on the treatment response of p53-proficient cancer cells. This suggests a potential for enhancement chemosensitization of p53-deficient tumors to DNA-damaging chemotherapy in vivo through synthetic lethality between p53 and MK2 (Morandell, S. et al., Cell Reports 5, 868-877, Nov. 27, 2013).

FasL and its receptor Fas (also known as APO-1 and CD95), play a key role in the regulation of apoptosis within the immune system. (Niehans, G A, et al. (2007) “Human lung carcinomas express Fas ligand,” Cancer Res. 57: 1007-12). Both proteins are highly expressed on activated T cells (Id. citing Nagata, S., Golstein, P. (1995) “The Fas death factor,” Science 267: 1449-56), with low levels of expression seen in resting T cells (Id. citing Trauth, BC (1989) “Monoclonal antibody-mediated tumor regression by induction of apoptosis,” Science 245: 301-305; Suda, T. et al. (1993) “Molecular cloning and expression of the Fas ligand, a novel member of the tumor necrosis factor family,” Cell 75: 1169-788; Owen-Schaub, L P, et al (1992) “DNA fragmentation and cell death is selectively triggered in activated human lymphocytes by Fas antigen engagement,” Cell Immunol. 140: 197-205; Klas, C. et al (1993) “Activation interferes with the APO-1 pathway in mature human T cells,” Int. Immunol 5(6): 625-630). FasL-Fas interactions have been shown to be required for activation-induced cell death in peripheral T cells (Id. citing Singer, G G and Abbas, A K (1994) “The Fas antigen is involved in peripheral but not thymic deletion of lymphocytes in T cell receptor transgenic mice,” Immunity 1: 365-71; Alderson, M R, et al (1995) “Fas ligand mediates activation-induced cell death in human T lymphocytes,” J. Exp. Med. 181: 71-77; Dhein, J. et al (1995) “Autocrine T-cell suicide mediated by APO-1/Fas/CD95,” Nature 373: 438-41; Brunner, T et al (1995) “Cell-autonomous Fas (CD95)/Fas ligand interaction mediates activation-induced apoptosis in T cell hybridomas,” Nature 373: 441-44; Ju S-T et al (1995) “Fas (CD95)/FasL interactions required for programmed cell death after T-cell activation,” Nature 373: 444-48), which may normally be needed to terminate an immune response at the end of infection and/or to peripherally delete autoreactive clones. FasL expression has been implicated in ocular tissues and Sertoli cells as a critical factor in maintaining immune privilege in the eye and testis, respectively.

It has been shown that neoplastic cells prevent a T-cell immune response by various means, including down-regulation of MHC class I molecules (Id. citing Cordon-Cardo, C. et al (1991) “Expression of HLA-A, B, C antigens on primary and metastatic tumor cell populations of human carcinomas,” Cancer Res. 51: 6372-80; Restifo, N P et al (1993) “Identification of human cancers deficient in antigen processing,” J. Exp. Med. 177: 265-72), lack of costimulatory signals, such as B7 (Id. citing Chen L et al. (1992) “Costimulation of antitumor immunity by the B7 counterreceptor for the T lymphocyte molecules CD28 and CTLA-4,” Cell 71: 1093-1102; Townsend, S., Allison, JP (1993) “Tumor rejection after direct costimulation of CD8+ T cells by B7-transfected melanoma cells,” Science 259: 368-70), secretion of immunoinhibitory proteins such as TGFβ (Id. citing Sulitzeanu, D (1993) “Immunosuppressive factors in human cancer,” Adv. Cancer Res. 60: 247-71), loss of ζ signal transducing chains from tumor-infiltrating lymphocytes (Id. citing Mizoguchi, H. et al (1992 “Alterations in signal transduction molecules in T lymphocytes from tumor-bearing mice,” Science 258: 1795-98; Nakagomi, H. et al (1993) “Decreased expression of the signal-transducing ζ chains in tumor-infiltrating T cells and NK cells of patients with colorectal carcinoma,” Cancer Res. 53: 5610-12; Finke, J. H. et al (1993) “loss of T-cell receptor ζ chain and p561ck in T-cells infiltrating human renal cell carcinomas,” Cancer Res. 53: 5613-16), and interaction of neoplastic cells with the inhibitory CTLA-4 receptor (Id. citing 50).

Well-characterized human lung carcinoma cell lines and primary human lung neoplasms were examined for evidence of FasL production, on the theory that expression of FasL might protect tumor cells from immune attack. Six NSCLC cell lines (H522, H1155, H2009, H2030, H2087 and H2172) and 10 SCLC cell lines (H69, H209, H417, H685, H689, H719, H774, H792, H865, and H1436) were tested. Cell line H2373 is a mesothelioma cell line. Whole cell lysates were prepared from 5×10⁶ cells in 1 ml lysis buffer, and 100 ug of total protein electroblotted onto nitrocellulose following separation on a 12.5% SDS-PAGE gel for FasL detection. Filters were blocked for 1 hr using 5% dry milk/1% BSA in PBS and incubated overnight at 4 C in a sealed bag with a 1:1000 dilution of the α human FasL murine monoclonal antibody (Transduction Laboratories, Lexington Ky.) and then incubated with a rabbit α murine IgG secondary antibody (PharMingen, San Diego, Calif.) and 2.5×10⁶ cpm 125I-protein A followed by autoradiography. Is.

Lung cancer cell lines H209, H2009, H522 and H841 were seeded at 1×10⁵ cells/ml into tissue culture chamber slides and growth for 2 days at 37 C. Cells were washed twice with PBS, fixed with 4% paraformaldehyde in PBS; permeabilized in 0.25% triton×100; washed with PBS; blocked with 1% BSA in PBS, and then stained for FasL with 5 ug/ml murine monoclonal α-human FasI antibody (NOK-1) (PharMingen) overnight at 37 C. Cells were washed three times in PBS/BSA and incubated with a 1:50 dilution of a goat α mouse FITC conjugate for 30 minutes at room temperature. Id.

In all cell lines analyzed, a protein corresponding to FasL was identified that comigrated with an extract of testicular tissue used as a positive control. Tissue samples from a resected NSCLC also demonstrated the presence of this protein. Immunofluorescent staining of two SCLC cell lines (H209 and H841) and two NSCLC cell lines (H522 and H2009) also demonstrated FasL expression by lung cancer cells. Normal alveolar lung tissue was negative for FasL. Subsequently a 565 bp product was identified in all lung cancer cell lines and in the primary tumor by nested RT-PCR consistent with human FasL. Id.

To evaluate the functional significance of FasL expression in lung cancer cells and tumors, coculture experiments of the lung cancer cell lines with the Fas-positive Jurkat cell line (of human T cell origin) as a target cell were conducted. Jurkat cells have been shown to be capable of apoptosis following exposure to FasL. Id. A marked decrease in viable Jurkat cells detected by trypan blue exclusion was found, compared to Jurkat cells grown in the absence of human lung cancer cells, which showed marked proliferation after 24 h. Although the cytotoxic capacity of human lung cancer cells was very reproducible, the extent of Jurkat cell killing was variable between experiments and cell lines. Id.

It was suggested that FasL expression by lung carcinomas may have effects on the immune system beyond the tumor site. Soluble FasL has been identified in sera from individuals with certain types of FasL positive leukemias and lymphomas (citing 36). It was hypothesized (1) that if circulating forms of FasL are also found in blood from lung cancer patients, this could contribute to the generalized depression of cellular immunity seen in patients with advanced neoplastic disease (Id. citing Trauth, B. et al. (1989) “Monoclonal antibody-mediated tumor regression by induction of apoptosis, Science 245: 301-305; Brugarolas, A., Takita, H. (1973) “Immunologic status in lung cancer,” Chest 64: 427-30), and (2) that FasL expression might facilitate tumor invasion by inducing apoptosis in surrounding Fas-positive tissue, allowing the tumor to grow into the resulting space.

Additionally, cell populations in the tumor microenvironment that contribute to local immunosuppression have been identified. Makkouk, A. and Weiner, G. (2015) “Cancer immunotherapy and breaking immune tolerance—new approaches to an old challenge,” Cancer Res. 75(1): 5-10. For example, myeloid derived suppressor cells (MDSCs) can be abundant in the tumor microenvironment, have profound suppressive effects on T cells, and can activate T regulatory cells. Id. The presence of MDSCs and Tregs at the tumor site or in peripheral blood has been shown to correlate with poor prognosis in several types of cancer. Id. Other substances produced by tumors, including IL-10 and VEGF, can block differentiation of myeloid DCs and lead to accumulation of immature DCs with reduced expression of costimulatory molecules (CD80/CD86) leading to T cell anergy. Id.

Exemplary chemotherapeutic agents for treating lung tumors include, without limitation, Abitrexate (Methotrexate), Abraxane (Paclitaxel Albumin-stabilized Nanoparticle Formulation), Afatinib Dimaleate, Alimta (Pemetrexed Disodium), Avastin (Bevacizumab), Bvacizumab, Carboplatin, Ceritinib, Cisplatin, Crizotinib, Cyramza (Ramucirumab), Docetaxel, Erlotinib Hydrochloride, Folex PFS (Methotrexate), Gefitinib, Gilotrif (Afatinib Dmaleate), Gemcitabine Hydrochloride, Gemzar (Gemcitabine Hydrochloride), Iressa (Gefitinib), Mechlorethamine Hydrochloride, Methotrexate, Methotrexate LPF (Methotrexate), Mexate (Methotrexate), Mexate-AQ (Methotrexate), Mustargen (mechlorethamine Hydrochloride), Navelbine (Vinorelbine Tartrate), Paclitaxel, Paclitaxel Albumin-stabilized nanoparticle Formulation, Paraplat (Carboplatin), Paraplatin (Carboplatin), Pemetrexed Disodium, Platinol (Cisplatin), Platinol-AQ (Cisplatin), Ramucirumab, Tarceva (Erlotinib Hydrochloride), Taxol (Paclitaxel), Taxotere (Docetaxel), Vinorelbine Tartrate, Xalkori (Crizotinib), Zykadia (Ceritinib), Carboplatin-Taxol, and Gemcitabine-Cisplatin.

A broad variety of agents that may impact immune tolerance induced by the tumor microenvironment have been used to tip the balance from immune tolerance to immune reactivity. Id. For example, interleukins (e.g., IL-2, IL-7, IL-12, and IL-15) have been investigated either as single agents or in combinatorial vaccine approaches. Id. Immune checkpoints, which tightly regulate the magnitude of the T cell response and are critical for avoiding autoimmunity, also limit the robustness and duration of desirable anti-tumor immune responses. Id. Molecules that play a key role in checkpoint regulation include the T cell surface molecules cytotoxic T-lymphocyte antigen 4 (CTLA-4), programmed death-1 (PD-1), T cell immunoglobulin and mucin domain-containing protein 3 (Tim-3) and lymphocyte activation gene-3 (LAG-3). Id. In the tumor microenvironment, the expression of these markers by intratumoral lymphocytes results in hyporesponsiveness sometimes described as immune exhaustion. Id. A number of these molecules are also highly expressed on Tregs, are employed to suppress effector T cells, and are potential targets for reversing immune tolerance. This has led to the development of checkpoint blockade mAbs that recognize receptor or ligand, interfere with their interaction, and can enhance the antitumor immune response (e.g., CTLA-4 mAb ipilimumab).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. LPS Challenge Study

Lipopolysaccharide (LPS) is an inflammatory factor found in the cell wall of Gram-negative bacteria. Inhaled LPS induces a dose-dependent, acute neutrophilic response in the airways of healthy volunteers that can be quantified in induced sputum. See, e.g., Leaker, B R et al. (2013), “Inhibition of LPS-induced airway neutrophilic inflammation in healthy volunteers with an oral CXCR2 antagonist,” Respir. Res. 14(1): 137). This method closely replicates key components of the inflammatory response associated with COPD, severe asthma, and CF. Id. Chemokines, such as CXCL1 and CXCL8, play an important role in neutrophilic inflammation in the lung through the activation of CXCR2 n acute airway neutrophilia. (Id.).

An LPS challenge therefore is a useful and convenient way to initially evaluate the effect of immunomodulatory drugs. Moreover, induced sputum can be used to sample the airways and quantify the inflammatory influx.

To explore the potential for MMI-0100 in pulmonary diseases with an inflammatory component, an LPS challenge was employed to produce an artificial, short-term but measureable inflammatory response that might be modulated by MMI-0100.

Smokers were chosen as the test population to provide sufficient sputum to conduct multiple biomarker analyses and because they already exhibit some inflammatory changes.

Clinical precedent with this approach suggested a reasonable likelihood of success. While LPS challenge responses produced by smokers may be blunted/more variable than those of non-smokers (See, e.g., Laan, M. et al. (2004) “Cigarette smoke inhibits lipolysaccharide-induced production of inflammatory cytokines by suppressing the activation of activator protein-1 in bronchial epithelial cells,” J. Immunol. 173: 4164-70), LPS inhalation in healthy smokers has been reported to be well-tolerated. Aul, R. et al. (2012), “Inhaled LPS challenges in smokers: a study of pulmonary and systemic effects,” Br. J. Clinical Pharmacol. 74(6): 1023-32) investigated LPS inhalation in healthy smokers as a model of COPD bacterial exacerbations. Twelve smokers inhaled 5 and 30 μg LPS and safety was monitored over 24 hours. The following biomarkers of inflammation were measured in serum samples collected at baseline, 4, 8 and 24 h: IL-6, C-reactive protein (CRP), pulmonary and activation-regulated chemokine (CCI-18/PARC), surfactant protein D (SP-D), Clara cell protein (CC-16) and β defensin 2. Significant increases occurred in sputum neutrophil counts with both doses. LPS increased sputum cell nuclear p65 translocation and phosphor-p65 expression. All of the serum biomarkers increased following challenge but with different temporal patterns. The authors concluded that since inhaled LPS challenge in smokers causes pulmonary and systemic inflammation that involves NFκB activation, this appears to be a suitable model for studying bacterial exacerbations of COPD.

In this LPS challenge study conducted in “healthy smokers”, MMI-0100's effects on biomarkers of MK2 target engagement and inflammation were investigated.

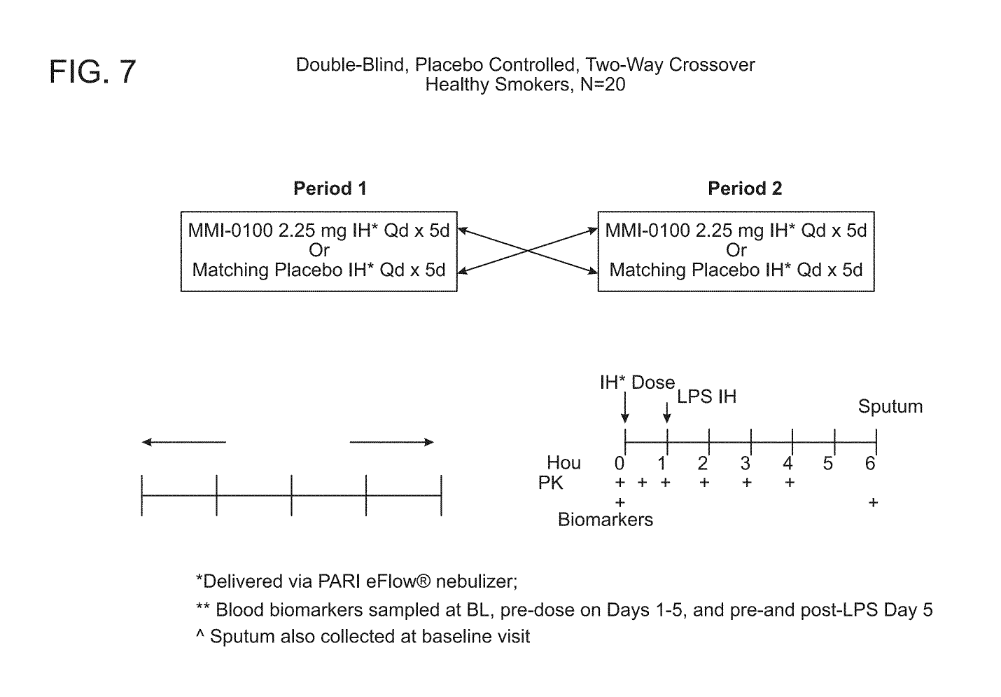

As shown in FIG. 7, a two-way cross-over design was selected to enable between-group (placebo vs. drug) but also to permit intra-subject comparisons of inflammatory markers. Period/sequence effects were analyzed to determine potential impact of treatment order.

First multi-day dosing in humans employed a single low dose of 2.25 mg, extrapolated from animal studies (37.5 μg/kg, based on 60 kg human). Anticipated variability predicts N=16 to provide 80% power to demonstrate 50% effect between drug and placebo groups.

The primary study endpoints were safety and tolerability following first repeat administration and induced sputum inflammatory biomarkers interleukin-6 (IL-6), interleukin-1β (IL-1β), interleukin-8 (IL-8) and tumor necrosis factor alpha (TNFα) following inhaled LPS (30 μg) challenge, day 5. Secondary study endpoints were: induced sputum cell counts (total cell; total neutrophil and differential (%) counts; total macrophage and differential (%) counts following inhaled LPS challenge; phosphorylation of MK2 protein in sputum leukocytes at day 3 each period (STAT1 surrogate), and pharmacokinetics of MMI-0100 in blood (buffy coat). Multiple exploratory biomarkers were interrogated in sputum and blood.

Biomarkers were evaluated in induced sputum (N=16) at screening, Day 3 and Day 5 post-LPS. Subjects were not re-baselined between treatment periods. Biomarkers were evaluated in blood (N=20) at Day 1 pre-treatment (baseline for each treatment period), Day 3 pre- and post-treatment and, on Day 5, both pre-treatment and at a single time point post-LPS challenge (5 hours) optimized to capture peak IL-6.

Demographics of the study subjects who completed the study (n=20)* are shown in table 2 below. 22 subjects were randomized; 16 subjects completed the protocol for sputum measurements

TABLE 2
Demographics of study subjects. The data below are reported
as mean (SD)(range) unless otherwise stated.

| Age (Years) | 40.3 | (11.6) [20-57] |
|---|---|---|
| Male (%) | 95% | |
| Race(%) | | |
| Caucasian | 65% | |
| African | 20% | |
| American | 15% | |
| Other | | |
| Weight (kg) | 79.3 | (13.8) [55-105] |
| BMI (kg/m²) | 25.9 | (3.8) [18.2-32.5] |
| Smoking History | | |
| Currently | 100% | |
| Smoking (%) | 24 | (10.7) [10-50] |
| # Cigarettes/Day | 30.5 | (25.2) [10-90] |
| Pack Years | | |

A summary of treatment emergent adverse effects (TEAE) (N=22) is shown in Table 3.

TABLE 3
Summary of treatment emergent adverse effects (TEAE) (N = 22).

| | | |
|---|---|---|
| Patients, N (%) | MMI-0100 | Placebo |

| | | | | |
|---|---|---|---|---|
| Any TEAE | 9 | (40.9%) | 7 | (31.8%) |
| Mild | 6 | (27.3%) | 6 | (27.3%) |
| Moderate | 4 | (18.2%) | 3 | (13.6%) |
| Severe or life threatening | 0 | (0%) | 0 | (0%) |
| Deaths | 0 | (0%) | 0 | (0%) |
| Any AE leading to | 0 | (0%) | 0 | (0%) |
| treatment D/C | | | | |

As shown in Table 4, there were no treatment-related serious adverse events (SAE). One unrelated SAE was reported during the placebo period.

TABLE 4
Treatment related serious adverse events (SAE)

Incidence of Serious

| Treatment Emergent | | | | | | |
|---|---|---|---|---|---|---|
| Adverse Events by System | | | | | | |
| Organ Class and Preferred | | | | | | |
| Term Safety Population | All Subjects | | MMI-0100 | | Placebo | |
| (N = 22) | (N = 22) | | (N = 22) | | (N = 22) | |
| Any serious treatment | 1 | (4.5%) | 0 | (0.5%) | 1 | (4.5%) |
| emergent adverse event | | | | | | |
| Number of serious | 3 | | 0 | | 3 | |
| treatment emergent | | | | | | |
| adverse events | | | | | | |
| Injury, poisoning and | 1 | (4.5%) | 0 | (0%) | 1 | (4.5%) |
| procedural complications | | | | | | |
| Concussion | 1 | (4.5%) | 0 | (0%) | 1 | (4.5%) |
| Joint dislocation | 1 | (4.5%) | 0 | (0%) | 1 | (4.5%) |
| Road traffic accident | | | | | | |

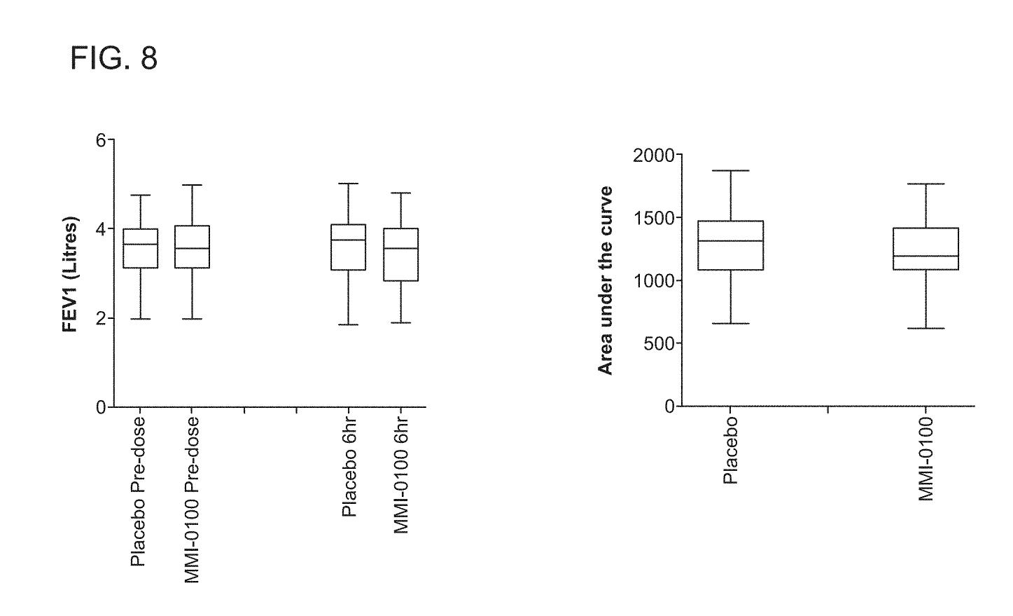

As shown in FIG. 8, no deleterious effect on lung function (FEV1) was observed following repeat dosing of MMI-0100.

Sputum biomarker results are shown in Table 5.

Group-level biomarkers of inflammation/matrix remodeling showed favorable changes in the MMI-0100 treated group compared with placebo.

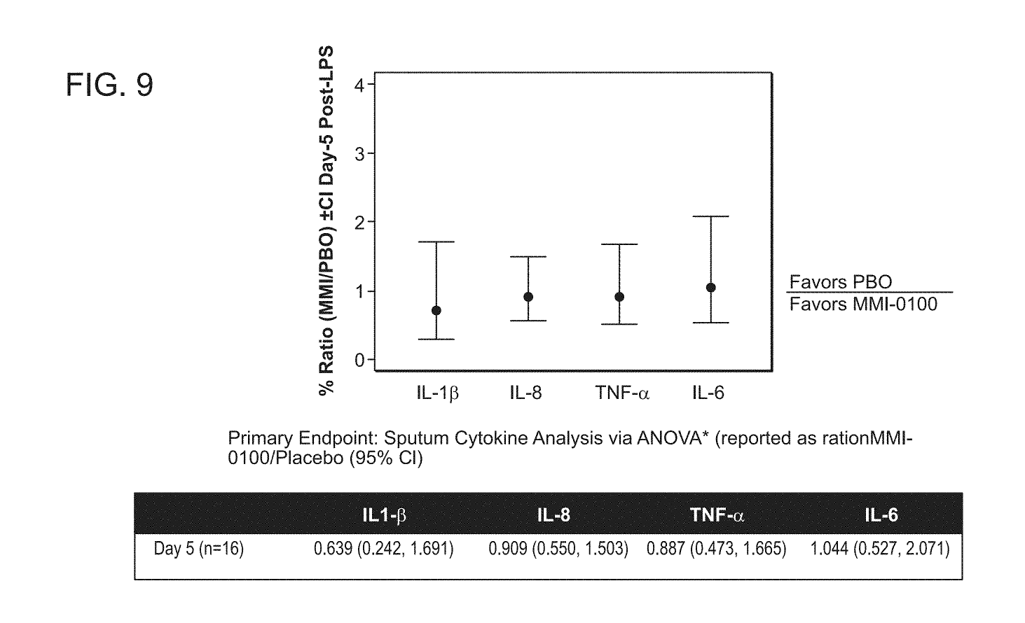

FIG. 9 shows primary endpoint data, sputum cytokine analysis (N=16) reported as the ratio of MMI-0100/Placebo (95% CI).

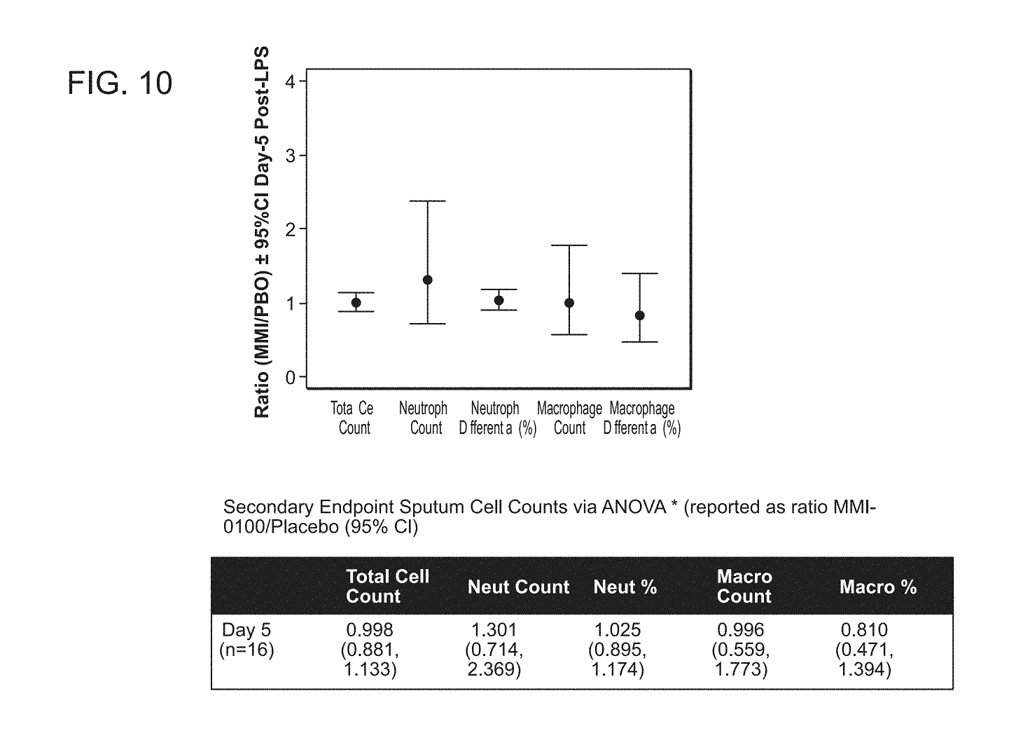

FIG. 10 shows secondary endpoint sputum cell counts (N=16) reported as the ratio of MMI-0100/Placebo (95% CI).

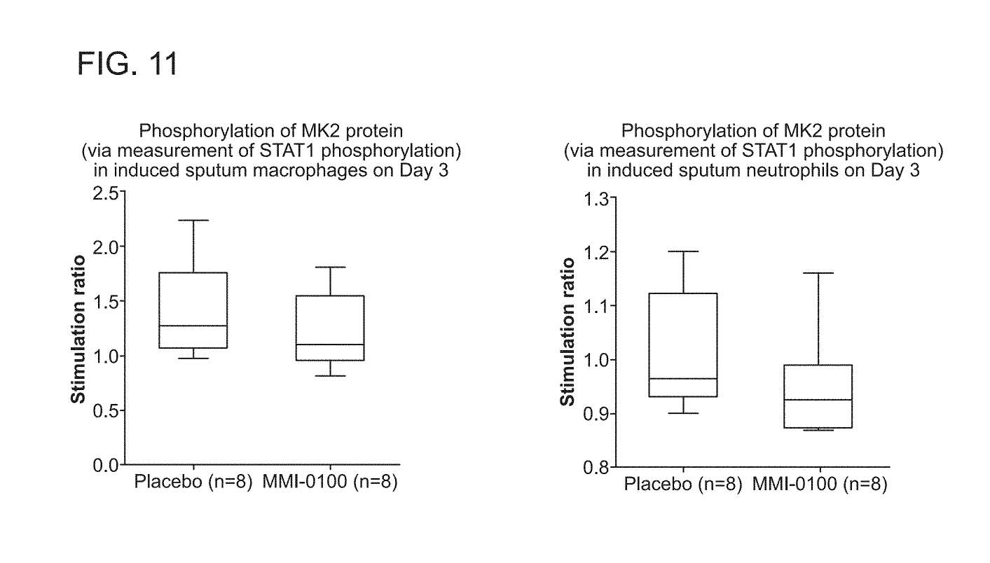

Phosphorylation of MK2 protein via measurement of STAT1 phosphorylation in induced sputum macrophages was measured on day 3. The data are shown in FIG. 11. 6/8 subjects had a reduction in MK2 phosphorylation post-MMI-0100 administration, compared with placebo. 4/4 subjects who produced a sputum sample on Day 3 during both treatment periods (‘true’ placebo rather than substituting in screen sputum for a missing placebo sputum) had a reduction in MK2 phosphorylation following administration of MMI-0100 compared with placebo.

The inflammatory cytokine 1l-1β was selected as an indicator of the effect of MMI-0100 following LPS challenge. A reduction in IL-1β concentration of >25% after MMI-0100 compared to placebo was required for the subject to be classified as a “responder”. IL-1β responders are highlighted in Table 5, and their response as measured via other biomarkers was assessed.

10/16 individual subjects were identified as “responders”, meaning they demonstrated an anti-inflammatory response (as measured by reductions in primary cytokine measures) to MMI-0100 vs. placebo. These were subjects who mounted a robust inflammatory response to LPS challenge in the placebo period. More subjects who were treated first with MMI-0100 displayed robust LPS challenge responses in the placebo period.

Table 6 shows that sputum cytokine responses delineate MMI-0100 “responder” (N=10) and “non-Responder” (N=6) Groups (FAS, N=16). MMI-0100 “responders” (shown in blue) showed a decrease on 22 inflammatory cytokine measures on drug vs. placebo. In subjects who mounted an inflammatory response to LPS, an anti-inflammatory response to MMI-0100 was observed. Subjects 036, 054, 057, and 064 were excluded for missing data points.

### Between-Group Conclusions

MMI-0100 was safe and well-tolerated following first repeat administration. There were no statistically significant differences in sputum biomarkers or cell counts between MMI-0100 and placebo groups post LPS challenge after 5 days of dosing in evaluable subjects, which suggests that the tested dose was too low and/or the study was underpowered given observed vs. anticipated variability.

Multiple statistically significant period and sequence effects were observed, which suggests that a cross-over study design with “healthy smokers” may not be ideal to study MMI-0100's impact on LPS challenge.

### Subject-Level Data Observations

The number of evaluable subjects in this study was 16. High variability was observed in baseline demographics (body weight, BMI), as well as sputum biomarker measures (greatly in excess of that assumed/anticipated in powering the study) as well as magnitude/direction of response to inflammatory challenge (LPS) in placebo treatment period. Aaron et al ((2010) “Multi-analyte profiling and variability of inflammatory markers in blood and induced sputum in patients with stable COPD,” Respiratory Res. 11:41) reported significant intra- and inter-patient variability in repeated measurement of inflammatory markers in induced sputum, with greater variation in sputum concentrations for most proteins (which can be attributed, in part, to variability in the amount of sputum yielded from the lower respiratory tract, its purulence and relative dilution from day to day in clinically stable subjects) compared to variations in serum protein concentrations.

Of the four primary endpoints (IL-8, TNF-α, IL-6 and IL-1β), only IL-8 was sufficiently powered to match the observed variability. For TNF-α, IL-6 and IL-1β, an n of 26, 29, and >50, respectively, were needed.

### Overall Sputum Data Conclusions

Group-level sputum primary and secondary endpoint data (N=16) suggest that MMI-0100 is safe and well-tolerated in first multi-day dosing. There was a trend toward anti-inflammatory activity on primary sputum cytokine endpoints on Day 5, which suggests that the chosen MMI-0100 dose was at the low end of its therapeutic range. pMK2 at Day 3 is directionally-supportive of target engagement. Because variability in the sputum biomarker measures was greater than anticipated, direction/magnitude of LPS challenge response suggest that the study may have been under-powered and/or smokers were tolerized to LPS.

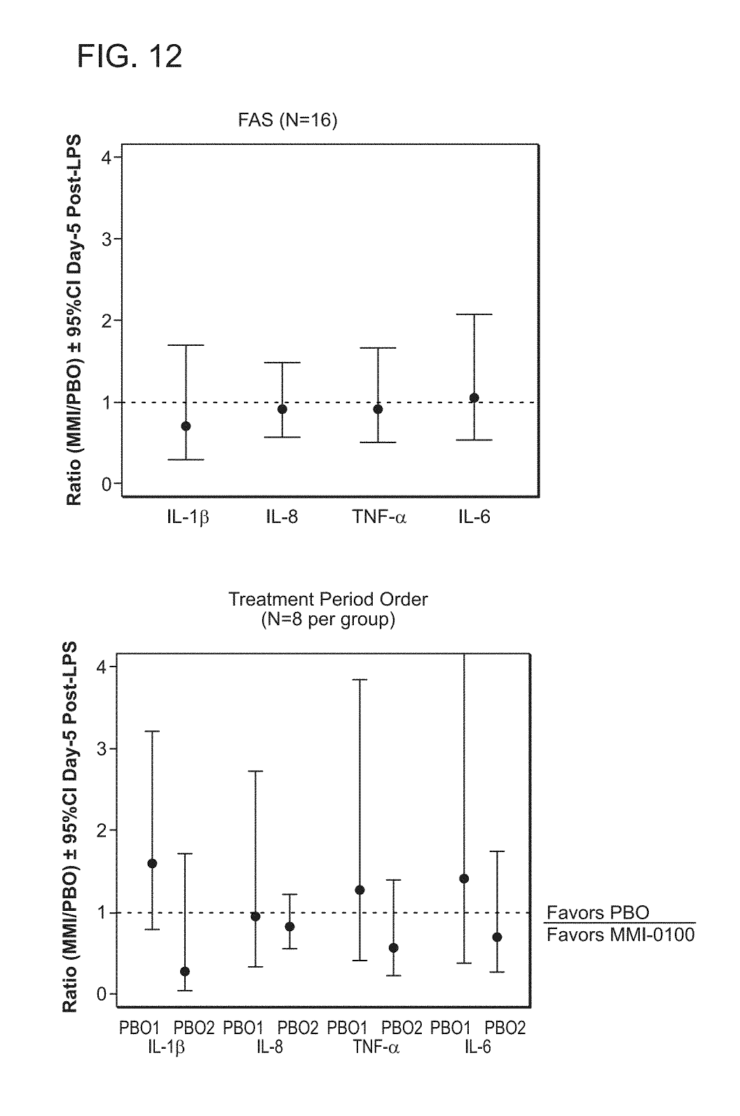

A period effect may explain the group-level data. As shown in FIG. 12, right panel, significant period and/or sequence effects were identified for IL6, IL-1β and IL-8 in sputum cytokine endpoints. For the IL1β period effect, p=0.0623 for Day 5 and p=0.0523 for change from baseline at Day 5 (recall IL1β was the most underpowered). For IL-6, p=0.0101 for a sequence effect Day 5-Day 3. For IL8, p=0.0368 for a sequence effect.

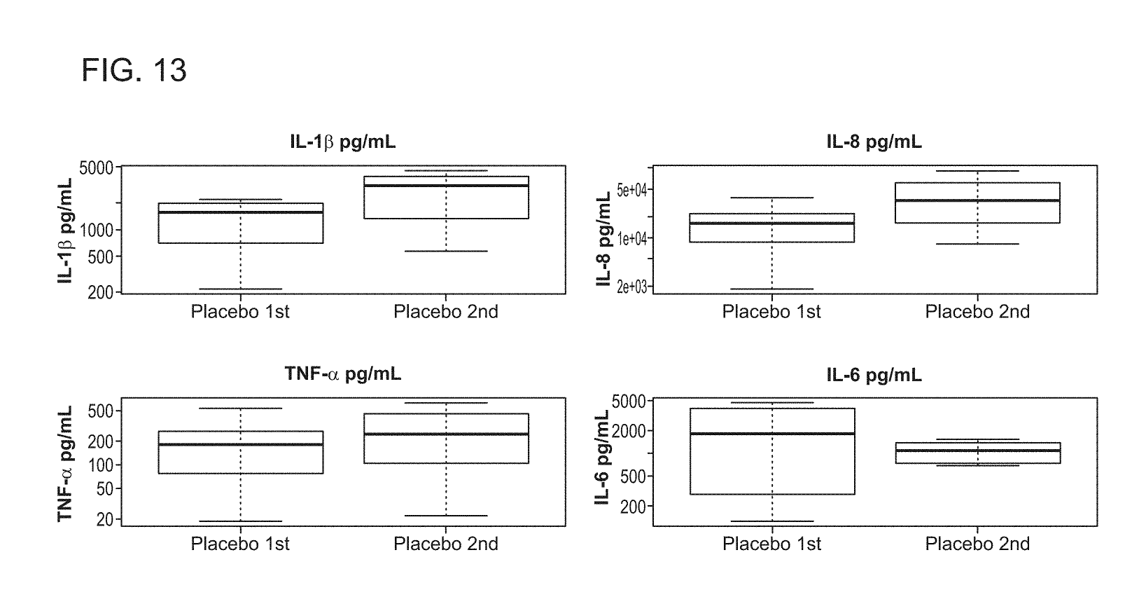

As shown in FIG. 13, when the treatment order was drug then placebo (when drug was no longer present), a big LPS inflammatory response was seen upon second challenge; when the treatment order was placebo then drug, a smaller LPS inflammatory response was seen followed by a somewhat larger LPS response with drug on board sensitizing the subject to LPS.

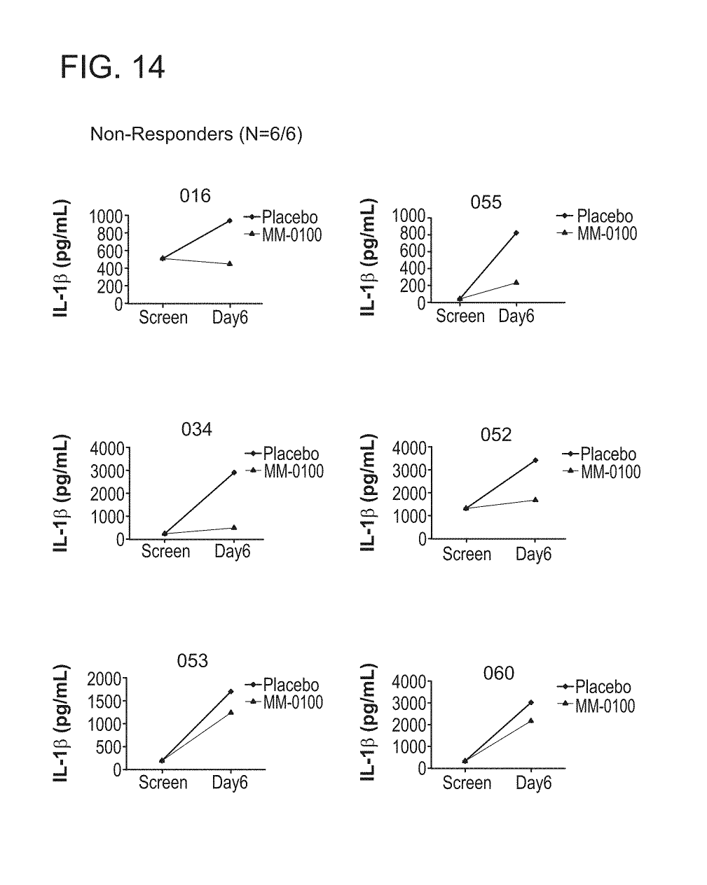

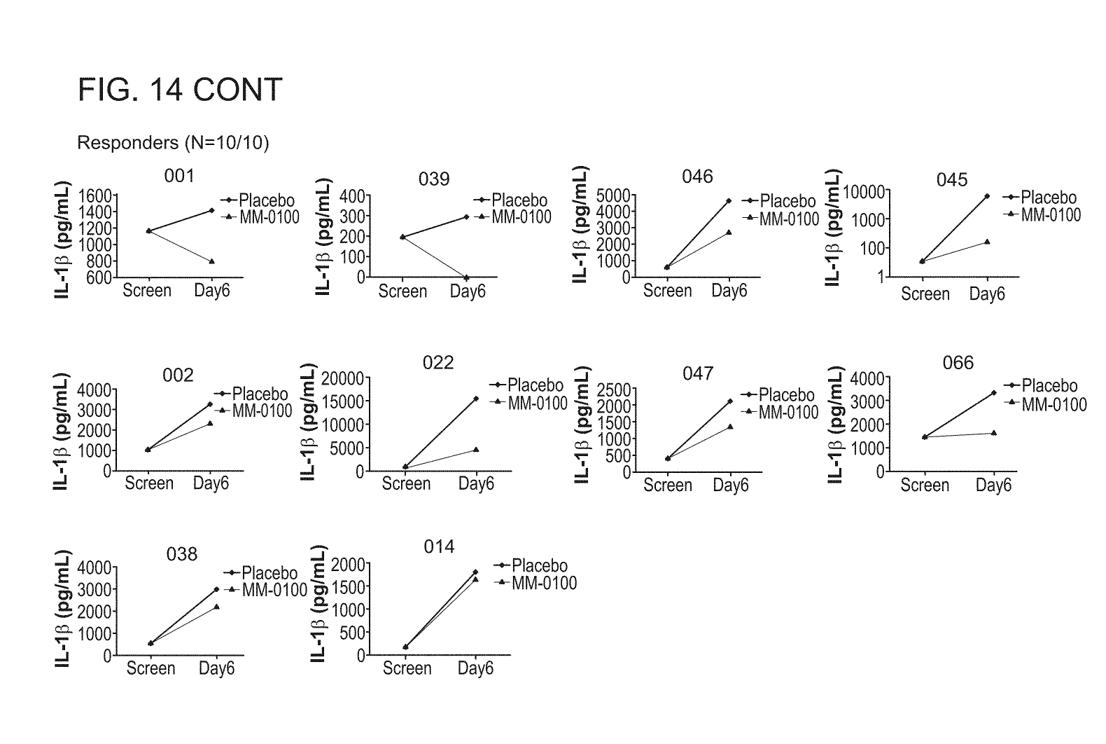

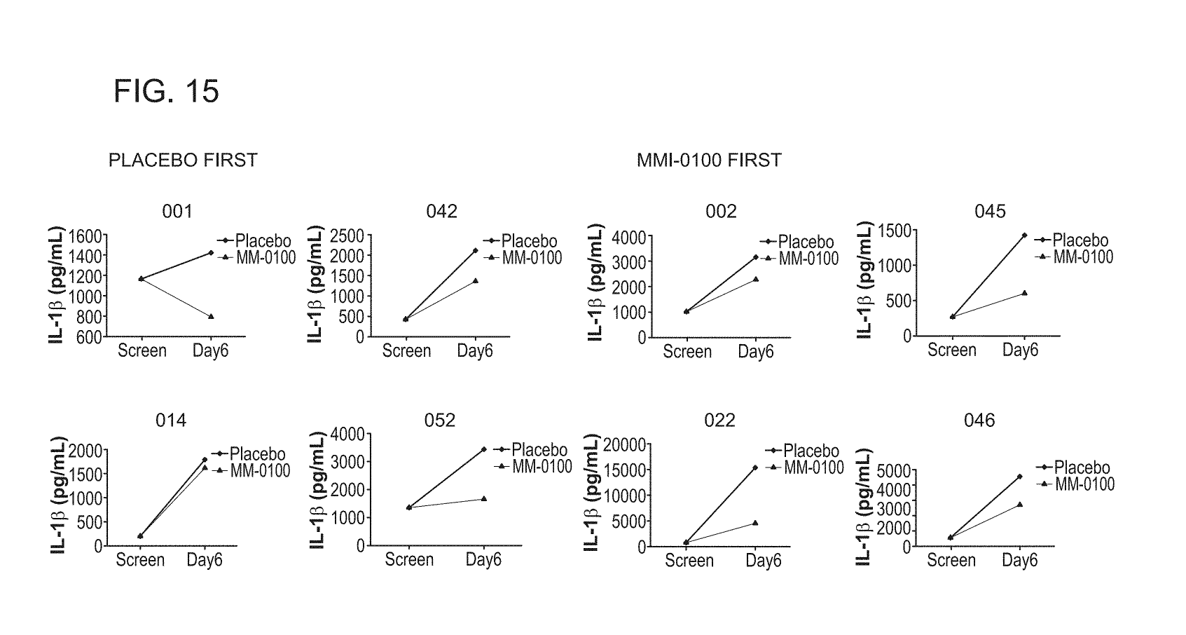

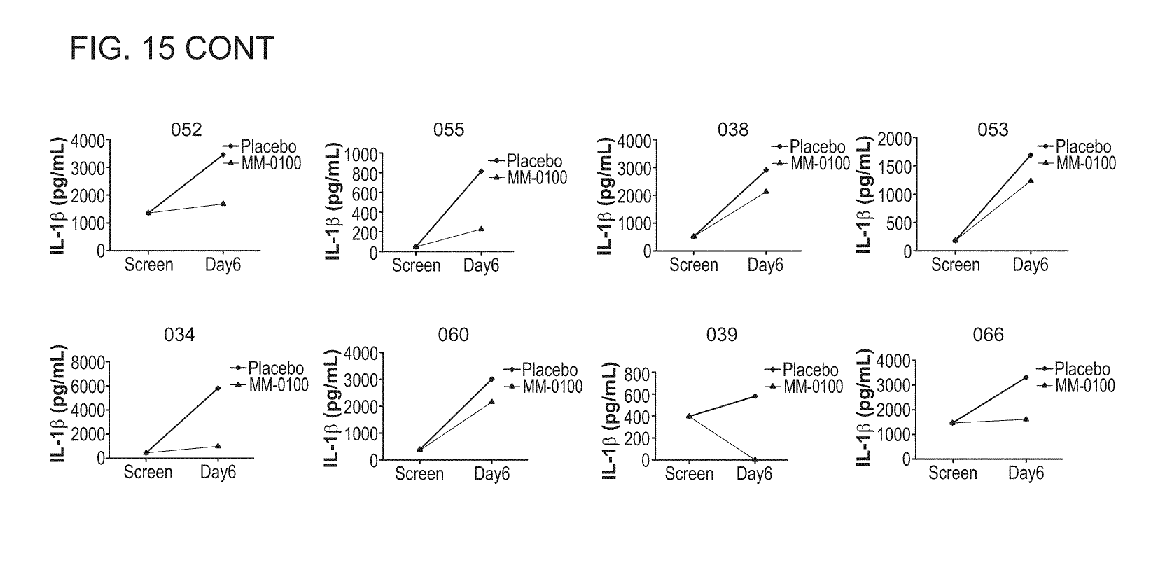

The subject-level sputum endpoint data analyses suggest that primary sputum cytokine responses define MMI-0100 responder (N=10) and non-responder (N=6) subgroups. As shown in FIG. 14 and FIG. 15, 7/10 responders were treated first with MMI-0100 in Period 1 (N=8), while 3/10 responders were treated first with placebo in Period 1 (N=8) and had robust inflammatory responses to LPS challenge. LPS challenge responses in subjects treated first with placebo were smaller in magnitude than in subjects treated with placebo second, in Period 2. The treatment sequence/responder analyses suggest that prior exposure to MMI-0100 re-sensitized LPS tolerant subjects upon subsequent LPS challenge.

### Blood Biomarker Results

The blood biomarker results are found in Table 7.

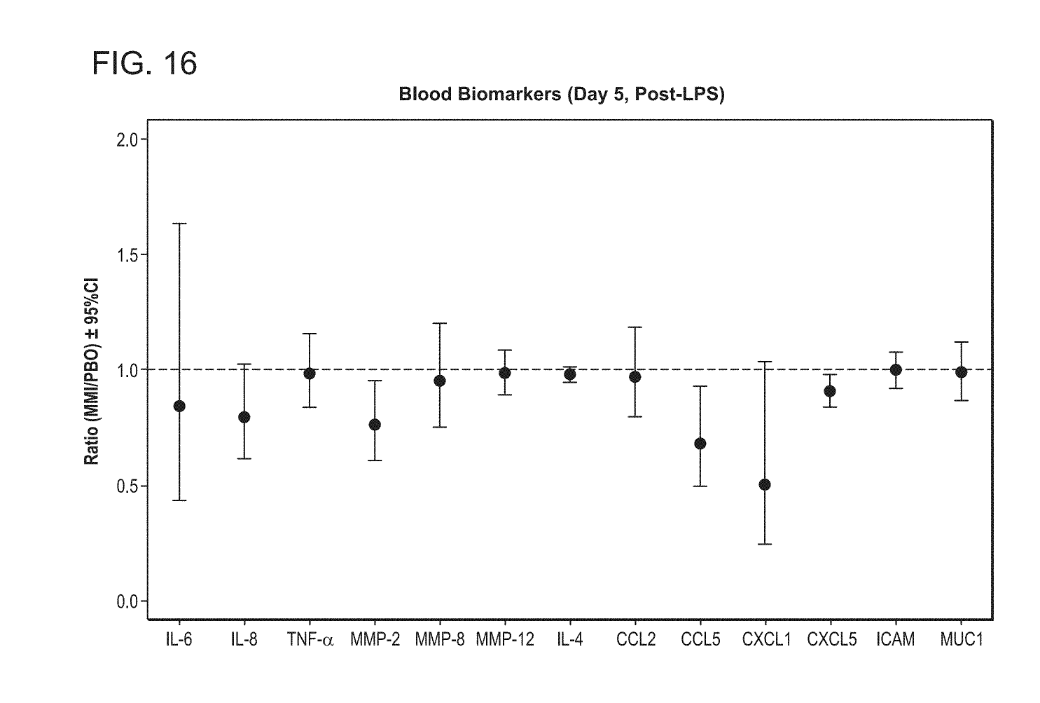

FIG. 16 shows blood biomarkers IL6, IL8, TNF-α, MMP-2, MMP-8, MMP-12, IL4, CCL2, CCL5, CXCL1, CXCL5, ICAM and MUC1 day 5, post-LPS plotted as the ratio MMI/PBO±95% confidence interval. Modulation of blood biomarkers was observed, even when MMI-0100 is locally administered; this mirrors the data obtained with inhaled MMI-0100 in the bleomycin animal model. (data not shown)

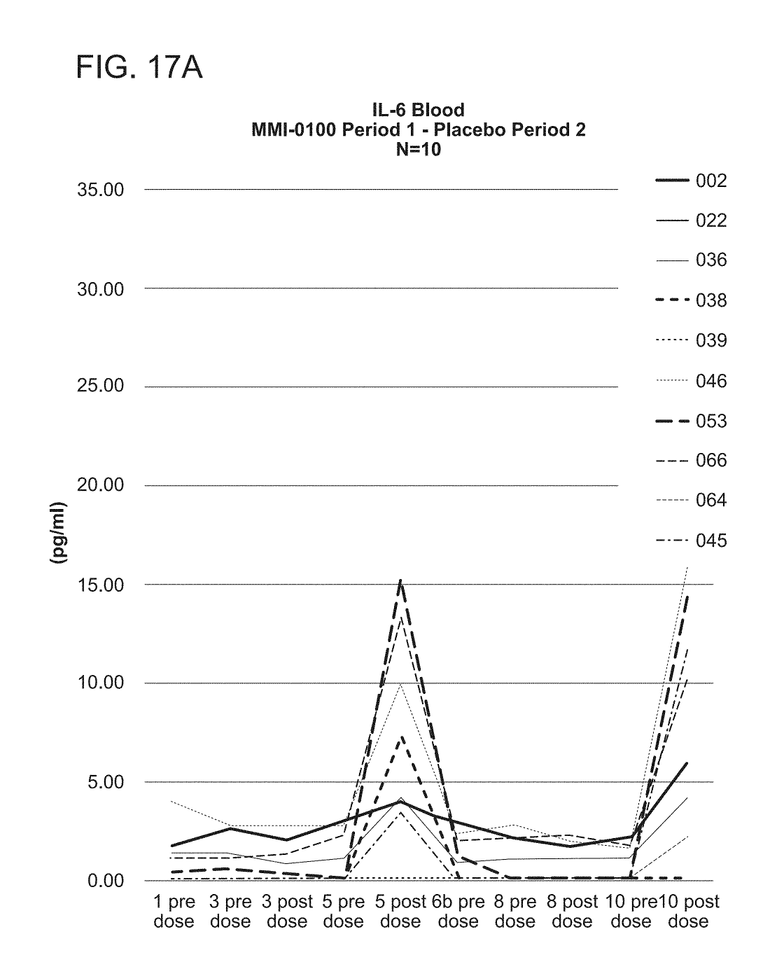

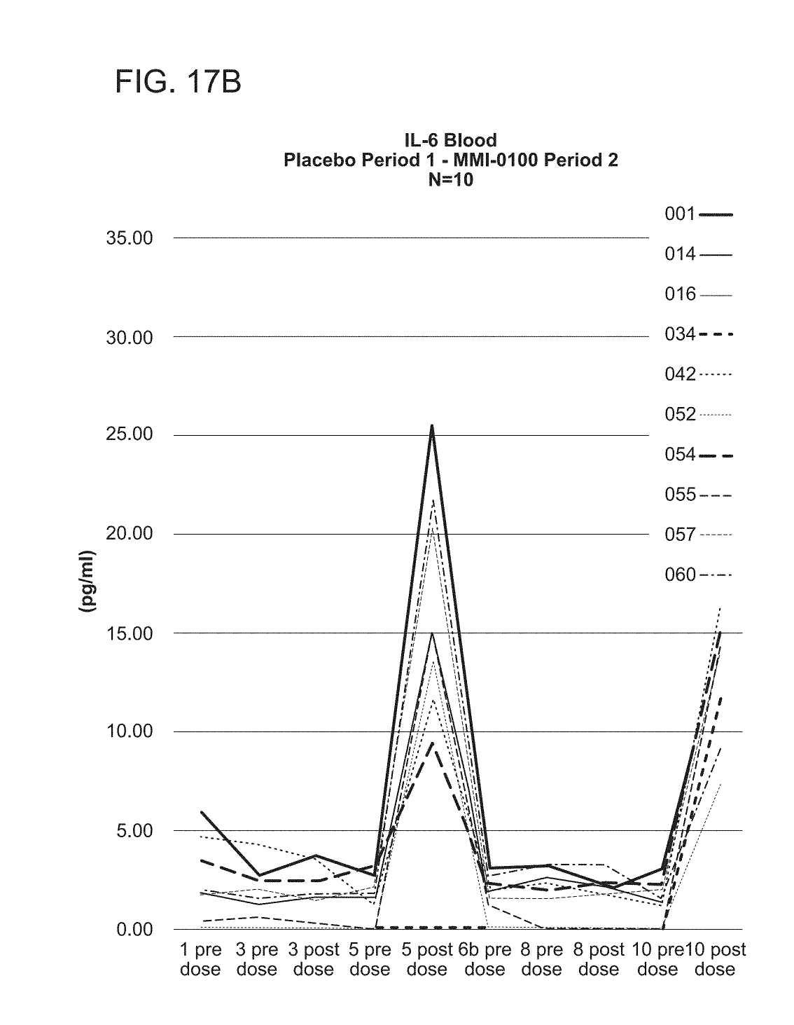

Subject-level IL-6 in blood on day 5 post-LPS showed a sequence effect (p=0.0010) (FIG. 17A and FIG. 17B)

Table 8 contains data quantifying this blood IL-6 treatment difference.

TABLE 8
Blood IL-6 treatment difference - ANOVA model, evaluable population (N =
20). A linear mixed effects model is fitted including fixed effects of treatment,
period and sequence, and a random effect of subject within sequence.

| | | | | Treatment |
|---|---|---|---|---|
| | Period | Sequence | Ratio | Difference |
| Parameter | Effect | Effect | MMI/PBO | (SE) |
| MMI/PBO ratio Day 5 | p = 0.0338 | p = 0.9574 | 1.139 | N/A |
| (Pre-Dose) | | | | |
| MMI/PBO ratio Day 5 | p = 0.6889 | p = 0.0184 | 0.840 | N/A |
| (Post-LPS Challenge) | | | | |
| Change from baseline at | p = 0.0537 | p = 0.6053 | N/A | 0.153 |
| Day 3 (Post-dose) | | | | (0.1361) |
| Change from Day 3 | p = 0.2435 | p = 0.0024 | N/A | −2.793 |
| (Post-dose) at Day 5 | | | | (1.8491) |
| (Post LPS Challenge) | | | | |
| Change from baseline at | p = 0.3016 | p = 0.0025 | N/A | −2.640 |
| Day 5 (Post-LPS | | | | (1.8320) |
| Challenge) | | | | |
| Change on Day 5 (Post- | p = 0.3201 | p = 0.0010 | N/A | −3.138 |
| dose minus Pre-dose) | | | | (1.8142) |
| Change from baseline at | p = 0.0777 | p = 0.6534 | N/A | 0.229 |
| Day 3 (pre-dose) | | | | (0.2015) |
| Change from baseline at | p = 0.7541 | p = 0.0595 | N/A | 0.499 |
| Day 5 (Pre-dose) | | | | (0.2939) |

### Analysis by Treatment Period

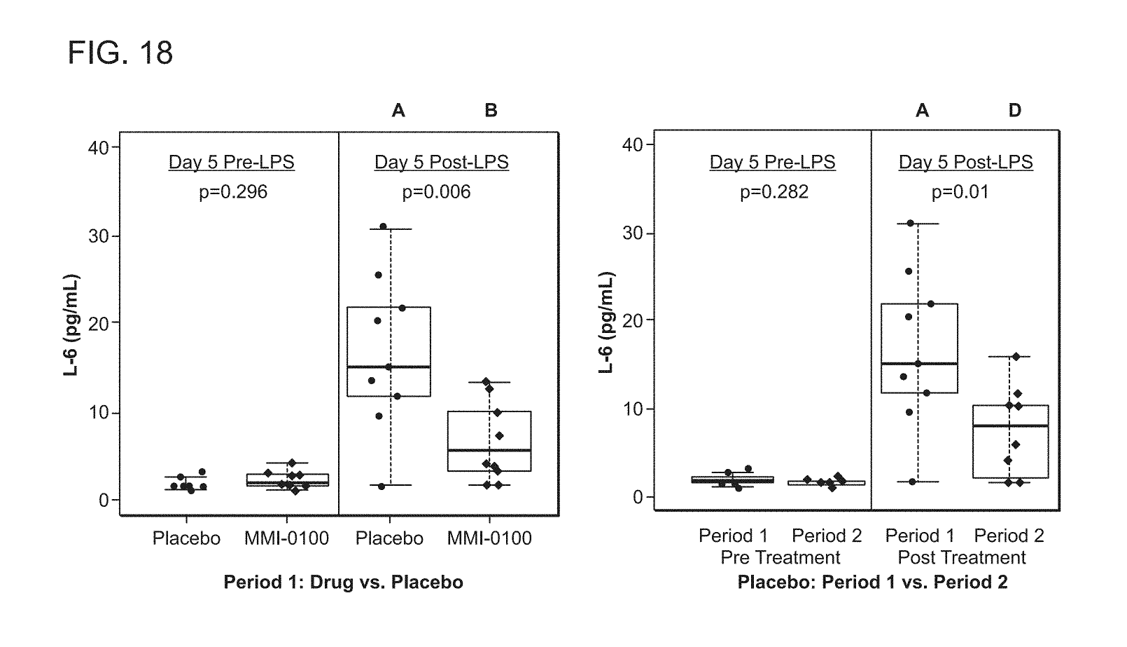

When analyzed by treatment period, it was found that MMI-0100 significantly (p=0.006) decreased serum IL-6 response to LPS challenge in Period 1 (N=10/group), with no difference pre-LPS challenge (FIG. 18). This effect on IL-6 unexpectedly persisted into Period 2, with Period 2 Placebo—previously exposed to MMI-0100—showing significantly (p=0.01) lower serum IL-6 response to LPS challenge than Period 1 Placebo (N=10/group). There was a significant (p=0.0184) sequence effect for IL-6 at Day 5 post-LPS.

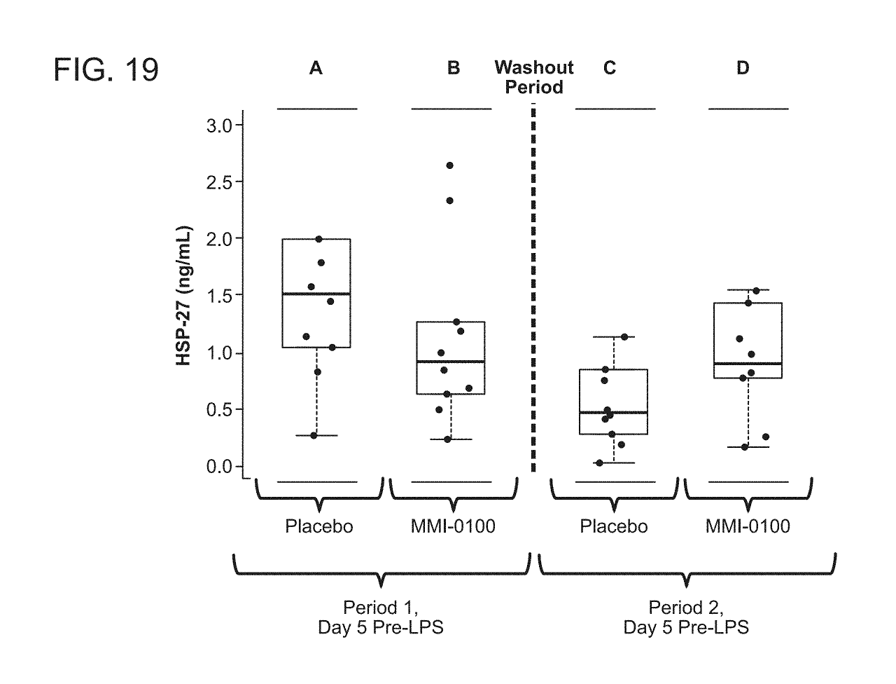

Although not statistically significant (p=0.153), MMI-0100 decreased pHSP27 in blood buffy coat after 5 days dosing, prior to LPS challenge (FIG. 19 N−10/group), with the observed decrease persisting into Period 2. There was a significant (p=0.0491) period effect for pHSP27 at Day 5 pre-LPS challenge. Decrease in pHSP27 following 5 days of MMI-0100 treatment appears to be reproducible upon first exposure to drug.

As described herein, significant differences were not observed on primary sputum cytokine endpoints when analyzed at a group level (N=16/group, pooled placebo and drug groups from both periods), with unexpectedly high biomarker variability observed.

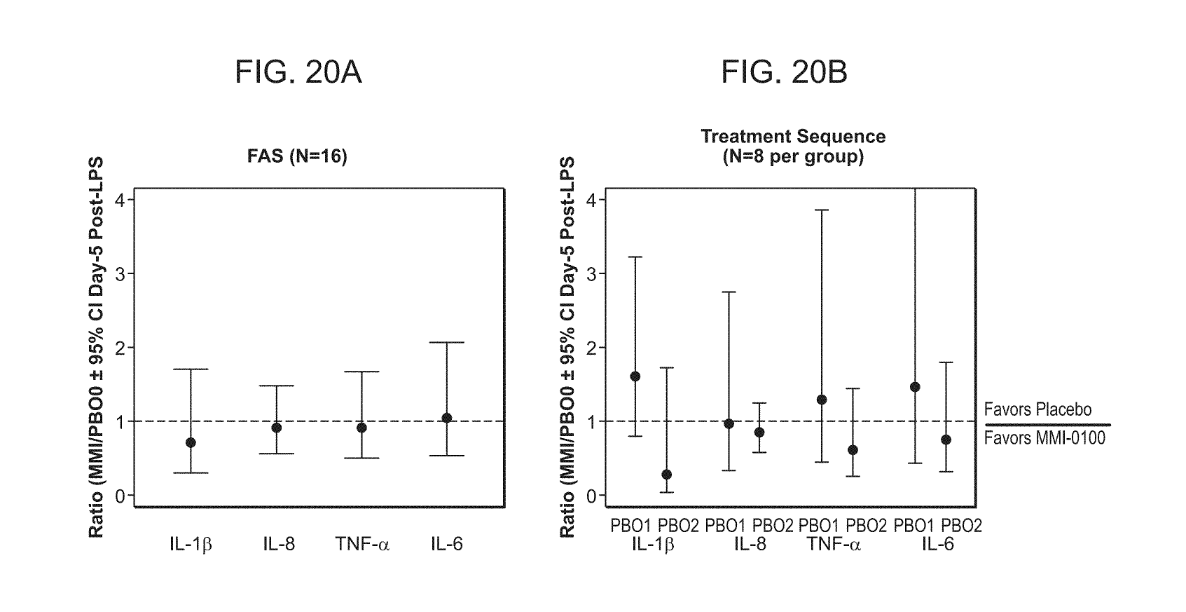

When analyzed by treatment period (N=8/group), 5 days of MMI-0100 pre-treatment appeared to enhance responsivity of smokers to LPS challenge in the lung compartment, with MMI-0100's PD effects persisting into Period 2 (FIG. 20A and FIG. 20N). There were statistically significant sequence effects for IL-8 at Day 5 (p=0.0368) and, for IL-6, change from Day 3 to Day 5 (p=0.0101).

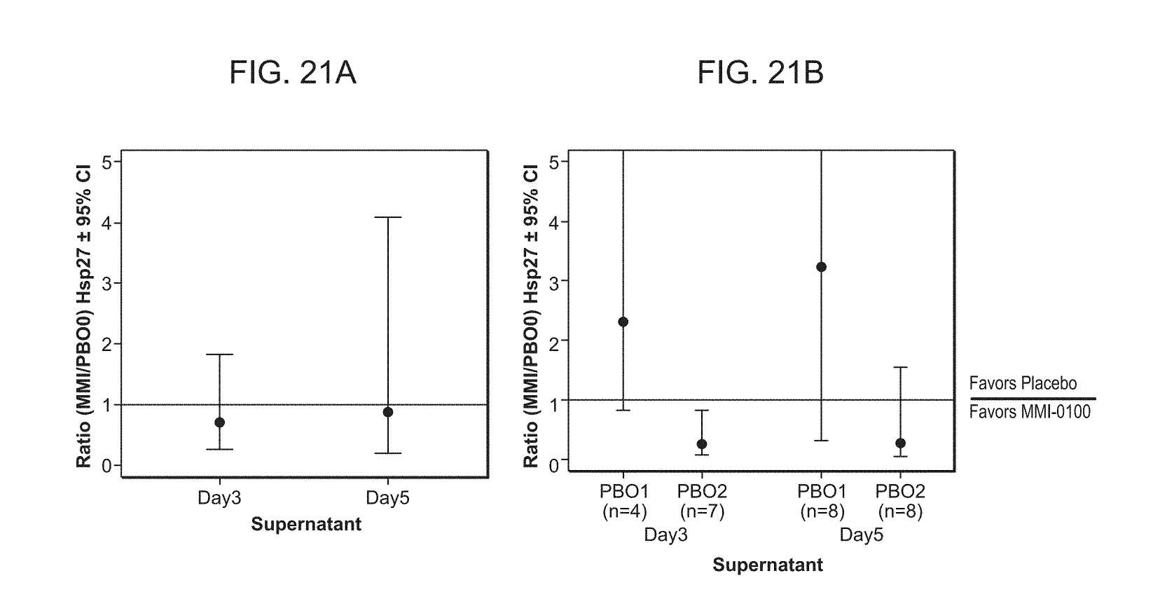

Similar patterns were observed on group-level and by treatment period sputum supernatant pHSP27 at Day 3 and at Day 5 post-LPS challenge (FIG. 21A and FIG. 21B). Significant period effects were observed at Day 3 (p=0.0209) and change from Day 3 to Day 5 (p=0.0021); at Day 5 post-LPS, period effect on pHSP27 approached significance (p=0.0592).

### Conclusions:

MMI was safe and well-tolerated in this first multi-day dosing.

The group-level analyses were complicated by variability in magnitude/direction of inflammatory response to LPS challenge in the placebo group; variability among subjects on biomarker endpoints, which was particularly pronounced in sputum; and by multiple period and sequence effects (many of which were statistically significant). Nonetheless, favorable trends were seen on multiple biomarkers of inflammation and ECM remodeling in sputum (N=16) and blood (N=20), several of which were statistically significant. When inflammatory responses to LPS challenge were produced, there were attenuated inflammatory responses in the MMI-0100 treatment group relative to placebo. Encouraging trends in inflammatory/ECM modulation biomarkers suggest clinically meaningful target engagement by MMI-0100 in a physiologic setting, delineating biomarkers for future clinical efficacy studies.

The sputum IL-1β data demonstrates that subjects receiving MMI-0100 first are more likely to display robust LPS challenge responses in the placebo period and demonstrate an anti-inflammatory response to MMI0100. These data indicate that MMI-0100 has the ability to resensitize to a previously-tolerized stimulus.

The cross-over study design described herein revealed prolonged MMI-0100 PD activity, with immunomodulatory properties impacting LPS response in sputum and blood, with effects of MMI-0100 exposure in Period 1 unexpectedly persisting into Period 2 and hindering ability to detect drug vs. placebo treatment differences with group-level (pooled) analyses. Although drug carry-over is possible, it is unlikely given the between-period washout of ˜6.5-13 times reported drug half-life ˜77 hours. Nonetheless, it is clear that the durability of MMI-0100's effect on the immune system is longer-lived.

As demonstrated in the studies described herein, MMI-0100 modifies basal pHSP27 and IL-6 response to LPS, demonstrating meaningful MK2 target engagement. Further, as demonstrated herein, buffy coat pHSP27, requiring only a readily-obtained blood sample, represents a candidate MMI-0100 PD biomarker reflecting modulation of MK2 activity, with baseline pHSP27 levels suitable for use in response monitoring and/or patient selection/stratification in clinical trials.

### Example 2. Inhibiting Mitogen-Activated Protein Kinase (MAPK)AP Kinase II (MK2) Using the MK2 Inhibitor MMI□0100 to Inhibit Fibrosis and Inflammation in Familial Heart Diseases

Proteotoxicity has recently been identified as an underlying mechanism of heart failure. The turnover of proteins in non-replicating cells such as the cardiomyocyte (CM) and brain rely heavily on the balance between protein synthesis, refolding of damaged proteins, and protein degradation if they are misfolded, mutated or damaged. Protein damage is common and secondary to post-translational modifications secondary to oxidative stress and part of many essential routine biological processes (e.g. ubiquitination, phosphorylation, acetylation). If this balance between protein synthesis, folding and refolding, and degradation is disrupted, accumulation of both large protein aggregates and amyloid-like-oligomers that are toxic to the cell can accumulate (Willis, M. S. & Patterson, C. (2013) “Proteotoxicity and cardiac dysfunction—Alzheimer's disease of the heart?” N Engl J Med 368: 455-464; Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007). These misfolded proteins are central to the pathophysiology of neurodegenerative diseases such as Huntington's disease, Parkinson's disease, and Alzheimer's disease.

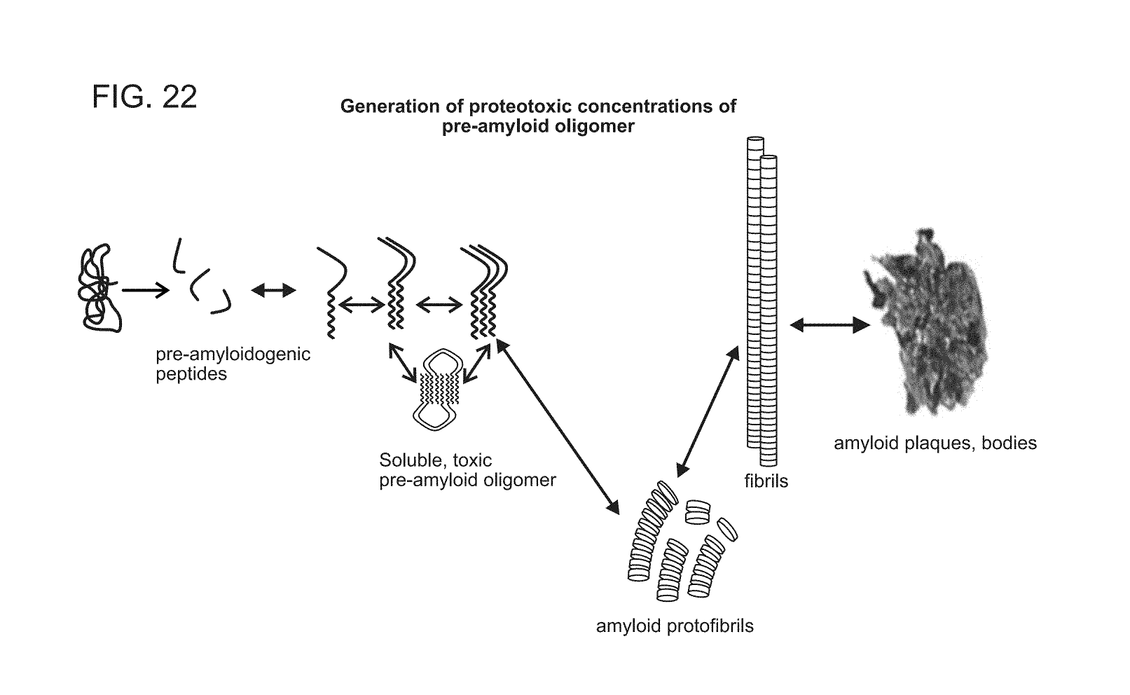

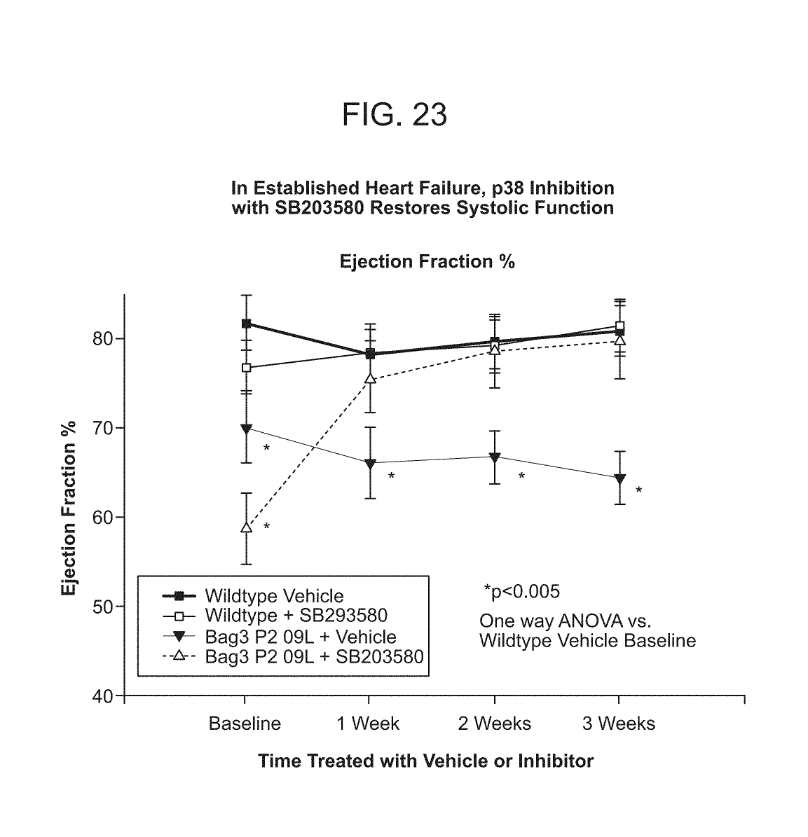

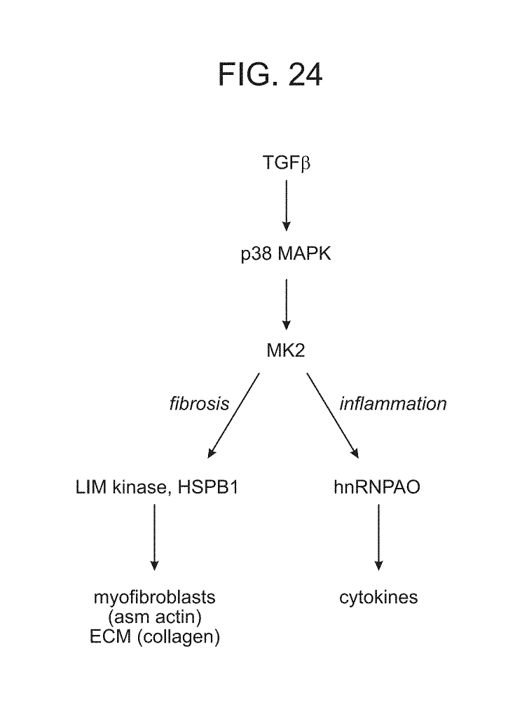

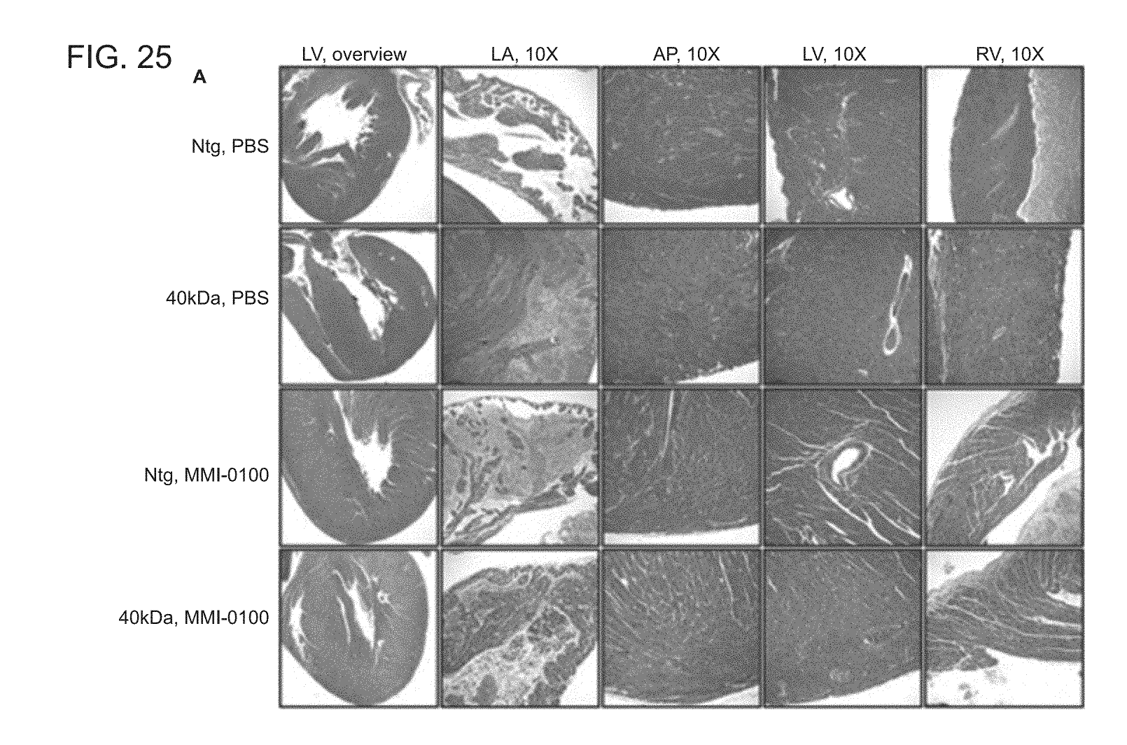

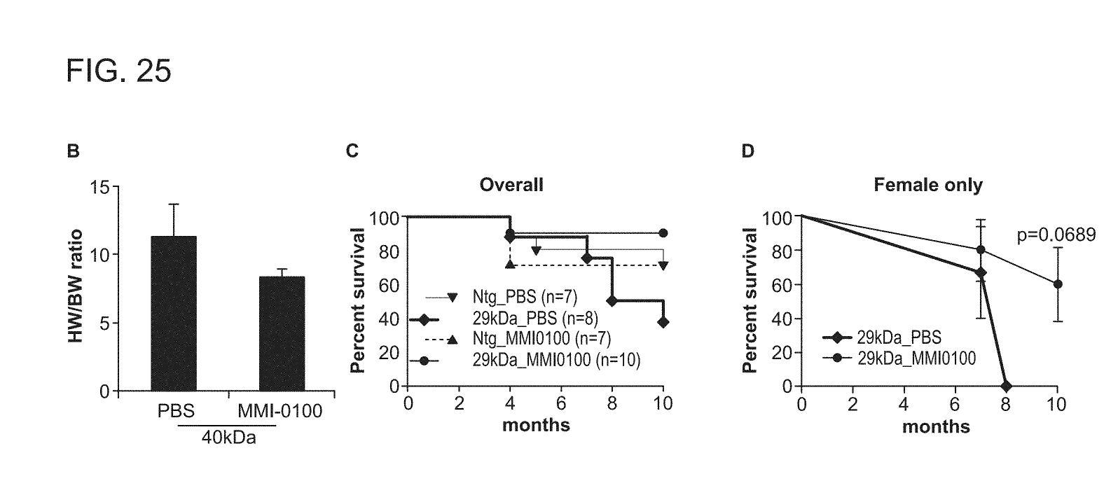

Proteotoxic proteins prone to misfolding are found in familial forms of heart failure, with pre-amyloid oligomers (PAOs) accumulating (see FIG. 22, McLendon, P. M. & Robbins, J. (2015) “Proteotoxicity and cardiac dysfunction,” Circ Res 116: 1863-1882). The direct causation of heart failure by PAOs has been demonstrated in mouse models with CM expression of misfolded prone poly-glutamine repeats (P83) and closely related proteins that that don't misfold (P19) (Pattison, J. S. et al. (2008) “CM expression of a polyglutamine preamyloid oligomer causes heart failure,” Circulation 117: 2743-2751. Remarkably, the misfolded prone cardiac P83 protein rapid heart failure and death, whereas the P19 non-misfolded protein does not differ from wildtype mice in vivo (Id.). A progressive cardiac dilation and fibrosis could be seen in the P83 hearts by 5 months, whereas the fibrosis was absent in P19 and wildtype hearts.

Proteotoxic proteins formed from hereditary mutations induce heart failure by activating the p38 MAPK and downstream MAPKAP Kinase II (MK2). Human hereditary single point mutations in small heat shock proteins have been shown to be prone to misfolding and form the proteotoxic PAOs (Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007); McLendon, P. M. & Robbins, J. (2015) “Proteotoxicity and cardiac dysfunction,” Circ Res 116: 1863-1882). We have created two models carrying CM-specific hereditary mutations, one with a disease-causing mutation in the human alpha B crystallin (CryAB) and the other in the BCL2-Associated Athanogene 3 (Bag3) (Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007); Pattison, J. S. et al. (2008) “CM expression of a polyglutamine preamyloid oligomer causes heart failure,” Circulation 117: 2743-2751).

### CM Expression of CryAB R120G.

Constitutive expression of CryAB R120G led to p38 activation, the development of extensive fibrosis, dilated heart failure, and premature death (Wang, X. et al. (2001) “Expression of R120G-alphaB-crystallin causes aberrant desmin and alphaB-crystallin aggregation and cardiomyopathy in mice,” Circ Res 89: 84-91); Sanbe, A. et al. (2004) “Desmin-related cardiomyopathy in transgenic mice: a cardiac amyloidosis,” Proc Natl Acad Sci USA 101: 10132-10136). A distinctive mitochondrial dysfunction, including altered organization and architecture, reduced maximal oxygen consumption with substrates utilizing complex I, and altered permeability transition pore and activation of apoptosis (Maloyan, A. et al. (2005) “Mitochondrial dysfunction and apoptosis underlie the pathogenic process in alpha-B-crystallin desmin-related cardiomyopathy,” Circulation 112: 3451-3461).

### CM Expression of Bag3 P209L.

Like the human disease, constitutive expression of Bag3 P209L lead to a slowly progressive systolic and diastolic heart failure first seen at 8 months and continue to worsen without any mortality (Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007)). PAOs could be seen at 12 months with the characteristic cellular pathologies, including increased mitochondrial fragmentation measured by by transmission electron microscopy and transcriptional alterations in mitochondrial fission and fusion (Id). Unexpectedly, there was extensive cardiac remodeling at 12 months, with Bag3 P209L hearts having increased numbers of activated cardiac fibroblasts; however, no increases in fibrosis were evident despite upregulation of MAPK p38 signaling (Id).

The common theme is that hereditary misfolded protein in CMs activates p38 signaling and inflammation. Both the CryAB R120G and the Bag3 P209L human mutations activate p38 signaling in vivo (Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007); Wang, X. et al. (2001) “Expression of R120G-alphaB-crystallin causes aberrant desmin and alphaB-crystallin aggregation and cardiomyopathy in mice,” Circ Res 89: 84-91). Misfolded tau proteins in Alzheimer's disease has similarly been linked to the activation of MAPK signaling (Kovac, A. et al. (2011) “Misfolded truncated protein tau induces innate immune response via MAPK pathway. J Immunol 187: 2732-2739). Attenuation of p38 activation in Alzheimer disease animal models reduces memory loss due to Ap and tau toxicities, identifying it as a direct mediator of the pathology (Giraldo, E., et al. (2014) “Abeta and tau toxicities in Alzheimer's are linked via oxidative stress-induced p38 activation: protective role of vitamin E,” Redox Biol 2: 873-877). Similarly, we recently treated 15 month old Bag3 P209L mice exhibiting systolic dysfunction: p38 activation (Quintana, M. T. et al. (2016) “CM-Specific Human Bcl2-Associated Anthanogene 3 P209L Expression Induces Mitochondrial Fragmentation, Bcl2-Associated Anthanogene 3 Haploinsufficiency, and Activates p38 Signaling,” Am J Pathol 186 (8): 1989-2007)) stably reversed the systolic deficit (FIG. 19). Bag3 P209L transgenic (Tg) hearts show an inflammatory response as measured both by an increase in inflammatory infiltrates and NF-κB activity (increased phospho-p65/p65 by Western blot) (Id.)

We've recently identified that the MAPKAP Kinase II (MK2) is downstream of TGFβ and p38 activation (FIG. 20) in both fibroblasts and CMs (Xu, L. et al. (2014) “MMI-0100 inhibits cardiac fibrosis in myocardial infarction by direct actions on CMs and fibroblasts via MK2 inhibition,” J Mol Cell Cardiol 77: 86-101). In these studies, we used the cell-permeant peptide inhibitor of MAPKAP kinase 2 (MK2), MMI-0100, which can inhibit MK2 and downstream fibrosis, and inflammation in pulmonary fibrosis (Id). We showed that daily MMI-0100 treatment started after permanent left anterior descending coronary artery ligation to induce myocardial infarction, significantly inhibited development of cardiac fibrosis and attenuated systolic dysfunction and inflammation (Id.). We subsequently demonstrated that we could aerosolize MMI-0100 using a nebulizer to similarly protect against the TGFβ-mediated fibrosis seen in myocardial infarction in vivo (Brown, D. I., et al. (2016) “Nebulized Delivery of the MAPKAP Kinase 2 Peptide Inhibitor MMI-0100 Protects Against Ischemia-Induced Systolic Dysfunction,” Intl J. Peptide Res. & Therapeutics In press: 1-8), as had previously been shown in a bleomycin-induced model of acute lung injury (Vittal, R. et al. (2013) “Peptide-mediated inhibition of mitogen-activated protein kinase-activated protein kinase-2 ameliorates bleomycin-induced pulmonary fibrosis,” Am J Respir Cell Mol Biol 49: 47-57).

### Hypertrophic Cardiomyopathy (CM-Specific Inducible Tg Myosin-Binding Protein C 40 kDa Fragment Mouse) Induces Fibrosis Via MK2

A stable 40 kDa fragment produced from the cleavage of cardiac myosin-binding protein C (by μ-calpain) when the heart is stressed is detected in both mouse and human hearts. Recent studies have shown that this 40 kDa protein can mediate heart failure, fibrosis, and sudden death in pre-clinical studies. Using an inducible Tg mouse with CM expression of the fragment, we found that mice developed heart failure at 12-17 weeks, along with cardiac hypertrophy and fibrosis with the activation of pathogenic MEK-ERK pathways (Razzaque, M. A. et al. (2013) “An endogenously produced fragment of cardiac myosin-binding protein C is pathogenic and can lead to heart failure,” Circ Res 113: 553-561. We found that inhibiting MK2 with MMI-0100 reduces fibrosis, cardiac hypertrophy, and death (FIG. 21).

Our test hypothesis is that inhaled MMI-0100 therapy can be applied therapeutically to A) chronic cardiac injuries involving proteotoxicity seen in aging military veterans (e.g. hereditary cardiomyopathies/heart failure); and B) acutely to μ-calpain-induced cardiac dysfunction, as seen in sepsis-induced cardiac dysfunction.

The role of MK2 in the pathogenesis of hereditary cardiomyopathies (CryAB R120G and Bag3 P209L Tg mouse lines) and an inducible CM-specific Tg mouse model of μ-calpain activation (cMyBP-C 40 kDaTg mouse line) using a nebulized cell permeant peptide inhibitor of MK2 (MMI-0100) will be evaluated.

Recent studies have found that the cell-permeant peptide MMI-0100 inhibits inflammation and fibrosis (intimal hyperplasia) in a mouse vein graft model (Muto, A. et al. (2012) “Inhibition of Mitogen Activated Protein Kinase Activated Protein Kinase II with MMI-0100 reduces intimal hyperplasia ex vivo and in vivo,” Vascul Pharmacol 56: 47-55), bleomycin-induced pulmonary fibrosis (Vittal, R. et al. (2013) “Peptide-mediated inhibition of mitogen-activated protein kinase-activated protein kinase-2 ameliorates bleomycin-induced pulmonary fibrosis,” Am J Respir Cell Mol Biol 49: 47-57), and abdominal adhesions post-surgery (Ward, B. C., et al. (2011) “Peptide inhibitors of MK2 show promise for inhibition of abdominal adhesions,” J Surg Res 169: e27-36). The MMI-0100 peptide is rapidly taken up by micropinocytosis and targeted to endosomal compartments, where it is retained for up to 7 days (Flynn, C. R. et al. (2010) “Internalization and intracellular trafficking of a PTD-conjugated anti-fibrotic peptide, AZX100, in human dermal keloid fibroblasts,” J Pharm Sci 99: 3100-3121).

A) MK2 Inhibition to Protect Against Cardiac Proteotoxicity.

The ability of the nebulized MMI-0100 (50 μg/kg/day) to inhibit proteotoxic-mediated heart failure, cardiac fibrosis, and death in two complementary models of familial cardiomyopathy will be studied: both are mediated by p38 activation (Brown, D. I., et al., (2016) “Nebulized Delivery of the MAPKAP Kinase 2 Peptide Inhibitor MMI-0100 Protects Against Ischemia-Induced Systolic Dysfunction,” Intl J. Peptide Res. & Therapeutics In press: 1-8).

We have identified MMI-0100's ability to attenuate the development of 1) cardiac hypertrophy; 2) cardiac fibrosis; 3) cardiac dysfunction (detecting systolic function by conscious echocardiography and diastolic function using Doppler analysis of the mitral and aortic valves); 4) dysrhythmias and sudden death (continuous ECG using telemetry implants); 5) activation of the inflammasome (inflammation); and 6) alterations in the cardiac vasculature. The CryAB R120G will be studied at Cincinnati Children's; the Bag3 P209L at the University of North Carolina at Chapel Hill.

B) MK2 Inhibition to Protect Against cMyBP-C 40 kDa Induced Heart Failure.

Cardiac dysfunction in sepsis is characterized by the activation of μ-calpain and the the cMyBP-C 40 kDa Tg mice accurately model this pathogenic process (Li, X. et al. (2014) “Cleavage of IkappaBalpha by calpain induces myocardial NF-kappaB activation, TNF-alpha expression, and cardiac dysfunction in septic mice,” Am J Physiol Heart Circ Physiol 306: H833-843) Here the ability of the nebulized MMI-0100 (50 μg/kg/day) to inhibit development of cardiac fibrosis and heart failure will be studied (Brown, D. I., et al., (2016) “Nebulized Delivery of the MAPKAP Kinase 2 Peptide Inhibitor MMI-0100 Protects Against Ischemia-Induced Systolic Dysfunction,” Intl J. Peptide Res. & Therapeutics In press: 1-8). The mechanism by which MMI-0100 has the ability to attenuate the development of 1) cardiac hypertrophy; 2) cardiac fibrosis; 3) cardiac dysfunction (detecting systolic function by conscious echocardiography and diastolic function using Doppler analysis of the mitral and aortic valves) will be defined. These studies will be primarily performed at Cincinnati Children's. Complementary studies will be performed at UNC, to leverage the cardiovascular pathology skillset on the anatomical aspects of both disease and treatment on arrhythmias. Preliminary data will be built upon by implanting telemetry units to perform continuous ECG monitoring to identify dysrhythmias and sudden death, in addition to any other more subtle defects in the ECG waveforms induced by fibrosis and/or other cardiac remodeling processes; 5) activation of the inflammasome (inflammation); and 6) alterations in the cardiac vasculature.

Impact on Congenital Heart Disease, Mitochondrial Disease (Proteotoxic alterations of mitochondrial fission/fusion (Rana, A., et al. (2013) “Parkin overexpression during aging reduces proteotoxicity, alters mitochondrial dynamics, and extends lifespan,” Proc Natl Acad Sci USA 110: 8638-8643), Diabetes (proteotoxicity of amylin (Despa, S. et al. (2012) “Hyperamylinemia contributes to cardiac dysfunction in obesity and diabetes: a study in humans and rats,” Circ Res 110: 598-608).

Short-term impact: to provide a proof of concept that targeting MK2 to inhibit proteotoxicity in hereditary cardiomyopathies/heart failure may decrease morbidity, improve survival and provide one of the first effective anti-fibrotic therapies in the heart.

Long-term impact: to point the way for providing an easily transportable, nebulized peptide therapy that could be applied to patients easily in the field in acute settings as well as in the the outpatient setting where nebulization is commonly performed by support staff. With MMI-0100's successful IND package and successful Phase Ia trial for Pulmonary Fibrosis, and its topical use for acute surgical adhesions, cutaneous scarring and intimal hyperplasia, the present study will accelerate its application to cardiac diseases where no current therapies exist.

Relevance/patient population(s): Cardiac proteotoxicity is a common mediator of heart failure, the most common cause of mortality and morbidity in the US, including aging veterans with or without human hereditary cardiomyopathies. The studies of the μ-calpain mediated cMyBP-c induced cardiac dysfunction seen in sepsis-induced cardiac dysfunction may have direct application to sepsis-associated cardiac dysfunction in a more acute setting.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method of treating a subject that is in an immunotolerant state with regard to an immune stimulating agent that is no longer therapeutically effective for treating a disease, disorder or condition of lung comprising, in order,
(a) administering (1) a first pharmaceutical formulation formulated for delivery by inhalation containing an immunomodulatory amount of a kinase-inhibiting peptide, and
(b) then administering a second pharmaceutical formulation containing a therapeutic amount of the immunostimulatory agent,
wherein the method is effective to resensitize the subject to the immune stimulating agent so that the subject is immunoresponsive to the immune stimulating agent upon its subsequent administration.

2. The method according to claim 1, wherein the immunotolerant state of the subject is characterized by an attenuated immune response to the immunostimulatory agent, compared to a normal control.

3. The method according to claim 1, wherein the immunotolerant state is characterized by one or more of a reduced level of synthesis, expression, or both of pro-inflammatory cytokines, anti-inflammatory cytokines, both pro-inflammatory and anti-inflammatory cytokines, or an altered balance between proinflammatory cytokines and anti-inflammatory cytokines, compared to a control.

4. The method according to claim 1, wherein the immunotolerant state is a result of repeated prior exposure to the immunostimulatory agent.

5. The method according to claim 4, wherein the immunostimulatory agent is a chemotherapeutic agent.

6. The method according to claim 4, wherein the immunostimulatory agent is lipopolysaccharide (LPS).

7. The method according to claim 1, wherein the kinase-inhibiting peptide is MMI0100, or a functional equivalent, a peptide mimetic or a variant of MMI0100.

8. The method according to claim 7, wherein the immunomodulatory amount of MMI0100 is effective to modulate MK2 signaling.

9. The method according to claim 8, wherein the immunomodulatory amount of MMI0100 is effective to modulate the MK2 signaling affecting an MAPK pathway, an NfκB pathway, an IFN α/β pathway or a combination thereof.

10. The method according to claim 8, wherein the immunomodulatory amount of MMI100 is effective to modulate one or more of autocrine signaling, paracrine signaling or hormonal signaling in an immune cell population.

11. The method according to claim 8, wherein the immunomodulatory amount of MMI0100 is effective to increase activation of a population of inflammatory cells selected from the group consisting of T cells, B cells, NK cells, CT cells, neutrophils, lymphocytes, macrophages, dendritic cells.

12. The method according to claim 8, wherein the immunomodulatory amount of MMI0100 is effective to increase one or more of autocrine signaling, paracrine signaling or hormonal signaling by immune cells.

13. The method according to claim 12, wherein the autocrine signaling, paracrine signaling or hormonal signaling by one or more immune cells comprises TLR-4 signaling.

14. The method according to claim 12, wherein the immune cells are one or more populations selected from T cells, B cells, NK cells, CT cells, neutrophils, lymphocytes, macrophages, dendritic cells.

15. The method according to claim 12, wherein as a result of the signaling the immune cells express, synthesize, or secrete one or more cytokines selected from the group consisting of IL-1α, IL-1β, IL-2, IL-3, IL-4, IL-5, IL-6, IL-7, IL-8, IL-9, IL-10, IL-11, IL-12/IL-23 P40, IL13, IL-17, IL-18, TGF-β, IFN-γ, GM-CSF, CXCL1, CXCL2, and TNF-α.

16. The method according to claim 12, wherein a level of cytokines expressed, synthesized or secreted is measurable in a body fluid.

17. The method according to claim 16, wherein the body fluid is sputum, blood or both.

18. The method according to claim 1, wherein the immunoresponsive immune response comprises restoration of expression, synthesis or both of inflammatory cytokines in immune cells of the lung without affecting immune cells systemically in an amount to cause unwanted systemic side effects.

19. The method according to claim 1, wherein the disease, disorder or condition is gram negative bacterial sepsis, cystic fibrosis, COPD, or lung cancer.

20. The method according to claim 1, wherein the subject is an immunocompromised subject.


SEQUENCE LISTING (27 sequences)

SEQ ID NO: 1 - protein, 22 aa, Artificial Sequence
  Synthetic peptide
YARAAARQARAKALARQLGVAA

SEQ ID NO: 2 - protein, 11 aa, Artificial Sequence
  Synthetic peptide
KALARQLGVAA

SEQ ID NO: 3 - protein, 21 aa, Artificial Sequence
  Synthetic peptide
FAKLAARLYRKALARQLGVAA

SEQ ID NO: 4 - protein, 23 aa, Artificial Sequence
  Synthetic peptide
KAFAKLAARLYRKALARQLGVAA

SEQ ID NO: 5 - protein, 21 aa, Artificial Sequence
  Synthetic peptide
YARAAARQARAKALARQLAVA

SEQ ID NO: 6 - protein, 21 aa, Artificial Sequence
  Synthetic peptide
YARAAARQARAKALARQLGVA

SEQ ID NO: 7 - protein, 22 aa, Artificial Sequence
  Synthetic peptide
HRRIKAWLKKIKALARQLGVAA

SEQ ID NO: 8 - protein, 10 aa, Artificial Sequence
  Synthetic peptide
KALARQLAVA

SEQ ID NO: 9 - protein, 10 aa, Artificial Sequence
  Synthetic peptide
KALARQLGVA

SEQ ID NO: 10 - protein, 11 aa, Artificial Sequence
  Synthetic peptide
KALARQLGVAA

SEQ ID NO: 11 - protein, 11 aa, Artificial Sequence
  Synthetic peptide
YARAAARQARA

SEQ ID NO: 12 - protein, 14 aa, Artificial Sequence
  Synthetic peptide
WLRRIKAWLRRIKA

SEQ ID NO: 13 - protein, 7 aa, Artificial Sequence
  Synthetic peptide
WLRRIKA

SEQ ID NO: 14 - protein, 11 aa, Artificial Sequence
  Synthetic peptide
YGRKKRRQRRR

SEQ ID NO: 15 - protein, 12 aa, Artificial Sequence
  Synthetic peptide
WLRRIKAWLRRI

SEQ ID NO: 16 - protein, 10 aa, Artificial Sequence
  Synthetic peptide
FAKLAARLYR

SEQ ID NO: 17 - protein, 12 aa, Artificial Sequence
  Synthetic peptide
KAFAKLAARLYR

SEQ ID NO: 18 - protein, 11 aa, Artificial Sequence
  Synthetic peptide
HRRIKAWLKKI

SEQ ID NO: 19 - protein, 21 aa, Artificial Sequence
  Synthetic peptide
YARAAARQARAKALNRQLGVA

SEQ ID NO: 20 - protein, 9 aa, Artificial Sequence
  Synthetic peptide
RKKRRQRRR

SEQ ID NO: 21 - protein, 17 aa, Artificial Sequence
  Synthetic peptide
  MISC_FEATURE (3)..(12): X is any amino acid
KKXXXXXXXXXXKRRKK

SEQ ID NO: 22 - protein, 7 aa, Artificial Sequence
  Synthetic peptide
LLKRRKK

SEQ ID NO: 23 - protein, 22 aa, Artificial Sequence
  Synthetic peptide
YARAAARDARAKALNRQLAVAA

SEQ ID NO: 24 - protein, 21 aa, Artificial Sequence
  Synthetic peptide
YARAAARQARAKALNRQLAVA

SEQ ID NO: 25 - protein, 11 aa, Artificial Sequence
  Synthetic peptide
KALNRQLAVAA

SEQ ID NO: 26 - protein, 10 aa, Artificial Sequence
  Synthetic peptide
KALNRQLAVA

SEQ ID NO: 27 - protein, 4 aa, Artificial Sequence
  Synthetic peptide
  MISC_FEATURE (1)..(1): X is any amino acid
  MISC_FEATURE (3)..(4): X is any amino acid
XRXX